SEQUENCE LISTING (44 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - DNA, 3813 bp, Severe acute respiratory syndrome coronavirus 2
  source 1..3813: mol_type = genomic DNA
atgttcgtgtttctggtgctgctgcccctggtgtccagccagtgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacaccccgatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgttccggaagtccaatctgaagcccttcgagcgggacatcagcaccgaaatctatcaggccggcaacaagccctgcaatggcgtggcaggcttcaactgctacttcccactgcggagctacagcttcagacccacatacggcgttggccaccagccttacagagtggtggtgctgtccttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgttccagaccagagccggctgtctgattggcgccgagtatgtgaacaacagctacgagtgcgacatccccatcggagccggcatctgtgccagctaccagacacagaccaagagccacagacgggctagaagcgtggccagccagagcatcattgcctacacaatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgacagcaccgagtgctccaatctgctgctgcagtacggcagcttctgcacccagctgaagagagccctgacagggattgccgtggaacaggacaagaacacccaagaggtgttcgcccaagtgaagcaaatctacaagaccccgcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgcctgggcgacattgcagccagggatctgatttgcgcccagaagttcaagggcctgacagtgctgcctcctctgctgacagatgagatgatcgcccagtacacaagcgccctgctggccggcacaatcacctctggatggacatttggagccggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgctctgggaaagctgcaggacgtggtcaaccacaatgcccaggcactgaacaccctggtcaagcagctgtcctccaagttcggcgccatctctagcgtgctgaatgacatcttctcccggctggaccctcctgaggccgaggtgcaaatcgacagactgatcaccggcagactgcagagcctccagacatacgtgacccagcagctgattagagccgccgagatcagagccagcgctaatctggccgccaccaagatgtctgagtgtgtgctgggccagagcaagagagtggacttttgcggcaagggctaccacctgatgagcttccctcagtctgctcctcacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaaaggcgtgaagctgcactacacctga

SEQ ID NO: 5 - DNA, 3822 bp, synthetic construct
  source 1..3822: mol_type = other DNA
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatga

SEQ ID NO: 6 - DNA, 3822 bp, synthetic construct
  source 1..3822: mol_type = other DNA
atgttcgtatttctcgtcctgctccccttggtgagctcccagtgcgtcaatcttacaacacgaacccagctccctcccgcttacactaacagttttactaggggtgtatattacccagataaggtattccggtcctccgtacttcacagcacacaagacctttttctcccatttttctcaaacgtgacatggttccacgccatccacgtctctggtaccaatggcaccaaacgattcgataaccccgtacttccctttaatgacggggtttatttcgcaagtactgagaagagcaacatcatccgaggatggatttttgggacaactttggatagcaaaactcaatctctgttgatagtcaacaacgctactaacgtcgtgattaaagtatgtgaattccaattctgtaatgacccctttctgggggtctactatcataaaaataacaagagctggatggagtcagagttccgcgtctactctagcgctaataattgcacttttgagtatgttagccagccattccttatggaccttgaagggaagcaggggaatttcaagaaccttagagagttcgttttcaagaatatagacggatattttaagatttacagtaaacacacaccaatcaacctggttcgcgatctcccacagggttttagtgctttggagcccctggttgacctccccataggaataaacataacacgctttcaaactctgttggctcttcataggagttatttgaccccaggtgattcaagctctggatggactgctggagcagccgcttactacgtggggtatctgcaaccaaggaccttcctgctgaaatacaacgaaaatgggaccattactgatgccgtagactgcgctctcgaccccctgtccgaaactaaatgcacccttaagtccttcactgtcgagaaaggaatttatcagactagtaactttcgagtccagccaacagaatcaatcgttcgatttcccaatatcactaatctctgcccattcggtgaagtgttcaatgcaactcgctttgcctccgtatatgcttggaataggaagcggatttctaactgcgtagcagattactccgttctgtataattcagccagcttcagcactttcaaatgttacggggtttctccaacaaaactgaacgatctgtgtttcactaatgtgtacgctgattcatttgtgattagaggggatgaagtccgccagattgccccaggtcagacagggaagatcgccgattacaactataaacttcccgacgacttcactggatgcgtaatagcatggaatagtaataatctggacagcaaggtaggtgggaactacaactatttgtataggttgtttagaaagtcaaacctcaagcccttcgagagagacatctctaccgagatttaccaggctggtagcacaccttgtaatggcgtagaaggctttaattgttactttcctctccaaagttacggctttcaacctactaatggggttggctaccagccctaccgagttgtagttcttagcttcgagctccttcacgcccccgctactgtttgtgggccaaagaagtccaccaatctcgtcaagaacaaatgtgtcaatttcaactttaacggcttgacaggtaccggcgtacttaccgaatctaacaaaaagttccttcccttccagcaatttggcagagatatcgcagatacaacagacgcagtacgagacccccagaccctggagattttggacatcacaccatgttcattcggaggggtcagcgtcatcactccaggaactaataccagcaaccaggtagcagttctctaccaagatgttaactgcactgaagtgccagttgcaattcacgctgaccaactgactcccacttggcgggtatactccactggttccaatgtctttcagacacgcgcaggatgtctgatcggggccgaacacgtaaataacagctatgaatgtgatatccctattggggccggcatttgcgctagctatcagacacaaacaaattctccccgacgagcacgctcagttgcatctcaatccataattgcctacaccatgagtctgggtgcagaaaactctgtagcttattctaacaattccattgctatacccaccaattttaccatctcagtaacaacagagatccttccagtgagcatgaccaagacctctgttgattgcactatgtacatctgcggtgattcaacagaatgttccaatcttttgcttcaatatggctcattctgtactcaactcaaccgagccctgaccggcattgctgtggaacaggataaaaatactcaagaggtctttgcccaagtcaaacagatttacaaaacaccaccaatcaaggattttggtggcttcaatttcagtcagatactccctgacccttccaagcctagcaagcggtcattcattgaagatttgcttttcaataaggtaaccttggcagacgccgggtttatcaagcagtacggcgactgtcttggcgacatagctgcccgggatttgatttgtgcccagaagtttaatggcctgactgtactccctcccctgctgacagatgaaatgatcgcacagtacactagcgctttgctcgccggtacaattacctctgggtggacatttggggctggtgccgccctccaaattcccttcgcaatgcaaatggcttaccgcttcaacggcataggcgtcactcagaacgtcttgtacgagaaccagaaacttatcgctaaccaatttaattccgcaatcgggaagattcaggatagccttagtagcactgcttcagctctgggcaagctgcaagatgtcgtcaatcaaaatgcacaggcacttaacacactggttaagcagttgtctagtaattttggggctatttcctctgtccttaatgatatcttgagtcgattggacaaagtagaagctgaggtccagatagatcggttgataactggacggttgcagagcctgcagacttacgtcacccagcagctcattcgcgctgcagagatccgcgcttcagctaacttggctgctacaaaaatgtcagaatgcgtcctggggcagtctaaacgggttgacttttgtggcaagggttatcacctgatgagctttcctcaaagtgcccctcacggtgtcgtcttcctccacgtaacttatgtccctgcccaggaaaaaaacttcaccacagcacccgctatatgccatgacgggaaagcacatttccctcgagagggtgtattcgtgagcaatggaacccattggtttgtaacacagcgaaacttttacgaacctcaaatcattactactgacaatactttcgtctcaggcaactgcgatgtagtaataggcatagttaacaatacagtttatgatcctctgcaaccagagctcgatagctttaaggaggagcttgacaagtactttaaaaatcatacatctcctgacgtagaccttggggatattagcggcatcaatgcttcagtcgttaatattcaaaaagagattgaccgcctgaatgaggtggccaaaaaccttaacgaaagcttgatagatttgcaagagctgggaaagtatgagcaatatataaaatggccttggtacatctggcttggcttcatcgccgggctcatcgccattgtcatggtaactataatgctttgttgcatgaccagctgttgcagctgtctgaagggctgttgttcatgcggaagttgttgcaaatttgatgaagacgactctgaaccagtactgaagggggtgaagcttcattacacctga

SEQ ID NO: 7 - protein, 159 aa, synthetic construct
MGSGPSNFVNHAPGEAIVTGLEKGADKVAGTISHTIFVEIVSSSTGIIIAVGIFAFIFSFLYKLLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 8 - DNA, 20 bp, synthetic construct
  source 1..20: mol_type = other DNA
ggtgcatcgatgcagggggg

SEQ ID NO: 9 - DNA, 114 bp, synthetic construct
  source 1..114: mol_type = other DNA
aaaacaggtcctccccatactctttcattgtacacaccgcaagctcgacaatcatcggattgaagcattgtcgcacacatcttccacacaggatcagtacctgctttcgctttt

SEQ ID NO: 10 - RNA, 20 nt, synthetic construct
  source 1..20: mol_type = other RNA
ggtgcatcgatgcagggggg

SEQ ID NO: 11 - RNA, 114 nt, synthetic construct
  source 1..114: mol_type = other RNA
aaaacaggtcctccccatactctttcattgtacacaccgcaagctcgacaatcatcggattgaagcattgtcgcacacatcttccacacaggatcagtacctgctttcgctttt

SEQ ID NO: 12 - protein, 23 aa, synthetic construct
IVSSSTGIIIAVGIFAFIFSFLY

SEQ ID NO: 13 - protein, 38 aa, synthetic construct
MGSGPSNFVNHAPGEAIVTGLEKGADKVAGTISHTIWE

SEQ ID NO: 14 - protein, 49 aa, synthetic construct
KLLQWYNRKSKNKKRKEQIREQIELGLLSYGAGVASLPLLNVIAHNPGS

SEQ ID NO: 15 - protein, 48 aa, synthetic construct
VISATPIYKGPCTGVPNSRLLQITSGTAEENTRILNHDGRNPDGSINV

SEQ ID NO: 16 - DNA, 26 bp, synthetic construct
  source 1..26: mol_type = other DNA
acaggtacgttaatagttaatagcgt

SEQ ID NO: 17 - DNA, 22 bp, synthetic construct
  source 1..22: mol_type = other DNA
atattgcagcagtacgcacaca

SEQ ID NO: 18 - DNA, 26 bp, synthetic construct
  source 1..26: mol_type = other DNA
acactagccatccttactgcgcttcg

SEQ ID NO: 19-20 - skipped (2 entries)

SEQ ID NO: 21 - protein, 159 aa, synthetic construct; identical to SEQ ID NO: 7

SEQ ID NO: 22 - protein, 125 aa, synthetic construct
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 23 - protein, 154 aa, synthetic construct
MGSGPSNFVNHAPGEAIVTGLEKGADKVAGTISHTIWEVIAGLVALLTFLAFGFWLFKYLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 24-30 - skipped (7 entries)

SEQ ID NO: 31 - protein, 1268 aa, Severe acute respiratory syndrome coronavirus 2
MFVFLVLLPLVSSQCVNLITRTQSYTNSFTRGVYYPDKVFRSSVLHSTQDLFLPFFSNVTWFHAISGTNGIKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 32 - DNA, 3807 bp, Severe acute respiratory syndrome coronavirus 2
  source 1..3807: mol_type = genomic DNA
atgttcgtgttcctggtgctgctgcctctggtgtccagccagtgtgtgaacctgatcacccggacacagagctacaccaacagcttcaccaggggcgtgtactaccccgacaaggtgttcagatccagcgtgctgcactctacccaggacctgttcctgcctttcttcagcaacgtgacctggttccacgccatcagcggcaccaacggcatcaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctgggcagagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaacttcgccccattcttcgccttcaagtgctacggcgtgtcccctaccaagctgaacgacctgtgcttcaccaatgtgtacgccgacagcttcgtgatccggggcaatgaggtgtcccagattgcccctggacagaccggcaatatcgccgactacaactacaagctgcccgacgacttcaccggctgtgtgatcgcctggaatagcaacaagctggactccaaagtcggcggcaactacaattaccggtacaggctgttccggaagtccaatctgaagcccttcgagcgggacatctccaccgagatctatcaggccggcaacaagccctgtaatggcgtggcaggcgtgaactgctacttcccactgcagtcctacggcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaccggcaccggcgtgctgacagagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgtttcagaccagagccggctgtctgattggcgccgagtatgtgaacaacagctacgagtgcgacatccccatcggagccggcatctgtgccagctaccagacacagaccaagagccacagacgggctagaagcgtggccagccagagcatcattgcctacacaatgtctctgggcgccgagaacagcgtggcctacagcaacaactctatcgctatccccaccaacttcaccatcagcgtgaccacagagatcctgccagtgtccatgaccaagaccagcgtggactgcaccatgtacatctgcggcgattccaccgagtgctccaacctgctgctgcagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagattctgcccgatcctagcaagcccagcaagcggagcttcatcgaggacctgctgttcaacaaagtgacactggccgacgccggcttcatcaagcagtatggcgattgcctgggcgacattgcagccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaatggcatcggagtgacccagaatgtgctgtacgagaaccagaagctgatcgccaaccagttcaacagcgccatcggcaagatccaggacagcctgagcagtacagccagcgctctgggaaagctgcaggacgtggtcaaccacaatgcccaggcactgaacaccctggtcaagcagctgagcagcaagttcggcgccatctctagcgtgctgaacgatatcctgagcagactggaccctcctgaggccgaggtgcagatcgacagactgatcacaggcagactgcagagcctccagacctacgtgacacagcagctgattagagccgccgagatcagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtccggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtatatcaagtggccttggtacatctggctgggctttatcgccggactgattgccatcgtgatggtcacaatcatgctgtgctgcatgaccagctgctgctcttgcctgaagggctgctgtagctgtggctcctgctgcaagttcgacgaggacgattctgagcccgtgctgaagggcgtgaagctgcactacacatga

SEQ ID NO: 33 - DNA, 3807 bp, Severe acute respiratory syndrome coronavirus 2
  source 1..3807: mol_type = genomic DNA
atgttcgtgtttctggtactccttccccttgtgtcttcacaatgcgttaatttgatcacacgaactcaatcatatactaattctttcacgcgaggcgtatactatccggataaggtcttccgctcttccgtactccacagtacacaggaccttttcctgccattctttagcaatgtcacttggttccatgcaatatctggaacaaatggtattaaacgatttgataatcctgtcttaccattcaatgacggcgtctacttcgctagtaccgagaaaagcaatatcatcagaggttggatcttcggtaccactcttgattctaagactcagagcttgcttattgtcaataatgccaccaatgttgttatcaaagtttgcgaatttcaattttgtaacgaccctttcttggacgtgtactatcacaagaacaacaagtcatggatggagagtgagttccgagtatattcttctgcaaacaactgtaccttcgagtatgtcagtcaacccttcttgatggacctggaagggaagcaaggcaactttaagaatcttcgcgaattcgtatttaagaacattgatggctacttcaagatctactccaaacatacccccatcaatcttgggcgtgatttgccccaaggctttagtgcgctcgagcctcttgtggatctcccaattggaatcaacattactcgctttcaaacgctgctggctttgcataggtcctatcttactcctggagactcttcatctggctggacggccggggcagcagcctactacgtgggatatcttcaacccaggacttttctgcttaaatacaacgaaaatggaactattacagatgcagttgactgcgctcttgatcccctctccgaaactaaatgtacactcaagagctttacagtagagaaagggatatatcaaacctcaaactttcgggtccaaccaacagaaagtatcgtgagattccccaacataacgaatttgtgtccattcgatgaagttttcaacgccaccaggttcgcgtccgtatatgcttggaaccgaaagcggattagtaactgtgtagccgactattctgtactgtataactttgctcctttctttgccttcaaatgttatggagtgagcccaaccaaactgaacgacttgtgctttactaacgtctacgccgacagttttgtcatcagagggaacgaggtttcccagatcgcaccaggacagactggtaatatagccgattacaattataaattgccagatgactttacaggctgcgtcatagcttggaattcaaataagctcgattccaaggtcggtggaaattataattatcgataccggttattcaggaaaagcaatctcaagccttttgaacgtgacatcagcactgagatctatcaagcaggcaataaaccctgtaatggcgtagccggtgtaaattgttatttccctctgcaaagttacggatttaggcccacctacggagtgggtcaccaaccataccgcgtagtcgttcttagttttgagctgctgcatgccccagcgacagtttgtggaccaaagaaatccaccaatctcgtcaagaacaaatgcgtgaattttaattttaacggtctcaccggcacaggggtcttaacagagtccaacaagaagtttctccctttccaacaattcggacgggatatagccgataccaccgatgcagtgcgagatccccaaaccttggagatccttgatataacaccttgctcattcggtggcgtctccgtcattactcctggaacgaacacctctaatcaagtggcagtcttgtatcaaggtgttaactgcacggaagtccctgtcgcaatccatgctgatcaattgacacccacatggcgggtctatagcaccggctcaaatgtatttcagacacgggcaggttgtctgattggtgcagagtatgttaataactcatatgaatgcgatatccctatcggagctggaatttgcgcttcctatcaaacccaaacaaaatcccatcggagagctagatccgttgcctctcaatctattattgcatatacaatgtctctgggagcggagaacagtgtggcctactccaataacagcattgcgattccaactaacttcactatcagtgtgactactgaaattttaccagtgtctatgaccaagacttctgtagattgtaccatgtatatatgcggtgactccaccgagtgtagcaacctgctgttgcagtacggcagtttctgtacccaattgaaacgtgccctgactggcatcgccgtggaacaggataagaatactcaagaggttttcgcccaagtaaaacaaatttataaaacaccacctatcaaatattttggcggattcaacttctcccagatcttacccgacccttctaaaccaagcaagcggtcattcatagaagatctgttgtttaataaagttacgctggcggacgctggattcatcaagcagtacggcgattgcttaggggatatcgccgctcgggatctgatctgtgcccaaaagtttaatggactcaccgttctgccaccattgttgactgacgaaatgattgcccaatacacatccgctttgctggcgggcacaattacctctggctggacgtttggtgccggggcagcacttcagatcccgttcgccatgcaaatggcctacagatttaatggcataggagtgacacaaaacgtcctgtatgaaaaccaaaagttgattgccaatcaatttaactccgctatagggaaaattcaggattcactgtcatcaactgcttccgcattggggaaactccaggacgtggtcaaccacaacgcacaggcactcaacacgttggtcaaacaattgtcttctaaattcggggctatatcttctgtgctcaatgatattctttcacgcctggatccacccgaagctgaagttcaaatcgataggctcataacgggtaggctccagtctcttcaaacttatgtgacgcaacaattaatccgcgcggcagaaattcgtgcgtccgctaatctcgcggccacaaagatgagtgaatgcgttctggggcagtctaagcgcgttgatttctgtggaaagggttatcatctcatgtctttcccccaatcagcaccacacggagttgttttccttcatgtaacttatgttcctgcacaagagaagaacttcacaacggctccagccatttgtcacgatggaaaagcacatttccctcgcgagggtgtctttgtcagtaacggaacccattggttcgtgactcaacgcaatttctatgaaccccaaattattacaaccgacaatacctttgttagcggaaattgtgacgtggtcataggcattgtcaataatacagtatacgacccgctccaacctgagttagactctttcaaggaagagctggataaatactttaagaatcatacatctcctgatgtggacttgggcgatattagtggtatcaatgctagcgtagttaatatccaaaaggaaattgacaggctgaatgaggttgcaaagaacctgaatgagagccttattgacttacaagagcttggaaaatacgaacaatacattaaatggccctggtacatctggctgggctttatagctggcttgatcgccatcgttatggtaaccatcatgttgtgttgtatgacctcatgttgtagttgccttaaaggatgttgttcatgtgggagttgttgcaagtttgatgaagatgatagtgaacctgttcttaaaggggttaaactgcactacacttag

SEQ ID NO: 34 - DNA, 3807 bp, Severe acute respiratory syndrome coronavirus 2
  source 1..3807: mol_type = genomic DNA
atgtttgtcttcctggtgctgttgcctctggtgagctctcagtgtgtcaaccttatcacccggacacagtcctacactaactctttcacgcgcggagtgtactatcctgacaaggtttttcggagtagtgtcctccactcgacacaagacctcttcctgccttttttttccaatgtgacatggttccacgccatctccggaacaaatgggataaaacgcttcgacaaccctgtgttgcctttcaacgatggcgtgtactttgcctcgactgagaagtctaatatcatccggggttggatcttcggtactaccctggacagtaaaactcaaagtctgctgattgtgaataatgccaccaatgtggtaatcaaagtgtgtgaattccagttttgtaacgatcctttcctggatgtgtactaccacaagaacaacaagagctggatggagtctgagttcagagtatattccagcgccaacaactgcacttttgagtacgtgagccagcccttcctcatggacttggaaggaaagcagggcaatttcaaaaacctgcgggaatttgtgttcaagaacatcgacggctatttcaaaatctacagtaaacacacacccattaacctgggccgggatctgcctcagggattctctgccctggagccactagtagacctacccatcggcatcaacattacccgctttcaaacacttctagccctccacaggagctacctcactcctggcgacagtagctccggctggaccgccggcgcggccgcgtactacgtggggtacctgcaacctcgtaccttcctgctcaagtataatgaaaatggcaccataacagatgctgtggactgcgcacttgatcccctgagcgagactaaatgcaccctcaaaagcttcaccgtggagaaggggatttaccagaccagcaactttagagtccagccgacagagagcatcgtgagattcccaaacataaccaacctgtgtccttttgacgaagtcttcaatgctacccggtttgcgagcgtgtacgcctggaataggaagaggatcagcaactgtgtggctgattactcggtgctgtacaacttcgcccctttctttgcctttaaatgctatggggtgtctcccactaagctgaatgatctgtgtttcactaacgtttatgccgactctttcgtcatcagaggcaatgaggtaagccaaattgcccctggacagacggggaacatcgccgactacaactacaaactccctgacgactttacaggatgtgtaatcgcgtggaactccaataaactggactctaaggtcgggggcaactataactatagatacagactgttcagaaagtccaacctgaagccctttgaaagagatattagcacagaaatatatcaggccggcaataaaccatgcaatggagttgccggggtgaactgctactttcctctgcaaagttatgggttcagacctacatacggggtagggcaccagccctacagagtggtggtactcagctttgagttgttgcacgcgcctgccactgtctgcggcccgaagaaaagcaccaacctggtgaagaacaaatgtgtgaacttcaacttcaacggcttaaccgggaccggcgttctgaccgagtcaaacaagaagttccttcctttccaacagtttggcagagacatcgctgacaccacggatgcagttcgcgacccacagactctggagatcctcgatatcaccccttgctcctttggcggcgtgagcgttatcacccctggtacaaatacttctaatcaggttgctgtcctgtaccaaggtgtgaattgtacagaggtgcccgtggccattcatgccgaccagttaacccctacctggagggtgtactccacaggttccaacgttttccagacaagagctggctgcctgattggagccgaatatgtcaacaacagttacgaatgcgatatacccattggagccggcatctgtgcctcataccaaacacagaccaaaagtcatcggagagccagatcagtggcctcccagtctatcatcgcctatacaatgagtctgggtgcagaaaacagtgtagcttactctaacaacagcatcgccatccctactaacttcacaatcagcgttaccaccgaaatcctgccagtgagcatgaccaagacaagcgtagattgcaccatgtacatctgcggagactcgacggagtgcagcaacctgttactgcagtatggcagcttctgcacacagcttaagcgagccctgaccggaattgctgtggagcaggataagaatacacaggaggtgttcgcacaggtaaagcaaatctacaagacccctccaattaagtacttcggtggcttcaacttttctcagattctccctgatccatcgaaacccagcaaaagatccttcatcgaggacctcctgttcaacaaggtgactttggcagacgcgggcttcattaagcagtacggagactgtctgggcgacatcgctgcccgagacctgatctgtgctcagaagtttaacgggctgaccgtgctgccccccctcctgactgatgagatgatagcacagtacaccagcgccctgttggccggtaccatcacatccggctggacatttggtgccggcgcagcattgcaaatccccttcgccatgcagatggcttaccggtttaacggcatcggcgtgacccagaatgtattgtacgagaaccagaagctgattgccaaccaattcaactcagccatcggaaagatccaggacagtctgagcagcacagctagcgccctcggcaagttgcaggatgtcgtgaaccacaacgcccaggctctcaacacactagtgaagcagttgagctccaagttcggagcaatttcgagcgtgctcaatgatatcttgagcagattggaccctcctgaggccgaagtccagatcgatcggctgattacagggagactgcagagcctgcagacctatgtaacccagcagctgataagagcagccgagattcgggcctctgcgaatctggctgccaccaagatgagcgagtgtgtcctgggtcagtctaagagagtggacttctgtggcaagggataccacctcatgtcttttccacaaagcgccccccatggggtggtgttcctgcacgtcacctacgtgcccgcccaggaaaaaaactttaccactgcgcctgccatctgccatgacgggaaagcccatttccctcgagaaggcgtgttcgtttctaacggcacgcactggtttgtgactcagaggaacttctacgagccccagatcatcaccacagacaataccttcgtgtccggcaactgtgatgtggtgattggcatcgtgaataacacagtgtatgaccctcttcagccagagctggattcgtttaaggaggaactggataaatatttcaagaatcacacatcccctgatgtggacctgggggatatctctgggatcaatgcttcagtggttaacattcagaaggaaatcgacagactgaacgaagtggctaagaatctcaacgaatccctcattgacctgcaggagctgggcaaatacgaacaatacatcaagtggccctggtacatctggctgggcttcatcgctggcttaatcgctatcgtcatggtgacaattatgctgtgttgcatgacttcttgctgctcctgcctcaagggctgttgcagttgtgggtcttgctgcaaatttgatgaagatgattctgagccagtgctcaagggcgtgaaactgcactacacctga

SEQ ID NO: 35 - DNA, 3810 bp, Severe acute respiratory syndrome coronavirus 2
  source 1..3810: mol_type = genomic DNA
atgttcgtgttcctggtgctgctgcccctggtgagctctcagtgcgtgaacctgatcacaagaacacagagctacaccaacagcttcacaagaggcgtgtactaccccgacaaggtgttcagaagcagcgtgctacacagcacccaagacctgtttctgcccttcttcagcaacgtgacctggttccacgccatcagcggcaccaacggcatcaagagattcgacaaccccgtgctgcccttcaacgacggcgtgtacttcgctagcaccgagaagagcaacatcatcagaggctggatcttcggcaccaccctggatagtaaaactcagagcctgctgatagtgaacaatgccaccaacgtggtgatcaaggtgtgcgagtttcagttctgcaacgaccccttcctggacgtgtactaccacaagaacaacaagagctggatggagagcgagttccgtgtgtatagctccgctaacaactgcaccttcgagtacgtgtctcagcccttcctgatggacctggagggcaagcaaggcaacttcaagaatctgagagagttcgtgttcaagaacatcgacggctacttcaagatctacagcaagcacacccccatcaacctgggcagagacctgccccaaggcttcagcgccctggagcccctggtggacctgcccatcggcatcaacatcacaagatttcagaccctgctggccctgcacagaagctatctgacccccggcgacagctcatcgggctggaccgctggcgcagcggcctactacgtgggctacctgcagcctagaaccttcctgctgaagtacaacgagaacggtacgattactgacgccgtggactgtgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcttggaacagaaagagaatcagcaactgcgtggccgactacagcgtgctgtacaacttcgcccccttcttcgccttcaagtgctatggcgtgagccccaccaagctgaacgacctgtgcttcaccaacgtgtacgccgacagcttcgtgatcagaggcaacgaggtgtctcagatcgcccccgggcagaccggcaacatcgccgactataactacaaactgcccgacgacttcaccggctgcgtgatcgcctggaacagcaacaagctggacagcaaggtgggcggcaactataattatagatacagactgttcagaaagagcaacctgaagcccttcgagagagacatcagcaccgagatctaccaagccggcaacaagccctgcaacggcgtggccggcgtaaactgctactttcccctgcagagctacggcttcagacccacctacggcgtgggccatcagccctacagagtggtggtgctgagcttcgagctgctgcacgcgcccgctactgtctgcggccccaagaagagcaccaacctggtgaagaacaagtgcgtgaacttcaactttaacggcctgaccggaaccggcgtgctgaccgagagcaacaagaagttcctgccatttcagcaattcggcagagacatcgccgacaccaccgatgccgtgcgtgaccctcagaccctggagatcctggacatcaccccgtgcagtttcggcggcgtgagcgtgatcacccccggcaccaacacaagcaaccaagtggccgtgctgtaccaaggcgtcaactgtacggaggtgcccgtggccatccacgccgatcagctgacccccacctggagagtatatagcaccggcagcaacgtgtttcagacaagagccggctgcctgatcggcgccgagtacgtgaacaacagctacgagtgcgacatccccatcggcgccggcatctgcgctagctatcagacacagaccaagagccacagaagagctagaagcgtggcttctcagagcatcatcgcctacaccatgagcctgggcgccgagaacagcgtggcctacagcaacaacagcatcgccatccccaccaacttcaccatcagcgtgaccaccgagatactgcccgtgagcatgaccaagacaagcgtggactgcaccatgtacatctgcggcgacagcaccgagtgcagcaacctgctgctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcctgaccctagcaagcctagcaagagaagcttcatcgaggacctgctgttcaacaaggtgaccctggccgacgccggcttcatcaagcagtacggcgactgcctgggggacatcgccgctagagacctgatctgcgctcagaagttcaacggcttaactgtgctcccccccctgctgaccgacgagatgatcgctcagtatacaagcgcgctactggccggtaccatcacctccggatggacctttggtgcgggggcggccctgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatctcgagcgtgttgaacgacatcctgagcagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcacaaccgcccccgcgatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggaatagttaataacacggtatacgatcccctgcagcccgagctggacagcttcaaggaggagctggacaagtactttaagaatcatacaagccccgacgtggacctgggcgacataagcgggatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 36 - protein, 1269 aa, Severe acute respiratory syndrome coronavirus 2
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - DNA, 3810 bp, Severe acute respiratory syndrome coronavirus 2
  source 1..3810: mol_type = genomic DNA
atgttcgtgttcctggtgctgctgcctctggtgtccagccagtgtgtgaacctgatcacccggacacagagctacaccaacagcttcaccaggggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgctctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtaccagaagaacaacaagagctggatggaaagcgagttccgggtgtacagcagcgccaacaactgcaccttcgagtacgtgtcccagcctttcctgatggacctggaaggcaaagagggcaacttcaagaacctgcgcgagttcgtgtttaagaacatcgacggctacttcaagatctacagcaagcacacccctatcaacctggaacgggatctgcctcagggcttctctgctctggaacccctggtggatctgcccatcggcatcaacatcacccggtttcagacactgctggccctgcaccggtcttacctgacacctgtggatagcagcagcggatggacagctggcgccgctgcttactatgtgggctacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccacattcgccagcgtgtacgcctggaaccggaagagaatcagcaactgcgtggccgactacagcgtgatctacaacttcgccccattcttcgccttcaagtgctacggcgtgtcccctaccaagctgaacgacctgtgcttcaccaatgtgtacgccgacagcttcgtgatccggggcaatgaggtgtcccagattgcccctggacagaccggcaatatcgccgattacaactacaagctgcccgacgacttcaccggctgtgtgatcgcctggaatagcaacaagctggactccaagcctagcggcaactacaactacctgtaccggctgttcagaaagagcaagctgaagcccttcgagcgggacatctccaccgagatctatcaggccggcaacaagccctgcaatggcgtggcaggccctaactgttatagcccactgcagagctacggcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaccggcaccggcgtgctgacagagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggccgttctgtaccagggcgtgaactgtacagaggtgccagtggccattcacgccgatcagctgacccctacttggcgggtgtactccacaggcagcaatgtgtttcagaccagagccggctgtctgattggcgccgagtatgtgaacaacagctacgagtgcgacatccccatcggagccggcatctgtgccagctaccagacacagaccaagagccacagacgggctagaagcgtggccagccagagcatcattgcctacacaatgtctctgggcgccgagaacagcgtggcctacagcaacaactctatcgctatccccaccaacttcaccatcagcgtgaccacagagatcctgcctgtgtccatgaccaagaccagcgtggactgcaccatgtacatctgcggcgatagcaccgagtgctccaacctgctgctgcagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagattctgcccgatcctagcaagcccagcaagcggagcttcatcgaggacctgctgttcaacaaagtgacactggccgacgccggcttcatcaagcagtatggcgattgcctgggcgacattgcagccagggatctgatttgcgcccagaagtttaacggactgaccgtgctgcctcctctgctgaccgatgagatgatcgcccagtacacatctgccctgctggccggcacaatcacaagcggctggacatttggagctggcgctgccctgcagatcccctttgctatgcagatggcctaccggttcaacggcatcggagtgacccagaacgtgctgtacgagaaccagaagctgatcgccaaccagttcaacagcgccatcggcaagatccaggacagcctgagcagtacagccagcgctctgggaaagctgcaggacgtggtcaaccacaatgcccaggcactgaacaccctggtcaagcagctgagcagcaagttcggcgccatctcctccgtgctgaacgatatcctgagcagactggaccctcctgaggccgaggtgcagatcgacagactgatcacaggacggctgcagtccctgcagacctacgttacacagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtatatcaagtggccttggtacatctggctgggctttatcgccggactgattgccatcgtgatggtcacaatcatgctgtgctgcatgaccagctgctgctcttgcctgaagggctgctgtagctgtggctcctgctgcaagttcgacgaggacgattctgagcctgtgctgaaaggcgtgaagctgcactacacatga

SEQ ID NO: 38 - DNA, 3810 bp, Severe acute respiratory syndrome coronavirus 2
  source 1..3810: mol_type = genomic DNA
atgttcgtgtttctggttctgcttccactggtatcctctcaatgcgtcaacttgattactcgaacacagtcatatactaattcttttactcgcggggtgtattatccagataaggtatttcgctccagcgtacttcattctacacaagacctcttcttacctttcttttccaacgttacgtggtttcatgcaatccatgtcagcggtacgaacggaactaaacggttcgacaatccagctctcccttttaacgacggcgtttatttcgcctctaccgaaaagtccaatataatacgcgggtggatatttggcactactctggatagcaaaactcaatccttgctcattgtgaataacgcaacaaatgtcgtaataaaggtttgcgaattccaattttgtaatgatccgtttctcgacgtctaccagaagaacaacaagtcctggatggagagtgagtttagggtgtacagcagtgccaacaattgtactttcgagtatgttagtcaaccatttcttatggaccttgaggggaaagagggcaactttaagaatctgagggagttcgtgtttaagaatattgacggttatttcaaaatttattctaagcatacccccataaatctcgagcgagaccttccccaaggcttttccgcgctggaaccgctcgtcgatctccctattggaattaacataacacggttccaaactctgctcgcattacaccgctcatacctcacgcccgttgactccagcagtggttggactgccggcgccgccgcgtactacgtaggctatctccaacctagaacttttctgctcaaatataatgaaaacggtactatcacagacgctgtggattgtgccttagatcctctgagcgagactaaatgcaccctcaaaagctttacggtagaaaaggggatctatcaaacatccaatttccgagtccagcctaccgaaagtattgtgcggttcccaaacatcaccaatctctgtccctttcacgaagtgtttaatgctacaacttttgcgagcgtgtatgcatggaatcgaaagagaatttccaattgtgttgcggactactctgttatttacaatttcgcacctttcttcgcctttaaatgttatggcgtcagcccgactaaactcaacgatctttgttttacaaacgtttacgccgattccttcgtgattaggggtaacgaagtcagccagattgcacccggacaaactggaaatatagcagattataactataaattgccagacgatttcactggctgcgttatcgcatggaatagcaacaagcttgactccaaacctagtggaaattataattatctttaccggctgtttcggaaatccaaactgaagccatttgagcgcgatatcagcacagagatttatcaagcggggaacaagccatgtaatggagttgctgggccaaattgctacagtccccttcaatcttacgggtttcgccctacctatggagtgggccatcaaccttaccgcgtggtggttttgagttttgaactgctccatgcccccgccacggtgtgcggccctaagaagtccaccaatctggttaagaataagtgcgtcaactttaactttaatggacttacaggaactggggttctgaccgaatctaacaagaaattccttccattccaacaatttggtagggatattgctgataccactgatgccgtaagggacccccaaacactggagattctcgatatcaccccttgctcctttggcggcgtctctgttatcacacccggtactaatacttcaaatcaagtggccgtcctgtatcagggcgtaaactgcacagaggtacctgtagcgattcacgccgatcaactcaccccaacctggcgggtgtattctacggggagtaatgtgtttcagacacgcgcggggtgtttgatcggggccgaatacgtaaataactcatacgagtgcgacatacctattggagctggcatctgtgcatcttatcaaacgcaaactaaatcccaccggcgggcaagatctgtggcatctcaatcaattatcgcctatactatgagcttaggcgctgaaaatagtgtggcatactcaaataacagtatcgcaatcccaaccaattttaccataagtgttaccaccgaaatactgcctgtctctatgacgaaaacctcagtggactgcactatgtatatttgtggcgattctacggagtgctccaacctgctgctccagtacggctccttttgcacccagttgaaacgcgcactcactggaattgctgtagaacaggacaagaatacacaagaggtgtttgcccaagtcaagcaaatctataaaacaccgcccatcaagtatttcggcggcttcaattttagccaaattcttccagatccctcaaagccttccaaacgaagctttattgaagacttgttatttaataaggttactctggcggatgctgggttcattaaacaatatggagattgtttgggcgatatcgccgcacgagatctgatctgcgctcagaaattcaatgggcttaccgtgctgccgcccttgttaacagatgaaatgatcgcgcaatacactagcgccttgcttgcaggcaccattacttccggttggacgttcggggcaggtgccgcactgcaaattcctttcgccatgcaaatggcctataggttcaatggtataggggtgactcagaatgtactctacgagaatcaaaagttaatagcaaatcagttcaatagtgccataggcaagattcaggattccctcagtagcacagcaagtgcactcggcaaattgcaggatgttgtgaaccacaatgcccaagcactgaacacgctcgtaaaacaactgagctcaaagtttggagctatttcaagcgtactcaatgatatactgtcccgattggatccacccgaggctgaggttcaaattgatagactgattaccggaaggttacagtctttgcagacctacgttacccaacagcttattcgcgccgctgaaattcgtgcttctgccaacctcgcggcaactaaaatgtccgaatgcgtccttggtcaaagtaaaagggtcgatttctgtgggaaggggtatcacctcatgtcctttccgcaatctgctccccacggggtagtgtttctccacgtcacgtacgttccagcacaagagaagaactttacaacagcacctgctatctgtcatgacggcaaagcccattttccacgagaaggagtgtttgtctccaacgggacgcattggtttgttactcagaggaatttctacgaaccgcagatcattactactgataacacttttgtttccggaaattgtgatgtggtcatcggtattgttaacaacactgtgtatgaccctctccagcctgaactggacagctttaaggaagagctcgataagtatttcaagaaccatactagccccgacgtggacctgggcgatatcagtgggattaatgcgtcagtagttaacatacagaaagaaatcgaccggcttaatgaagttgcaaagaacttaaatgagtctctgattgatttgcaagaactcggaaagtacgagcaatacattaaatggccctggtacatatggctcgggtttatcgctggcctcatcgcaatagtcatggtgacgattatgctgtgttgcatgaccagttgttgcagctgtttgaaaggatgttgctcatgtgggagctgctgcaagtttgatgaagacgactcagaacctgttttgaaaggcgttaaattacattacacgtaa

SEQ ID NO: 39 - DNA, 3810 bp, Severe acute respiratory syndrome coronavirus 2
  source 1..3810: mol_type = genomic DNA
atgtttgtgttcttggtgttattgcctctcgtctcctcccagtgtgtgaatctgatcacaagaactcaaagctacaccaacagctttactcgcggagtttattacccagacaaagtgttcagaagcagcgtcttgcacagcactcaagacctgttcctacctttctttagcaatgtcacatggttccacgcaatccatgtgagcggtacaaacgggacaaaacggttcgataaccctgcactccccttcaatgatggagtttatttcgcgagcacggaaaagagcaatataattagaggctggatcttcggcaccacgttagactccaagacccaatctctgctgatcgtcaacaacgcgaccaacgtggtgatcaaggtgtgtgagttccaattctgtaacgacccctttctggatgtctaccaaaaaaacaacaagtcctggatggagagcgaattccgggtgtactcctcagccaataactgcacctttgagtacgttagccagccgttcctcatggacctggaggggaaggaaggaaacttcaagaacttacgggaatttgtctttaaaaacattgatggatatttcaaaatctacagcaaacacacacctattaacctggagagagacctcccacagggattcagtgctctcgagccgctggtggacctgcccatcggcatcaacatcaccagatttcagacgctgctggctctgcaccggagttacctgactcctgtcgatagcagctcggggtggacagccggcgctgctgcgtactatgtcggctatctgcagccaagaacctttctgctgaagtataatgaaaacggtaccatcaccgatgccgtggactgcgccctggatccactgagcgaaactaagtgcacgctgaaaagcttcactgtcgaaaaaggaatctaccaaacatctaacttccgagtccagcctaccgagagtatcgtgaggttccctaacatcacaaacctttgcccctttcatgaagtgttcaatgccacaaccttcgcatctgtgtacgcatggaacaggaagagaatcagcaattgtgtcgccgattactccgtcatctataacttcgcccccttcttcgccttcaaatgttacggcgtaagtcccaccaagctaaacgatctatgcttcaccaatgtgtatgctgacagcttcgtgattcgtggcaatgaagtatcccagatcgcgcctggacagacgggtaatattgcagactacaactacaagctgccggacgacttcactggctgcgtcatcgcctggaattccaacaagctcgacagcaaaccttcaggaaactacaattacttgtaccgcttatttagaaaaagcaagctgaagccctttgaaagggatatctctacggagatctaccaagccggcaacaagccctgcaacggggtggcaggccccaactgttacagccctctgcaatcgtacggctttagacccacgtacggcgtagggcaccagccctaccgggtggtagtgctgagcttcgagttactacatgccccagccaccgtgtgtggacccaagaaaagcaccaacctggtgaagaataaatgtgttaacttcaacttcaatggcctgacaggaacaggtgtgctgaccgagagcaacaagaagttccttccattccagcagtttggcagggatattgctgacacaaccgacgccgtgagagatccccagacacttgagatcctggacatcactccttgcagtttcggcggtgtgtctgtgatcacccctggcacaaacacctctaaccaggtggctgtgctgtatcagggcgtgaactgcacggaggtgcctgtggctatccacgccgaccagttgaccccaacctggagagtgtacagcaccggcagcaacgtgttccaaacccgcgctggctgtcttatcggcgcggagtacgttaataactcctacgagtgcgacatcccaatcggggcagggatctgcgcttcatatcagacccagacaaagtcccacagacgggcacgatctgtggcgtcccagtcaatcattgcatatacaatgtccttgggagctgagaatagtgtggcctactccaataacagcatcgccattcccaccaacttcaccatctctgtcaccactgagatcctgccggtgtctatgaccaagacatctgtggactgcaccatgtacatttgtggagattccacggaatgcagtaatctgctgttacagtacggctccttctgcacgcagcttaaaagagccttgaccggaatcgccgtggagcaggacaagaacacccaggaagtgtttgcgcaggtcaagcaaatttacaaaacaccacctattaagtacttcggtggctttaatttcagccagattctgccagacccatccaagccatccaagagatcgttcatcgaggacctgctgttcaacaaggtcaccctggccgacgccggctttattaaacagtatggagactgcctgggggacatcgccgcccgagatctgatctgcgcccaaaagttcaatgggctgaccgtgcttcctcctctgctgacagatgagatgatcgcccagtacactagcgcccttctggctggtaccattacaagcggctggactttcggcgctggggctgcacttcagattccctttgccatgcagatggcctacagattcaacggaataggcgtgacacagaatgtactttacgaaaatcaaaagctgatcgcgaaccagttcaattctgctatcggtaagatacaggacagcctttcatcgacggcctcggccctgggcaaactgcaagacgtggttaaccacaacgctcaggccctcaacactttggtaaagcagctaagtagtaaatttggggccatcagctcagtgctgaatgacatactctctcgtctggaccctcctgaggctgaggtccagatagatagactgatcaccggccggctccagtccctgcagacttatgtgacccagcagctgatcagagccgccgagatccgggcctctgctaaccttgccgccaccaaaatgtctgaatgtgtattgggccaaagcaagcgtgtagacttttgtggtaagggctaccatctgatgtctttccctcagagcgcccctcacggggtggttttcttgcacgtgacgtacgtacctgcacaggagaagaactttacaaccgcccccgccatatgccacgatgggaaggctcacttcccgcgggagggggtcttcgtctctaacggcacccactggtttgtgactcagagaaatttttatgagccccagatcatcaccaccgacaacacatttgtttccggcaactgtgatgtggtaattgggatcgttaacaacaccgtgtatgaccctctgcagcctgaactggacagttttaaagaagagttggataaatactttaagaaccacacctcccctgatgttgacctgggagatattagcggcatcaatgcctccgtggtgaacatccagaaggagattgaccggctgaacgaggtcgccaagaacctcaacgagtccctcatcgatctccaagagctgggcaaatatgagcagtatataaagtggccctggtacatctggctcggcttcatcgcaggcctgatagccatagtgatggtgacaatcatgctgtgctgcatgacgtcgtgctgctcctgcttgaagggctgctgttcctgcggcagctgttgcaagtttgacgaggatgatagtgagcctgtgctgaaaggggtgaagctacactacacatga

SEQ ID NO: 40 - DNA, 3813 bp, Severe acute respiratory syndrome coronavirus 2
  source 1..3813: mol_type = genomic DNA
atgttcgtgttcctggtgctgctgcccctggtgagctctcagtgcgtgaacctgatcacaagaacacagagctacaccaacagcttcacaagaggcgtgtactaccccgacaaggtgttcagaagcagcgtgttgcattctacccaagacctgtttctgcccttcttcagcaacgtgacctggttccacgccatccacgtgagcggcaccaacggcaccaagagattcgacaaccccgccctgcccttcaacgacggcgtgtacttcgctagcaccgagaagagcaacatcatcagaggctggatcttcggcaccaccctcgacagcaaaacacagagcctgctgatcgtgaacaatgccaccaacgtggtgatcaaggtgtgcgagtttcagttctgcaacgaccccttcctggacgtgtatcagaagaacaacaagagctggatggagagcgagttccgtgtgtatagctccgctaacaactgcaccttcgagtacgtgtctcagcccttcctgatggacctggagggcaaggagggcaacttcaaaaacctgagagagttcgtgttcaagaacatcgacggctacttcaagatctacagcaagcacacccccatcaacctggagagagacctgccccaaggcttcagcgccctggagcccctggtggacctgcccatcggcatcaacatcacaagatttcagaccctgctggccctgcacagatcataccttactcctgtggatagcagctcgggttggaccgccggtgccgccgcctactacgtgggctacctgcagcctagaaccttcctgctgaagtacaacgagaacggtacgattactgacgccgtggactgtgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttccacgaggtgttcaacgccaccaccttcgctagcgtgtacgcctggaacagaaagagaatcagcaactgcgtggccgactacagcgtgatctacaacttcgcccccttcttcgccttcaagtgttacggcgtgagccccaccaagctgaacgacctgtgcttcaccaacgtgtacgccgacagcttcgtgatcagaggcaacgaggtgtctcagatcgcccccgggcagaccggcaacatcgccgactacaactacaagctgcccgacgacttcaccggctgcgtgatcgcttggaacagcaataagctggacagcaagccgtccggcaactacaactacctgtacagactgttcagaaagagcaagctgaagcccttcgagagagacatcagcaccgagatctaccaagccggcaacaagccctgcaacggcgtggccggccccaactgctacagccccctgcagagctacggcttcagacccacctacggcgtgggccatcagccctacagagtggtggtgctgagcttcgagctgctgcacgcccccgctaccgtgtgcggccccaagaagagcaccaacctggtgaagaacaagtgcgtgaacttcaactttaacggtctgacgggcaccggcgtgctgaccgagagcaacaagaagttcctgccatttcagcaattcggcagagacatcgccgacaccaccgatgccgtgcgtgaccctcagaccctggagatcctggacatcaccccctgtagtttcggaggcgtgagcgtgatcacccccggcaccaacacaagcaaccaagtggccgtgctgtaccaaggcgtgaactgcaccgaggtgcccgtggccatccacgccgatcagctgacccccacctggagagtatatagcaccggcagcaacgtgtttcagacaagagccggctgcctgatcggcgccgagtacgtgaacaacagctacgagtgcgacatccccatcggcgccggcatctgcgctagctatcagacacagaccaagagccacagaagagctagaagcgtggcttctcagagcatcatcgcctacaccatgagcctgggcgccgagaacagcgtggcctacagcaacaacagcatcgccatccccaccaacttcaccatcagcgtgaccaccgagattctgcctgtgagcatgaccaagacaagcgtggactgcaccatgtacatctgcggcgacagcaccgagtgcagcaacctgctgctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcgtagcttcatcgaggacctgctgttcaacaaggtgaccctggccgacgccggcttcatcaagcagtacggcgactgcctgggcgacattgccgctagagacctgatctgcgctcagaagttcaatgggttaacggtgctcccccccctgctgaccgacgagatgatcgctcagtacactagcgctctgctggctggcactatcacctccggttggaccttcggagccggcgcggccctgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgttaaatgacatcctgagcagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcactacggcacctgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggtatcgttaataataccgtgtacgaccccctgcagcctgagctggacagcttcaaggaggagctggacaagtacttcaagaaccatactagccccgacgtggacctaggcgacattagcggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 41 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 42 - DNA, 670 bp, Severe acute respiratory syndrome coronavirus 2
  source 1..670: mol_type = genomic DNA
atggccgatagcaacggcaccatcaccgtggaagaactgaagaaactgctggaacagtggaacctcgtgatcggcttcctgttcctgacctggatctgcctgctgcagttcgcctacgccaaccggaacagattcctgtatattatcaagctgatcttcctgtggctgctgtggcccgtgacactggcctgttttgtgctggccgccgtgtaccggatcaactggatcacaggcggaatcgccattgccatggcctgtctcgttggcctgatgtggctgagctactttatcgccagcttccggctgttcgcccggaccagatccatgtggtccttcaatcccgagacaaacatcctgctgaacgtgcccctgcacggcacaatcctgacaagacctctgctggaaagcgagctggttatcggcgccgtgatcctgagaggccacctgagaattgccggacaccacctgggcagatgcgacatcaaggacctgcctaaagaaatcacagtggccaccagcagaaccctgtcctactataagctgggcgccagccagagagtggccggcgattctggatttgccgcctacagcagataccggatcggcaactacaagctgaacaccgaccacagctccagcagcgacaatatcgcactgctggtgcagtgat

SEQ ID NO: 43 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 44 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

(19) United States
(12) Patent Application Publication
(10) Pub. No.: US 2024/0398935 A1
(43) Pub. Date: Dec. 5, 2024

(54) SARS COV-2 VACCINES, ASSOCIATED POLYNUCLEOTIDES, AND METHODS OF USE

(71) Applicants: Aegis Life, Inc., San Diego, CA (US); Entos Pharmaceuticals, Inc., Edmonton (CA)

(72) Inventors: John LEWIS, Encinitas, CA (US); Arun Raturi, Del Mar, CA (US); Roy Duncan, Halifax (CA); Ping Wee, San Diego, CA (US); Prakash Bhandari, San Diego, CA (US); Thornton Thompson, Seattle, WA (US); Alex Sevy, San Diego, CA (US)

(21) Appl. No.: 18/678,619

(22) Filed: May 30, 2024

Related U.S. Application Data
(60) Provisional application No. 63/505,368, filed on May 31, 2023.

(51) Int. Cl. A61K 39/215; A61K 9/50; A61K 39/00; A61P 37/04

(52) U.S. Cl. CPC A61K 39/215; A61K 9/5015; A61K 9/5052; A61P 37/04; A61K 2039/53; A61K 2039/54; A61K 2039/545

(57) ABSTRACT

The present disclosure relates to a vaccines and related polynucleotides useful in eliciting an immune response to the SARS-CoV-2 virus and related methods of use.

### CROSS REFERENCE

This application claims the benefit of U.S. Provisional Application No. 63/505,368 filed May 31, 2023 the content of which is incorporated herein by reference in its entirety.

### SEQUENCE LISTING

The instant application contains a Sequence Listing which has been submitted electronically in XML file format and is hereby incorporated by reference in its entirety. Said XML copy, created on May 24, 2024, is named 94529-0016_704201US_SL.xml and is 84,001 bytes in size.

### BRIEF SUMMARY

In some aspects provided herein are SARS-CoV-2 vaccines which comprises vectors encoding a SARS-CoV-2 protein (e.g. the spike protein) which contain optimized codons for enhanced immunogenicity. In some embodiments, these improved vectors can be used as monovalent vaccines (e.g., there is only one vector in the vaccine encoding the SARS-CoV-2 protein such as the spike protein) or such vectors can be used in multi-valent vaccines as described herein.

In certain aspects provided herein are multi-valent (e.g., bi-valent) vaccines, vectors, and other polynucleotides useful in the treatment, prevention, and or mitigation of the SARS-CoV-2 virus or it associated diseases and indications. In some embodiments, provided herein are DNA vaccines which comprise vectors encoding multiple SARS-CoV-2 proteins. In some embodiments, administration of such a vaccine elicits a greatly enhanced immune response as compared to a single antigen strategy.

In some embodiments, the SARS-CoV-2 vaccines provided herein comprise DNA plasmids comprised in proteolipid vehicles which comprise a fusogenic membrane protein, thereby facilitating fusion of the proteolipid vehicles with target cells in order to deliver the DNA cargo. In some embodiments, the fusogenic membrane proteins are fusion-associated small transmembrane (FAST) proteins (e.g., those of the Fusogenix proteolipid vehicle (PLV) platform provided by Entos Pharmaceuticals). Further provided herein are methods of treating, preventing, reducing risk of, and/or reducing the severity of SARS-CoV-2 infection by administering a vaccine as provided herein.

In one aspect provided herein is a SARS-CoV-2 DNA vaccine, comprising: a) a first DNA vector comprising a polynucleotide sequence encoding a first SARS-CoV-2 spike protein or a portion thereof, b) a second DNA vector comprising a polynucleotide sequence encoding a second SARS-CoV-2 protein or a portion thereof; and wherein the first DNA vector and the second DNA vector are encapsulated in proteolipid vehicles that comprise a fusogenic membrane protein, optionally comprising an adjuvant or a polynucleotide sequence encoding an adjuvant.

In another aspect herein is a SARS-CoV-2 DNA vaccine, comprising: a) a first DNA vector comprising a polynucleotide sequence encoding a first SARS-CoV-2 spike protein or a portion thereof, wherein the first SARS-CoV-2 spike protein or portion thereof comprises 1) at least one amino acid substitution selected from a D614G amino acid substitution, a K986P amino acid substitution and a V987P amino acid substitution, and 2) an additional amino acid substitution at another residue of the first SARS-CoV-2 spike protein, wherein residue position numbering is based on SEQ ID NO: 1 as a reference sequence; b) an adjuvant or a polynucleotide sequence encoding an adjuvant; wherein the first DNA vector is encapsulated in proteolipid vehicles that comprise a fusogenic membrane protein.

In another aspect described herein is a SARS-CoV-2 DNA vaccine, comprising: a first DNA vector comprising a polynucleotide sequence encoding a first SARS-CoV-2 spike protein or a portion thereof, wherein the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence; a second DNA vector comprising a polynucleotide sequence encoding a second SARS-CoV-2 protein or a portion thereof; and an adjuvant or a polynucleotide sequence encoding an adjuvant; wherein the first DNA vector and the second DNA vector are encapsulated in proteolipid vehicles that comprise a fusogenic membrane protein.

In some embodiments, the first SARS-CoV-2 spike protein or portion thereof comprises a D614G amino acid substitution, a K986P amino acid substitution, and a V987P amino acid substitution. In some embodiments, the first SARS-CoV-2 spike protein or the portion thereof is a full-length SARS-CoV-2 spike protein. In some embodiments, the first SARS-CoV-2 spike protein comprises an amino acid sequence having at least 90%, at least 95%, at least 96%, at least 97%, at least 98%, or at least 99% sequence identity with the sequence set forth in SEQ ID NO: 1. In some embodiments, the first SARS-CoV-2 spike protein further comprises a T19I, 24-26Del, A27S, A67V, 69-70Del, T95I, 137-145Del, G142D, 143-145Del, Y145H, 211Del, L212I, V213G, ins214EPE, ins214TDR, A222V, G339D, G339H, R346K, R346S, V367F, L368I, S371F, S371L, S373P, S375F, T376A, P384L, N394S, D405N, R408S, Q414K, K417N, K417T, N439K, N440K, V445P, G446S, Y449H, Y449N, N450K, L452R, L452Q, N460K, S477N, T478K, V483A, E484A, E484K, E484Q, E484Del, F486P, F486V, F490R, F490S, Q493K, Q493R, S494P, G496S, Q498R, N501T, N501Y, Y505H, E516Q, T547K, Q613H, A653V, H655Y, G669S, Q677H, N679K, ins679GIAL, P681H, P681R, A701V, S704L, N764K, D796Y, N856K, Q954H, N969K, or L981F modification, or any combination thereof. In some embodiments, the first SARS-CoV-2 spike protein comprises an amino acid sequence of SEQ ID NO: 2, 3, 31, or 36. In some embodiments, the first SARS-CoV-2 spike protein comprises an amino acid sequence of SEQ ID NO: 36. In some embodiments, the polynucleotide sequence encoding the first SARS-CoV-2 spike protein comprises a polynucleotide sequence having at least 80%, at least 85%, at least 90%, at least 95%, at least 97%, at least 98%, or at least 99% sequence identity with the sequence set forth in SEQ ID NO: 4, 5, 32-35, or 37-40. In some embodiments, the polynucleotide sequence encoding the first SARS-CoV-2 spike protein comprises a polynucleotide sequence having at least 80%, at least 85%, at least 90%, at least 95%, at least 97%, at least 98%, or at least 99% sequence identity with the sequence set forth in SEQ ID NO: 40. In some embodiments, the polynucleotide sequence encoding the first SARS-CoV-2 spike protein comprises a polynucleotide sequence of SEQ ID NO: 4, 5, 32-35, or 37-40. In some embodiments, the polynucleotide sequence encoding the first SARS-CoV-2 spike protein comprises a polynucleotide sequence of SEQ ID NO: 40.

In some embodiments, the second SARS-CoV-2 protein is a second SARS-CoV-2 spike protein. In some embodiments, the second SARS-CoV-2 spike protein comprises at least 5, at least 10, at least 15, at least 20, at least 25, or at least 30 amino acid modifications relative to the first SARS-CoV-2 spike protein. In some embodiments, the second SARS-CoV-2 spike protein further comprises a T19I, 24-26Del, A27S, A67V, 69-70Del, T95I, 137-145Del, G142D, 143-145Del, Y145H, 211Del, L212I, V213G, ins214EPE, ins214TDR, A222V, G339D, G339H, R346K, R346S, V367F, L368I, S371F, S371L, S373P, S375F, T376A, P384L, N394S, D405N, R408S, Q414K, K417N, K417T, N439K, N440K, V445P, G446S, Y449H, Y449N, N450K, L452R, L452Q, N460K, S477N, T478K, V483A, E484A, E484K, E484Q, E484Del, F486P, F486V, F490R, F490S, Q493K, Q493R, S494P, G496S, Q498R, N501T, N501Y, Y505H, E516Q, T547K, Q613H, D614G, A653V, H655Y, G669S, Q677H, N679K, ins679GIAL, P681H, P681R, A701V, S704L, N764K, D796Y, N856K, Q954H, N969K, L981F, K986P, or V987P modification, or any combination thereof. In some embodiments, the second SARS-CoV-2 spike protein comprises a K986P amino acid substitution and a V987P amino acid substitution. In some embodiments, the second SARS-CoV-2 spike protein comprises an amino acid sequence having at least 90%, at least 95%, at least 96%, at least 97%, at least 98%, or at least 99% sequence identity with the sequence set forth in SEQ ID NO: 31 or 36. In some embodiments, the second SARS-CoV-2 spike protein comprises an amino acid sequence of SEQ ID NO: 31 or 36. In some embodiments, the polynucleotide sequence encoding the second SARS-CoV-2 spike protein comprises a polynucleotide sequence having at least 80%, at least 85%, at least 90%, at least 95%, at least 97%, at least 98%, or at least 99% sequence identity with the sequence set forth in SEQ ID NO: 4, 32-35, or 37-40. In some embodiments, the polynucleotide sequence encoding the second SARS-CoV-2 spike protein comprises a polynucleotide sequence of SEQ ID NO: 4, 32-35 or 37-40. In some embodiments, the first SARS-CoV-2 spike protein and the second SARS-CoV-2 spike protein are derived from different variants of the SARS-CoV-2 virus.

In some embodiments, the first SARS-CoV-2 spike protein is derived from the Wuhan strain of SARS-CoV-2. In some embodiments, the second SARS-CoV-2 spike protein is derived from an omicron variant of SARS-CoV-2.

In some embodiments, the second SARS-CoV-2 protein comprises a SARS-CoV-2 envelope protein, a SARS-CoV-2 membrane protein, or a SARS-CoV-2 nucleocapsid protein, or a portion of any of these.

In some embodiments, the second SARS-CoV-2 protein is a full-length SARS-CoV-2 envelope protein. In some embodiments, the SARS-CoV-2 envelope protein comprises an amino acid sequence having at least about 80%, 85%, 90%, 95%, 97%, or 98% sequence identity to the sequence MYSFVSEETGTLIVNSVLLFLAFVVFLLVTLAILTALRLCAYCCNIVNVSLVKPSFYV YSRVKNLNSSRVPDLLV (Uniprot ID P0DTC4) (SEQ ID NO: 44). In some embodiments, the SARS-CoV-2 envelope protein comprises the sequence SEQ ID NO: 44.

In some embodiments, the second SARS-CoV-2 protein is a SARS-CoV-2 membrane protein, or a portion thereof. In some embodiments, the second SARS-CoV-2 protein is a full-length SARS-CoV-2 membrane protein. In some embodiments, the SARS-CoV-2 membrane protein comprises an amino acid sequence having at least about 80%, 85%, 90%, 95%, 97%, 98%, or 99% sequence identity to the sequence MADSNGTITVEELKKLLEQWNLVIGFLFLTWICLLQFAYANRNRFLYIIKLIFLWLLW PVTLACFVLAAVYRINWITGGIAIAMACLVGLMWLSYFIASFRLFARTRSMWSFNPE TNILLNVPLHIGTILTRPLLESELVIGAVILRGHLRIAGH HLGRCDIKDLPKEITVATSRT LSYYKLGASQRVAGDSGFAAYSRYRIGNYKLNTDHSSSSDNIALLVQ (UniProt ID P0DTC5) (SEQ ID NO: 41). In some embodiments, the SARS-CoV-2 membrane protein comprises the sequence SEQ ID NO: 41.

In some embodiments, the second SARS-CoV-2 protein is a SARS-CoV-2 nucleocapsid protein, or a portion thereof. In some embodiments, the second SARS-CoV-2 protein is a full-length SARS-CoV-2 nucleocapsid protein. In some embodiments, the SARS-CoV-2 nucleocapsid protein comprises an amino acid sequence having at least about 80%, 85%, 90%, 95%, 97%, 98%, or 99% sequence identity to the sequence MSDNGPQNQRNAPRITFGGPSDSTGSNQNGERSGARSKQRRPQGLPNNTASWFTAL TQHGKEDLKFPRGQGVPINTNSSPDDQIGYYRRATRRIRGGDGKMKDLSPRWYFYY LGTGPEAGLPYGANKDGIIWVATEGALNTPKDHIGTRNPANNAAIVLQLPQGTTLPK GFYAEGSRGGSQASSRSSSRSRNSSRNSTPGSSRGTSPARMAGNGGDAALALLLLDR LNQLESKMSGKGQQQQGQTVTKKSAAEASKKPRQKRTATKAYNVTQAFGRRGPEQ TQGNFGDQELIRQGTDYKHWPQIAQFAPSASAFFGMSRIGMEVTPSGTWLTYTGAIK LDDKDPNFKDQVILLNKHIDAYKTFPPTEPKKDKKKKADETQALPQRQKKQQTVTL LPAADLDDFSKQLQQSMSSADSTQA (UniProt ID P0DTC9) (SEQ ID NO: 43). In some embodiments, the SARS-CoV-2 nucleocapsid protein comprises the sequence of SEQ ID NO: 43. In some embodiments, the first SARS-CoV-2 spike protein comprises an amino acid sequence having at least 90%, at least 95%, at least 96%, at least 97%, at least 98%, or at least 99% sequence identity with the sequence set forth in SEQ ID NO: 3, 31, or 36. In some embodiments, the second SARS-CoV-2 spike protein comprises an amino acid sequence of SEQ ID NO: 3, 31, or 36. In some embodiments, the first SARS-CoV-2 spike protein is derived from an omicron variant of SARS-CoV-2.

In some embodiments, the DNA vectors are each double-stranded DNA vectors. In some embodiments, the DNA vectors are each plasmids. In some embodiments, the DNA vectors are present in the vaccine in about equal amounts.

In some embodiments, the proteolipid vehicle comprises an ionizable lipid. In some embodiments, the fusogenic membrane protein is a fusion-associated small transmembrane (FAST) protein. In some embodiments, the FAST protein comprises domains from one or more FAST proteins selected from p10, p14, p15, and p22. In some embodiments, the FAST protein comprises an amino acid sequence having at least 80% sequence identity to the sequence: MGSGPSNFVNHAPGEAIVTGLEKGADKVAGTISHTIFVEIVSSSTGIIIAVGIFAFIFSFL YKLLQWYNRKSKNKKRKEQIREQIELGLLSYGAGVASLPLLNVIAHNPGSVISATPIY KGPCTGVPNSRLLQITSGTAEENTRILNHDGRNPDGSINV (SEQ ID NO: 7).

In some embodiments, the first DNA vector and/or the second DNA vector comprise the polynucleotide sequence encoding the adjuvant. In some embodiments, both the first DNA vector and the second DNA vector comprises copies of the polynucleotide sequence encoding the adjuvant. In some embodiments, the polynucleotide encoding the adjuvant is positioned on the DNA vector in which it is comprised such that the adjuvant is included in the 3′-UTR of an mRNA transcribed from the polynucleotide encoding the SARS-CoV-2 protein. In some embodiments, the adjuvant comprises a pathogen-associated molecular pattern (PAMP). In some embodiments, the adjuvant comprises an RNA CpG motif, a retinoic acid-inducible gene I (RIGI) agonist, a melanoma differentiation-associated protein 5 (MDA5) agonist, or a combination thereof. In some embodiments, the first DNA vector and/or the second DNA vector comprise a second polynucleotide sequence encoding a second adjuvant. In some embodiments, both the first DNA vector and the second DNA vector comprises copies of a second polynucleotide sequence encoding a second adjuvant. In some embodiments, the second polynucleotide encoding the second adjuvant is positioned on the DNA vector such that the second adjuvant is included in the 3′-UTR of an mRNA transcribed from the polynucleotide encoding the SARS-CoV-2 protein. In some embodiments, the adjuvant and the second adjuvant each independently comprise PAMPs. In some embodiments, the adjuvant comprises an RNA CpG motif and the second adjuvant comprises a RIGI agonist.

In some embodiments, the SARS-CoV-2 DNA vaccine is stable at a temperature of from about 2° C. to about 8° C. for a period of at least about 1 month, at least about 2 months, at least about 3 months, at least about 6 months, at least about 9 months, or at least about 12 months. In some embodiments, the SARS-CoV-2 DNA vaccine is stable at room temperature for a period of at least about 7 days, at least about 14 days, at least about 21 days, or at least about 28 days.

In some embodiments, administration of the SARS-CoV-2 DNA vaccine to a subject induces at least 2-fold, at least 3-fold, at least 4-fold, at least 5-fold, at least 6-fold, at least 7-fold, at least 8-fold, at least 9-fold, or at least 10-fold higher anti-spike protein antibody concentration as compared to a SARS-CoV-2 DNA vaccine which contains only one of the vectors. In some embodiments, the higher anti-spike protein antibody concentration is determined as against the spike protein from which the second SARS-CoV-2 spike protein is derived. In some embodiments, the higher anti-spike protein concentration is determined as against a third SARS-CoV-2 spike protein. In some embodiments, the third SARS-CoV-2 spike protein comprises at least 1, at least 2, at least 3, at least 4, or at least 5 amino acid modification as compared to the first and second SARS-CoV-2 spike proteins. In some embodiments, the third SARS-CoV-2 spike protein is a different sub-variant of the same variant from which the first or second SARS-CoV-2 spike protein is derived.

Also provided herein in an aspect is a method of eliciting an anti-SARS-CoV-2 immune response in a subject, the method comprising administering to the subject a therapeutically effective amount of the SARS-CoV-2 DNA vaccine provided herein to the subject. In some embodiments, the administering comprises administering two doses of the therapeutically effective amount of the SARS-CoV-2 DNA vaccine. In some embodiments, the two doses are administered at an interval of from about 2 weeks to about 8 weeks apart. In some embodiments, the therapeutically effective amount of the SARS-CoV-2 DNA vaccine is from about 0.050 mg to about 0.500 mg. In some embodiments, the SARS-CoV-2 DNA vaccine is administered intramuscularly. In some embodiments, the SARS-CoV-2 DNA vaccine is administered without electroporation. In some embodiments, the subject is a mammal. In some embodiments, the subject is a human. In some embodiments, antibodies induced by the administration of the SARS-CoV-2 DNA vaccine exhibit specific binding for two or more variants of the SARS-CoV-2 spike protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1A shows an illustration of a mechanism of a SARS-CoV-2 DNA vaccine as provided herein activating an immune response.

FIG. 1B shows a schematic representation of a SARS-CoV-2 spike protein.

FIG. 1C shows a western blot analysis of 293T cell extracts, un-transfected (UnTx), or transiently transfected with empty Nanoplasmid vector (Ctrl NP), Nanoplasmid encoding Spike (Spike NP), or p10 backbone encoding Spike (Spike p10) using SARS-CoV-2 Spike rabbit mAb (upper) and alpha-tubulin mouse mAb (lower).

FIG. 1D shows a Western blot of 293T cell lysates from cells transfected with 0, 1, 2 and 5 microgram of PLVs carrying various plasmid DNA encoded antigens; control (Ctrl) lanes are SARS-CoV-2 Nucleocapsid, CpG lanes are SARS-CoV-2 Nucleocapsid+CpG, Spike lanes are SARS-CoV-2 full length Spike. The primary antibodies used were RBD mouse mAb (upper) and alpha-actin mouse mAb (lower).

FIG. 1E shows Western blot analysis of secreted Receptor Binding Domain from 293T cell extracts from cells transfected with either empty p10 vector, p10 backbone encoding sRBD or p10 encoding full length Spike. The primary antibodies used were SARS-CoV-2 Spike RBD mouse mAb (upper) and alpha-tubulin mouse mAb (lower).

FIG. 2A shows the fold change in anti-Spike antibodies from vaccinated animals versus control animals as measured from serum by indirect electro-chemiluminescence immunoassay (ECLIA) 21 days after initial dose (7 days post boost).

FIG. 2B shows interferon gamma ELISpot assay conducted on splenocytes from immunized and control mice. Splenocytes were stimulated at 21 days post immunization (7 days post boost).

FIG. 3A shows the fold change in anti-Spike binding of serum from immunized mice 14 days post boosting as measured by ELISA.

FIG. 3B shows anti-Spike titers (U/mL) on the same serum samples generated in FIG. 3A using a standardized assay performed by a reference lab.

FIG. 3C shows 50% neutralizing antibody titers (IC50) in sera of immunized animals as measured by PSVN assay.

FIG. 3D shows IC50 in sera of immunized animals as measured by a standardized PSVN assay performed by a reference lab.

FIG. 3E shows IC50 in sera of immunized animals as measured by inhibition of ACE2 binding to the RBD domain of SARS-CoV-2 spike.

FIG. 3F shows relative neutralizing capacity of sera from immunized mice as measure by inhibition of ACE2 binding to the S1 domain of SARS-CoV-2 Spike.

FIG. 3G shows antibody binding to Wuhan Spike as measured by ECLIA following PLV-DNA vaccination. Animals (n=10 per group) were immunized on days 0 and 21 with the indicated doses of NP-S-2P or 100 μg of NP-Vector control. Reciprocal titer of anti-Spike IgG binding is depicted by bars, **** denotes P<0.0001, ns denotes P>0.05 by Kruskal-Wallis Test with Dunn's multiple comparison test versus NP-vector control.

FIG. 3H shows 50% neutralizing antibody titers (IC50) in serum of immunized animals in (E) as measured by PSVN assay using SARS CoV-2 Spike protein-pseudotyped (Wuhan) lentivirus-expressing firefly luciferase (FLuc). For comparison convalescent human serum (CHS) samples (n=28) and Comirnaty vaccinated patient serum (n=52) were tested in parallel. Mean PSVN IC50 is depicted by bars * denotes P<0.05, ** denotes P<0.01, ns denotes P>0.05 by Kruskal-Wallis Test with Dunn's multiple comparison test versus control. There was no detectable difference between mean PSVN IC50 for NP-S-2P 10 μg, 25 μg, 100 μg and 250 μg groups and the Comirnaty® group, ns in magenta denotes P>0.05 by Kruskal-Wallis Test with Dunn's multiple comparison test versus Comirnaty® group.

FIG. 3I shows the same as in FIG. 3G measuring binding to Delta Spike protein. Reciprocal titer of anti-Spike IgG binding is depicted by bars, ** denotes P<0.01 **** denotes P<0.0001, ns denotes P>0.05 by Kruskal-Wallis Test with Dunn's multiple comparison test versus NP-vector control

FIG. 3J shows the same as in FIG. 3H with Delta Spike-protein pseudotyped lentivirus expressing firefly luciferase.

FIG. 3K shows Relative neutralizing capacity of sera from immunized mice in (F) as measured by inhibition of ACE2 binding to the S1 domain of SARS-Cov2 Wuhan Spike (magenta) or Delta Spike (green). Mean % inhibition of ACE2 binding is depicted by bars. Mean % ACE2 inhibition of Wuhan or Delta spike is not significantly different from control at the 1 μg dose (P>0.05), at all other doses mean % ACE2 inhibition is significantly different from control (P<0.0001) by one-way ANNOVA and Dunnett's post test (not shown). No significant difference in mean % ACE2 inhibition between Wuhan Spike and Delta Spike were detected, ns denotes P>0.01 by two-way ANNOVA and Sidak's post test.

FIG. 4A shows frequency of interferon gamma producing CD4+ T-cells in splenocytes isolated at day 35 (14 post boost) from immunized mice.

FIG. 4B shows frequency of interferon gamma producing CD8+ T-cells in splenocytes isolated at day 35 (14 post boost) from immunized mice.

FIG. 4C shows frequency of blasting lymphocytes per are determined by enumerating cells with a blasting phenotype from H&E-stained sections of spleen 8 days post infection.

FIG. 4D shows evidence of splenomegaly quantified by organ weight 8 days post immunization.

FIG. 4E shows evidence of splenomegaly quantified by organ weight 21 days post immunization.

FIG. 4F shows a representative H&E staining in spleens from control and NP-S immunized mice 8 days after dosing.

FIG. 4G shows a representative H&E staining in spleens from control NP-S immunized mice 8 days after dosing from draining lymph node.

FIG. 5A shows an illustration of activated CD8+ T-cells recognizing SARS-CoV-2 infected cells by binding of the T-cell receptor to MHC-I presented SARS-CoV-2 antigens.

FIG. 5B shows Annexin V and 7-AAD flow cytometry used to confirm apoptosis induction in B16F10 cells cultured with splenocytes isolated from NP-S immunized and control mice.

FIG. 5C shows percentage of dead target cells represented as Annexin V+/7-AAD+ double populations summed with AnnexinV and 7-AAD single positive populations, gated on B16F10-Spike cells.

FIG. 5D shows percentage of dead target cells represented as in FIG. 5C following co-culture of B16 target cells expressing WT or variant of concern (VOC) spike with purified CD*+ T-cells isolated from vaccinated animals.

FIG. 6A shows timeline of a hamster immunization regimen.

FIG. 6B shows pre-challenge anti-spike antibody titers as measured by endpoint ELISA.

FIG. 6C shows body weight changes post challenge.

FIG. 6D shows percent of starting weight at peak weight loss (day 7). Each dot represents one animal.

FIG. 6E shows viral genome copies present in nasal washes at the indicated time points.

FIG. 6F shows CXCL10 expression in nasal turbinates as determined by qRT-PCR.

FIG. 6G shows IL-6 expression in nasal turbinates as determined by qRT-PCR.

FIG. 6H shows interferon gamma expression in nasal turbinates as determined by qRT-PCR.

FIG. 6I shows viral genome copies present in lung tissue at the indicated time points.

FIG. 6J shows CXCL10 expression in lung as determined by qRT-PCR.

FIG. 6K shows IL-6 expression in lung as determined by qRT-PCR.

FIG. 6L shows interferon gamma expression in lung as determined by qRT-PCR.

FIG. 7A shows a non-human primate (NHP) immunization regimen. Groups of 6 NHPs received either saline alone (control), one 250 microgram dose of NP-S (IM) on day 0 (single dose) or two 250 microgram doses of NP-S (IM) at days 0 and 28.

FIG. 7B shows IC50 values from serum at the indicated days obtained from NHPs immunized according to the protocol of FIG. 7A.

FIG. 7C shows Day 42 IC50s for neutralizing titers for each animal compared to convalescent human serum.

FIG. 7D shows injection site irritation for each animal as measured by Primary Dermal Irritation Index by analyzing erythema and edema at the injection site for each animal. Animals with no observable edema or erythema were assigned a value of 0.

FIG. 8A shows viral load in the indicated tissues 3 days post challenge as determined by TCID₅₀ assay, expressed as TCID₅₀ equivalents per gram of tissue.

FIG. 8B shows the same as FIG. 8A as measured by RT-qPCR and expressed as genome copies/gram of tissue.

FIG. 8C shows viral load in the indicated tissues 7 days post challenge.

FIG. 8D shows viral load in the indicated tissues 14 days post challenge.

FIG. 9 shows experimental data generated for a number of vaccine candidates.

FIG. 10 shows a vector map of the vector used in the NP-S-2P vaccine candidate.

FIG. 11A shows anti-spike IgG levels in NHPs at various time points after receiving 250 microgram doses of NP-S-2P vaccine at time points ranging from 0 to 56 days.

FIG. 11B shows neutralizing titers against Wuhan and Delta variant strains at various time points after receiving 250 microgram doses of NP-S-2P vaccine at time points ranging from 0 to 56 days.

FIG. 11C shows a time course of 50% neutralizing antibody titers (IC50) in serum of NHP subjects as measured by PSVN assay using SARS-CoV-2/Wuhan Spike (left) or Delta Spike (right) pseudotyped lentivirus-expressing firefly luciferase (Fluc). Mean PSVN IC50 is denoted by bars, each data point represents one animal subject.

FIG. 11D shows a time course of relative neutralizing capacity of sera from immunized NHP subjects as measured by inhibition of ACE2 binding to the S1 domain of SARS-Cov2/Wuhan Spike (left) or Delta Spike (right). Mean % inhibition of ACE2 denoted by bars.

FIG. 12A shows Reciprocal titer of anti-Wuhan Spike antibody following immunization with the PLV-based vaccines shown, on day 35 (14 days after booster dose). *** denotes P<0.001, ** denotes P<0.01, * denotes P<0.05, ns denotes P>0.05 by one way ANNOVA with Kruskal-Wallis test compared to vector control.

FIG. 12B shows the same as in FIG. 12A measuring anti-Omicron B.1.351 antibody.

FIG. 12C shows the same as in FIG. 12A measuring anti-Omicron BA.2 antibody.

FIG. 12D shows the same as in FIG. 12A measuring anti-Omicron BA.3 antibody.

FIG. 12E shows the same as in FIG. 12A measuring anti-Omicron BA.4 antibody.

FIG. 12F shows the same as in FIG. 12A measuring anti-Omicron BA.5 antibody.

FIG. 13 shows in vitro transfection results of selected codon optimized vaccine candidates encoding omicron variant spike proteins.

FIG. 14 shows Day 35 anti-spike IgG titers obtained in C57BL/6 mice receive a prime and boost immunization with the indicated vaccine candidates on Day 1 and Day 21. For each spike protein against which IgG titer is shown, results shown in the FIG. 14 from left to right are for NP-S-2P, NP-Omicron-2P, XBB.1.5-2P_AZ, XBB.1.5-2P_GS, and XBB.1.5-2P_TF.

FIG. 15 shows antibody titer in C57BL/6 mice as measured by ECLIA following PLV-DNA immunization. Mice were administered a single dose of the indicated vaccine and dose level on Day 1. Fold change in anti-Spike binding to the indicated SARS-CoV-2 variant (X-axis) of Day 36 serum compared to baseline (pre-immunization) serum is plotted. Bars indicate geometric mean values, symbols represent data for individual animals. For each spike protein against which serum anti-spike IgG levels were measured, results shown from left to right are for PBS, NP-S-CPG-RIGI (100 ug), NP-S-2P (25 ug), NP-S-2P (100 ug), VAX-002 (25 ug), and VAX-002 (100 ug).

FIG. 16A shows neutralizing antibody response as measured by a validated PsVN assay for serum from mice immunized with the indicated vaccine candidate and dose level (N=10 per group). PsVN was conducted against pseudovirus expressing XBB.1.5 Spike protein. Bars indicate geometric mean and symbols represent data for individual animals. Dotted line indicates lowest dilution tested.

FIG. 16B shows the same as in FIG. 16A but using pseudovirus expressing D614G Spike protein. (**, P<0.01; ****, P<0.0001 by Kruskal-Wallis test with Dunn's post-test).

FIG. 16C shows inhibition of ACE2-Spike binding by antibodies in serum of vaccinated animals using the same serum samples as in FIG. 16A. Spike protein from SARS-CoV-2 XBB.1.5 strain was used. (**, P<0.01; ***, P<0.001 by Kruskal-Wallis test with Dunn's post-test). Bars indicate mean values and symbols represent data for individual animals.

FIG. 16D shows the same as in FIG. 16C but using Spike protein from SARS-CoV-2 Wuhan strain.

FIG. 17 shows splenocytes from animals administered the indicated vaccine dose level were isolated 35 days after dosing, followed by ex vivo stimulation with spike peptide pools spanning the SARS-CoV-2 spike ORF, strain XBB.1.5. IFN-γ release by SARS-CoV-2-specific T-cells was assessed by ELISpot. Mean frequency of IFN-γ positive cells is indicated by bars, symbols represent individual animals (n=10 animals per group); ns denotes no significant difference from control (P>0.05); asterisks indicate significant difference from unstimulated control (*** P<0.001; **** P<0.0001 by Kruskal-Wallis test with uncorrected Dunn's post-test).

### DETAILED DESCRIPTION

Ending the COVID-19 pandemic requires a global vaccination effort leveraging scalable, globally deployable vaccine platforms that are also rapidly adaptable to emerging variants. DNA-based vaccines can satisfy these criteria, but inefficient intracellular DNA delivery has limited their development and efficacy. Herein is reported rapid prototyping of DNA vaccine candidates utilizing an intracellular delivery platform where plasmid DNA vaccines are encapsulated in proteolipid vehicles (PLVs) formulated with a fusion-associated small transmembrane (FAST) protein and well-tolerated lipids. Rapid prototyping of SARS-CoV2 vaccine candidates identified full-length SARS-CoV-2 Spike protein (Wuhan strain) combined with two genetic adjuvants (CpG motifs, RIG-I agonist, termed NP-S-CpG-RIGI) could elicit both potent neutralizing antibody responses in mice and nonhuman primates comparable to COVID-19 convalescent patients, and Spike-specific T cell responses including functional cytotoxic T lymphocyte responses. In some instances, it was observed that a single dose of NP-S protects hamsters from morbidity following SARS-CoV-2 challenge. The FAST-PLV vaccine platform is ideally suited to develop countermeasures against emerging infections. It was further observed that the immune response to the NP-S-CpG-RIGI construct, including responses to delta and omicron SARS-CoV-2 variants, could be unexpectedly improved by the introduction of selected mutations to the Wuhan strain spike protein sequence as provided herein.

Several features of nucleic acid vaccines have propelled them to the forefront of COVID-19 vaccination efforts, including rapid prototyping of vaccine candidates for improved immune responses and protection from emerging variants, the absence of an immunogenic delivery system, and the case of nucleic acid vaccine manufacturing. Nucleic acid vaccine payloads must be expressed and translated intracellularly to yield antigens that elicit a host immune response. The value of mRNA-based vaccines to prevent COVID-19 has been clearly demonstrated, but the requirement for cold-storage hinders their use in lower middle-income countries. By contrast, the inherent stability of DNA allows DNA vaccines to be stored and transported at normal refrigeration temperatures. DNA vaccines are relatively inexpensive to manufacture at scale and can be programmed with multiple antigens and/or immunostimulatory factors on the same molecule. In addition, cytoplasmic DNA activates innate immune responses leading to IFN production through the cGAS-STING pathway, and cytokine production through the AIM2 inflammasome pathway. Recently, DNA vaccines have been shown to generate both innate cytokine signaling and antigen-specific B and T cell responses via the STING pathway but independent of the cGAS sensor in vivo, indicating the presence of a redundant DNA sensor. For these reasons, DNA-based vaccines may be ideally suited for rapid prototyping and global deployment.

DNA vaccines are safe in animals and humans and generate balanced cellular and humoral immune responses in non-human primates (NHPs). These vaccines are effective and licensed for animal use but not yet approved for use in humans. Recently, Zydus Cadila has received approval of its three-dose COVID-19 vaccine, ZyCoV-D, that showed efficacy of 66.6% in an interim study, and is the worlds' first DNA vaccine for humans.

Inefficient intracellular plasmid delivery has been a major barrier limiting the clinical application of DNA vaccines to date. Intracellular DNA vaccine delivery has traditionally relied on viral vectors, cationic lipid nanoparticles (LNPs) or electroporation. However, these methods each suffer from notable limitations; the immunogenicity of adenovirus- and adeno-associated virus (AAV)-based vectors limits their repeated administration, cationic lipid nanoparticles can generate immunogenic toxicity and acute proinflammatory cytokine secretion, and electroporation requires a device to deliver an electric charge at the injection site. These limitations prompted the development of FAST-PLVs as a DNA vaccine delivery platform described herein.

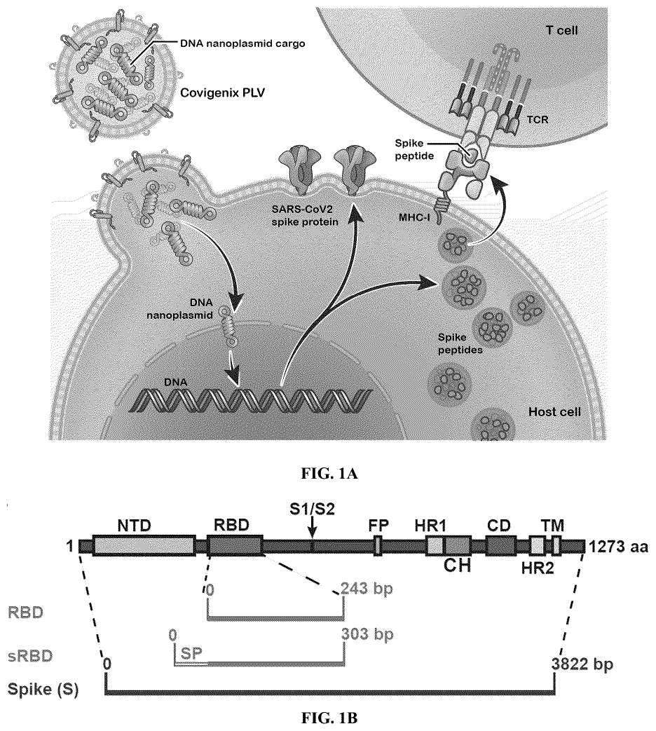

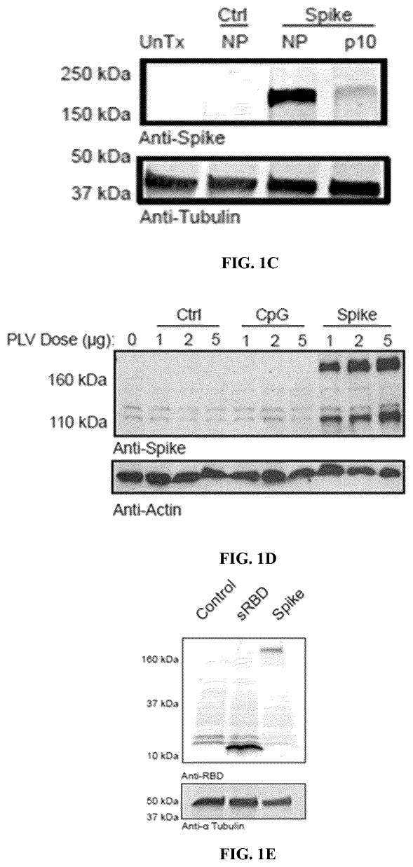

FAST-PLVs are a fusogenic proteolipid vehicle (PLV) based on well-tolerated lipids and, in preferred instances, a recombinant fusion-associated small transmembrane (FAST) protein that catalyzes fusion between the PLV membrane and cell membrane to promote intracellular DNA delivery. FAST-PLVs demonstrated low toxicity and improved intracellular delivery of both mRNA and plasmid DNA in mouse and non-human primate models with no diminished transgene expression following repeat injections. The utility of this delivery platform was also demonstrated in a gene therapy mouse model for muscle wasting disorders, showing increased hindlimb muscle size and grip strength (Brown, D., et al. (2021). Safe and effective delivery of nucleic acids using proteolipid vehicles formulated with fusion-associated small transmembrane proteins (In Submission)). The inventors herein leveraged the efficacy and highly favorable safety profile of FAST-PLVs as a DNA delivery platform to develop a DNA-based COVID-19 vaccine (FIG. 1A). Rapid prototyping of multiple antigen candidates, including full-length SARS-CoV-2 Spike(S), receptor binding domain (RBD), and secreted RBD (sRBD) configurations, combined with an optimized traditional plasmid backbone, or a Nanoplasmid (NP) backbone with two genetically encoded adjuvants (CpG motifs and a RIGI agonist). Initial screening identified a preferred vaccine candidate; full-length Spike in a Nanoplasmid backbone with both CpG and RIGI encoded adjuvants (NP-S). This vaccine stimulated spike-specific humoral and cellular immunity manifested by neutralizing antibody titers and a functional cytotoxic T cell response, and importantly protected against SARS-CoV-2 in a hamster challenge study.

In some instances, the data described herein indicates that a single-dose of NP-S was protective in a hamster SARS-CoV-2 challenge model as demonstrated by reduced weight loss during infection, reduced viral shedding and burden in the lungs, as well as a reduced proinflammatory response in the airways. Furthermore, in some cases, vaccination with a single dose of NP-S produced neutralizing antibodies in the NHP model, and the full human dose was well tolerated with no detectable injection site manifestations. These features are in stark contrast to most of the conventional DNA vaccine delivery platforms used to date. The FAST-PLV platform and antigen encoding plasmid DNA payload strategy employed for these SARS-CoV-2 vaccine candidates could also be rapidly and broadly applied to future emerging infections.

Zydus Cadila recently received emergency authorization in India for use of ZyCoV-D, its three-dose COVID-19 vaccine (3 mg/dose) administered with the PharmaJet® Tropis® Needle-free device. This will be the worlds' first human DNA vaccine to enter the marketplace and will have a large impact on driving further development and innovation in the DNA-based vaccine space. Furthermore, INOVIO is entering into a Phase 3 study with its DNA-based vaccine candidate, INO-4800, which is administered intradermally as a 2 mg dose in a two-dose regimen, followed by electroporation with their proprietary hand-held CELLECTRA® device. Based on the data presented here, the PLV-plasmid DNA platform could demonstrate immunogenicity with 8-30 times less DNA and eliminate the need for electroporation.

Studies with convalescent patient sera show that SARS-CoV-2 elicits a robust but potentially short to medium-lived humoral antibody response followed by more durable cell mediated CD4+ and CD8+ responses across all levels of disease severity. SARS-CoV-2 memory T cells have also been detected in blood samples stored before the COVID-19 pandemic, indicating an effect of cross-reactivity between the common cold-causing coronaviruses and SARS-CoV-2. SARS-CoV-2 Spike protein-encoding nucleic acid vaccines that elicit a neutralizing antibody response and a CD8+ T cell-based response are predicted to provide longer-lasting immunity. The DNA vaccine candidates NP-S and NP-S-2P provided herein demonstrate Spike-specific CD8+ T cell-mediated killing of target cells expressing both Wuhan Spike and Spike from alpha, beta and delta variants consistent with this notion. The potent CTL response likely plays a key role in the control of viral shedding and viral load in hamsters following challenge, as well as mediating a key effector mechanism against reinfection events. Without being bound by any theory, it is suspected that the enhanced CTL response induced by NP-S/NP-S-2P is due to direct intracellular delivery of the DNA payload encoding the antigen and two genetic adjuvants, CpG and RIGI, on the same DNA molecule. Furthermore, this is in addition to the response induced by recognition of the plasmid DNA payload by the STING and AIM2 pathways. Adjuvants such as CpG and RIGI enhance adaptive immunity through innate signaling; thus all cells expressing antigen will also express the adjuvants mobilizing innate immune cells to help shape the adaptive response.

Since it was observed herein that enhanced antibody, neutralizing antibody, and T cell responses from the Spike candidate with CpG and RIGI but no cell-mediated immune response without CpG-RIGI, it seems evident that the genetically encoded adjuvants enhanced cell-mediated immunity.

A neutralizing antibody response is certainly critical for protection against SARS-CoV-2 infection, but vulnerable to emerging variants. Synergy with a potent cellular immune response, will have a greater likelihood of providing long-term immunity. Early evidence in patients undergoing B cell-depleting cancer therapies indicates that cell-mediated immunity is sufficient to reduce COVID-19 disease severity. The findings described herein showing FAST-PLV delivery of a plasmid DNA encoding Spike, CpG and RIGI generated a potent and orchestrated CD8+ T cell response independent of mutations found in VOCs that limit nAb potency, suggesting that this vaccine candidate could be effective at reducing COVID-19 disease severity in humans. The vast majority of neutralizing antibodies against SARS-CoV-2 target the RBD of the Spike protein, the region where most mutations have arisen in the emerging variants of concern and circumvent neutralizing antibodies against the Wuhan strain. The findings provided herein demonstrate that an optimized combination of antigen, adjuvant, and dose induces a robust SARS-CoV-2 specific cell-mediated response and neutralizing antibodies, with the potential to confer strong immunity against COVID-19 infection and/or disease (or disease severity), and that preferably, such effect can be broadened by combining multiple variant of concern (VOC) sequences as bi-valent vaccines as described herein.

In some instances, it was observed that a single dose of NP-S is sufficient to meet or exceed the predicted threshold nAb values shown to be protective in NHPs. This appears promising for translation into the clinic, especially when combined with the induction of a robust anti-spike T cell response. Notably, the FAST-PLV delivery system is immunogenic and protective in the hamster SARS-CoV-2 challenge model with 20-50 times less plasmid DNA per dose compared with conventional strategies for delivering DNA-based vaccines such as electroporation.

Herein, the FAST-PLV delivery system was adapted as a plasmid DNA vaccine platform capable of stimulating a protective immune response consistent with other nucleic acid vaccines targeting SARS-CoV-2 Spike, ideally with a single dose. However, in contrast to conventional nucleic acid vaccine strategies, the approach described herein yields an inexpensive, rapidly scalable platform with durable stability at 2-8° C. that is not constrained by cold-chain processes. These attributes demonstrate how the FAST-PLV vaccine platform is ideally suited for rapid prototyping and global distribution of countermeasures against emerging pathogens.

Additionally, and unexpectedly, it has been identified herein that use of the FAST-PLV delivery system to deliver two separate vectors (e.g., plasmid DNA vectors) which encode two distinct versions of the SARS-CoV-2 spike protein (or a portion thereof) is associated with substantially increased immune response in individuals, both to each of the two distinct versions of the SARS-CoV-2 spike protein as well as to alternative SARS-CoV-2 spike proteins. In some embodiments, such a bi-valent approach (e.g., administering multiple vectors encoding multiple different variants of concern (VOCs)) shows immense potential as a therapeutic for the treatment or prevention of SARS-CoV-2.

Additionally, also provided herein multi-valent SARS-CoV-2 vaccines which comprises vectors encoding a SARS-CoV-2 spike protein and a second SARS-CoV-2 protein (e.g., an envelope (E) protein, a membrane protein, or a nucleocapsid protein). In some embodiments, use of multiple different antigens enhances the immune response to the vaccine to one or both components as compared to a vaccine encoding only one of the components.

Further additionally, in some embodiments, newly codon optimized vectors encoding a SARS-CoV-2 spike protein (or a portion thereof) are provided herein which are used in SARS-CoV-2 vaccines provided herein. In some embodiments, use of these newly codon optimized vectors leads to enhanced immune response as compared to other vectors.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### I. Vaccines

Provided herein are vaccines for eliciting an immune response to the SARS-CoV-2 virus in a subject. In some embodiments, the immune response is sufficient to prevent infection by the SARS-CoV-2 virus, or to prevent or minimize symptoms associated with being infected by the SARS-CoV-2 virus. In some preferred embodiments, a DNA vector (or, in some embodiments, multiple DNA vectors) as provided herein is administered as a vaccine. In other embodiments, an RNA polynucleotide (or multiple RNA polynucleotides) as provided herein is administered as a vaccine. In some embodiments, administration of a vaccine as provided herein comprising vectors encoding multiple SARS-CoV-2 proteins (e.g., encoding two different SARS-CoV-2 spike proteins, or encoding a SARS-CoV-2 spike protein and a second protein (e.g., nucleocapsid, membrane protein, or envelope protein) results in substantially enhanced efficacy (e.g., as measured by T cell activity, neutralizing antibody titers, reduction in risk of infection by SARS-CoV-2 virus, or reduced risk/severity of symptoms associated with SARS-CoV-2 infection) as compared to a vaccine which utilizes only a single encoded spike protein. In some embodiments, administration of a vaccine as provided herein comprising vectors encoding multiple SARS-CoV-2 proteins (e.g., encoding two different SARS-CoV-2 spike proteins) results in substantially enhanced efficacy (e.g., as measured by T cell activity, neutralizing antibody titers, reduction in risk of infection by SARS-CoV-2 virus, or reduced risk/severity of symptoms associated with SARS-CoV-2 infection) as compared to a vaccine which utilizes only a single encoded spike protein.

In an aspect herein is a SARS-CoV-2 DNA vaccine, comprising a first DNA vector comprising a polynucleotide sequence encoding a first SARS-CoV-2 spike protein or a portion thereof. In some embodiments, the first SARS-CoV-2 spike protein or portion thereof comprises 1) at least one amino acid substitution selected from a D614G amino acid substitution, a K986P amino acid substitution and a V987P amino acid substitution (based on SEQ ID NO: 1 as a reference sequence). In some embodiments, the first SARS-CoV-2 spike protein or portion thereof comprises an additional amino acid substitution at another residue of the first SARS-CoV-2 spike protein. In some embodiments, the vaccine comprises an adjuvant or a polynucleotide sequence encoding an adjuvant. In some embodiments, the first DNA vector is encapsulated in proteolipid vehicles that comprise a fusogenic membrane protein.

In one aspect, provided herein, is a SARS-CoV-2 DNA vaccine, comprising: a) a first DNA vector comprising a polynucleotide sequence encoding a first SARS-CoV-2 spike protein or a portion thereof; and b) a second DNA vector comprising a polynucleotide sequence encoding a second SARS-CoV-2 spike protein or a portion thereof, wherein the second SARS-CoV-2 spike protein has a different sequence from the first SARS-CoV-2 spike protein. In some embodiments, the first DNA vector and the second DNA vector are encapsulated in proteolipid vehicles that comprise a fusogenic membrane protein. In some embodiments, the SARS-CoV-2 DNA vaccine further comprises an adjuvant or a polynucleotide sequence encoding an adjuvant.

In one aspect herein is a SARS-CoV-2 DNA vaccine, comprising: a) a first DNA vector comprising a polynucleotide sequence encoding a first SARS-CoV-2 spike protein or a portion thereof, wherein the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence; and b) a second DNA vector comprising a polynucleotide sequence encoding a second SARS-CoV-2 spike protein or a portion thereof, wherein the second SARS-CoV-2 spike protein has a different sequence from the first SARS-CoV-2 spike protein. In some embodiments, the SARS-CoV-2 DNA vaccine comprises an adjuvant or a polynucleotide sequence encoding an adjuvant. In some embodiments, the first DNA vector and the second DNA vector are encapsulated in proteolipid vehicles that comprise a fusogenic membrane protein.

In some embodiments, after administration of a SARS-CoV-2 vaccine (e.g., DNA vaccine) provided herein to a subject, the subject displays a strong T-cell mediated immune response and a strong antibody response. The T-cell mediated immune response and antibody response can be any of the activities provided herein. In some embodiments, the strong T-cell mediated immune response and the strong antibody response are measured according to a method provided herein.

### Vectors

Provided herein are vectors which encode SARS-CoV-2 spike proteins or other SARS-CoV-2 proteins provided herein which are useful in eliciting an immune response to the SARS-CoV-2 protein when administered as a vaccine (e.g., as part of a SARS-CoV-2 vaccine described herein). The vectors and related polynucleotides provided herein can be delivered to a relevant cell of a subject and induce an immune response to the SARS-CoV-2 protein provided herein. In some embodiments, the vectors and related polynucleotides provided herein are formulated to be administered to a subject and deliver the vector or other polynucleotide to a cell of the subject.

Also provided herein are SARS-CoV-2 vaccines which comprise multiple of the vectors provided herein. In some embodiments, provided herein are SARS-CoV-2 vaccines which encode two of the vectors described herein, wherein each of the two vectors can be the same (except for the sequence encoding the SARS-CoV-2 protein or portion thereof, which will vary as described herein), or the two vectors can be different (e.g., comprise or encode different adjuvants, be comprised on different plasmid backbones, be controlled by different promoter elements, etc.). In some embodiments, a multi-valent SARS-CoV-2 DNA vaccine comprises vectors described herein which are substantially identical except for the sequence encoding the SARS-CoV-2 protein or portion thereof.

### DNA Vectors

In some embodiments, a vector as provided herein comprises a polynucleotide sequence encoding a SARS-CoV-2 protein, or a portion thereof. In some embodiments, a vector as provided herein comprises a polynucleotide sequence encoding a SARS-CoV-2 spike protein, or a portion thereof. In some embodiments, a vector as provided herein comprises a polynucleotide sequence encoding a SARS-CoV-2 protein selected from a nucleocapsid, an envelope protein, or a membrane protein.

In one aspect, provided herein, is a vector comprising a polynucleotide sequence encoding a SARS-CoV-2 spike protein, or a portion thereof, and a polynucleotide sequence encoding an adjuvant.

In some embodiments, the vector is a DNA vector. In some embodiments, the DNA vector is a plasmid, a viral vector, a cosmid, or an artificial chromosome. In some embodiments, the DNA vector is a plasmid.

In some embodiments herein is a DNA vector comprising a polynucleotide sequence encoding a SARS-CoV-2 spike protein or a portion thereof, wherein the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence.

In some embodiments herein is a DNA vector comprising a polynucleotide sequence encoding a SARS-CoV-2 spike protein or a portion thereof, wherein the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence, and a polynucleotide sequence encoding an adjuvant.

In some embodiments herein is a DNA vector comprising a polynucleotide sequence encoding a SARS-CoV-2 spike protein or a portion thereof, wherein the SARS-CoV-2 spike protein or portion thereof comprises 1) at least one amino acid substitution selected from a D614G amino acid substitution, a K986P amino acid substitution and a V987P amino acid substitution, and 2) an additional amino acid substitution at another residue of the first SARS-CoV-2 spike protein, wherein residue position numbering is based on SEQ ID NO: 1 as a reference sequence. In some embodiments, the DNA vector comprises a polynucleotide sequence encoding an adjuvant.

In some embodiments herein is a DNA vector comprising a polynucleotide sequence encoding a SARS-CoV-2 protein or a portion thereof, wherein the SARS-CoV-2 protein is an envelope protein, a membrane protein, or a nucleocapsid protein, or a portion of any of these.

In embodiments wherein the vector is a plasmid, it may be advantageous that the plasmid be at or below a certain size. In some embodiments, a smaller plasmid provided the advantage of better loading the vector into a desired formulation (e.g., a proteolipid vehicle as provided herein), as well as enhanced expression due to the lessor potential of cross reactivity owing to a larger size. In some embodiments, the plasmid comprises at most about 50,000, at most about 45,000, at most about 40,000, at most about 35,000, at most about 30,000, at most about 25,000, at most about 20,000, at most about 15,000, at most about 10,000, at most about 9,000, at most about 8,000, at most about 7,000, or at most about 6,500 nucleotides or nucleotide pairs (for double stranded DNA plasmids).

In some embodiments, the plasmid is or is derived from a bacterial or fungal plasmid. In some embodiments, the plasmid is or is derived from a yeast plasmid. In some embodiments, the plasmid is or is derived from bacteria.

In some embodiments, the plasmid backbone is derived from a bacteria or a fungus. In some embodiments, the plasmid backbone is derived from a bacteria. In some embodiments, the plasmid backbone is derived from a yeast.

In some embodiments, the plasmid backbone is a NTC9385R plasmid. The NTC9835R plasmid is an expression vector that contains a bacterial backbone comprising a 140 bp RNA-based sucrose selectable antibiotic free marker (RNA-OUT). The NTC9385R plasmid is described in U.S. Pat. No. 9,550,998, which is hereby incorporated by reference as if set forth herein in its entirety. NTC9835R is sold commercially by Nature Technology Corporation under the trade name Nanoplasmid™.

In some embodiments, the DNA vectors provided herein encode one or more adjuvants as provided herein. In some embodiments, the adjuvant is an immunostimulatory protein or a polynucleotide sequence. In some embodiments, the adjuvant polynucleotide sequence is comprised on an mRNA transcribed from the vector.

In some embodiments, the adjuvant is an immunostimulatory protein. In some embodiments, the vector co-expresses the immunostimulatory protein with the SARS-CoV-2 spike protein, thereby resulting in a more robust immune response by the subject. In some embodiments, the section of the vector which encodes the immunostimulatory protein is in a distinct region of the vector which encodes the SARS-CoV-2 spike protein. In some embodiments, the immunostimulatory protein is CRM197, interleukin-12, interleukin-15, NF-κB subunit p65/RelA, T-bet transcription factor, PPE44/pCI-OVA, Cholera Toxin Subunit A, C-terminal Hsp70, GM-CSF, MyD88, TRIF, IRF1, ΔIRF1, IRF3, IRF7, Flagellin, TBK1, HMGB1, DAI/ZBP1, or chMHD5. In some embodiments, the immunostimulatory protein is CRM197.

In some embodiments, DNA vector encodes an adjuvant polynucleotide sequence. In some embodiments, the adjuvant comprises a pathogen-associated molecular pattern (PAMP). In some embodiments, the adjuvant comprises an RNA CpG motif, a retinoic acid-inducible gene I (RIGI) agonist, a melanoma differentiation-associated protein 5 (MDA5) agonist, or a combination thereof. In some embodiments, the adjuvant comprises an RNA CpG motif. In some embodiments, the adjuvant comprises a RIGI agonist.

In some embodiments, the portion of the DNA vector which encodes the adjuvant (e.g., a polynucleotide adjuvant) is disposed on the DNA vector such that the adjuvant is incorporated into an mRNA transcript of the SARS-CoV-2 protein (e.g., the spike protein). In some embodiments, the polynucleotide encoding the adjuvant is positioned on the DNA vector such that the adjuvant is included in the 3′-UTR of an mRNA transcribed from the polynucleotide encoding the SARS-CoV-2 protein.

In some embodiments, the DNA vector encodes a second adjuvant. In some embodiments, the DNA vector comprises a second polynucleotide sequence encoding a second adjuvant. In some embodiments, the second adjuvant is a PAMP. In some embodiments, the second adjuvant comprises an RNA CpG motif, a retinoic acid-inducible gene I (RIGI) agonist, or a melanoma differentiation-associated protein 5 (MDA5) agonist. In some embodiments, the adjuvant and the second adjuvant each independently are an RNA CpG motif, a retinoic acid-inducible gene I (RIGI) agonist, or a melanoma differentiation-associated protein 5 (MDA5) agonist. In some embodiments, the adjuvant is a CpG motif and the second adjuvant is a RIGI agonist. In some embodiments, the second polynucleotide encoding the second adjuvant is positioned on the DNA vector such that the second adjuvant is included in the 3′-UTR of an mRNA transcribed from the polynucleotide encoding the SARS-CoV-2 protein.

In some embodiments, the vector comprises a polynucleotide encoding a SARS-CoV-2 spike protein as provided herein. In some embodiments, the polynucleotide encoding the SARS-CoV-2 spike protein comprises a nucleotide sequence having at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% sequence identity with the sequence set forth in SEQ ID NO: 5. In some embodiments, the polynucleotide encoding the SARS-CoV-2 spike protein comprises the sequence set forth in SEQ ID NO: 5.

In some embodiments, the polynucleotide encoding the SARS-CoV-2 spike protein comprises a nucleotide sequence having at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% sequence identity with the sequence set forth in SEQ ID NO: 4, 32-35, or 37-40 (e.g., any one of the indicated sequences). In some embodiments, the polynucleotide encoding the SARS-CoV-2 spike protein comprises the sequence set forth in SEQ ID NO: 4, 32-35, or 37-40. In some embodiments, the polynucleotide encoding the SARS-CoV-2 spike protein comprises a sequence having the sequence identity specified above but encodes a SARS-CoV-2 spike protein having the same amino acid sequence of the reference polynucleotide sequence (e.g., the polynucleotide sequence having, for example, 80% sequence identity to the sequence set forth in SEQ ID NO: 32 encodes a SARS-CoV-2 spike protein having the sequence set forth in SEQ ID NO: 31).

In some embodiments, a SARS-CoV-2 vaccine as provided herein (e.g., a bi-valent SARS-CoV-2 vaccine as described herein) will comprises multiple vectors provided herein. In some embodiments, the SARS-CoV-2 vaccine comprises two vectors provided herein, wherein each vector is identical or substantially identical except for the portion encoding the SARS-CoV-2 spike protein. For example, a SARS-CoV-2 vaccine as described herein can comprise two DNA plasmids which encode a different SARS-CoV-2 spike protein, but each of which comprises or encodes the same adjuvants (e.g., CpG and/or RIGI) and comprises the same promotors, enhancers, backbone, etc.

In some embodiments, SARS-CoV-2 vaccines comprising two vectors (e.g., DNA vectors) described herein will contain the two vectors at a desired ratio. In some embodiments, the two vectors are present in the vaccine in about the same amount (e.g., the amount of the first and second vector in the vaccine will differ by no more than 10%). In some embodiments, the two vectors are each present in the vaccine in the same amount. In some embodiments, the two vectors are present in the vaccine in a molar ratio of about 1:1. In some embodiments, the two vectors are present in the vaccine in a mass ratio of about 1:1. In some embodiments, the two vectors can be present in different amounts (e.g., a ratio of about 10:1 to about 1:1). In preferred embodiments, the two vectors are present in about the same amount.

### RNA Polynucleotides

Also provided herein are RNA polynucleotides which encode a SARS-CoV-2 protein as provided herein. The RNA polynucleotides can either be delivered directly to the cell (e.g., using a lipid vesicle known in the art to deliver mRNA) or can be formed in situ from transcription of a DNA vector as provided herein. Also provided herein are SARS-CoV-2 DNA vaccines which comprise vectors which encode the RNA polynucleotides provided herein.

In some embodiments, provided herein are SARS-CoV-2 DNA vaccines which encode two of the RNA polynucleotides described herein, wherein each of the two RNA polynucleotides can be the same (except for the sequence encoding the SARS-CoV-2 protein or portion thereof, which will vary as described herein), or the two RNA polynucleotides can be different (e.g., comprise different adjuvants). In preferred embodiments, a multi-valent SARS-CoV-2 DNA vaccine comprises vectors which encode two RNA polynucleotides described herein which are substantially identical except for the sequence encoding the SARS-CoV-2 protein (e.g., the spike protein or other SARS CoV-2 protein) or portion thereof.

In one aspect, provided herein, is an RNA polynucleotide which comprises an open reading frame encoding a SARS-CoV-2 protein. The SARS-CoV-2 protein can be any SARS-CoV-2 protein provided herein. In some embodiments, the RNA polynucleotide further comprises one or more adjuvants disposed in the 3′UTR of the RNA polynucleotide. In some embodiments, each RNA polynucleotide encoded by a multi-valent SARS-CoV-2 vaccine (e.g., a bi-valent SARS-CoV-2 vaccine) provided herein comprises an open reading frame according to the described embodiments.

In some embodiments, provided herein, is an RNA polynucleotide, comprising an open reading frame encoding a SARS-CoV-2 protein (e.g., a SARS-CoV-2 spike protein), or a portion thereof, and a 3′ untranslated region (UTR) comprising an adjuvant polynucleotide.

In some embodiments, provided herein, is an RNA polynucleotide, comprising an open reading frame encoding a SARS-CoV-2 spike protein, or a portion thereof, wherein the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence, and a 3′ untranslated region (UTR) comprising an adjuvant polynucleotide.

In some embodiments, the RNA polynucleotide is mRNA. In some embodiments, the mRNA is produced in situ in a cell after administration of a DNA vector provided herein to a subject. In some embodiments, the RNA polynucleotide is transcribed from a template nucleic acid in a cell of a subject. In some embodiments, the template nucleic acid is a DNA vector as provided herein. In some embodiments, the mRNA is prepared outside the body of the subject and delivered intracellularly.

In some embodiments, the RNA polynucleotide comprises one or more adjuvants in the 3′UTR of the RNA polynucleotide. In some embodiments, the adjuvant is an adjuvant polynucleotide. In some embodiments, the adjuvant polynucleotide comprises a CpG motif, a retinoic acid-inducible gene I (RIGI) agonist, or a melanoma differentiation-associated protein 5 (MDA5) agonist.

In some embodiments, the adjuvant polynucleotide comprises a CpG motif. In some embodiments, the CpG motif is a D type CpG, a Class B CpG, or a Class C CpG. In some embodiments, the CpG motif is a D type CpG. In some embodiments, the CpG motif comprises a nucleotide sequence having at least about 70%, at least about 75%, at least about 80%, at least about 85%, at least about 90%, or at least about 95% sequence identity with the sequence of 5′-GGUGCAUCGAUGCAGGGGGG-3′ (SEQ ID NO: 10). In some embodiments, the CpG motif comprises a nucleotide sequence of 5′-GGUGCAUCGAUGCAGGGGGG-3′ (SEQ ID NO: 10).

In some embodiments, the adjuvant comprises a RIGI agonist. In some embodiments, the RIGI agonist comprises a nucleotide sequence having at least about 70%, at least about 75%, at least about 80%, at least about 85%, at least about 90%, at least about 95%, at least about 96%, at least about 97%, at least about 98%, or at least about 99% sequence identity with the of sequence 5′AAAACAGGUCCTCCCCAUACUCUUUCAUUGUACACACCGCAAGCUCGACAAU CAUCGGAUUGAAGCAUUGUCGCACACAUCUUCCACACAGGAUCAGUACCUGCU UUCGCUUUU 3′ (SEQ ID NO: 11). In some embodiments, the RIGI agonist comprises a nucleotide sequence of 5′AAAACAGGUCCTCCCCAUACUCUUUCAUUGUACACACCGCAAGCUCGACAAU CAUCGGAUUGAAGCAUUGUCGCACACAUCUUCCACACAGGAUCAGUACCUGCU UUCGCUUUU 3′ (SEQ ID NO: 11).

In some embodiments, the RNA polynucleotide further comprises a second adjuvant polynucleotide. In some embodiments, the adjuvant polynucleotide and the second adjuvant polynucleotide are different. In some embodiments, the second adjuvant polynucleotide is comprised in the 3′-UTR. In some embodiments, the second adjuvant polynucleotide is upstream of the adjuvant. In some embodiments, the second adjuvant polynucleotide is downstream of the polynucleotide adjuvant. In some embodiments, the second adjuvant polynucleotide comprises a CpG motif, RIGI agonist, or a MDA5 agonist. In some embodiments, the adjuvant polynucleotide comprises a CpG motif and the second adjuvant polynucleotide comprises a RIGI agonist.

In some embodiments, the RNA polynucleotide comprises an open reading frame encoding a SARS-CoV-2 spike protein. In some embodiments, the open reading frame encoding the SARS-CoV-2 spike protein comprises a nucleotide sequence having at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% sequence identity with an RNA sequence transcribed from SEQ ID NO: 5. In some embodiments, the open reading frame encoding the SARS-CoV-2 spike protein comprises an RNA sequence transcribed from SEQ ID NO: 5.

In some embodiments, a SARS-CoV-2 vaccine as provided herein (e.g., a bi-valent SARS-CoV-2 vaccine as described herein) will comprises vectors which encode multiple (e.g., 2) of the RNA polynucleotides provided herein. In some embodiments, the SARS-CoV-2 vaccine comprises vectors which encode 2 of the RNA polynucleotides provided herein, wherein each RNA polynucleotide encoded is identical or substantially identical except for the SARS-CoV-2 protein encoded by each RNA polynucleotide. For example, a SARS-CoV-2 vaccine as described herein can comprise two DNA plasmids which encode two RNA polynucleotides, each of which comprises the same RIGI agonist and/or CpG motif in the same orientation relative to the portion encoding the SARS-CoV-2 protein, but each will encode a different SARS-CoV-2 spike protein, or a SARS-CoV-2 spike protein and a second SARS-CoV-2 protein as provided herein.

### SARS-CoV-2 Proteins

The vectors and RNA polynucleotides provided herein encode proteins, or portions thereof, derived from the SARS-CoV-2 virus. In some embodiments, the vaccine introduces into a cell of a subject the vector or RNA polynucleotide encoding the SARS-CoV-2 protein in a context in which the immune system recognizes the SARS-CoV-2 protein as a foreign antigen, thereby starting the immune response against the SARS-CoV-2 protein. In some embodiments, the SARS-CoV-2 protein is a spike protein, envelope protein, membrane protein, or nucleocapsid protein of a SARS-CoV-2 virus.

### SARS-CoV-2 Spike Proteins

In some embodiments, the SARS-CoV-2 protein of the instant disclosure is SARS-CoV-2 spike protein, or a portion thereof. The sequence of the spike protein of Wuhan SARS-CoV-2 originally isolated in December of 2019 is set forth in SEQ ID NO: 1 (QHD43416, UniProt P0DTC2). Since the first discovery, many different variants of the spike protein have been identified, some of which enable the virus to more readily infect host cells.

In some embodiments, the SARS-CoV-2 spike protein comprises one or more modifications to the sequence set forth in SEQ ID NO: 1. The modifications can comprise amino acid substitutions, deletions, insertions, or any combination thereof. In some embodiments, the SARS-CoV-2 spike protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more modifications. In some embodiments, the SARS-CoV-2 spike protein comprises at least 1, 2, 3, 4, 5, 6, 7, 8, 9, or 10 modifications.

In some embodiments, the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence. In some embodiments, the SARS-CoV-2 spike protein or portion thereof comprises a D614G amino acid substitution. In some embodiments, the SARS-CoV-2 spike protein or portion thereof comprises a K986P amino acid substitution. In some embodiments, the SARS-CoV-2 spike protein or portion thereof comprises a V987P amino acid substitution. In some embodiments, the SARS-CoV-2 spike protein or portion thereof comprises two or three mutations selected from D614G, K986P, and V987P. In some embodiments, the SARS-CoV-2 spike protein or portion thereof comprises a D614G amino acid substitution, a K986P amino acid substitution, and a V987P amino acid substitution.

In some embodiments, the SARS-CoV-2 protein is the full length SARS-CoV-2 spike protein. In some embodiments, the SARS-CoV-2 protein is a portion of the SARS-CoV-2 spike protein. In some embodiments, the portion of the SARS-CoV-2 spike protein comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 85%, at least 90%, at least 95%, at least 96%, at least 97%, at least 98%, or at least 99% of the amino acids of the full length spike protein. In some embodiments, the portion of the SARS-CoV-2 spike protein comprises one or more subunits of the SARS-CoV-2 spike protein. In some embodiments, the one or more subunits of the SARS-CoV-2 spike protein are selected from the N-terminal domain (NTD), the receptor binding domain (RBD), the S1 domain, the S2 domain, the fusion peptide domain, the heptad repeat domain 1 (HR1), the heptad repeat domain 2 (HR2), and the transmembrane domain (TM), or any combination thereof. In some embodiments, the portion of the SARS-CoV-2 spike protein comprises the RBD. In some embodiments, the portion of the SARS-CoV-2 protein is part of a fusion protein (e.g., an Fc or IgG fusion).

In some embodiments, the SARS-CoV-2 protein further comprises an additional modification (e.g., in addition to the 1, 2, or 3 substitutions selected from D614G, K986P, and V987P). In some embodiments, the additional modification is a modification identified in a variant form of the SARS-CoV-2 virus (e.g., the beta, gamma, delta, or omicron variants). In some embodiments, the additional modification is in the RBD of the variant form of the virus. Exemplary modifications in the RBD in selected variants can be found in Tables 1A and 1B below. In some embodiments, the additional modification is outside of the RBD. In some embodiments, the additional modifications are inside of the RBD and outside of the RBD.

TABLE 1A
Select RBD (residues 319-541 of SEQ ID
NO: 1) Mutations of Certain Variants

| Variant | Lineage | Spike Receptor Binding Domain Mutations |
|---|---|---|
| Alpha | B.1.1.7 | N501Y. |
| Beta | B.1.351 | K417N, E484K, N501Y. |
| Gamma | P.1 | K417T, E484K, and N501Y. |
| Delta | B.1.617.2 | L452R, T478K. |
| Epsilon | B.1.427 | L452R. |
| | and | |
| | B.1.429 | |
| Zeta | P.2 | E484K. |
| Eta | B.1.525 | E484K. |
| Iota | B.1.526 | E484K. |
| Theta | P.3 | E484K; N501Y. |
| Kappa | B.1.617.1 | L452R, E484Q. |
| Lambda | C.37 | L452Q, F490S. |

TABLE 1B

| | | |
|---|---|---|
| Omicron | B.1.1.529 | G339D, S371L, S373P, S375F, K417N, N440K, G446S, S477N, |
| | | T478K, E484A, Q493R, G496S, Q498R, N501Y, Y505H |
| Omicron | B.1.1.529.5 | G339D, S371F, S373P, S375F, T376A, D405N, R408S, K417N, |
| | (BA.5) | N440K, L452R, S477N, T478K, E484A, F486V, Q493R, Q498R, |
| | | N501Y, Y505H |
| Omicron | XBB.1.5 | G339H, R346T, L368I, S371F, S373P, S375F, T376A, D405N, |
| | | R408S, K417N, N440K, V445P, G446S, N460K, S477N, T478K, |
| | | E484A, F486P, F490S, Q498R, N501Y, Y505H |

In some embodiments, the SARS-CoV-2 spike protein comprises one or more mutations found in the alpha, beta, gamma, delta, epsilon, zeta, eta, iota, theta, kappa, lambda, or omicron variants. In some embodiments, the SARS-CoV-2 spike protein comprises one or more mutations found in the RBD of the alpha, beta, gamma, delta, epsilon, zeta, eta, iota, theta, kappa, lambda, or omicron variants. In some embodiments, the SARS-CoV-2 spike protein comprises each of the mutations found in the RBD of the alpha, beta, gamma, delta, epsilon, zeta, eta, iota, theta, kappa, lambda, or omicron variants.

In some embodiments, the SARS-CoV-2 spike protein comprises one or more mutations found in the beta, gamma, delta, or omicron variants. In some embodiments, the SARS-CoV-2 spike protein comprises one or more mutations found in the RBD of the beta, gamma, delta, or omicron variants. In some embodiments, the SARS-CoV-2 spike protein comprises each of the mutations found in the RBD of the beta, gamma, delta, or omicron variants. In some embodiments, the SARS-CoV-2 spike protein comprises each of the mutations found in the RBD of the beta variant. In some embodiments, the SARS-CoV-2 spike protein comprises each of the mutations found in the RBD of the gamma variant. In some embodiments, the SARS-CoV-2 spike protein comprises each of the mutations found in the RBD of the delta variant. In some embodiments, the SARS-CoV-2 spike protein comprises each of the mutations found in the RBD of the omicron variant. In some embodiments, the SARS-CoV-2 spike protein comprises 1, 2, 3, 4, 5, 6, 7, 8, 9, 10, 11, 12, or 13 of the mutations found in the RBD of the omicron variant (e.g., any one of the omicron variants, including those provided herein).

In some embodiments, the SARS-CoV-2 spike protein further comprises a T19I, 24-26Del, A27S, A67V, 69-70Del, T95I, 137-145Del, G142D, 143-145Del, Y145H, 211Del, L212I, V213G, ins214EPE, ins214TDR, A222V, G339D, G339H, R346K, R346S, V367F, L368I, S371F, S371L, S373P, S375F, T376A, P384L, N394S, D405N, R408S, Q414K, K417N, K417T, N439K, N440K, V445P, G446S, Y449H, Y449N, N450K, L452R, L452Q, N460K, S477N, T478K, V483A, E484A, E484K, E484Q, E484Del, F486P, F486V, F490R, F490S, Q493K, Q493R, S494P, G496S, Q498R, N501T, N501Y, Y505H, E516Q, T547K, Q613H, A653V, H655Y, G669S, Q677H, N679K, ins679GIAL, P681H, P681R, A701V, S704L, N764K, D796Y, N856K, Q954H, N969K, or L981F modification, or any combination thereof. In some embodiments, the SARS-CoV-2 spike protein further comprises 1, 2, 3, 4, 6, 7, 8, 9, 10, 11, 12, 13, 14, 15, or more of T19I, 24-26Del, A27S, A67V, 69-70Del, T95I, 137-145Del, G142D, 143-145Del, Y145H, 211Del, L212I, V213G, ins214EPE, ins214TDR, A222V, G339D, G339H, R346K, R346S, V367F, L368I, S371F, S371L, S373P, S375F, T376A, P384L, N394S, D405N, R408S, Q414K, K417N, K417T, N439K, N440K, V445P, G446S, Y449H, Y449N, N450K, L452R, L452Q, N460K, S477N, T478K, V483A, E484A, E484K, E484Q, E484Del, F486P, F486V, F490R, F490S, Q493K, Q493R, S494P, G496S, Q498R, N501T, N501Y, Y505H, E516Q, T547K, Q613H, A653V, H655Y, G669S, Q677H, N679K, ins679GIAL, P681H, P681R, A701V, S704L, N764K, D796Y, N856K, Q954H, N969K, or L981F modifications.

In some embodiments, the SARS-CoV-2 spike protein further comprises a A67V, 69-70Del, T95I, 137-145Del, G142D, 143-145Del, Y145H, 211Del, L212I, ins214EPE, A222V, G339D, R346K, R346S, S371L, S373P, S375F, N394S, K417N, K417T, N440K, G446S, Y449H, Y449N, L452R, L452Q, S477N, T478K, E484A, E484K, E484Del, F490R, F490S, Q493K, G496S, Q498R, N501Y, T547K, Q613H, H655Y, Q677H, N679K, P681H, P681R, A701V, N764K, D796Y, N856K, Q954H, N969K, or L981F modification, or any combination thereof. In some embodiments, the SARS-CoV-2 spike protein further comprises 1, 2, 3, 4, 5, 6, 7, 8, 9, 10, 11, 12, 13, 14, 15, or more of A67V, 69-70Del, T95I, 137-145Del, G142D, 143-145Del, Y145H, 211Del, L212I, ins214EPE, A222V, G339D, R346K, R346S, S371L, S373P, S375F, N394S, K417N, K417T, N440K, G446S, Y449H, Y449N, L452R, L452Q, S477N, T478K, E484A, E484K, E484Del, F490R, F490S, Q493K, G496S, Q498R, N501Y, T547K, Q613H, H655Y, Q677H, N679K, P681H, P681R, A701V, N764K, D796Y, N856K, Q954H, N969K, and L981F modifications.

In some embodiments, the SARS-CoV-2 spike protein further comprises a A67V, 69-70Del, T95I, G142D, 143-145Del, Y145H, 211Del, L212I, ins214EPE, A222V, G339D, R346K, S371L, S373P, S375F, K417N, K417T, N440K, G446S, L452R, L452Q, S477N, T478K, E484A, E484K, F490S, Q493K, G496S, Q498R, N501Y, Y505H, T547K, H655Y, N679K, P681H, P681R, A701, N764K, D796Y, N856K, Q954H, N969K, or L981F modification, or any combination thereof. In some embodiments, the SARS-CoV-2 spike protein further comprises 1, 2, 3, 4, 5, 6, 7, 8, 9, 10, 11, 12, 13, 14, 15, or more of A67V, 69-70Del, T95I, G142D, 143-145Del, Y145H, 211Del, L212I, ins214EPE, A222V, G339D, R346K, S371L, S373P, S375F, K417N, K417T, N440K, G446S, L452R, L452Q, S477N, T478K, E484A, E484K, F490S, Q493K, G496S, Q498R, N501Y, Y505H, T547K, H655Y, N679K, P681H, P681R, A701, N764K, D796Y, N856K, Q954H, N969K, and L981F modifications.

In some embodiments, the SARS-CoV-2 spike protein further comprises a A67V, 69-70Del, T95I, G142D, 143-145Del, 211Del, L212I, ins214EPE, G339D, S371L, S373P, S375F, K417N, K417T, N440K, G446S, S477N, L452R, T478K, E484A, E484K, Q493K, G496S, Q498R, N501Y, Y505H, T547K, H655Y, N679K, P681H, P681R, A701V, N764K, D796Y, N856K, Q954H, N969K, or L981F modifications, or any combination thereof. In some embodiments, the SARS-CoV-2 spike protein further comprises 1, 2, 3, 4, 5, 6, 7, 8, 9, 10, 11, 12, 13, 14, 15, or more of A67V, 69-70Del, T95I, G142D, 143-145Del, 211Del, L212I, ins214EPE, G339D, S371L, S373P, S375F, K417N, K417T, N440K, G446S, S477N, L452R, T478K, E484A, E484K, Q493K, G496S, Q498R, N501Y, Y505H, T547K, H655Y, N679K, P681H, P681R, A701V, N764K, D796Y, N856K, Q954H, N969K, and L981F modifications.

In some embodiments, the SARS-CoV-2 spike protein at least partially aligns with the sequence set forth in SEQ ID NO: 1. In some embodiments, the SARS-CoV-2 spike protein comprises an amino acid sequence having at least 90%, at least 95%, at least 96%, at least 97%, at least 98%, or at least 99% sequence identity with the sequence set forth in SEQ ID NO: 1. In some embodiments, the SARS-CoV-2 spike protein comprises an amino acid sequence having at least 99%, at least 99.1%, at least 99.2%, at least 99.3%, at least 99.4%, at least 99.5%, at least 99.6%, at least 99.7%, at least 99.8%, or at least 99.9% sequence identity with the sequence set forth in SEQ ID NO: 1. In some embodiments, the SARS-CoV-2 spike protein comprises an amino acid sequence having at least 99.5% sequence identity with the sequence set forth in SEQ ID NO: 1. In some embodiments, the SARS-CoV-2 spike protein comprises an amino acid sequence having at least 99.6% sequence identity with the sequence set forth in SEQ ID NO: 1. In some embodiments, the SARS-CoV-2 spike protein comprises an amino acid sequence having at least 99.7% sequence identity with the sequence set forth in SEQ ID NO: 1. In some embodiments, the SARS-CoV-2 spike protein comprises an amino acid sequence having at least 99.8% sequence identity with the sequence set forth in SEQ ID NO: 1. In some embodiments, the SARS-CoV-2 spike protein comprises an amino acid sequence having at least 99.9% sequence identity with the sequence set forth in SEQ ID NO: 1. In some embodiments, the SARS-CoV-2 spike protein comprises an amino acid sequence as set forth in SEQ ID NO: 1.

In some embodiments, the SARS-CoV-2 spike protein comprises an amino acid sequence having at least 90%, at least 95%, at least 96%, at least 97%, at least 98%, or at least 99% sequence identity with the sequence set forth in SEQ ID NO: 1. In some embodiments, the SARS-CoV-2 spike protein comprises an amino acid sequence having at least 99%, at least 99.1%, at least 99.2%, at least 99.3%, at least 99.4%, at least 99.5%, at least 99.6%, at least 99.7%, at least 99.8%, or at least 99.9% sequence identity with the sequence set forth in SEQ ID NO: 2, 3, 31, or 36. In some embodiments, the SARS-CoV-2 spike protein comprises each of the modifications relative to SEQ ID NO: 1 contained in SEQ ID NO: 2, 3, 31, or 36 and comprises the indicated percent identity. In some embodiments, the SARS-CoV-2 spike protein comprises an amino acid sequence as set forth in SEQ ID NO: 2. In some embodiments, the SARS-CoV-2 spike protein comprises an amino acid sequence as set forth in SEQ ID NO: 3. In some embodiments, the SARS-CoV-2 spike protein comprises an amino acid sequence as set forth in SEQ ID NO: 31. In some embodiments, the SARS-CoV-2 spike protein comprises an amino acid sequence as set forth in SEQ ID NO: 36.

In some embodiments, a SARS-CoV-2 vaccine as provided herein (e.g., a bi-valent SARS-CoV-2 vaccine as described herein) will comprises vectors which encode multiple (e.g., 2) of the spike proteins (or portions thereof) provided herein. In some embodiments, the SARS-CoV-2 vaccine comprises vectors which encode two of the spike proteins or portions thereof provided herein (i.e., a first spike protein and a second spike protein).

In some embodiments, the first and the second SARS-CoV-2 spike proteins are different proteins (i.e., include at least one modification to the sequence of one relative to the other, such as a substitution, deletion, or insertion). In some embodiments, the second SARS-CoV-2 spike protein comprises at least 2, at least 3, at least 4, least 5, at least 10, at least 15, at least 20, at least 25, or at least 30 amino acid modifications relative to the first SARS-CoV-2 spike protein. The amino acid modifications can be any of those provided herein, or can be any other suitable modification. In some embodiments, both spike proteins will comprise the “2P” substitution (i.e., a K986P amino acid substitution and a V987P amino acid substitution). In some embodiments, the first SARS-CoV-2 spike protein and the second SARS-CoV-2 spike protein are derived from different variants of the SARS-CoV-2 virus (e.g., one from an omicron strain and one from the Wuhan strain). In some embodiments, the first spike protein and the second spike protein are derived from different sub-variants of the SARS-CoV-2 virus (e.g., the omicron BA.5 and omicron XBB.1.4 sub-variants).

### SARS-CoV-2 Envelope Proteins

In some embodiments, the SARS-CoV-2 protein encoded by a vector described herein is a SARS-CoV-2 envelope protein, or a portion thereof. In some embodiments, the SARS-CoV-2 protein is a full-length SARS-CoV-2 envelope protein. In some embodiments, the envelope protein is truncated (e.g., at least about 1, 2, 3, 4, 5, 6, 7, 8, 9, 10, 11, 12, 13, 14, 15, or more amino acids from the N or C-terminus are removed).

In some embodiments, the SARS-CoV-2 envelope protein comprises an amino acid sequence having at least about 80%, 85%, 90%, 95%, 97%, or 98% sequence identity to the sequence MYSFVSEETGTLIVNSVLLFLAFVVFLLVTLAILTALRLCAYCCNIVNVSLVKPSFYV YSRVKNLNSSRVPDLLV (Uniprot ID P0DTC4) (SEQ ID NO: 44). In some embodiments, the SARS-CoV-2 envelope protein comprises the sequence of SEQ ID NO: 44. In some embodiments, the envelope protein is the envelope protein associated with a desired variant of the SARS-CoV-2 virus (e.g., an omicron variant as described herein).

### SARS-CoV-2 Membrane Proteins

In some embodiments, the SARS-CoV-2 protein encoded by a vector described herein is a SARS-CoV-2 membrane protein, or a portion thereof. In some embodiments, the SARS-CoV-2 protein is a full-length SARS-CoV-2 membrane protein. In some embodiments, the membrane protein is truncated (e.g., at least about 1, 2, 3, 4, 5, 6, 7, 8, 9, 10, 11, 12, 13, 14, 15, 16, 17, 18, 19, or 20% of the amino acids from the N or C-terminus are removed).

In some embodiments, the SARS-CoV-2 membrane protein comprises an amino acid sequence having at least about 80%, 85%, 90%, 95%, 97%, 98%, or 99% sequence identity to the sequence MADSNGTITVEELKKLLEQWNLVIGFLFLTWICLLQFAYANRNRFLYIIKLIFLWLLW PVTLACFVLAAVYRINWITGGIAIAMACLVGLMWLSYFIASFRLFARTRSMWSFNPE TNILLNVPLHGTILTRPLLESELVIGAVILRGHLRIAGHHLGRCDIKDLPKEITVATSRT LSYYKLGASQRVAGDSGFAAYSRYRIGNYKLNTDHSSSSDNIALLVQ (UniProt ID P0DTC5) (SEQ ID NO: 41). In some embodiments, the membrane protein comprises the amino acid sequence set forth in SEQ ID NO: 41. In some embodiments, the membrane protein is the membrane protein associated with a desired variant of the SARS-CoV-2 virus (e.g., an omicron variant as described herein).

In some embodiments, the SARS-CoV-2 membrane protein is encoded by a polynucleotide sequence having at least 80%, 85%, 90%, 95%, 97%, 98%, or 99% identity to the forth in 42 sequence set SEQ ID NO: (atggccgatagcaacggcaccatcaccgtggaagaactgaagaaactgctggaacagtggaacctcgtgatcggcttcctgttcctg acctggatctgcctgctgcagttcgcctacgccaaccggaacagattcctgtatattatcaagctgatcttcctgtggctgctgtggcccg tgacactggcctgttttgtgctggccgccgtgtaccggatcaactggatcacaggcggaatcgccattgccatggcctgtctcgttggc ctgatgtggctgagctactttatcgccagcttccggctgttcgcccggaccagatccatgtggtccttcaatcccgagacaaacatcctg ctgaacgtgcccctgcacggcacaatcctgacaagacctctgctggaaagcgagctggttatcggcgccgtgatcctgagaggccac ctgagaattgccggacaccacctgggcagatgcgacatcaaggacctgcctaaagaaatcacagtggccaccagcagaaccctgtc ctactataagctgggcgccagccagagagtggccggcgattctggatttgccgcctacagcagataccggatcggcaactacaagct gaacaccgaccacagctccagcagcgacaatatcgcactgctggtgcagtgat). In some embodiments, the SARS-CoV-2 membrane protein is encoded by a polynucleotide set forth in SEQ ID NO: 42.

### SARS-CoV-2 Nucleocapsid Proteins

In some embodiments, the SARS-CoV-2 protein encoded by a vector described herein is a SARS-CoV-2 nucleocapsid protein, or a portion thereof. In some embodiments, the SARS-CoV-2 protein is a full-length SARS-CoV-2 nucleocapsid protein. In some embodiments, the nucleocapsid protein is truncated (e.g., at least about 1, 2, 3, 4, 5, 6, 7, 8, 9, 10, 11, 12, 13, 14, 15, 16, 17, 18, 19, or 20% of the amino acids from the N or C-terminus are removed).

In some embodiments, the SARS-CoV-2 nucleocapsid protein comprises an amino acid sequence having at least about 80%, 85%, 90%, 95%, 97%, 98%, or 99% sequence identity to the sequence MSDNGPQNQRNAPRITFGGPSDSTGSNQNGERSGARSKQRRPQGLPNNTASWFTAL TQHGKEDLKFPRGQGVPINTNSSPDDQIGYYRRATRRIRGGDGKMKDLSPRWYFYY LGTGPEAGLPYGANKDGIIWVATEGALNTPKDHIGTRNPANNAAIVLQLPQGTTLPK GFYAEGSRGGSQASSRSSSRSRNSSRNSTPGSSRGTSPARMAGNGGDAALALLLLDR LNQLESKMSGKGQQQQGQTVTKKSAAEASKKPRQKRTATKAYNVTQAFGRRGPEQ TQGNFGDQELIRQGTDYKHWPQIAQFAPSASAFFGMSRIGMEVTPSGTWLTYTGAIK LDDKDPNFKDQVILLNKHIDAYKTFPPTEPKKDKKKKADETQALPQRQKKQQTVTL LPAADLDDFSKQLQQSMSSADSTQA (UniProt ID P0DTC9) (SEQ ID NO: 43). In some embodiments, the nucleocapsid protein comprises the amino acid sequence set forth in SEQ ID NO: 41. In some embodiments, the nucleocapsid protein is the membrane protein associated with a desired variant of the SARS-CoV-2 virus (e.g., an omicron variant as described herein).

### Adjuvants

In some embodiments, the SARS-CoV-2 DNA vaccines, vectors, or RNA polynucleotides provided herein include or encode one or more adjuvants. An adjuvant is a substance which enhances a subject's immune response to an antigen. Adjuvants are commonly utilized in many vaccines and many are known in the art, though which adjuvant or combination of adjuvants will give rise to a desired immune response to a particular antigen is generally difficult to predict. The adjuvants provided herein may be incorporated in an appropriate manner, such as directly (e.g., the adjuvant is included in a co-formulation) or indirectly (e.g., the adjuvant is encoded by a polynucleotide such that it is expressed in situ). In multi-valent embodiments of a vaccine as provided herein, it is preferred that each vector comprises the same adjuvants or nucleotides encoding the adjuvants, though other configurations (e.g., each vector having different adjuvants or polynucleotides encoding adjuvants) are also contemplated herein.

In some embodiments, SARS-CoV-2 DNA vaccines described herein comprise two different vectors (e.g., two different vectors encoding different SARS-CoV-2 proteins as described herein). In such embodiments, each of the two vectors can encode adjuvants which are the same or different, or one vector can encode and adjuvant and the other does not. In some embodiments, a multi-valent SARS-CoV-2 DNA vaccine comprises vectors which each encode the same adjuvants.

In some embodiments, a SARS-CoV-2 DNA vaccine provided herein comprises an adjuvant.

In some embodiments, the adjuvant is included as part of the formulation of a SARS-CoV-2 DNA vaccine provided herein (e.g., encased within the proteolipid vehicle, within the lipid layer of the proteolipid vehicle, or outside of the proteolipid vehicle). In some embodiments, the adjuvant comprises an immunostimulatory protein (e.g., CRM197), alum, squalene oil-in-water emulsions, liposomes, TLR ligands, or polysaccharides. In some embodiments, the adjuvant comprises CRM197, glucopyranosyl lipid adjuvant (GLA-AF), dsRNA analog poly (I:C), CpG 1018 (Dynavax), CpG 7909 (Dynavax), cationic antimicrobial polypeptide (IC31®), alum-absorbed GLA/SLA, lipopolypeptide Pam2/Pam3, Alum-absorbed phosphonate BZN compounds (SMIP7.10), imidazoquinolines, inulin; chitosan, 3-O-desacyl-4-monophosphoryl lipid A, squalene, sorbitan oleate (MONTANE™80 PPI), eumulgin B1 PH, monophosphoryl lipid A (MPL®), saponin, polyoxyethylenesorbitan monooleate (TWEEN® 80), sorbitan triolate, glycerol egg phosphatidylcholine, poloxamer, ammonium phosphate buffer, α-tocopherol, dimethyldioctadecylammonium bromide (DDAR), or trehalose-6,6-dibchenate (TDB®). In some embodiments, the adjuvant comprises alum, and 3-O-desacyl-4-monophosphoryl lipid A. In some embodiments, the adjuvant comprises squalene, sorbitan oleate (MONTANE™80 PPI), and eumulgin B1 PH. In some embodiments, the adjuvant comprises squalene, monophosphoryl lipid A (MPL®), and saponin. In some embodiments, the adjuvant comprises squalene, polyoxyethylenesorbitan monooleate (TWEEN® 80), and sorbitan triolate. In some embodiments, the adjuvant comprises squalene, glycerol, egg phosphatidylcholine, poloxamer, and ammonium phosphate buffer (stable emulsion, SE). In some embodiments, the adjuvant comprises squalene, polyoxyethylenesorbitan monooleate (TWEEN® 80), and α-tocopherol. In some embodiments, the adjuvant comprises monophosphoryl lipid A (MPL®), and saponin. In some embodiments, the adjuvant comprises dimethyldioctadecylammonium bromide (DDA®), and trehalose-6,6-dibchenate (TDB®). In some embodiments, the adjuvant comprise CRM197.

In some embodiments, the adjuvant comprises an immunostimulatory protein. In some embodiments, the adjuvant immunostimulatory protein is included directly in the formulation. In some embodiments, the adjuvant immunostimulatory protein is encoded on a polynucleotide included with the vaccine (e.g., on the same vector as the SARS-CoV-2 protein, or on a separate vector). In some embodiments, the immunostimulatory protein is CRM197, interleukin-12, interleukin-15, NF-κB subunit p65/RelA, T-bet transcription factor, PPE44/pCI-OVA, Cholera Toxin Subunit A, C-terminal Hsp70, GM-CSF, MyD88, TRIF, IRF1, ΔIRF1, IRF3, IRF7, Flagellin, TBK1, HMGB1, DAI/ZBP1, or chMHD5. In some embodiments, the immunostimulatory protein is CRM197.

In some embodiments, the adjuvant is an adjuvant polynucleotide. In some embodiments, the adjuvant polynucleotide is comprised in the DNA vector or is encoded in the DNA vector and expressed in situ. In some embodiments, the adjuvant polynucleotide is included on the mRNA transcript encoding the SARS-CoV-2 protein. In some embodiments, the adjuvant polynucleotide is included in the 3′UTR of the mRNA transcript encoding the SARS-CoV-2 protein. In some embodiments, adjuvant polynucleotide comprises a CpG motif, a retinoic acid-inducible gene I (RIGI) agonist, or a melanoma differentiation-associated protein 5 (MDA5) agonist.

In some embodiments, the adjuvant polynucleotide is a CpG motif. In some embodiments, the CpG motif is a D type CpG, a Class B CpG, or a Class C CpG. In some embodiments, the CpG motif is a D type CpG. In some embodiments, the CpG motif is an RNA CpG. In some embodiments, the CpG motif is encoded by a nucleotide sequence having at least about 70%, at least about 75%, at least about 80%, at least about 85%, at least about 90%, or at least about 95% sequence identity with the sequence of 5′-GGTGCATCGATGCAGGGGGG-3′ (SEQ ID NO: 8). In some embodiments, the CpG motif is encoded by a nucleotide sequence of 5′-GGTGCATCGATGCAGGGGGG-3′ (SEQ ID NO: 8). In some embodiments, the CpG motif comprises a nucleotide sequence having at least about 70%, at least about 75%, at least about 80%, at least about 85%, at least about 90%, or at least about 95% sequence identity with the sequence of 5′-GGUGCAUCGAUGCAGGGGGG-3′ (SEQ ID NO: 10). In some embodiments, the CpG motif comprises nucleotide sequence of 5′-GGUGCAUCGAUGCAGGGGGG-3′ (SEQ ID NO: 10).

In some embodiments, the adjuvant comprises a RIGI agonist. In some embodiments, the RIGI agonist is encoded by nucleotide sequence having at least about 70%, at least about 75%, at least about 80%, at least about 85%, at least about 90%, at least about 95%, at least about 96%, at least about 97%, at least about 98%, or at least about 99% sequence identity with the sequence of 5′AAAACAGGTCCTCCCCATACTCTTTCATTGTACACACCGCAAGCTCGACAATCA TCGGATTGAAGCATTGTCGCACACATCTTCCACACAGGATCAGTACCTGCTTTCG CTTTT 3′ (SEQ ID NO: 9). In some embodiments, the RIGI agonist is encoded by a nucleotide sequence of 5′AAAACAGGTCCTCCCCATACTCTTTCATTGTACACACCGCAAGCTCGACAATCA TCGGATTGAAGCATTGTCGCACACATCTTCCACACAGGATCAGTACCTGCTTTCG CTTTT 3′ (SEQ ID NO: 9). In some embodiments, the RIGI agonist comprises a nucleotide sequence having at least about 70%, at least about 75%, at least about 80%, at least about 85%, at least about 90%, at least about 95%, at least about 96%, at least about 97%, at least about 98%, or at least about 99% sequence identity with the sequence of 5′AAAACAGGUCCTCCCCAUACUCUUUCAUUGUACACACCGCAAGCUCGACAAU CAUCGGAUUGAAGCAUUGUCGCACACAUCUUCCACACAGGAUCAGUACCUGCU UUCGCUUUU 3′ (SEQ ID NO: 11). In some embodiments, the RIGI agonist comprises a nucleotide sequence of 5′AAAACAGGUCCTCCCCAUACUCUUUCAUUGUACACACCGCAAGCUCGACAAU CAUCGGAUUGAAGCAUUGUCGCACACAUCUUCCACACAGGAUCAGUACCUGCU UUCGCUUUU 3′ (SEQ ID NO: 11).

### Exemplary Monovalent Vaccine Vectors

In some embodiments, monovalent vaccine according to the instant disclosure comprises a single DNA vector encoding an omicron spike protein (e.g., as in SEQ ID NO: 3, 31, or 36).

In some embodiments, a monovalent vaccine provided herein comprises a DNA vector encoding a SARS-CoV-2 spike protein of SEQ ID NO: 3. In some embodiments, the polynucleotide sequence encoding the SARS-CoV-2 spike protein comprises the sequence of SEQ ID NO: 4.

In some embodiments, a monovalent vaccine provided herein comprises a DNA vector encoding a SARS-CoV-2 spike protein of SEQ ID NO: 31. In some embodiments, the polynucleotide sequence encoding the SARS-CoV-2 spike protein comprises the sequence of SEQ ID NO: 32, SEQ ID NO: 33, SEQ ID NO: 34, or SEQ ID NO: 35.

In some embodiments, a monovalent vaccine comprises a DNA vector encoding a SARS-CoV-2 spike protein of SEQ ID NO: 36. In some embodiments, the polynucleotide sequence encoding the SARS-CoV-2 spike protein comprises the sequence of SEQ ID NO: 37, SEQ ID NO: 38, SEQ ID NO: 39, or SEQ ID NO: 40.

### Exemplary Multi-Valent Vaccine Vectors

In some embodiments, a bi-valent vaccine according to the instant disclosure comprises two vectors, the first vector encoding a first SARS-CoV-2 spike protein and a second vector encoding a second SARS-CoV-2 spike protein.

In some embodiments, a bi-valent vaccine provided herein comprises a first vector encoding a first SARS-CoV-2 spike protein of SEQ ID NO: 2 (optionally encoded by SEQ ID NO: 5) and a second vector encoding a second SARS-CoV-2 spike protein of SEQ ID NO: 3, 31, or 36. In some embodiments, the second SARS-CoV-2 spike protein has a sequence of SEQ ID NO: 3. In some embodiments, the second SARS-CoV-2 spike protein has a sequence of SEQ ID NO: 31 (and, optionally, encoded by a sequence set forth in one of SEQ NOs: 32-35). In some embodiments, the second SARS-CoV-2 spike protein has a sequence of SEQ ID NO: 36 (and, optionally, encoded by a sequence set forth in one of SEQ ID NOs: 37-40).

In some embodiments, a bi-valent vaccine provided herein comprises a first vector encoding a first SARS-CoV-2 spike protein of SEQ ID NO: 3 (optionally encoded by SEQ ID NO: 4) and a second vector encoding a second SARS-CoV-2 spike protein of SEQ ID NO: 31 or 36. In some embodiments, the second SARS-CoV-2 spike protein has a sequence of SEQ ID NO: 31 (and, optionally, encoded by a sequence set forth in one of SEQ NOs: 32-35). In some embodiments, the second SARS-CoV-2 spike protein has a sequence of SEQ ID NO: 36 (and, optionally, encoded by a sequence set forth in one of SEQ ID NOs: 37-40).

In some embodiments, a bi-valent vaccine provided herein comprises a first vector encoding a first SARS-CoV-2 spike protein of SEQ ID NO: 31 (and, optionally, encoded by a sequence set forth in one of SEQ NOs: 32-35) and a second vector encoding a second SARS-CoV-2 spike protein of SEQ ID NO: 36 (and, optionally, encoded by a sequence set forth in one of SEQ ID NOs: 37-40).

In some embodiments, a bi-valent vaccine according to the instant disclosure comprises two vectors, the first vector encoding a first SARS-CoV-2 spike protein and a second vector encoding a SARS-CoV-2 envelope protein. In some embodiments, the envelope protein comprises the sequence set forth in SEQ ID NO: 44. In some embodiments, the SARS-CoV-2 spike protein comprises the sequence set forth in SEQ ID NO: 2 (optionally encoded by a polynucleotide sequence of SEQ ID NO: 5). In some embodiments, the SARS-CoV-2 spike protein comprises the sequence set forth in SEQ ID NO: 3 (optionally encoded by a polynucleotide sequence of SEQ ID NO: 4). In some embodiments, the SARS-CoV-2 spike protein comprises the sequence set forth in SEQ ID NO: 31 (optionally encoded by a polynucleotide sequence of one of SEQ ID NOs: 32-35). In some embodiments, the SARS-CoV-2 spike protein comprises the sequence set forth in SEQ ID NO: 36 (optionally encoded by a polynucleotide sequence of one of SEQ ID NOs: 37-40).

In some embodiments, a bi-valent vaccine according to the instant disclosure comprises two vectors, the first vector encoding a first SARS-CoV-2 spike protein and a second vector encoding a SARS-CoV-2 membrane protein. In some embodiments, the membrane protein comprises the sequence set forth in SEQ ID NO: 41 (and optionally encoded by a polynucleotide sequence of SEQ ID NO: 42). In some embodiments, the SARS-CoV-2 spike protein comprises the sequence set forth in SEQ ID NO: 2 (optionally encoded by a polynucleotide sequence of SEQ ID NO: 5). In some embodiments, the SARS-CoV-2 spike protein comprises the sequence set forth in SEQ ID NO: 3 (optionally encoded by a polynucleotide sequence of SEQ ID NO: 4). In some embodiments, the SARS-CoV-2 spike protein comprises the sequence set forth in SEQ ID NO: 31 (optionally encoded by a polynucleotide sequence of one of SEQ ID NOs: 32-35). In some embodiments, the SARS-CoV-2 spike protein comprises the sequence set forth in SEQ ID NO: 36 (optionally encoded by a polynucleotide sequence of one of SEQ ID NOs: 37-40).

In some embodiments, a bi-valent vaccine according to the instant disclosure comprises two vectors, the first vector encoding a first SARS-CoV-2 spike protein and a second vector encoding a SARS-CoV-2 nucleocapsid protein. In some embodiments, the membrane protein comprises the sequence set forth in SEQ ID NO: 43. In some embodiments, the SARS-CoV-2 spike protein comprises the sequence set forth in SEQ ID NO: 2 (optionally encoded by a polynucleotide sequence of SEQ ID NO: 5). In some embodiments, the SARS-CoV-2 spike protein comprises the sequence set forth in SEQ ID NO: 3 (optionally encoded by a polynucleotide sequence of SEQ ID NO: 4). In some embodiments, the SARS-CoV-2 spike protein comprises the sequence set forth in SEQ ID NO: 31 (optionally encoded by a polynucleotide sequence of one of SEQ ID NOs: 32-35). In some embodiments, the SARS-CoV-2 spike protein comprises the sequence set forth in SEQ ID NO: 36 (optionally encoded by a polynucleotide sequence of one of SEQ ID NOs: 37-40).

### Proteolipid Vehicles

In some embodiments, the pharmaceutical compositions provided herein comprise proteolipid vehicles. A proteolipid vehicle comprises a lipid and one or more protein components contacting or disposed at least partially within the lipid. In some embodiments, the proteolipid vehicle encapsulates one or more other parts of the pharmaceutical composition (e.g., the DNA vector, such as any DNA vector provided herein).

In certain aspects the vaccine systems provided herein comprise proteolipid vehicles. A proteolipid vehicle comprises one or more lipid components which can encapsulate a vector provided herein (e.g., a DNA plasmid, or multiple DNA plasmids for a multi-valent composition). In some embodiments, the proteolipid vehicle comprises one or more protein components contacting or disposed at least partially within the lipid. In some embodiments, the proteolipid vehicle encapsulates a polynucleotide construct (e.g., a vector as provided herein, such as plasmid DNA, or multiple such plasmids).

In some embodiments, vaccine compositions comprising a plasmid DNA (or multiple plasmids) encapsulated with a proteolipid vehicle formulation are non-toxic and non-immunogenic in animals at doses of >15 mg/kg and exhibit an efficiency in excess of 80× greater than that achievable with neutral lipid compositions and 2-5× greater than that achievable with cationic lipid compositions. In some embodiments, proteolipid vehicle cargo is deposited directly into the cytoplasm, thereby bypassing the endocytic pathway.

Examples of proteolipid vehicles compatible with the disclosure herein are described in U.S. Pat. No. 8,252,901, U.S. Pat. Pub. No. 2019/0367566, and Patent Cooperation Treaty (PCT) Pub. No. WO2022/067446A1, each of which is incorporated herein by reference.

Within further embodiments, the present disclosure includes proteolipid vehicles for the targeted delivery of a vector encoding a SARS-CoV-2 protein (e.g., spike protein) within a target cell, which proteolipid vehicle composition comprises: (a) a lipid component, and (b) one or more fusogenic membrane protein(s).

Proteolipid vehicle compositions according to certain aspects of these embodiments include one or more lipid(s) at a concentration ranging from 1 mM to 100 mM, or from 5 mM to 50 mM, or from 10 mM to 30 mM, or from 15 mM to 25 mM. Lipid vesicle formulations exemplified herein can include one or more lipid(s) at a concentration of about 20 mM.

Within certain illustrative lipid vesicle compositions, one or more lipid(s) is selected from 1,2-dioleoyl-3-dimethylammonitim-propane (DODAP), 1,2-diolcoyl-3-trimethylammonium-propane (DOTAP), 1,2-dioleoyl-sn-glycero-3-phosphoethanolamine (DOPE), Cholesterol, and 1,2-dimyristoyl-rac-glycero-3-methoxypolyethylene glycol (DMG-PEG). LNP compositions may contain two or more lipids selected from the group consisting of DODAP, DOTAP, DOPE, Cholesterol, and DMG-PEG.

Exemplified herein are lipid compositions including DODAP, DOTAP, DOPE, Cholesterol, and DMG-PEG at a molar ratio of 35-55 mole % DODAP: 10-20 mole % DOTAP: 22.5-37.5 mole % DOPE: 4-8 mole % Cholesterol: 3-5 mole % DMG-PEG; or at a molar ratio of about 45 mole % DODAP:about 15:mole % DOTAP about 30 mole % DOPE:about 6 mole % Cholesterol about 4 mole % DMG-PEG. Within certain aspects, the lipid vesicle compositions include DODAP, DOTAP, DOPE, Cholesterol, and DMG-PEG at a molar ratio of 45 mole % DODAP: 5 mole % DOTAP: 30 mole % DOPE: 6 mole % Cholesterol: 4 mole % DMG-PEG.

Lipid vesicle formulations according to other aspects of these embodiments include one or more fusogenic membrane protein(s) at a concentration ranging from 0.5 μM to 20 μM, or from 1 μM to 10 μM, or from 3 μM to 4 μM. Exemplified herein are lipid vesicle formulations wherein fusogenic membrane protein(s) are present at a concentration of about 3.5 μM, about 5μ, about 7.5μ, about 10μ, about 12.5μ, about 15μ, about 20μ. Exemplary, suitable fusogenic membrane protein(s) include those provided herein, including a p15x fusogenic membrane protein (SEQ ID NO: 21), a p14 fusogenic membrane protein (SEQ ID NO: 22), and a p14c15 fusogenic membrane protein (SEQ ID NO: 23).

Within additional aspects of these embodiments, lipid vesicle formulations include vectors comprising polynucleotide sequences encoding one or more antibody or antigen binding fragment as set forth above.

In some embodiments, the pharmaceutical compositions provided herein comprise proteolipid vehicles (PLV). In some embodiments, the proteolipid vehicle encapsulates one or more other parts of the pharmaceutical composition (e.g., the DNA vector, such as any DNA vector provided herein).

Exemplified herein are lipid vesicle formulations including vectors (e.g., DNA plasmids) at a concentration ranging from 20 μg/mL to 1.5 mg/mL, of from 100 μg/mL to 500 μg/mL, or at a concentration of about 250 μg/mL.

A suitable exemplary lipid vesicle formulation includes the following: for each 1 mL of lipid vesicle, the lipid concentration is about 20 mM, the DNA content is about 250 μg, and the fusogenic protein (e.g, p14 or p14e15) is at about 3.5 μM wherein the lipid formulation comprises DODAP:DOTAP:DOPE:Cholesterol:DMG-PEG at a mole % ratio of about 45:15:30:6:4, respectively.

The lipid vesicle comprises one or more lipid components. In some embodiments, the lipids of the lipid vesicle are non-immunogenic lipids. In some embodiments, the lipids of the lipid vesicle comprise naturally occurring lipids. In some embodiments, the lipids of the lipid vesicle comprise naturally occurring mammalian lipids. In some embodiments, the lipids of the lipid vesicle comprise naturally occurring human lipids.

In some embodiments, the lipid vesicle comprises a minimal amount of cationic lipid. cationic lipids are used in certain lipid vesicle formulations in order to facilitate the fusion of the lipid vesicle with another desired membrane. However, in some embodiments, proteolipid vesicles provided herein use alternative strategies for the fusion of the lipid vesicle with a desired cell membrane (e.g., a fusogenic membrane protein). Thus, the lipid vesicles provided herein in some instances use less cationic lipids than other preparations, which makes the lipid vesicles provided herein less toxic. Due to their positive charge, cationic lipids have been employed for condensing negatively charged DNA molecules and to facilitate the encapsulation of DNA into liposomes. In some embodiments, the lipid vesicle comprises less than 10%, less than 5%, less than 4%, less than 3%, less than 2%, less than 1%, less than 0.5%, less than 0.4%, less than 0.3%, less than 0.2%, or less than 0.1% cationic lipid content in the proteolipid vehicle (w/w of total lipid content).

### Fusogenic Membrane Proteins

In some embodiments, the proteolipid vehicles comprises a fusogenic membrane protein. A fusogenic membrane protein is membrane bound or associated protein which facilitates lipid to lipid membrane fusion of two separate lipid membranes. Many such fusogenic membrane proteins are known in the art.

In some embodiments, the fusogenic membrane protein is derived from a virus. Examples of such virus derived fusogenic membrane proteins include influenza virus hemagglutinin (HA) proteins, Sendai virus F proteins, Filoviridae family ebolavirus glycoproteins, Retroviridae family glycoprotein 41, Togaviridae family alphaviruse envelope protein E1, Flaviviridae family Flavivirus envelope protein, Herpesviridae family Herpesvirus glycoprotein B, Rhabdoviridae family SVS G proteins, Reoviridae family fusion-associated small transmembrane proteins (FAST), and derivatives thereof.

Preferred fusogenic membrane proteins are those which are non-immunogenic (e.g., do not produce an immune response specific to the fusogenic membrane protein upon administration to a subject). In some cases, such fusogenic membrane proteins allow for repeated administration of the pharmaceutical compositions provided herein and/or for enhanced delivery of enclosed material (e.g., DNA vectors as provided herein) to target cells.

In some embodiments, the fusogenic membrane protein is a FAST protein. Examples of preferred FAST proteins are described in U.S. Pat. No. 8,252,901, U.S. Pat. Pub. No. 2019/0367566, and Patent Cooperation Treaty (PCT) Pub. No. WO2022/067446A1, each of which is incorporated by reference as if set forth herein in its entirety. Other descriptions of suitable fusogenic membrane proteins are described in PCT Patent Publication Nos. WO2012/040825A1 and WO2002/044206A2, Lau, Biophys. J. g£: 272 (2004), Nesbitt, Master of Science Thesis (2012), Zijlstra, AACR (2017), Mrlouah, PAACRAM 77/13Supnn: Abst 5143 (2017), Krabbe, Cancers 10:216 (2018), Sanchez-Garria, ChemComm 53:4565 (2017), Clancy/Virology 83/71:2941 (2009), Sudo, J Control Release 255:1 (2017), Wong, Cancer Gene Therapy 23:355 (2016), and Corcoran, JBC 281/421:31778 (2006), each of which is incorporated by reference as if set forth herein in its entirety. Within some embodiments, proteolipid vehicles provided herein comprise a fusogenic membrane protein, such as a fusogenic p14 FAST membrane fusion protein from reptilian reovirus to catalyze lipid mixing between the lipid vesicle and target cell plasma membrane.

FAST proteins are a unique family of membrane fusion proteins encoded by the fusogenic retroviruses. FAST proteins include: p10, p14, p15 and p22. At 95 to 198 amino acids in size, the FAST proteins are the smallest known viral membrane fusion proteins. Rather than mediating virus-cell fusion, the FAST proteins are non-structural viral proteins that are expressed on the surfaces of virus-infected or -transfected cells, where they induce cell-cell fusion and the formation of multinucleated syncytia. A purified FAST protein, when reconstituted into liposome membranes, induces liposome-cell and liposome-liposome fusion, indicating the FAST proteins are bona fide membrane fusion proteins.

In contrast to most enveloped viral fusion proteins in which the cytoplasmic tail is extremely short relative to the overall size of the protein, the FAST proteins all have an unusual topology that partitions the majority of the protein to the membrane and cytoplasm, exposing ectodomains of just 20 to 43 residues to the extracellular milieu. Despite the diminutive size of their ectodomains, both p14 and p10 encode patches of hydrophobicity (HP) hypothesized to induce lipid mixing analogously to the fusion peptides encoded by enveloped viral fusion proteins. The p14 HP is comprised of the N-terminal 21 residues of the protein, but peptides corresponding to this sequence require the inclusion of the N-terminal myristate moiety to mediate lipid mixing. Nuclear magnetic resonance (NMR) spectroscopy revealed that two proline residues within the p14 HP form a protruding loop structure presenting valine and phenylalanine residues at the apex and connected to the rest of the protein by a flexible linker region. The p10 HP on the other hand, flanked by two cysteine residues that form an intramolecular disulfide bond, may have more in common with the internal fusion peptides of the Ebola virus and avian leukosis and sarcoma virus (ALSV) glycoproteins, and likely adopts a cystine-noose structure that forces solvent exposure of conserved valine and phenylalanine residues for membrane interactions. In contrast to p14 and p10, the 20 residue ectodomain of p15 completely lacks a hydrophobic sequence that could function as a traditional fusion peptide. In the absence of such a motif, the p15 ectodomain instead encodes a polyproline helix that has been proposed to function as a membrane destabilizing motif.

FAST proteins with improved properties for facilitating membrane fusion in the context of synthetic lipid vehicles (e.g., the proteolipid vehicles of the instant disclosure) have been previously described (e.g., U.S. Pat. No. 10,227,386). Examples of fusogenic FAST proteins include the p15 and p14e15 proteins having the amino acid sequences presented in the table below. In some embodiments, the FAST protein of a lipid vesicle provided herein comprises a sequence having at least 80%, at least 85%, at least 90%, at least 95%, at least 98%, or 100% sequence identity with the sequence of p15x set forth below. In some embodiments, the FAST protein of a lipid vesicle provided herein comprises a sequence having at least 80%, at least 85%, at least 90%, at least 95%, at least 98%, or 100% sequence identity with the sequence of p14 set forth below. In some embodiments, the FAST protein of a lipid vesicle provided herein comprises a sequence having at least 80%, at least 85%, at least 90%, at least 95%, at least 98%, or 100% sequence identity with the sequence of p14e15 set forth below.

TABLE of
FAST Proteins

| | | |
|---|---|---|
| p15x | MGSGPSNFVNHAPGEAIVTGLEKG | SEQ ID NO: |
| | ADKVAGTISHTIFVEIVSSSTGIIIAV | 21 |
| | GIFAFIFSFLYKLLQWYNRKSKNK | |
| | KRKEQIREQIELGLLSYGAGVASLP | |
| | LLNVIAHNPGSVISATPIYKGPCTG | |
| | VPNSRLLQITSGTAEENTRILNHDG | |
| | RNPDGSINV | |
| p14 | MGSGPSNFVNHAPGEAIVTGLEKG | SEQ ID NO: |
| | ADKVAGTISHTIWEVIAGLVALLT | 22 |
| | FLAFGFWLFKYLQKRRERRRQLTE | |
| | FQKRYLRNSYRLSEIQRPISQHEYE | |
| | DPYEPPSRRKPPPPPYSTYVNIDNV | |
| | SAI | |
| p14e15 | MGSGPSNFVNHAPGEAIVTGLEKG | SEQ ID NO: |
| | ADKVAGTISHTIWEVIAGLVALLT | 23 |
| | FLAFGFWLFKYLQWYNRKSKNKK | |
| | RKEQIREQIELGLLSYGAGVASLPL | |
| | LNVIAHNPGSVISATPIYKGPCTGV | |
| | PNSRLLQITSGTAEENTRILNHDGR | |
| | NPDGSINV | |

In some embodiments, the FAST protein comprises domains from one or more FAST proteins selected from p10, p14, p15, and p22. In some embodiments, the FAST protein comprises an ectodomain, a transmembrane domain, and an endodomain.

In some embodiments, the FAST protein comprises an endodomain having at least about 80%, at least about 85%, at least about 90%, or at least about 95% sequence identity with an endodomain from p10, p14, p15, or p22. In some embodiments, the FAST protein comprises an endodomain from p10, p14, p15, or p22. In some embodiments, the FAST protein comprises an endodomain having at least about 80%, at least about 85%, at least about 90%, or at least about 95% sequence identity with an endodomain from p15. In some embodiments, the FAST protein comprises an endodomain from p15.

In some embodiments, the FAST protein comprises a transmembrane domain from a wild type FAST protein, or a derivative thereof. In some embodiments, the FAST protein comprise a transmembrane domain having at least about 80%, at least about 85%, at least about 90%, or at least about 95% sequence identity with a transmembrane domain from p10, p14, p15, or p22. In some embodiments, the transmembrane domain comprises 23 amino acid residues, at least two hydrophobic, β-branched residues adjacent the ectodomain, three consecutive serine residues immediately adjacent the at least two hydrophobic, β-branched residues, and a glycine residue at positions 7 and 13 from the junction between the ectodomain and the first hydrophobic, β-branched residue. In some embodiments, the transmembrane domain comprises an amino acid sequence having at least about 80%, at least about 85%, at least about 90%, or at least about 95% sequence identity with the sequence of IVSSSTGIIIAVGIFAFIFSFLY (SEQ ID NO: 12).

In some embodiments, the FAST protein comprises an ectodomain from a wild type FAST protein, or a derivative thereof. In some embodiments, the FAST protein comprises an ectodomain having at least about 80%, at least about 85%, at least about 90%, or at least about 95% sequence identity with an ectodomain from p10, p14, p15, or p22. In some embodiments, the FAST protein comprises an ectodomain from p10, p14, p15, or p22. In some embodiments, the FAST protein comprises an endodomain having at least about 80%, at least about 85%, at least about 90%, or at least about 95% sequence identity with an ectodomain from p14. In some embodiments, the FAST protein comprises an ectodomain from p14.

In some embodiments, a FAST protein as provided herein comprises an ectodomain comprising a sequence with at least 80% sequence identity that of a p14 FAST protein (e.g., the sequence defined by the sequence MGSGPSNFVNHAPGEAIVTGLEKGADKVAGTISHTIWE (SEQ ID NO: 13)) and comprising a functional myristoylation motif; a transmembrane domain comprising 23 amino acid residues, at least two hydrophobic, β-branched residues adjacent the ectodomain, three consecutive serine residues immediately adjacent the at least two hydrophobic, β-branched residues, and a glycine residue at positions 7 and 13 from the junction between the ectodomain and the first hydrophobic, β-branched residue; and an endodomain comprising a sequence with at least 80% sequence identity with the sequence a p15 endodomain (e.g., as sequence defined by KLLQWYNRKSKNKKRKEQIREQIELGLLSYGAGVASLPLLNVIAHNPGS (SEQ ID NO: 14) or VISATPIYKGPCTGVPNSRLLQITSGTAEENTRILNHDGRNPDGSINV (SEQ ID NO: 15)).

In some embodiments, the FAST protein comprises an amino acid having at least about at least about 80%, at least about 85%, at least about 90%, at least about 95%, at least about 96%, at least about 97%, at least about 98%, at least about 99%, or 100% sequence identity with the of sequence

(SEQ ID NO: 7)
MGSGPSNFVNHAPGEAIVTGLEKGADKVAGTISHTIFVEIVSSSTGIII
AVGIFAFIFSFLYKLLQWYNRKSKNKKRKEQIREQIELGLLSYGAGVAS
LPLLNVIAHNPGSVISATPIYKGPCTGVPNSRLLQITSGTAEENTRILN
HDGRNPDGSINV.

In some embodiments, the FAST protein is provided from a commercial vendor. In some embodiments, the FAST protein is part of the Fusogenix platform prepared by Entos Pharmaceuticals.

### Stability

The SARS-CoV-2 DNA vaccines provided herein preferably have a favorable stability profile. In some cases, the SARS-CoV-2 DNA vaccines are more stable at elevated temperatures than other vaccines, such as mRNA vaccines (e.g., the Pfizer/BioNTech COVID-19 vaccine Comirnaty®), which generally require frozen storage, in some cases at temperatures as low as −80° C. In some embodiments, the SARS-CoV-2 DNA vaccines provided herein are stable at standard refrigeration temperatures (e.g., ˜2-8° C.) or even room temperature (e.g., ˜20-25° C.) for extended periods of time, which can simplify supply chains and ensure that the SARS-CoV-2 vaccine is delivered in an active and non-degraded state.

Stability of the SARS-CoV-2 DNA vaccine can be measured in a variety of ways. In some instances, the stability is measured in a functional assay of the SARS-CoV-2 DNA vaccine (e.g., an activity above a threshold value remains present after the specified period of time, such as a threshold of 80%, 85%, 90%, 95%, 96%, 97%, 98%, or 99%). In some instances, the stability is measured by an assay which measures for concentrations of one or more components of the vaccine or degradants thereof (e.g., a DNA quantitation assay examining the amount of vector present after an indicated period of time).

In some embodiments, the SARS-CoV-2 DNA vaccines is stable at a temperature of about 2° C. to about 8° C. In some embodiments, the SARS-CoV-2 DNA vaccine is stable at a temperature of from about 2° C. to about 8° C. for a period of at least about 1 month, at least about 2 months, at least about 3 months, at least about 6 months, at least about 9 months, or at least about 12 months. In some embodiments, the SARS-CoV-2 DNA vaccine is stable at a temperature of from about 2° C. to about 8° C. for a period of at least about 6 months. In some embodiments, the SARS-CoV-2 DNA vaccine is stable at a temperature of from about 2° C. to about 8° C. for a period of at least about 9 months. In some embodiments, the SARS-CoV-2 DNA vaccine is stable at a temperature of from about 2° C. to about 8° C. for a period of at least about 12 months.

In some embodiments, the SARS-CoV-2 DNA vaccine is stable at room temperature (e.g., 20-25° C.). In some embodiments, the SARS-CoV-2 DNA vaccine is stable at room temperature for a period of at least about 7 days, at least about 14 days, at least about 21 days, or at least about 28 days. In some embodiments, the SARS-CoV-2 DNA vaccine is stable at room temperature for a period of at least about 7 days. In some embodiments, the SARS-CoV-2 DNA vaccine is stable at room temperature for a period of at least about 14 days. In some embodiments, the SARS-CoV-2 DNA vaccine is stable at room temperature for a period of at least about 21 days. In some embodiments, the SARS-CoV-2 DNA vaccine is stable at room temperature for a period of at least about 28 days.

### II. Methods of Treatment

The present disclosure also relates to methods of eliciting a response to a SARS-CoV-2 virus. In some embodiments, a DNA vaccine, a vector (or vectors), or an RNA polynucleotide (or RNA polynucleotides) as provided herein is administered to a subject. In some embodiments, after administration to the subject, the subject displays enhanced resistance to infection by the SARS-CoV-2 virus. In some embodiments, after administration to the subject, the subject displays a reduced risk of infection by the SARS-CoV-2 virus. In some embodiments, after administration to the subject, the subject displays reduced risk of developing one or more symptoms associated with SARS-CoV-2 infection. In some embodiments, after administration to the subject, the subject displays reduced risk of developing severe symptoms associated with SARS-CoV-2 infection.

In one aspect, provided herein, is a method of eliciting an anti-SARS-CoV-2 immune response in a subject, the method comprising administering to the subject a therapeutically effective amount of a SARS-CoV-2 DNA vaccine provided herein, a vector provided herein, or an RNA polynucleotide provided herein.

In one aspect, provided herein, is a method of eliciting an anti-SARS-CoV-2 immune response in a subject, the method comprising administering to the subject a therapeutically effective amount of an RNA polynucleotide provided herein.

In one aspect, provided herein, is a method of eliciting an anti-SARS-CoV-2 immune response in a subject, the method comprising administering to the subject a therapeutically effective amount of a vector provided herein.

In one aspect, provided herein, is a method of eliciting an anti-SARS-CoV-2 immune response in a subject, the method comprising administering to the subject a therapeutically effective amount of a SARS-CoV-2 DNA vaccine provided herein.

### Doses

In some embodiments, a subject is administered a therapeutically effective amount of a SARS-CoV-2 DNA vaccine provided herein, a vector provided herein, or an RNA polynucleotide provided herein. In some embodiments, the subject is administered a therapeutically effective amount of an RNA polynucleotide provided herein. In some embodiments, the subject is administered a therapeutically effective amount of a vector provided herein. In some embodiments, the subject is administered a therapeutically effective amount of a SARS-CoV-2 DNA vaccine provided herein. Where doses are described herein, unless otherwise specified, it is intended that the dose of the vaccine refers to the dose of the vector (or total dose of multiple vectors for multi-valent vaccines) in the vaccine (i.e., the other components of the vaccine are not calculated in determining the dose).

In some embodiments, the subject is administered an amount of a SARS-CoV-2 DNA vaccine effective to elicit a desired immune response. In some embodiments, the therapeutically effective amount of the SARS-CoV-2 DNA vaccine is measured as the amount of DNA vector administered as part of the vaccine.

In some embodiments, the therapeutically effective amount of the SARS-CoV-2 DNA vaccine is about 50 micrograms to about 500 micrograms. In some embodiments, the therapeutically effective amount of the SARS-CoV-2 DNA vaccine is about 50 micrograms to about 100 micrograms, about 50 micrograms to about 150 micrograms, about 50 micrograms to about 200 micrograms, about 50 micrograms to about 250 micrograms, about 50 micrograms to about 300 micrograms, about 50 micrograms to about 350 micrograms, about 50 micrograms to about 400 micrograms, about 50 micrograms to about 450 micrograms, about 50 micrograms to about 500 micrograms, about 100 micrograms to about 150 micrograms, about 100 micrograms to about 200 micrograms, about 100 micrograms to about 250 micrograms, about 100 micrograms to about 300 micrograms, about 100 micrograms to about 350 micrograms, about 100 micrograms to about 400 micrograms, about 100 micrograms to about 450 micrograms, about 100 micrograms to about 500 micrograms, about 150 micrograms to about 200 micrograms, about 150 micrograms to about 250 micrograms, about 150 micrograms to about 300 micrograms, about 150 micrograms to about 350 micrograms, about 150 micrograms to about 400 micrograms, about 150 micrograms to about 450 micrograms, about 150 micrograms to about 500 micrograms, about 200 micrograms to about 250 micrograms, about 200 micrograms to about 300 micrograms, about 200 micrograms to about 350 micrograms, about 200 micrograms to about 400 micrograms, about 200 micrograms to about 450 micrograms, about 200 micrograms to about 500 micrograms, about 250 micrograms to about 300 micrograms, about 250 micrograms to about 350 micrograms, about 250 micrograms to about 400 micrograms, about 250 micrograms to about 450 micrograms, about 250 micrograms to about 500 micrograms, about 300 micrograms to about 350 micrograms, about 300 micrograms to about 400 micrograms, about 300 micrograms to about 450 micrograms, about 300 micrograms to about 500 micrograms, about 350 micrograms to about 400 micrograms, about 350 micrograms to about 450 micrograms, about 350 micrograms to about 500 micrograms, about 400 micrograms to about 450 micrograms, about 400 micrograms to about 500 micrograms, or about 450 micrograms to about 500 micrograms. In some embodiments, the therapeutically effective amount of the SARS-CoV-2 DNA vaccine is about 50 micrograms, about 100 micrograms, about 150 micrograms, about 200 micrograms, about 250 micrograms, about 300 micrograms, about 350 micrograms, about 400 micrograms, about 450 micrograms, or about 500 micrograms. In some embodiments, the therapeutically effective amount of the SARS-CoV-2 DNA vaccine is at least about 50 micrograms, about 100 micrograms, about 150 micrograms, about 200 micrograms, about 250 micrograms, about 300 micrograms, about 350 micrograms, about 400 micrograms, or about 450 micrograms. In some embodiments, the therapeutically effective amount of the SARS-CoV-2 DNA vaccine is at most about 100 micrograms, about 150 micrograms, about 200 micrograms, about 250 micrograms, about 300 micrograms, about 350 micrograms, about 400 micrograms, about 450 micrograms, or about 500 micrograms. In some embodiments, the therapeutically effective amount of the SARS-CoV-2 DNA vaccine is about 25 micrograms. In some embodiments, the therapeutically effective amount of the SARS-CoV-2 DNA vaccine is about 50 micrograms. In some embodiments, the therapeutically effective amount of the SARS-CoV-2 DNA vaccine is about 100 micrograms. In some embodiments, the therapeutically effective amount of the SARS-CoV-2 DNA vaccine is about 150 micrograms. In some embodiments, the therapeutically effective amount of the SARS-CoV-2 DNA vaccine is about 200 micrograms. In some embodiments, the therapeutically effective amount of the SARS-CoV-2 DNA vaccine is about 250 micrograms. In some embodiments, the therapeutically effective amount of the SARS-CoV-2 DNA vaccine is about 300 micrograms. In some embodiments, the therapeutically effective amount of the SARS-CoV-2 DNA vaccine is about 350 micrograms. In some embodiments, the therapeutically effective amount of the SARS-CoV-2 DNA vaccine is about 400 micrograms. In some embodiments, the therapeutically effective amount of the SARS-CoV-2 DNA vaccine is about 450 micrograms. In some embodiments, the therapeutically effective amount of the SARS-CoV-2 DNA vaccine is about 500 micrograms.

In some embodiments, the therapeutically effective amount of the SARS-CoV-2 DNA vaccine is administered in a specified volume. In some embodiments, the therapeutically effective amount of the SARS-CoV-2 DNA vaccine is delivered in a volume of from about 0.1 mL to about 1.0 mL. In some embodiments, the therapeutically effective amount of the SARS-CoV-2 DNA vaccine is delivered in a volume of about 0.1 mL. about 0.2 mL, about 0.3 mL, about 0.4 mL, about 0.5 mL, about 0.6 mL, about 0.7 mL, about 0.8 mL, about 0.9 mL, or about 1.0 mL. In some embodiments, the therapeutically effective amount of the SARS-CoV-2 DNA vaccine is delivered in a volume of about 0.5 mL.

### Dosing Regimens

The SARS-CoV-2 DNA vaccines, vectors, or RNA polynucleotides provided herein may be dosed according to any number of dosing regimens. As used herein, a dosing regimen refers to an interval at which doses of the indicated therapeutics is administered, as well as how much of the indicated therapeutic is administered at a given interval.

In some embodiments, only a single therapeutically effective dose of the SARS-CoV-2 DNA vaccine is required in order to elicit a desired immune response. In some embodiments, a single dose of the SARS-CoV-2 DNA vaccine is administered, and a booster dose of the SARS-CoV-2 DNA vaccine is not administered for an extended period of time. In some embodiments, the administering consists of administering a single dose of the therapeutically effective amount of the SARS-CoV-2 DNA vaccine in a 6-month period. In some embodiments, the administering consists of administering a single dose of the therapeutically effective amount of the SARS-CoV-2 DNA vaccine in a 6-month period, a 9-month period, or a 12-month period.

In some embodiments, a first dose of the SARS-CoV-2 DNA vaccine is administered and a booster SARS-CoV-2 DNA vaccine is not administered for a period of at least about 6-months, at least about 9 months, at least about 12 months, at least about 15 months, at least about 18 months, at least about 21 months, or at least about 24 months. In some embodiments, a first dose of the SARS-CoV-2 DNA vaccine is administered and a booster SARS-CoV-2 DNA vaccine is not administered for at least about 6 months.

In some embodiments, administration of the SARS CoV-2 DNA vaccine comprises a single first dose followed by a second dose after a relatively short period of time. In some embodiments, the administering comprises administering two doses of the therapeutically effective amount of the SARS-CoV-2 DNA vaccine. In some embodiments, the two doses are the same. In some embodiments, the two doses are different amounts of the SARS-CoV-2 DNA vaccine. In some embodiments, the two doses are administered at an interval of about 2 weeks apart to about 8 weeks apart. In some embodiments, the two doses are administered at an interval of about 2 weeks apart to about 3 weeks apart, about 2 weeks apart to about 4 weeks apart, about 2 weeks apart to about 5 weeks apart, about 2 weeks apart to about 6 weeks apart, about 2 weeks apart to about 8 weeks apart, about 3 weeks apart to about 4 weeks apart, about 3 weeks apart to about 5 weeks apart, about 3 weeks apart to about 6 weeks apart, about 3 weeks apart to about 8 weeks apart, about 4 weeks apart to about 5 weeks apart, about 4 weeks apart to about 6 weeks apart, about 4 weeks apart to about 8 weeks apart, about 5 weeks apart to about 6 weeks apart, about 5 weeks apart to about 8 weeks apart, or about 6 weeks apart to about 8 weeks apart. In some embodiments, the two doses are administered at an interval of about 2 weeks apart, about 3 weeks apart, about 4 weeks apart, about 5 weeks apart, about 6 weeks apart, or about 8 weeks apart. In some embodiments, the two doses are administered at an interval of at least about 2 weeks apart, about 3 weeks apart, about 4 weeks apart, about 5 weeks apart, or about 6 weeks apart. In some embodiments, the two doses are administered at an interval of at most about 3 weeks apart, about 4 weeks apart, about 5 weeks apart, about 6 weeks apart, or about 8 weeks apart. In some embodiments, the two doses are administered at an interval of about 2 weeks apart, about 3 weeks apart, or about 4 weeks apart. In some embodiments, the two doses are administered at an interval of about 2 weeks apart. In some embodiments, the two doses are administered at an interval of about 3 weeks apart. In some embodiments, the two doses are administered at an interval of about 4 weeks apart. In some embodiments, the two doses are administered at an interval of about 5 weeks apart. In some embodiments, the two doses are administered at an interval of about 6 weeks apart. In some embodiments, a third dose is administered after an additional period of time following the second dose (e.g., 6 or more months later).

### Routes of Administration

The SARS-CoV-2 DNA vaccines, vectors, and RNA polynucleotides provided herein are administered by a route of administration which allows the SARS-CoV-2 DNA to be delivered to the relevant part of a subject (e.g., where it will come into contact with the immune system and elicit and immune response). In some embodiments, the SARS-CoV-2 DNA vaccines, vectors, or RNA polynucleotide is administered by intramuscular administration.

In some embodiments, the SARS-CoV-2 DNA vaccine is administered orally, nasally, sublingually, intravenously, intramuscularly, subcutaneously, or intradermally. In some embodiments, the SARS-CoV-2 DNA vaccine is administered intramuscularly.

In some embodiments, the SARS-CoV-2 DNA vaccine is administered intramuscularly to a specific part of the body of the subject. In some embodiments, the SARS-CoV-2 DNA vaccine is administered to the arm of the subject. In some embodiments, the SARS-CoV-2 DNA vaccine is administered to the deltoid muscle of the subject.

In some embodiments, the SARS-CoV-2 DNA vaccine is administered without electroporation. In some embodiments, the SARS-CoV-2 DNA vaccine is administered intramuscularly without electroporation. In some embodiments, the SARS-CoV-2 DNA vaccine is administered without any specialized equipment. In some embodiments, the SARS-CoV-2 DNA vaccine is administered with a standard needle and syringe. In some embodiments, the SARS-CoV-2 DNA vaccine is not administered with a jet injector.

### Subjects

In some embodiments, the SARS-CoV-2 DNA vaccines, vectors, and/or RNA polynucleotides provided herein are administered to a subject. The subject can be any animal at risk of contracting the SARS-CoV-2 virus, or any animal on which the SARS-CoV-2 DNA vaccine, vector, or RNA polynucleotide is desired to be tested.

In some embodiments, the subject is a mammal. In some embodiments, the subject is a rodent. In some embodiments, the subject is a mouse, a rat, or a hamster. In some embodiments, the subject is a rodent, a canine, a feline, a bovine, an equine, or a primate. In some embodiments, the subject is a primate. In some embodiments, the subject is a human.

In some embodiments, the subject is a human within a desired age range. In some embodiments, the human is above the age of 18. In some embodiments, the human is above the age of 18, 25, 35, 45, 55, 65, or 75. In some embodiments, the human is in the age range of 5 to 18. In some embodiments, the subject is in the age range of 3 to 5. In some embodiments, the subject is in the age range of 3-18. In some embodiments, the subject is in the age range of 0-18.

### Activity

In some embodiments, the SARS-CoV-2 DNA vaccine, vector, or RNA polynucleotide elicits an immune response to the SARS-CoV-2 virus in the subject after administration. In some embodiments, the immune response comprises increased antibody production. In some embodiments, the immune response comprises increased T-cell activation. In some embodiments, the immune response comprises increased antibody production and increased T-cell activation.

In some embodiments, the immune response is effective to eliminate or reduce the likelihood of being infected by a SARS-CoV-2 virus, the likelihood or severity of an infection by a SARS-CoV-2 virus, the likelihood of death from infection by a SARS-CoV-2 virus, the likelihood of hospitalization from infection by a SARS-CoV-2 virus, the likelihood of transmitting the SARS-CoV-2 virus to another individual if the subject is infected, or any combination thereof.

In some embodiments, comparison of an activity of a SARS-CoV-2 DNA vaccine to another value (e.g., an increased likelihood or decreased likelihood of a symptom or outcome of SARS-CoV-2 infection) refers to the value reflected in a subject who has received no SARS-CoV-2 vaccine. In some embodiments, comparison of an activity of a SARS-CoV-2 DNA vaccine to another value (e.g., an increased likelihood or decreased likelihood of a side effect) refers to the value reflected in the subject prior to being administered the SARS-CoV-2 DNA vaccine, preferably in a subject who has not received any previous vaccination for SARS-CoV-2.

In some embodiments, after administration of SARS-CoV-2 DNA vaccine, vector, or RNA polynucleotide provided herein to the subject, antibodies which bind the SARS-CoV-2 spike protein are increased by at least 10-fold in the subject. In some embodiments, antibodies which bind the SARS-CoV-2 spike protein are increased by at least 10-fold, 15-fold, 20-fold, 25-fold, 30-fold, 35-fold, 40-fold, 45-fold, 50-fold, 60-fold, 70-fold, 80-fold, 90-fold, or 100-fold. In some embodiments, the antibodies are IgG antibodies. In some embodiments, the increase in antibody levels is measured about 1 week, about 2 weeks, about 3 weeks, about 4 weeks, about 5 weeks, or about 6 weeks after a first dose. In some embodiments, the increase in antibody levels is measured about 1 week, about 2 weeks, about 3 weeks, about 4 weeks, about 5 weeks, or about 6 weeks after a second dose. In some embodiments, the antibodies also display specific binding for one or more variants of the SARS-CoV-2 spike protein. In some embodiments, the one or more variants of the SARS-CoV-2 spike protein comprise a spike protein from at least one variant selected from the beta variant, the delta variant, and the omicron variant. In some embodiments, the antibodies display specific binding for both the omicron variant and the delta variant.

In some embodiments, after administration of the SARS-CoV-2 DNA vaccine, vector, or RNA polynucleotide provided herein to the subject, T-cell activation in the subject is increased. In some embodiments, T-cells of the subject are activated specifically towards the SARS-CoV-2 spike protein.

In some embodiments, increased T-cell activation is measured by a percentage of the subject's CD4+ and/or CD8+ T-cells which produce interferon-gamma (IFN-γ) after stimulation with one or more peptide fragments of the SARS-CoV-2 spike protein. In some embodiments, a percentage of the subject's CD4+ and/or CD8+ T-cells which produce IFN-γ by a factor of at least 3-fold after stimulation with one or more peptide fragments of the SARS-CoV-2 spike protein. In some embodiments, a percentage of the subject's CD4+ and/or CD8+ T-cells which produce IFN-γ by a factor of at least 3-fold, at least 4-fold, at least 5-fold, at least 6-fold, at least 7-fold, at least 8-fold, at least 9-fold, or at least 10-fold after stimulation with one or more peptide fragments of the SARS-CoV-2 spike protein. In some embodiments, a percentage of the subject's CD4⁺ T-cells which produce IFN-γ by a factor of at least 3-fold after stimulation with one or more peptide fragments of the SARS-CoV-2 spike protein. In some embodiments, a percentage of the subject's CD4⁺ T-cells which produce IFN-γ by a factor of at least 3-fold, at least 4-fold, at least 5-fold, at least 6-fold, at least 7-fold, at least 8-fold, at least 9-fold, or at least 10-fold after stimulation with one or more peptide fragments of the SARS-CoV-2 spike protein. In some embodiments, a percentage of the subject's CD8+ T-cells which produce IFN-γ by a factor of at least 3-fold after stimulation with one or more peptide fragments of the SARS-CoV-2 spike protein. In some embodiments, a percentage of the subject's CD8+ T-cells which produce IFN-γ by a factor of at least 3-fold, at least 4-fold, at least 5-fold, at least 6-fold, at least 7-fold, at least 8-fold, at least 9-fold, or at least 10-fold after stimulation with one or more peptide fragments of the SARS-CoV-2 spike protein. In some embodiments, the increase in the percentage of the subject's CD4⁺ and/or CD8⁺ T-cells which produce IFN-γ is measured about 1 week, about 2 weeks, about 3 weeks, about 4 weeks, about 5 weeks, or about 6 weeks after a first dose. In some embodiments, the increase in the percentage of the subject's CD4⁺ and/or CD8+ T-cells which produce IFN-γ is measured about 1 week, about 2 weeks, about 3 weeks, about 4 weeks, about 5 weeks, or about 6 weeks after a second dose.

In some embodiments, increased T-cell activation comprises activation of cytotoxic T lymphocytes (CTLs). In some embodiments, increased activation of CTLs results in CTLs which display enhanced killing of cells comprising the SARS-CoV-2 spike protein (e.g., cells expressing the SARS-CoV-2 spike protein, such as a cell line engineered to express the SARS-CoV-2 spike protein for assay purposes). In some embodiments, T-cell activation is measured by the ability of CD8⁺ T-cells from the subject to kill cells comprising the SARS-CoV-2 protein compared to CD8⁺ T-cells from a subject who did not receive a dose of one or more of a SARS-CoV-2 DNA vaccine provided herein. In some embodiments, the subject has not received any SARS-CoV-2 vaccine. In some embodiments, after administration to the subject, CD8+ T-cells from the subject kill at least 50% more cells comprising the SARS-CoV-2 protein compared to CD8⁺ T-cells from a subject who did not receive the SARS-CoV-2 DNA vaccine. In some embodiments, after administration to the subject, CD8+ T-cells from the subject kill at least 50%, 60%, 70%, 80%, 90%, or 100% more cells comprising the SARS-CoV-2 protein compared to CD8+ T-cells from a subject who did not receive the SARS-CoV-2 DNA vaccine. In some embodiments, after administration to the subject, CD8⁺ T-cells from the subject kill at least 2-fold, 2.5-fold, 3-fold, 3.5-fold, 4-fold, 4.5-fold, 5-fold, 6-fold, 7-fold, 8-fold, 9-fold, or 10-fold more cells comprising the SARS-CoV-2 protein compared to CD8+ T-cells from a subject who did not receive the SARS-CoV-2 DNA vaccine. In some embodiments, cell killing is measured as outlined in the “CTL Killing Assay” as described in Example 7.

In some embodiments, the subject has an improved likelihood of a positive outcome or reduction in likelihood of a negative outcome related to SARS-CoV-2 infection. In some embodiments, the likelihood is made in comparison to a corresponding subject who has not received an intervention provided herein, or, where appropriate, to an aggregate of corresponding subjects who have not received an intervention provided herein. In some embodiments, the corresponding subjects have not received any SARS-CoV-2 related intervention (e.g., no other SARS-CoV-2 vaccine).

In some embodiments, after administration of the SARS-CoV-2 DNA vaccine, vector, or RNA polynucleotide provided herein to the subject, the subject displays a reduced likelihood of being infected by a SARS-CoV-2 virus. In some embodiments, the subject displays at least a 5%, at least a 10%, at least a 15%, at least a 20%, at least a 25%, at least a 30%, at least a 40%, at least a 50%, at least a 60%, at least a 70%, at least a 80%, or at least a 90% reduced likelihood of being infected by a SARS-CoV-2 virus.

In some embodiments, after administration of the SARS-CoV-2 DNA vaccine, vector, or RNA polynucleotide provided herein to the subject, the subject displays a reduced severity of an infection by a SARS-CoV-2 virus. In some embodiments, the reduced severity is determined by a reduced likelihood of developing one or more symptoms of an infection by a SARS-CoV-2 virus. In some embodiments, the reduced severity is determined by a reduced severity of one or more symptoms compared to average subjects who do not receive an intervention. Examples of symptoms of SARS-CoV-2 infection include fever, chills, cough, shortness of breath, difficulty breathing, fatigue, muscle or body aches, headache, loss of taste, loss of smell, sore throat, congestion, runny nose, nausea or vomiting, diarrhea, or any combination thereof.

In some embodiments, after administration of the SARS-CoV-2 DNA vaccine vector, or RNA polynucleotide provided herein to the subject, subjects displays a reduced recovery time from an infection by a SARS-CoV-2 virus. In some embodiments, the reduced recovery time is measured as an average dissipation of one or more symptoms of SARS-CoV-2 infection. In some embodiments, the reduced recovery time is at least 1 day, at least 2 days, at least 5 days, at least 10 days, at least 15 days, at least 20 days, at least 25 days, or at least 30 days compared to the average recovery time in individuals who did not receive the intervention.

In some embodiments, after administration of the SARS-CoV-2 DNA vaccine, vector, or RNA polynucleotide provided herein to the subject, the subject displays likelihood of death from infection by a SARS-CoV-2 virus. In some embodiments, the subject displays at least 10%, at least 20%, at least 30%, at least 40%, at least 50%, at least 60%, at least 70%, at least 75%, at least 80%, at least 85%, at least 90%, or at least 95% less change of dying from infection by a SARS-CoV-2 virus.

In some embodiments, after administration of the SARS-CoV-2 DNA vaccine, vector, or RNA polynucleotide provided herein to the subject, the subject displays a reduced likelihood of hospitalization from infection by a SARS-CoV-2 virus. In some embodiments, the subject's likelihood of being hospitalized due to infection from a SARS-CoV-2 virus is reduced by at least 10%, at least 20%, at least 30%, at least 40%, at least 50%, at least 60%, at least 70%, at least 75%, at least 80%, at least 85%, at least 90%, or at least 95%.

In some embodiments, after administration of the SARS-CoV-2 DNA vaccine, vector, or RNA polynucleotide provided herein to the subject, the subject displays a reduced likelihood of transmitting the SARS-CoV-2 virus to another individual if the subject is infected. In some embodiments, the likelihood of transmitting the SARS-CoV-2 virus to another individual if the subject becomes infected is reduced by at least 5%, at least 10%, at least 15%, at least 20%, at least 25%, at least 30%, at least 40%, at least 50%, at least 60%, at least 70%, at least 80%, or at least 90%.

In some embodiments, administration of a bi-valent vaccine as described herein (e.g., a vaccine containing two vectors encoding different spike proteins or a vaccine encoding two vectors, one vector encoding a spike protein and the other vector encoding a membrane, nucleocapsid, or envelope protein) exhibits enhanced activity compared to a corresponding monovalent vaccine. In some embodiments, administration of the SARS-CoV-2 DNA vaccine to a subject induces at least 2-fold, at least 3-fold, at least 4-fold, at least 5-fold, at least 6-fold, at least 7-fold, at least 8-fold, at least 9-fold, or at least 10-fold higher anti-spike protein antibody concentration as compared to a SARS-CoV-2 DNA vaccine which contains only one of the vectors (e.g., only one vector encoding one spike protein).

In embodiments where the vaccine contains vectors encoding two spike proteins, the higher anti-spike protein antibody concentration can be determined against the spike protein from which one of the SARS-CoV-2 spike protein is derived (e.g., a corresponding spike protein which does not contain a “2P” mutation as provided herein). In some embodiments, the higher anti-spike protein concentration can be determined as against a third SARS-CoV-2 spike protein. In some embodiments, the third SARS-CoV-2 spike protein comprises at least 1, at least 2, at least 3, at least 4, or at least 5 amino acid modification as compared to the first and second SARS-CoV-2 spike proteins. In some embodiments, the third SARS-CoV-2 spike protein is a different sub-variant of the same variant from which one of the administered SARS-CoV-2 spike proteins is derived.

### III. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

Used herein are references to insertions and/or deletions of one or more nucleotides or amino acids from a sequence. As used herein in reference to a sequence, the term “ins” placed before a number followed by a nucleotide or amino acid sequence means that the listed nucleotide or amino acid sequence is inserted into the sequence after the indicated residue. For example, “ins214TDR” indicates that the sequence “TDR” is inserted after residue 214 of the referenced sequence. As used herein, the term “del” following a number or range of numbers indicates that the nucleotide(s) or amino acid(s) at the indicated position numbers of the reference sequence are deleted from the sequence. For example, 137-145del indicates that residues 137, 138, 139, 140, 141, 142, 143, 144, and 145 are deleted from the reference sequence.

As used herein, the vaccine candidate “NP-S-CpG-RIGI” refers to the vaccine described herein with the plasmid which encodes the Wuhan Spike protein of SEQ ID NO: 1 with the nucleotide sequence defined by SEQ ID NO: 6. The vaccine candidate “NP-S-2P” refers to a substantially identical vaccine as that of NP-S-CpG-RIGI (e.g., comprising the same adjuvants), but differs in that the vector of NP-S-2P encodes the spike protein of SEQ ID NO: 2 (having the D614G, K986P, V987P spike protein mutations) encoded by the nucleotide sequence of SEQ ID NO: 5. “VAX-002” likewise refers to the analogous vaccine candidate (e.g., comprising the same adjuvants), but encodes the spike protein of SEQ ID NO: 36, encoded by the nucleotide sequence of SEQ ID NO: 40.

In some instances, nucleotide sequences encoding proteins or peptide sequences as provided herein are depicted with the stop codon as part of the sequence. Where the stop codon is included, it is contemplated herein that alternative stop codons could be used in the alternative to the one depicted. Where percent sequence identities of such nucleotide sequences encoding proteins or peptides are described herein, it is also contemplated that the % sequence identity can refer to only the portion of the nucleotide sequence which encodes amino acids.

IV. Sequences

| SEQ | | |
|---|---|---|
| ID | | |
| NO | Comment | Sequence |
| 1 | Wuhan Spike | MFVFLVLLPLVSSQCVNLTTRTQLPPAYTNSFTRGVYYPDKVFRSSVLHSTQDLFLPFF |
| | Protein | S |
| | | NVTWFHAIHVSGTNGTKRFDNPVLPFNDGVYFASTEKSNIIRGWIFGTTLDSKTQSLLIV |
| | | NNATNVVIKVCEFQFCNDPFLGVYYHKNNKSWMESEFRVYSSANNCTFEYVSQPFLM |
| | | DLE |
| | | GKQGNFKNLREFVFKNIDGYFKIYSKHTPINLVRDLPQGFSALEPLVDLPIGINITRFQT |
| | | LLALHRSYLTPGDSSSGWTAGAAAYYVGYLQPRTFLLKYNENGTITDAVDCALDPLSE |
| | | TK |
| | | CTLKSFTVEKGIYQTSNFRVQPTESIVRFPNITNLCPFGEVFNATRFASVYAWNRKRISN |
| | | CVADYSVLYNSASFSTFKCYGVSPTKLNDLCFTNVYADSFVIRGDEVRQIAPGQTGKIA |
| | | D |
| | | YNYKLPDDFTGCVIAWNSNNLDSKVGGNYNYLYRLFRKSNLKPFERDISTEIYQAGST |
| | | PC |
| | | NGVEGENCYFPLQSYGFQPTNGVGYQPYRVVVLSFELLHAPATVCGPKKSTNLVKNK |
| | | CVN |
| | | FNFNGLTGTGVLTESNKKFLPFQQFGRDIADTTDAVRDPQTLEILDITPCSFGGVSVITP |
| | | GTNTSNQVAVLYQDVNCTEVPVAIHADQLTPTWRVYSTGSNVFQTRAGCLIGAEHVN |
| | | NSY |
| | | ECDIPIGAGICASYQTQTNSPRRARSVASQSIIAYTMSLGAENSVAYSNNSIAIPTNFTI |
| | | SVTTEILPVSMTKTSVDCTMYICGDSTECSNLLLQYGSFCTQLNRALTGIAVEQDKNTQ |
| | | E |
| | | VFAQVKQIYKTPPIKDFGGFNFSQILPDPSKPSKRSFIEDLLFNKVTLADAGFIKQYGDC |
| | | LGDIAARDLICAQKFNGLTVLPPLLTDEMIAQYTSALLAGTITSGWTFGAGAALQIPFA |
| | | M |
| | | QMAYRFNGIGVTQNVLYENQKLIANQFNSAIGKIQDSLSSTASALGKLQDVVNQNAQA |
| | | LN |
| | | TLVKQLSSNFGAISSVLNDILSRLDKVEAEVQIDRLITGRLQSLQTYVTQQLIRAAEIRA |
| | | SANLAATKMSECVLGQSKRVDFCGKGYHLMSFPQSAPHGVVFLHVTYVPAQEKNFTT |
| | | APAICHDGKAHFPREGVFVSNGTHWFVTQRNFYEPQIITTDNTFVSGNCDVVIGIVNNT |
| | | VYDP |
| | | LQPELDSFKEELDKYFKNHTSPDVDLGDISGINASVVNIQKEIDRLNEVAKNLNESLIDL |
| | | QELGKYEQYIKWPWYIWLGFIAGLIAIVMVTIMLCCMTSCCSCLKGCCSCGSCCKFDE |
| | | DDSEPVLKGVKLHYT |
| 2 | D614G, K986P, | MFVFLVLLPLVSSQCVNLTTRTQLPPAYTNSFTRGVYYPDKVFRSSVLHSTQDLFLPFF |
| | V987P | SNVTWFHAIHVSGTNGTKRFDNPVLPFNDGVYFASTEKSNIIRGWIFGTTLDSKTQSLLI |
| | mutant | VNNATNVVIKVCEFQFCNDPFLGVYYHKNNKSWMESEFRVYSSANNCTFEYVSQPFL |
| | | MDLEGKQGNFKNLREFVFKNIDGYFKIYSKHTPINLVRDLPQGFSALEPLVDLPIGINIT |
| | | RFQTLLALHRSYLTPGDSSSGWTAGAAAYYVGYLQPRTFLLKYNENGTITDAVDCAL |
| | | DPLSETKCTLKSFTVEKGIYQTSNFRVQPTESIVRFPNITNLCPFGEVFNATRFASVYAW |
| | | NRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYLYRLFRKSNLKPFERDIST |
| | | EIYQAGSTPCNGVEGENCYFPLQSYGFQPTNGVGYQPYRVVVLSFELLHAPATVCGPK |
| | | KSTNLVKNKCVNFNFNGLTGTGVLTESNKKFLPFQQFGRDIADTTDAVRDPQTLEILDI |
| | | TPCSFGGVSVITPGTNTSNQVAVLYQGVNCTEVPVAIHADQLTPTWRVYSTGSNVFQT |
| | | RAGCLIGAEHVNNSYECDIPIGAGICASYQTQTNSPRRARSVASQSIIAYTMSLGAENSV |
| | | AYSNNSIAIPTNFTISVTTEILPVSMTKTSVDCTMYICGDSTECSNLLLQYGSFCTQLNRA |
| | | LTGIAVEQDKNTQEVFAQVKQIYKTPPIKDFGGFNFSQILPDPSKPSKRSFIEDLLFNKVT |
| | | LADAGFIKQYGDCLGDIAARDLICAQKFNGLTVLPPLLTDEMIAQYTSALLAGTITSGW |
| | | TFGAGAALQIPFAMQMAYRFNGIGVTQNVLYENQKLIANQFNSAIGKIQDSLSSTASAL |
| | | GKLQDVVNQNAQALNTLVKQLSSNFGAISSVLNDILSRLDPPEAEVQIDRLITGRLQSL |
| | | QTYVTQQLIRAAEIRASANLAATKMSECVLGQSKRVDFCGKGYHLMSFPQSAPHGVV |
| | | FLHVTYVPAQEKNFTTAPAICHDGKAHFPREGVFVSNGTHWFVTQRNFYEPQIITTDNT |
| | | FVSGNCDVVIGIVNNTVYDPLQPELDSFKEELDKYFKNHTSPDVDLGDISGINASVVNI |
| | | QKEIDRLNEVAKNLNESLIDLQELGKYEQYIKWPWYIWLGFIAGLIAIVMVTIMLCCMT |
| | | SCCSCLKGCCSCGSCCKFDEDDSEPVLKGVKLHYT |
| 3 | Omicron | MFVFLVLLPLVSSQCVNLTTRTQLPPAYTNSFTRGVYYPDKVFRSSVLHSTQDLFLPFF |
| | Spike 2P | SNVTWFHVISGTNGTKRFDNPVLPFNDGVYFASIEKSNIIRGWIFGTTLDSKTQSLLIVN |
| | (B.1.1.5 | NATNVVIKVCEFQFCNDPFLDHKNNKSWMESEFRVYSSANNCTFEYVSQPFLMDLEG |
| | 29 2P | KQGNFKNLREFVFKNIDGYFKIYSKHTPIIVREPEDLPQGFSALEPLVDLPIGINITRFQTL |
| | variant) | LALHRSYLTPGDSSSGWTAGAAAYYVGYLQPRTFLLKYNENGTITDAVDCALDPLSET |
| | | KCTLKSFTVEKGIYQTSNFRVQPTESIVRFPNITNLCPFDEVFNATRFASVYAWNRKRIS |
| | | NCVADYSVLYNLAPFFTFKCYGVSPTKLNDLCFTNVYADSFVIRGDEVRQIAPGQTGNI |
| | | ADYNYKLPDDFTGCVIAWNSNKLDSKVSGNYNYLYRLFRKSNLKPFERDISTEIYQAG |
| | | NKPCNGVAGFNCYFPLRSYSFRPTYGVGHQPYRVVVLSFELLHAPATVCGPKKSTNLV |
| | | KNKCVNFNFNGLKGTGVLTESNKKFLPFQQFGRDIADTTDAVRDPQTLEILDITPCSFG |
| | | GVSVITPGTNTSNQVAVLYQGVNCTEVPVAIHADQLTPTWRVYSTGSNVFQTRAGCLI |
| | | GAEYVNNSYECDIPIGAGICASYQTQTKSHRRARSVASQSIIAYTMSLGAENSVAYSNN |
| | | SIAIPTNFTISVTTEILPVSMTKTSVDCTMYICGDSTECSNLLLQYGSFCTQLKRALTGIA |
| | | VEQDKNTQEVFAQVKQIYKTPPIKYFGGFNFSQILPDPSKPSKRSFIEDLLFNKVTLADA |
| | | GFIKQYGDCLGDIAARDLICAQKFKGLTVLPPLLTDEMIAQYTSALLAGTITSGWTFGA |
| | | GAALQIPFAMQMAYRFNGIGVTQNVLYENQKLIANQFNSAIGKIQDSLSSTASALGKL |
| | | QDVVNHNAQALNTLVKQLSSKFGAISSVLNDIFSRLDPPEAEVQIDRLITGRLQSLQTY |
| | | VTQQLIRAAEIRASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYEPQIITTDNTFVS |
| | | GNCDVVIGIVNNTVYDPLQPELDSFKEELDKYFKNHTSPDVDLGDISGINASVVNIQKEI |
| | | DRLNEVAKNLNESLIDLQELGKYEQYIKWPWYIWLGFIAGLIAIVMVTIMLCCMTSCCS |
| | | CLKGCCSCGSCCKFDEDDSEPVLKGVKLHYT |
| 4 | Nucleotide | atgttcgtgtttctggtgctgctgcccctggtgtccagccagtgtgtgaacctgaccaccagaacacagctgcctccagcctacaccaaca |
| | Sequence A | gcttcaccagaggcgtgtactaccccgacaaggtgttcagatccagcgtgctgcactctacccaggacctgttcctgcctttcttcagcaac |
| | encoding | gtgacctggttccacgtgatcagcggcaccaatggcaccaagagattcgacaaccccgtgctgcccttcaacgacggggtgtactttgcc |
| | Omicron | agcatcgagaagtccaacatcatccgcggctggatcttcggcaccacactggatagcaagacccagagcctgctgatcgtgaacaacgc |
| | Spike 2P | caccaacgtggtcatcaaagtgtgcgagttccagttctgcaacgacccattcctggaccacaagaacaacaagagctggatggaaagcg |
| | (B.1.1.5 | agttccgggtgtacagcagcgccaacaactgcaccttcgagtacgtgtcccagcctttcctgatggacctggaaggcaagcagggcaac |
| | 29 2P | ttcaagaacctgcgcgagttcgtgttcaagaacatcgacggctacttcaaaatctacagcaagcacaccccgatcatcgtgcgcgagcct |
| | variant) | gaggatctgcctcagggcttttctgccctggaacctctggtggatctgcccatcggcatcaacatcacccggtttcagacactgctggccct |
| | (including | gcacagaagctacctgacacctggcgatagcagcagcggatggacagctggtgccgccgcttactatgtgggctacctgcagcctaga |
| | stop codon) | accttcctgctgaagtacaacgagaacggcaccatcaccgacgccgtggattgtgcccttgatcctctgagcgagacaaagtgcaccctg |
| | | aagtccttcaccgtggaaaagggcatctaccagaccagcaacttccgggtgcagcccaccgaatccatcgtgcggttccccaatatcacc |
| | | aatctgtgccccttcgatgaggtgttcaatgccaccagattcgccagcgtgtacgcctggaaccggaagagaatcagcaactgcgtggcc |
| | | gactactccgtgctgtacaatctggccccattcttcaccttcaagtgctacggcgtgtcccctaccaagctgaacgacctgtgcttcaccaat |
| | | gtgtacgccgacagcttcgtgatccggggagatgaagtgcggcagattgcccctggacagaccggcaatatcgccgactacaactaca |
| | | agctgcccgacgacttcaccggctgtgtgatcgcctggaatagcaacaagctggacagcaaggtgtccggcaactacaattacctgtacc |
| | | ggctgttccggaagtccaatctgaagcccttcgagcgggacatcagcaccgaaatctatcaggccggcaacaagccctgcaatggcgtg |
| | | gcaggcttcaactgctacttcccactgcggagctacagcttcagacccacatacggcgttggccaccagccttacagagtggtggtgctgt |
| | | ccttcgagctgctgcatgctcctgccacagtgtgcggccctaagaaaagcaccaacctcgtgaagaacaaatgcgtgaacttcaacttca |
| | | acggcctgaaaggcaccggcgtgctgaccgagagcaacaagaagttcctgccattccagcagttcggccgggacattgccgataccac |
| | | agacgccgttagagatccccagacactggaaatcctggacatcaccccttgcagcttcggcggagtgtctgtgatcacccctggcaccaa |
| | | caccagcaatcaggtggcagtgctgtaccagggcgtgaactgtacagaggtgccagtggccattcacgccgatcagctgacccctactt |
| | | ggcgggtgtactccacaggcagcaatgtgttccagaccagagccggctgtctgattggcgccgagtatgtgaacaacagctacgagtgc |
| | | gacatccccatcggagccggcatctgtgccagctaccagacacagaccaagagccacagacgggctagaagcgtggccagccagag |
| | | catcattgcctacacaatgtctctgggcgccgagaacagcgtggcctacagcaacaactctatcgctatccccaccaacttcaccatcagc |
| | | gtgaccaccgagatcctgcctgtgtccatgaccaagaccagcgtggactgcaccatgtacatctgcggcgacagcaccgagtgctccaa |
| | | tctgctgctgcagtacggcagcttctgcacccagctgaagagagccctgacagggattgccgtggaacaggacaagaacacccaagag |
| | | gtgttcgcccaagtgaagcaaatctacaagaccccgcctatcaagtacttcggcggcttcaatttcagccagattctgcccgatcctagcaa |
| | | gcccagcaagcggagcttcatcgaggacctgctgttcaacaaagtgacactggccgacgccggcttcatcaagcagtatggcgattgcc |
| | | tgggcgacattgcagccagggatctgatttgcgcccagaagttcaagggcctgacagtgctgcctcctctgctgacagatgagatgatcg |
| | | cccagtacacaagcgccctgctggccggcacaatcacctctggatggacatttggagccggcgctgccctgcagatcccatttgctatgc |
| | | agatggcctaccggttcaacggcatcggagtgacccagaatgtgctgtacgagaaccagaagctgatcgccaaccagttcaacagcgc |
| | | catcggcaagatccaggacagcctgagcagcacagcaagcgctctgggaaagctgcaggacgtggtcaaccacaatgcccaggcact |
| | | gaacaccctggtcaagcagctgtcctccaagttcggcgccatctctagcgtgctgaatgacatcttctcccggctggaccctcctgaggcc |
| | | gaggtgcaaatcgacagactgatcaccggcagactgcagagcctccagacatacgtgacccagcagctgattagagccgccgagatca |
| | | gagccagcgctaatctggccgccaccaagatgtctgagtgtgtgctgggccagagcaagagagtggacttttgcggcaagggctacca |
| | | cctgatgagcttccctcagtctgctcctcacggcgtggtgtttctgcacgtgacctacgtgcccgctcaagagaagaatttcaccaccgctc |
| | | cagccatctgccacgacggcaaagcccactttcctagagaaggcgtgttcgtgtccaacggcacccattggttcgtgacacagcggaact |
| | | tctacgagccccagatcatcaccaccgacaacaccttcgtgtctggcaactgcgacgtcgtgatcggcattgtgaacaataccgtgtacga |
| | | ccctctgcagcccgagctggactccttcaaagaggaactggataagtactttaagaaccacacaagccccgacgtggacctgggcgata |
| | | tcagcggaatcaatgccagcgtcgtgaacatccagaaagagatcgaccggctgaacgaggtggccaagaatctgaacgagagcctgat |
| | | cgacctgcaagaactggggaagtacgagcagtacatcaagtggccttggtacatctggctgggctttatcgccggactgattgccatcgt |
| | | gatggtcacaatcatgctgtgttgcatgaccagctgctgtagctgcctgaagggctgttgtagctgtggctcctgctgcaagttcgacgagg |
| | | acgattctgagcccgtgctgaaaggcgtgaagctgcactacacctga |
| 5 | Optimized | atgttcgtgttcctggtgctgctgcctctggtgtccagccagtgtgtgaacctgaccaccagaacacagctgc |
| | codons for | ctccagcctacaccaacagctttaccagaggcgtgtactaccccgacaaggtgttcagatccagcgtgctgca |
| | D614G, K986P, | ctctacccaggacctgttcctgcctttcttcagcaacgtgacctggttccacgccatccacgtgtccggcacca |
| | V987P mutant | atggcaccaagagattcgacaaccccgtgctgcccttcaacgacggggtgtactttgccagcaccgagaagt |
| | in NP-S-2P | ccaacatcatcagaggctggatcttcggcaccacactggacagcaagacccagagcctgctgatcgtgaac |
| | vector | aacgccaccaacgtggtcatcaaagtgtgcgagttccagttctgcaacgaccccttcctgggcgtctactacc |
| | | acaagaacaacaagagctggatggaaagcgagttccgggtgtacagcagcgccaacaactgcaccttcga |
| | | gtacgtgtcccagcctttcctgatggacctggaaggcaagcagggcaacttcaagaacctgcgcgagttcgt |
| | | gtttaagaacatcgacggctacttcaagatctacagcaagcacacccctatcaacctcgtgcgggatctgcct |
| | | cagggcttctctgctctggaacccctggtggatctgcccatcggcatcaacatcacccggtttcagacactgct |
| | | ggccctgcacagaagctacctgacacctggcgatagcagcagcggatggacagctggtgccgccgcttacta |
| | | tgtgggctacctgcagcctagaaccttcctgctgaagtacaacgagaacggcaccatcaccgacgccgtgga |
| | | ttgtgctctggatcctctgagcgagacaaagtgcaccctgaagtccttcaccgtggaaaagggcatctaccag |
| | | accagcaacttccgggtgcagcccaccgaatccatcgtgcggttccccaatatcaccaatctgtgccccttcg |
| | | gcgaggtgttcaatgccaccagattcgcctctgtgtacgcctggaaccggaagcggatcagcaattgcgtgg |
| | | ccgactactccgtgctgtacaactccgccagcttcagcaccttcaagtgctacggcgtgtcccctaccaagctg |
| | | aacgacctgtgcttcacaaacgtgtacgccgacagcttcgtgatccggggagatgaagtgcggcagattgcc |
| | | cctggacagacaggcaagatcgccgactacaactacaagctgcccgacgacttcaccggctgtgtgattgcc |
| | | tggaacagcaacaacctggactccaaagtcggcggcaactacaattacctgtaccggctgttccggaagtcc |
| | | aatctgaagcccttcgagcgggacatctccaccgagatctatcaggccggcagcaccccttgtaacggcgtg |
| | | gaaggcttcaactgctacttcccactgcagtcctacggctttcagcccacaaatggcgtgggctatcagccct |
| | | acagagtggtggtgctgagcttcgaactgctgcatgcccctgccacagtgtgcggccctaagaaaagcacca |
| | | atctcgtgaagaacaaatgcgtgaacttcaacttcaacggcctgaccggcaccggcgtgctgacagagagc |
| | | aacaagaagttcctgccattccagcagtttggccgggatatcgccgataccacagacgccgttagagatccc |
| | | cagacactggaaatcctggacatcaccccttgcagcttcggcggagtgtctgtgatcacccctggcaccaaca |
| | | ccagcaatcaggtggcagtgctgtaccagggcgtgaactgtaccgaagtgcccgtggccattcacgccgatc |
| | | agctgacacctacatggcgggtgtactccaccggcagcaatgtgtttcagaccagagccggctgtctgatcg |
| | | gagccgagcacgtgaacaatagctacgagtgcgacatccccatcggcgctggaatctgcgccagctaccag |
| | | acacagacaaacagccctcggagagccagaagcgtggccagccagagcatcattgcctacacaatgtctct |
| | | gggcgccgagaacagcgtggcctactccaacaactctatcgctatccccaccaacttcaccatcagcgtgac |
| | | cacagagatcctgcctgtgtccatgaccaagaccagcgtggactgcaccatgtacatctgcggcgattccacc |
| | | gagtgctccaacctgctgctgcagtacggcagcttctgcacccagctgaatagagccctgacagggatcgcc |
| | | gtggaacaggacaagaacacccaagaggtgttcgcccaagtgaagcagatctacaagacccctcctatcaa |
| | | ggacttcggcggcttcaatttcagccagattctgcccgatcctagcaagcccagcaagcggagcttcatcgag |
| | | gacctgctgttcaacaaagtgacactggccgacgccggcttcatcaagcagtatggcgattgtctgggcgac |
| | | attgccgccagggatctgatttgcgcccagaagtttaacggactgacagtgctgcctcctctgctgaccgatg |
| | | agatgatcgcccagtacacatctgccctgctggccggcacaatcacaagcggctggacatttggagcaggcg |
| | | ccgctctgcagatcccctttgctatgcagatggcctaccggttcaacggcatcggagtgacccagaatgtgct |
| | | gtacgagaaccagaagctgatcgccaaccagttcaacagcgccatcggcaagatccaggacagcctgagc |
| | | agcacagcaagcgccctgggaaagctgcaggacgtggtcaaccagaatgcccaggcactgaacaccctgg |
| | | tcaagcagctgtcctccaacttcggcgccatcagctctgtgctgaacgatatcctgagcagactggaccctcct |
| | | gaggccgaggtgcagatcgacagactgatcacaggcagactgcagagcctccagacatacgtgacccagc |
| | | agctgatcagagccgccgagattagagcctctgccaatctggccgccaccaagatgtctgagtgtgtgctgg |
| | | gccagagcaagagagtggacttttgcggcaagggctaccacctgatgagcttccctcagtctgcccctcacg |
| | | gcgtggtgtttctgcacgtgacatatgtgcccgctcaagagaagaatttcaccaccgctccagccatctgcca |
| | | cgacggcaaagcccactttcctagagaaggcgtgttcgtgtccaacggcacccattggttcgtgacacagcg |
| | | gaacttctacgagccccagatcatcaccaccgacaacaccttcgtgtctggcaactgcgacgtcgtgatcggc |
| | | attgtgaacaataccgtgtacgaccctctgcagcccgagctggacagcttcaaagaggaactggacaagtac |
| | | tttaagaaccacacaagccccgacgtggacctgggcgatatcagcggaatcaatgccagcgtcgtgaacatc |
| | | cagaaagagatcgaccggctgaacgaggtggccaagaatctgaacgagagcctgatcgacctgcaagaac |
| | | tggggaagtacgagcagtacatcaagtggccctggtacatctggctgggctttatcgccggactgattgccat |
| | | cgtgatggtcacaatcatgctgtgttgcatgaccagctgctgtagctgcctgaagggctgttgtagctgtggca |
| | | gctgctgcaagttcgacgaggacgattctgagcccgtgctgaagggcgtgaaactgcactacacatga |
| 6 | Optimized | atgttcgtatttctcgtcctgctccccttggtgagctcccagtgcgtcaatcttacaacacgaacccagctccct |
| | codon for | cccgcttacactaacagttttactaggggtgtatattacccagataaggtattccggtcctccgtacttcacag |
| | Wuhan Spike | cacacaagacctttttctcccatttttctcaaacgtgacatggttccacgccatccacgtctctggtaccaatgg |
| | protein | caccaaacgattcgataaccccgtacttccctttaatgacggggtttatttcgcaagtactgagaagagcaac |
| | in NP-S-CpG- | atcatccgaggatggatttttgggacaactttggatagcaaaactcaatctctgttgatagtcaacaacgcta |
| | RIGI | ctaacgtcgtgattaaagtatgtgaattccaattctgtaatgacccctttctgggggtctactatcataaaaata |
| | | acaagagctggatggagtcagagttccgcgtctactctagcgctaataattgcacttttgagtatgttagccag |
| | | ccattccttatggaccttgaagggaagcaggggaatttcaagaaccttagagagttcgttttcaagaatatag |
| | | acggatattttaagatttacagtaaacacacaccaatcaacctggttcgcgatctcccacagggttttagtgct |
| | | ttggagcccctggttgacctccccataggaataaacataacacgctttcaaactctgttggctcttcataggag |
| | | ttatttgaccccaggtgattcaagctctggatggactgctggagcagccgcttactacgtggggtatctgcaac |
| | | caaggaccttcctgctgaaatacaacgaaaatgggaccattactgatgccgtagactgcgctctcgaccccct |
| | | gtccgaaactaaatgcacccttaagtccttcactgtcgagaaaggaatttatcagactagtaactttcgagtc |
| | | cagccaacagaatcaatcgttcgatttcccaatatcactaatctctgcccattcggtgaagtgttcaatgcaac |
| | | tcgctttgcctccgtatatgcttggaataggaagcggatttctaactgcgtagcagattactccgttctgtataa |
| | | ttcagccagcttcagcactttcaaatgttacggggtttctccaacaaaactgaacgatctgtgtttcactaatgt |
| | | gtacgctgattcatttgtgattagaggggatgaagtccgccagattgccccaggtcagacagggaagatcgc |
| | | cgattacaactataaacttcccgacgacttcactggatgcgtaatagcatggaatagtaataatctggacagc |
| | | aaggtaggtgggaactacaactatttgtataggttgtttagaaagtcaaacctcaagcccttcgagagagac |
| | | atctctaccgagatttaccaggctggtagcacaccttgtaatggcgtagaaggctttaattgttactttcctctc |
| | | caaagttacggctttcaacctactaatggggttggctaccagccctaccgagttgtagttcttagcttcgagct |
| | | ccttcacgcccccgctactgtttgtgggccaaagaagtccaccaatctcgtcaagaacaaatgtgtcaatttc |
| | | aactttaacggcttgacaggtaccggcgtacttaccgaatctaacaaaaagttccttcccttccagcaatttgg |
| | | cagagatatcgcagatacaacagacgcagtacgagacccccagaccctggagattttggacatcacaccat |
| | | gttcattcggaggggtcagcgtcatcactccaggaactaataccagcaaccaggtagcagttctctaccaag |
| | | atgttaactgcactgaagtgccagttgcaattcacgctgaccaactgactcccacttggcgggtatactccact |
| | | ggttccaatgtctttcagacacgcgcaggatgtctgatcggggccgaacacgtaaataacagctatgaatgt |
| | | gatatccctattggggccggcatttgcgctagctatcagacacaaacaaattctccccgacgagcacgctcag |
| | | ttgcatctcaatccataattgcctacaccatgagtctgggtgcagaaaactctgtagcttattctaacaattcc |
| | | attgctatacccaccaattttaccatctcagtaacaacagagatccttccagtgagcatgaccaagacctctgt |
| | | tgattgcactatgtacatctgcggtgattcaacagaatgttccaatcttttgcttcaatatggctcattctgtact |
| | | caactcaaccgagccctgaccggcattgctgtggaacaggataaaaatactcaagaggtctttgcccaagtc |
| | | aaacagatttacaaaacaccaccaatcaaggattttggtggcttcaatttcagtcagatactccctgacccttc |
| | | caagcctagcaagcggtcattcattgaagatttgcttttcaataaggtaaccttggcagacgccgggtttatca |
| | | agcagtacggcgactgtcttggcgacatagctgcccgggatttgatttgtgcccagaagtttaatggcctgact |
| | | gtactccctcccctgctgacagatgaaatgatcgcacagtacactagcgctttgctcgccggtacaattacctc |
| | | tgggtggacatttggggctggtgccgccctccaaattcccttcgcaatgcaaatggcttaccgcttcaacggc |
| | | ataggcgtcactcagaacgtcttgtacgagaaccagaaacttatcgctaaccaatttaattccgcaatcggga |
| | | agattcaggatagccttagtagcactgcttcagctctgggcaagctgcaagatgtcgtcaatcaaaatgcaca |
| | | ggcacttaacacactggttaagcagttgtctagtaattttggggctatttcctctgtccttaatgatatcttgagt |
| | | cgattggacaaagtagaagctgaggtccagatagatcggttgataactggacggttgcagagcctgcagact |
| | | tacgtcacccagcagctcattcgcgctgcagagatccgcgcttcagctaacttggctgctacaaaaatgtcag |
| | | aatgcgtcctggggcagtctaaacgggttgacttttgtggcaagggttatcacctgatgagctttcctcaaagt |
| | | gcccctcacggtgtcgtcttcctccacgtaacttatgtccctgcccaggaaaaaaacttcaccacagcacccg |
| | | ctatatgccatgacgggaaagcacatttccctcgagagggtgtattcgtgagcaatggaacccattggtttgt |
| | | aacacagcgaaacttttacgaacctcaaatcattactactgacaatactttcgtctcaggcaactgcgatgta |
| | | gtaataggcatagttaacaatacagtttatgatcctctgcaaccagagctcgatagctttaaggaggagcttg |
| | | acaagtactttaaaaatcatacatctcctgacgtagaccttggggatattagcggcatcaatgcttcagtcgtt |
| | | aatattcaaaaagagattgaccgcctgaatgaggtggccaaaaaccttaacgaaagcttgatagatttgcaa |
| | | gagctgggaaagtatgagcaatatataaaatggccttggtacatctggcttggcttcatcgccgggctcatcg |
| | | ccattgtcatggtaactataatgctttgttgcatgaccagctgttgcagctgtctgaagggctgttgttcatgcg |
| | | gaagttgttgcaaatttgatgaagacgactctgaaccagtactgaagggggtgaagcttcattacacctga |

### V. Codon Optimized Omicron Sequences

TABLE 2
Exemplary Omicron 2P Sequences and Nucleotides Encoding Same

| | | |
|---|---|---|
| 31 | Omicron | MFVFLVLLPLVSSQCVNLITRTQSYTNSFTRGVYYPDKVFRSSVLHSTQDLFL |
| | BA.5 2P | PFFSNVTWFHAISGTNGIKRFDNPVLPFNDGVYFASTEKSNIIRGWIFGTTLDS |
| | Spike | KTQSLLIVNNATNVVIKVCEFQFCNDPFLDVYYHKNNKSWMESEFRVYSSAN |
| | protein | NCTFEYVSQPFLMDLEGKQGNFKNLREFVFKNIDGYFKIYSKHTPINLGRDLP |
| | | QGFSALEPLVDLPIGINITRFQTLLALHRSYLTPGDSSSGWTAGAAAYYVGYL |
| | | QPRTFLLKYNENGTITDAVDCALDPLSETKCTLKSFTVEKGIYQTSNFRVQPT |
| | | ESIVRFPNITNLCPFDEVFNATRFASVYAWNRKRISNCVADYSVLYNFAPFFAF |
| | | KCYGVSPTKLNDLCFTNVYADSFVIRGNEVSQIAPGQTGNIADYNYKLPDDFT |
| | | GCVIAWNSNKLDSKVGGNYNYRYRLFRKSNLKPFERDISTEIYQAGNKPCNG |
| | | VAGVNCYFPLQSYGFRPTYGVGHQPYRVVVLSFELLHAPATVCGPKKSTNLV |
| | | KNKCVNFNFNGLTGTGVLTESNKKFLPFQQFGRDIADTTDAVRDPQTLEILDI |
| | | TPCSFGGVSVITPGTNTSNQVAVLYQGVNCTEVPVAIHADQLTPTWRVYSTG |
| | | SNVFQTRAGCLIGAEYVNNSYECDIPIGAGICASYQTQTKSHRRARSVASQSII |
| | | AYTMSLGAENSVAYSNNSIAIPTNFTISVTTEILPVSMTKTSVDCTMYICGDST |
| | | ECSNLLLQYGSFCTQLKRALTGIAVEQDKNTQEVFAQVKQIYKTPPIKYFGGF |
| | | NFSQILPDPSKPSKRSFIEDLLFNKVTLADAGFIKQYGDCLGDIAARDLICAQK |
| | | FNGLTVLPPLLTDEMIAQYTSALLAGTITSGWTFGAGAALQIPFAMQMAYRF |
| | | NGIGVTQNVLYENQKLIANQFNSAIGKIQDSLSSTASALGKLQDVVNHNAQA |
| | | LNTLVKQLSSKFGAISSVLNDILSRLDPPEAEVQIDRLITGRLQSLQTYVTQQLI |
| | | RAAEIRASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYEPQIITT |
| | | DNTFVSGNCDVVIGIVNNTVYDPLQPELDSFKEELDKYFKNHTSPDVDLGDIS |
| | | GINASVVNIQKEIDRLNEVAKNLNESLIDLQELGKYEQYIKWPWYIWLGFIAG |
| | | LIAIVMVTIMLCCMTSCCSCLKGCCSCGSCCKFDEDDSEPVLKGVKLHYT |
| 32 | Omicron | ATGTTCGTGTTCCTGGTGCTGCTGCCTCTGGTGTCCAGCCAGTGTGTGAACCTGATCACCCG |
| | BA.5 2P | GACACAGAGCTACACCAACAGCTTCACCAGGGGCGTGTACTACCCCGACAAGGTGTTCAGA |
| | Codon | TCCAGCGTGCTGCACTCTACCCAGGACCTGTTCCTGCCTTTCTTCAGCAACGTGACCTGGTTC |
| | Strategy | CACGCCATCAGCGGCACCAACGGCATCAAGAGATTCGACAACCCCGTGCTGCCCTTCAACG |
| | 1 (TF) | ACGGGGTGTACTTTGCCAGCACCGAGAAGTCCAACATCATCAGAGGCTGGATCTTCGGCAC |
| | | CACACTGGACAGCAAGACCCAGAGCCTGCTGATCGTGAACAACGCCACCAACGTGGTCATC |
| | | AAAGTGTGCGAGTTCCAGTTCTGCAACGACCCCTTCCTGGACGTGTACTATCACAAGAACAA |
| | | CAAGAGCTGGATGGAAAGCGAGTTCCGGGTGTACAGCAGCGCCAACAACTGCACCTTCGA |
| | | GTACGTGTCCCAGCCTTTCCTGATGGACCTGGAAGGCAAGCAGGGCAACTTCAAGAACCTG |
| | | CGCGAGTTCGTGTTTAAGAACATCGACGGCTACTTCAAGATCTACAGCAAGCACACCCCTAT |
| | | CAACCTGGGCAGAGATCTGCCTCAGGGCTTCTCTGCTCTGGAACCCCTGGTGGATCTGCCCA |
| | | TCGGCATCAACATCACCCGGTTTCAGACACTGCTGGCCCTGCACAGAAGCTACCTGACACCT |
| | | GGCGATAGCAGCAGCGGATGGACAGCTGGTGCCGCCGCTTACTATGTGGGCTACCTGCAG |
| | | CCTAGAACCTTCCTGCTGAAGTACAACGAGAACGGCACCATCACCGACGCCGTGGATTGTG |
| | | CTCTGGATCCTCTGAGCGAGACAAAGTGCACCCTGAAGTCCTTCACCGTGGAAAAGGGCAT |
| | | CTACCAGACCAGCAACTTCCGGGTGCAGCCCACCGAATCCATCGTGCGGTTCCCCAATATCA |
| | | CCAATCTGTGCCCCTTCGATGAGGTGTTCAATGCCACCAGATTCGCCAGCGTGTACGCCTGG |
| | | AACCGGAAGAGAATCAGCAACTGCGTGGCCGACTACTCCGTGCTGTACAACTTCGCCCCAT |
| | | TCTTCGCCTTCAAGTGCTACGGCGTGTCCCCTACCAAGCTGAACGACCTGTGCTTCACCAAT |
| | | GTGTACGCCGACAGCTTCGTGATCCGGGGCAATGAGGTGTCCCAGATTGCCCCTGGACAGA |
| | | CCGGCAATATCGCCGACTACAACTACAAGCTGCCCGACGACTTCACCGGCTGTGTGATCGCC |
| | | TGGAATAGCAACAAGCTGGACTCCAAAGTCGGCGGCAACTACAATTACCGGTACAGGCTGT |
| | | TCCGGAAGTCCAATCTGAAGCCCTTCGAGCGGGACATCTCCACCGAGATCTATCAGGCCGG |
| | | CAACAAGCCCTGTAATGGCGTGGCAGGCGTGAACTGCTACTTCCCACTGCAGTCCTACGGC |
| | | TTCAGACCCACATACGGCGTGGGCCACCAGCCTTATAGAGTGGTGGTGCTGAGCTTCGAGC |
| | | TGCTGCATGCTCCTGCCACAGTGTGCGGCCCTAAGAAAAGCACCAACCTCGTGAAGAACAA |
| | | ATGCGTGAACTTCAACTTCAACGGCCTGACCGGCACCGGCGTGCTGACAGAGAGCAACAAG |
| | | AAGTTCCTGCCATTCCAGCAGTTCGGCCGGGACATTGCCGATACCACAGATGCTGTCAGAG |
| | | ATCCCCAGACACTGGAAATCCTGGACATCACCCCTTGCAGCTTCGGCGGAGTGTCTGTGATC |
| | | ACCCCTGGCACCAACACCAGCAATCAGGTGGCAGTGCTGTACCAGGGCGTCAACTGTACAG |
| | | AGGTGCCAGTGGCCATTCACGCCGATCAGCTGACCCCTACTTGGCGGGTGTACTCCACAGG |
| | | CAGCAATGTGTTTCAGACCAGAGCCGGCTGTCTGATTGGCGCCGAGTATGTGAACAACAGC |
| | | TACGAGTGCGACATCCCCATCGGAGCCGGCATCTGTGCCAGCTACCAGACACAGACCAAGA |
| | | GCCACAGACGGGCTAGAAGCGTGGCCAGCCAGAGCATCATTGCCTACACAATGTCTCTGGG |
| | | CGCCGAGAACAGCGTGGCCTACAGCAACAACTCTATCGCTATCCCCACCAACTTCACCATCA |
| | | GCGTGACCACAGAGATCCTGCCAGTGTCCATGACCAAGACCAGCGTGGACTGCACCATGTA |
| | | CATCTGCGGCGATTCCACCGAGTGCTCCAACCTGCTGCTGCAGTACGGCAGCTTCTGCACCC |
| | | AGCTGAAGAGAGCCCTGACAGGGATTGCCGTGGAACAGGACAAGAACACCCAAGAGGTGT |
| | | TCGCCCAAGTGAAGCAGATCTACAAGACCCCTCCTATCAAGTACTTCGGCGGGTTCAACTTC |
| | | TCCCAGATTCTGCCCGATCCTAGCAAGCCCAGCAAGCGGAGCTTCATCGAGGACCTGCTGTT |
| | | CAACAAAGTGACACTGGCCGACGCCGGCTTCATCAAGCAGTATGGCGATTGCCTGGGCGAC |
| | | ATTGCAGCCAGGGATCTGATTTGCGCCCAGAAGTTTAACGGACTGACAGTGCTGCCTCCTCT |
| | | GCTGACCGATGAGATGATCGCCCAGTACACATCTGCCCTGCTGGCCGGCACAATCACAAGC |
| | | GGCTGGACATTTGGAGCTGGCGCCGCTCTGCAGATCCCCTTTGCTATGCAGATGGCCTACC |
| | | GGTTCAATGGCATCGGAGTGACCCAGAATGTGCTGTACGAGAACCAGAAGCTGATCGCCA |
| | | ACCAGTTCAACAGCGCCATCGGCAAGATCCAGGACAGCCTGAGCAGTACAGCCAGCGCTCT |
| | | GGGAAAGCTGCAGGACGTGGTCAACCACAATGCCCAGGCACTGAACACCCTGGTCAAGCA |
| | | GCTGAGCAGCAAGTTCGGCGCCATCTCTAGCGTGCTGAACGATATCCTGAGCAGACTGGAC |
| | | CCTCCTGAGGCCGAGGTGCAGATCGACAGACTGATCACAGGCAGACTGCAGAGCCTCCAG |
| | | ACCTACGTGACACAGCAGCTGATTAGAGCCGCCGAGATCAGAGCCTCTGCCAATCTGGCCG |
| | | CCACCAAGATGTCTGAGTGTGTGCTGGGCCAGAGCAAGAGAGTGGACTTTTGCGGCAAGG |
| | | GCTACCACCTGATGAGCTTCCCTCAGTCTGCACCACACGGCGTGGTGTTTCTGCACGTGACA |
| | | TACGTGCCCGCTCAAGAGAAGAATTTCACCACCGCTCCAGCCATCTGCCACGACGGCAAAG |
| | | CCCACTTTCCTAGAGAAGGCGTGTTCGTGTCCAACGGCACCCATTGGTTCGTGACCCAGCGG |
| | | AACTTCTACGAGCCCCAGATCATCACCACCGACAACACCTTCGTGTCCGGCAACTGCGACGT |
| | | CGTGATCGGCATTGTGAACAATACCGTGTACGACCCTCTGCAGCCCGAGCTGGACAGCTTC |
| | | AAAGAGGAACTGGATAAGTACTTTAAGAACCACACAAGCCCCGACGTGGACCTGGGCGAT |
| | | ATCAGCGGAATCAATGCCAGCGTCGTGAACATCCAGAAAGAGATCGACCGGCTGAACGAG |
| | | GTGGCCAAGAATCTGAACGAGAGCCTGATCGACCTGCAAGAACTGGGGAAGTACGAGCAG |
| | | TATATCAAGTGGCCTTGGTACATCTGGCTGGGCTTTATCGCCGGACTGATTGCCATCGTGAT |
| | | GGTCACAATCATGCTGTGCTGCATGACCAGCTGCTGCTCTTGCCTGAAGGGCTGCTGTAGCT |
| | | GTGGCTCCTGCTGCAAGTTCGACGAGGACGATTCTGAGCCCGTGCTGAAGGGCGTGAAGCT |
| | | GCACTACACATGA |
| 33 | Omicron | ATGTTCGTGTTTCTGGTACTCCTTCCCCTTGTGTCTTCACAATGCGTTAATTTGATCACACGA |
| | BA.5 2P | ACTCAATCATATACTAATTCTTTCACGCGAGGCGTATACTATCCGGATAAGGTCTTCCGCTCT |
| | Codon | TCCGTACTCCACAGTACACAGGACCTTTTCCTGCCATTCTTTAGCAATGTCACTTGGTTCCAT |
| | Strategy | GCAATATCTGGAACAAATGGTATTAAACGATTTGATAATCCTGTCTTACCATTCAATGACGG |
| | 2 (TW) | CGTCTACTTCGCTAGTACCGAGAAAAGCAATATCATCAGAGGTTGGATCTTCGGTACCACTC |
| | | TTGATTCTAAGACTCAGAGCTTGCTTATTGTCAATAATGCCACCAATGTTGTTATCAAAGTTT |
| | | GCGAATTTCAATTTTGTAACGACCCTTTCTTGGACGTGTACTATCACAAGAACAACAAGTCA |
| | | TGGATGGAGAGTGAGTTCCGAGTATATTCTTCTGCAAACAACTGTACCTTCGAGTATGTCAG |
| | | TCAACCCTTCTTGATGGACCTGGAAGGGAAGCAAGGCAACTTTAAGAATCTTCGCGAATTC |
| | | GTATTTAAGAACATTGATGGCTACTTCAAGATCTACTCCAAACATACCCCCATCAATCTTGGG |
| | | CGTGATTTGCCCCAAGGCTTTAGTGCGCTCGAGCCTCTTGTGGATCTCCCAATTGGAATCAA |
| | | CATTACTCGCTTTCAAACGCTGCTGGCTTTGCATAGGTCCTATCTTACTCCTGGAGACTCTTC |
| | | ATCTGGCTGGACGGCCGGGGCAGCAGCCTACTACGTGGGATATCTTCAACCCAGGACTTTT |
| | | CTGCTTAAATACAACGAAAATGGAACTATTACAGATGCAGTTGACTGCGCTCTTGATCCCCT |
| | | CTCCGAAACTAAATGTACACTCAAGAGCTTTACAGTAGAGAAAGGGATATATCAAACCTCA |
| | | AACTTTCGGGTCCAACCAACAGAAAGTATCGTGAGATTCCCCAACATAACGAATTTGTGTCC |
| | | ATTCGATGAAGTTTTCAACGCCACCAGGTTCGCGTCCGTATATGCTTGGAACCGAAAGCGG |
| | | ATTAGTAACTGTGTAGCCGACTATTCTGTACTGTATAACTTTGCTCCTTTCTTTGCCTTCAAAT |
| | | GTTATGGAGTGAGCCCAACCAAACTGAACGACTTGTGCTTTACTAACGTCTACGCCGACAGT |
| | | TTTGTCATCAGAGGGAACGAGGTTTCCCAGATCGCACCAGGACAGACTGGTAATATAGCCG |
| | | ATTACAATTATAAATTGCCAGATGACTTTACAGGCTGCGTCATAGCTTGGAATTCAAATAAG |
| | | CTCGATTCCAAGGTCGGTGGAAATTATAATTATCGATACCGGTTATTCAGGAAAAGCAATCT |
| | | CAAGCCTTTTGAACGTGACATCAGCACTGAGATCTATCAAGCAGGCAATAAACCCTGTAATG |
| | | GCGTAGCCGGTGTAAATTGTTATTTCCCTCTGCAAAGTTACGGATTTAGGCCCACCTACGGA |
| | | GTGGGTCACCAACCATACCGCGTAGTCGTTCTTAGTTTTGAGCTGCTGCATGCCCCAGCGAC |
| | | AGTTTGTGGACCAAAGAAATCCACCAATCTCGTCAAGAACAAATGCGTGAATTTTAATTTTA |
| | | ACGGTCTCACCGGCACAGGGGTCTTAACAGAGTCCAACAAGAAGTTTCTCCCTTTCCAACAA |
| | | TTCGGACGGGATATAGCCGATACCACCGATGCAGTGCGAGATCCCCAAACCTTGGAGATCC |
| | | TTGATATAACACCTTGCTCATTCGGTGGCGTCTCCGTCATTACTCCTGGAACGAACACCTCTA |
| | | ATCAAGTGGCAGTCTTGTATCAAGGTGTTAACTGCACGGAAGTCCCTGTCGCAATCCATGCT |
| | | GATCAATTGACACCCACATGGCGGGTCTATAGCACCGGCTCAAATGTATTTCAGACACGGG |
| | | CAGGTTGTCTGATTGGTGCAGAGTATGTTAATAACTCATATGAATGCGATATCCCTATCGGA |
| | | GCTGGAATTTGCGCTTCCTATCAAACCCAAACAAAATCCCATCGGAGAGCTAGATCCGTTGC |
| | | CTCTCAATCTATTATTGCATATACAATGTCTCTGGGAGCGGAGAACAGTGTGGCCTACTCCA |
| | | ATAACAGCATTGCGATTCCAACTAACTTCACTATCAGTGTGACTACTGAAATTTTACCAGTGT |
| | | CTATGACCAAGACTTCTGTAGATTGTACCATGTATATATGCGGTGACTCCACCGAGTGTAGC |
| | | AACCTGCTGTTGCAGTACGGCAGTTTCTGTACCCAATTGAAACGTGCCCTGACTGGCATCGC |
| | | CGTGGAACAGGATAAGAATACTCAAGAGGTTTTCGCCCAAGTAAAACAAATTTATAAAACA |
| | | CCACCTATCAAATATTTTGGCGGATTCAACTTCTCCCAGATCTTACCCGACCCTTCTAAACCA |
| | | AGCAAGCGGTCATTCATAGAAGATCTGTTGTTTAATAAAGTTACGCTGGCGGACGCTGGAT |
| | | TCATCAAGCAGTACGGCGATTGCTTAGGGGATATCGCCGCTCGGGATCTGATCTGTGCCCA |
| | | AAAGTTTAATGGACTCACCGTTCTGCCACCATTGTTGACTGACGAAATGATTGCCCAATACA |
| | | CATCCGCTTTGCTGGCGGGCACAATTACCTCTGGCTGGACGTTTGGTGCCGGGGCAGCACT |
| | | TCAGATCCCGTTCGCCATGCAAATGGCCTACAGATTTAATGGCATAGGAGTGACACAAAAC |
| | | GTCCTGTATGAAAACCAAAAGTTGATTGCCAATCAATTTAACTCCGCTATAGGGAAAATTCA |
| | | GGATTCACTGTCATCAACTGCTTCCGCATTGGGGAAACTCCAGGACGTGGTCAACCACAAC |
| | | GCACAGGCACTCAACACGTTGGTCAAACAATTGTCTTCTAAATTCGGGGCTATATCTTCTGT |
| | | GCTCAATGATATTCTTTCACGCCTGGATCCACCCGAAGCTGAAGTTCAAATCGATAGGCTCA |
| | | TAACGGGTAGGCTCCAGTCTCTTCAAACTTATGTGACGCAACAATTAATCCGCGCGGCAGA |
| | | AATTCGTGCGTCCGCTAATCTCGCGGCCACAAAGATGAGTGAATGCGTTCTGGGGCAGTCT |
| | | AAGCGCGTTGATTTCTGTGGAAAGGGTTATCATCTCATGTCTTTCCCCCAATCAGCACCACA |
| | | CGGAGTTGTTTTCCTTCATGTAACTTATGTTCCTGCACAAGAGAAGAACTTCACAACGGCTC |
| | | CAGCCATTTGTCACGATGGAAAAGCACATTTCCCTCGCGAGGGTGTCTTTGTCAGTAACGGA |
| | | ACCCATTGGTTCGTGACTCAACGCAATTTCTATGAACCCCAAATTATTACAACCGACAATACC |
| | | TTTGTTAGCGGAAATTGTGACGTGGTCATAGGCATTGTCAATAATACAGTATACGACCCGCT |
| | | CCAACCTGAGTTAGACTCTTTCAAGGAAGAGCTGGATAAATACTTTAAGAATCATACATCTC |
| | | CTGATGTGGACTTGGGCGATATTAGTGGTATCAATGCTAGCGTAGTTAATATCCAAAAGGA |
| | | AATTGACAGGCTGAATGAGGTTGCAAAGAACCTGAATGAGAGCCTTATTGACTTACAAGAG |
| | | CTTGGAAAATACGAACAATACATTAAATGGCCCTGGTACATCTGGCTGGGCTTTATAGCTGG |
| | | CTTGATCGCCATCGTTATGGTAACCATCATGTTGTGTTGTATGACCTCATGTTGTAGTTGCCT |
| | | TAAAGGATGTTGTTCATGTGGGAGTTGTTGCAAGTTTGATGAAGATGATAGTGAACCTGTT |
| | | CTTAAAGGGGTTAAACTGCACTACACTTAG |
| 34 | Omicron | ATGTTTGTCTTCCTGGTGCTGTTGCCTCTGGTGAGCTCTCAGTGTGTCAACCTTATCACCCGG |
| | BA.5 2P | ACACAGTCCTACACTAACTCTTTCACGCGCGGAGTGTACTATCCTGACAAGGTTTTTCGGAG |
| | Codon | TAGTGTCCTCCACTCGACACAAGACCTCTTCCTGCCTTTTTTTTCCAATGTGACATGGTTCCAC |
| | Strategy | GCCATCTCCGGAACAAATGGGATAAAACGCTTCGACAACCCTGTGTTGCCTTTCAACGATGG |
| | 3 (GS) | CGTGTACTTTGCCTCGACTGAGAAGTCTAATATCATCCGGGGTTGGATCTTCGGTACTACCC |
| | | TGGACAGTAAAACTCAAAGTCTGCTGATTGTGAATAATGCCACCAATGTGGTAATCAAAGT |
| | | GTGTGAATTCCAGTTTTGTAACGATCCTTTCCTGGATGTGTACTACCACAAGAACAACAAGA |
| | | GCTGGATGGAGTCTGAGTTCAGAGTATATTCCAGCGCCAACAACTGCACTTTTGAGTACGT |
| | | GAGCCAGCCCTTCCTCATGGACTTGGAAGGAAAGCAGGGCAATTTCAAAAACCTGCGGGA |
| | | ATTTGTGTTCAAGAACATCGACGGCTATTTCAAAATCTACAGTAAACACACACCCATTAACCT |
| | | GGGCCGGGATCTGCCTCAGGGATTCTCTGCCCTGGAGCCACTAGTAGACCTACCCATCGGC |
| | | ATCAACATTACCCGCTTTCAAACACTTCTAGCCCTCCACAGGAGCTACCTCACTCCTGGCGAC |
| | | AGTAGCTCCGGCTGGACCGCCGGCGCGGCCGCGTACTACGTGGGGTACCTGCAACCTCGTA |
| | | CCTTCCTGCTCAAGTATAATGAAAATGGCACCATAACAGATGCTGTGGACTGCGCACTTGAT |
| | | CCCCTGAGCGAGACTAAATGCACCCTCAAAAGCTTCACCGTGGAGAAGGGGATTTACCAGA |
| | | CCAGCAACTTTAGAGTCCAGCCGACAGAGAGCATCGTGAGATTCCCAAACATAACCAACCT |
| | | GTGTCCTTTTGACGAAGTCTTCAATGCTACCCGGTTTGCGAGCGTGTACGCCTGGAATAGGA |
| | | AGAGGATCAGCAACTGTGTGGCTGATTACTCGGTGCTGTACAACTTCGCCCCTTTCTTTGCC |
| | | TTTAAATGCTATGGGGTGTCTCCCACTAAGCTGAATGATCTGTGTTTCACTAACGTTTATGCC |
| | | GACTCTTTCGTCATCAGAGGCAATGAGGTAAGCCAAATTGCCCCTGGACAGACGGGGAACA |
| | | TCGCCGACTACAACTACAAACTCCCTGACGACTTTACAGGATGTGTAATCGCGTGGAACTCC |
| | | AATAAACTGGACTCTAAGGTCGGGGGCAACTATAACTATAGATACAGACTGTTCAGAAAGT |
| | | CCAACCTGAAGCCCTTTGAAAGAGATATTAGCACAGAAATATATCAGGCCGGCAATAAACC |
| | | ATGCAATGGAGTTGCCGGGGTGAACTGCTACTTTCCTCTGCAAAGTTATGGGTTCAGACCTA |
| | | CATACGGGGTAGGGCACCAGCCCTACAGAGTGGTGGTACTCAGCTTTGAGTTGTTGCACGC |
| | | GCCTGCCACTGTCTGCGGCCCGAAGAAAAGCACCAACCTGGTGAAGAACAAATGTGTGAAC |
| | | TTCAACTTCAACGGCTTAACCGGGACCGGCGTTCTGACCGAGTCAAACAAGAAGTTCCTTCC |
| | | TTTCCAACAGTTTGGCAGAGACATCGCTGACACCACGGATGCAGTTCGCGACCCACAGACT |
| | | CCATTCATGCCGACCAGTTAACCCCTACCTGGAGGGTGTACTCCACAGGTTCCAACGTTTTC |
| | | CTGGAGATCCTCGATATCACCCCTTGCTCCTTTGGCGGCGTGAGCGTTATCACCCCTGGTAC |
| | | AAATACTTCTAATCAGGTTGCTGTCCTGTACCAAGGTGTGAATTGTACAGAGGTGCCCGTGG |
| | | CAGACAAGAGCTGGCTGCCTGATTGGAGCCGAATATGTCAACAACAGTTACGAATGCGATA |
| | | TACCCATTGGAGCCGGCATCTGTGCCTCATACCAAACACAGACCAAAAGTCATCGGAGAGC |
| | | CAGATCAGTGGCCTCCCAGTCTATCATCGCCTATACAATGAGTCTGGGTGCAGAAAACAGT |
| | | GTAGCTTACTCTAACAACAGCATCGCCATCCCTACTAACTTCACAATCAGCGTTACCACCGAA |
| | | ATCCTGCCAGTGAGCATGACCAAGACAAGCGTAGATTGCACCATGTACATCTGCGGAGACT |
| | | CGACGGAGTGCAGCAACCTGTTACTGCAGTATGGCAGCTTCTGCACACAGCTTAAGCGAGC |
| | | CCTGACCGGAATTGCTGTGGAGCAGGATAAGAATACACAGGAGGTGTTCGCACAGGTAAA |
| | | GCAAATCTACAAGACCCCTCCAATTAAGTACTTCGGTGGCTTCAACTTTTCTCAGATTCTCCC |
| | | TGATCCATCGAAACCCAGCAAAAGATCCTTCATCGAGGACCTCCTGTTCAACAAGGTGACTT |
| | | TGGCAGACGCGGGCTTCATTAAGCAGTACGGAGACTGTCTGGGCGACATCGCTGCCCGAG |
| | | ACCTGATCTGTGCTCAGAAGTTTAACGGGCTGACCGTGCTGCCCCCCCTCCTGACTGATGAG |
| | | ATGATAGCACAGTACACCAGCGCCCTGTTGGCCGGTACCATCACATCCGGCTGGACATTTG |
| | | GTGCCGGCGCAGCATTGCAAATCCCCTTCGCCATGCAGATGGCTTACCGGTTTAACGGCATC |
| | | GGCGTGACCCAGAATGTATTGTACGAGAACCAGAAGCTGATTGCCAACCAATTCAACTCAG |
| | | CCATCGGAAAGATCCAGGACAGTCTGAGCAGCACAGCTAGCGCCCTCGGCAAGTTGCAGG |
| | | ATGTCGTGAACCACAACGCCCAGGCTCTCAACACACTAGTGAAGCAGTTGAGCTCCAAGTT |
| | | CGGAGCAATTTCGAGCGTGCTCAATGATATCTTGAGCAGATTGGACCCTCCTGAGGCCGAA |
| | | GTCCAGATCGATCGGCTGATTACAGGGAGACTGCAGAGCCTGCAGACCTATGTAACCCAGC |
| | | AGCTGATAAGAGCAGCCGAGATTCGGGCCTCTGCGAATCTGGCTGCCACCAAGATGAGCG |
| | | AGTGTGTCCTGGGTCAGTCTAAGAGAGTGGACTTCTGTGGCAAGGGATACCACCTCATGTC |
| | | TTTTCCACAAAGCGCCCCCCATGGGGTGGTGTTCCTGCACGTCACCTACGTGCCCGCCCAGG |
| | | AAAAAAACTTTACCACTGCGCCTGCCATCTGCCATGACGGGAAAGCCCATTTCCCTCGAGAA |
| | | GGCGTGTTCGTTTCTAACGGCACGCACTGGTTTGTGACTCAGAGGAACTTCTACGAGCCCCA |
| | | GATCATCACCACAGACAATACCTTCGTGTCCGGCAACTGTGATGTGGTGATTGGCATCGTGA |
| | | ATAACACAGTGTATGACCCTCTTCAGCCAGAGCTGGATTCGTTTAAGGAGGAACTGGATAA |
| | | ATATTTCAAGAATCACACATCCCCTGATGTGGACCTGGGGGATATCTCTGGGATCAATGCTT |
| | | CAGTGGTTAACATTCAGAAGGAAATCGACAGACTGAACGAAGTGGCTAAGAATCTCAACGA |
| | | ATCCCTCATTGACCTGCAGGAGCTGGGCAAATACGAACAATACATCAAGTGGCCCTGGTAC |
| | | ATCTGGCTGGGCTTCATCGCTGGCTTAATCGCTATCGTCATGGTGACAATTATGCTGTGTTG |
| | | CATGACTTCTTGCTGCTCCTGCCTCAAGGGCTGTTGCAGTTGTGGGTCTTGCTGCAAATTTG |
| | | ATGAAGATGATTCTGAGCCAGTGCTCAAGGGCGTGAAACTGCACTACACCtga |
| 35 | Omicron | ATGTTCGTGTTCCTGGTGCTGCTGCCCCTGGTGAGCTCTCAGTGCGTGAACCTGATCACAAG |
| | BA.5 2P | AACACAGAGCTACACCAACAGCTTCACAAGAGGCGTGTACTACCCCGACAAGGTGTTCAGA |
| | Codon | AGCAGCGTGCTACACAGCACCCAAGACCTGTTTCTGCCCTTCTTCAGCAACGTGACCTGGTT |
| | Strategy | CCACGCCATCAGCGGCACCAACGGCATCAAGAGATTCGACAACCCCGTGCTGCCCTTCAAC |
| | 4 (AZ) | GACGGCGTGTACTTCGCTAGCACCGAGAAGAGCAACATCATCAGAGGCTGGATCTTCGGCA |
| | | CCACCCTGGATAGTAAAACTCAGAGCCTGCTGATAGTGAACAATGCCACCAACGTGGTGAT |
| | | CAAGGTGTGCGAGTTTCAGTTCTGCAACGACCCCTTCCTGGACGTGTACTACCACAAGAACA |
| | | ACAAGAGCTGGATGGAGAGCGAGTTCCGTGTGTATAGCTCCGCTAACAACTGCACCTTCGA |
| | | GTACGTGTCTCAGCCCTTCCTGATGGACCTGGAGGGCAAGCAAGGCAACTTCAAGAATCTG |
| | | AGAGAGTTCGTGTTCAAGAACATCGACGGCTACTTCAAGATCTACAGCAAGCACACCCCCA |
| | | TCAACCTGGGCAGAGACCTGCCCCAAGGCTTCAGCGCCCTGGAGCCCCTGGTGGACCTGCC |
| | | CATCGGCATCAACATCACAAGATTTCAGACCCTGCTGGCCCTGCACAGAAGCTATCTGACCC |
| | | CCGGCGACAGCTCATCGGGCTGGACCGCTGGCGCAGCGGCCTACTACGTGGGCTACCTGCA |
| | | GCCTAGAACCTTCCTGCTGAAGTACAACGAGAACGGTACGATTACTGACGCCGTGGACTGT |
| | | GCCCTGGACCCCCTGAGCGAGACCAAGTGCACCCTGAAGAGCTTCACCGTGGAGAAGGGC |
| | | ATCTATCAGACAAGCAACTTCAGAGTGCAGCCCACCGAGAGCATCGTGAGATTCCCCAACA |
| | | TCACCAACCTGTGCCCCTTCGACGAGGTGTTCAACGCCACAAGATTCGCTAGCGTGTACGCT |
| | | TGGAACAGAAAGAGAATCAGCAACTGCGTGGCCGACTACAGCGTGCTGTACAACTTCGCCC |
| | | CCTTCTTCGCCTTCAAGTGCTATGGCGTGAGCCCCACCAAGCTGAACGACCTGTGCTTCACC |
| | | AACGTGTACGCCGACAGCTTCGTGATCAGAGGCAACGAGGTGTCTCAGATCGCCCCCGGGC |
| | | AGACCGGCAACATCGCCGACTATAACTACAAACTGCCCGACGACTTCACCGGCTGCGTGAT |
| | | CGCCTGGAACAGCAACAAGCTGGACAGCAAGGTGGGCGGCAACTATAATTATAGATACAG |
| | | ACTGTTCAGAAAGAGCAACCTGAAGCCCTTCGAGAGAGACATCAGCACCGAGATCTACCAA |
| | | GCCGGCAACAAGCCCTGCAACGGCGTGGCCGGCGTAAACTGCTACTTTCCCCTGCAGAGCT |
| | | ACGGCTTCAGACCCACCTACGGCGTGGGCCATCAGCCCTACAGAGTGGTGGTGCTGAGCTT |
| | | CGAGCTGCTGCACGCGCCCGCTACTGTCTGCGGCCCCAAGAAGAGCACCAACCTGGTGAAG |
| | | AACAAGTGCGTGAACTTCAACTTTAACGGCCTGACCGGAACCGGCGTGCTGACCGAGAGCA |
| | | ACAAGAAGTTCCTGCCATTTCAGCAATTCGGCAGAGACATCGCCGACACCACCGATGCCGT |
| | | GCGTGACCCTCAGACCCTGGAGATCCTGGACATCACCCCGTGCAGTTTCGGCGGCGTGAGC |
| | | GTGATCACCCCCGGCACCAACACAAGCAACCAAGTGGCCGTGCTGTACCAAGGCGTCAACT |
| | | GTACGGAGGTGCCCGTGGCCATCCACGCCGATCAGCTGACCCCCACCTGGAGAGTATATAG |
| | | CACCGGCAGCAACGTGTTTCAGACAAGAGCCGGCTGCCTGATCGGCGCCGAGTACGTGAA |
| | | CAACAGCTACGAGTGCGACATCCCCATCGGCGCCGGCATCTGCGCTAGCTATCAGACACAG |
| | | ACCAAGAGCCACAGAAGAGCTAGAAGCGTGGCTTCTCAGAGCATCATCGCCTACACCATGA |
| | | GCCTGGGCGCCGAGAACAGCGTGGCCTACAGCAACAACAGCATCGCCATCCCCACCAACTT |
| | | CACCATCAGCGTGACCACCGAGATACTGCCCGTGAGCATGACCAAGACAAGCGTGGACTGC |
| | | ACCATGTACATCTGCGGCGACAGCACCGAGTGCAGCAACCTGCTGCTGCAGTACGGCAGCT |
| | | TCTGCACACAGCTGAAGAGAGCCCTGACCGGCATCGCCGTGGAGCAAGACAAGAACACCC |
| | | AAGAGGTGTTCGCCCAAGTGAAGCAGATCTACAAGACCCCCCCCATCAAGTACTTCGGCGG |
| | | CTTCAACTTCAGCCAAATCCTGCCTGACCCTAGCAAGCCTAGCAAGAGAAGCTTCATCGAGG |
| | | ACCTGCTGTTCAACAAGGTGACCCTGGCCGACGCCGGCTTCATCAAGCAGTACGGCGACTG |
| | | CCTGGGGGACATCGCCGCTAGAGACCTGATCTGCGCTCAGAAGTTCAACGGCTTAACTGTG |
| | | CTCCCCCCCCTGCTGACCGACGAGATGATCGCTCAGTATACAAGCGCGCTACTGGCCGGTAC |
| | | CATCACCTCCGGATGGACCTTTGGTGCGGGGGCGGCCCTGCAAATCCCCTTCGCCATGCAG |
| | | ATGGCCTACAGATTCAACGGCATCGGCGTGACACAGAACGTGCTGTACGAGAATCAGAAG |
| | | CTGATCGCCAATCAGTTCAACAGCGCCATCGGCAAGATCCAAGACAGCCTGAGCAGCACCG |
| | | CTAGCGCCCTGGGCAAGCTGCAAGACGTGGTGAACCACAACGCCCAAGCCCTGAACACCCT |
| | | GGTGAAGCAGCTGAGCAGCAAGTTCGGCGCCATCTCGAGCGTGTTGAACGACATCCTGAG |
| | | CAGACTGGACCCCCCCGAGGCCGAGGTGCAGATCGACAGACTGATCACCGGCAGACTGCA |
| | | GAGCCTGCAGACCTACGTGACACAGCAGCTGATCAGAGCCGCCGAGATCAGAGCTAGCGC |
| | | CAACCTGGCCGCCACCAAGATGAGCGAGTGCGTGCTGGGGCAGAGCAAGAGAGTGGACTT |
| | | CTGCGGCAAGGGCTACCACCTGATGAGCTTCCCTCAGAGCGCCCCCCACGGCGTGGTGTTC |
| | | CTGCACGTGACCTACGTGCCCGCCCAAGAGAAGAACTTCACAACCGCCCCCGCGATCTGCC |
| | | ACGACGGCAAGGCCCACTTCCCTAGAGAGGGCGTGTTCGTGAGCAACGGCACCCACTGGTT |
| | | CGTGACACAGAGAAACTTCTACGAGCCTCAGATCATCACCACCGACAACACCTTCGTGAGC |
| | | GGCAACTGCGACGTGGTGATCGGAATAGTTAATAACACGGTATACGATCCCCTGCAGCCCG |
| | | AGCTGGACAGCTTCAAGGAGGAGCTGGACAAGTACTTTAAGAATCATACAAGCCCCGACGT |
| | | GGACCTGGGCGACATAAGCGGGATCAACGCTAGCGTGGTGAACATTCAGAAGGAGATCGA |
| | | CCGGCTGAATGAGGTGGCCAAGAACCTGAACGAGAGCCTGATCGACCTGCAAGAGCTGGG |
| | | CAAGTACGAGCAGTACATCAAGTGGCCCTGGTACATCTGGCTGGGCTTCATCGCCGGCCTG |
| | | ATCGCCATCGTGATGGTGACCATCATGCTGTGCTGCATGACAAGCTGTTGCAGCTGCCTGAA |
| | | AGGTTGCTGCAGCTGTGGCTCGTGCTGCAAGTTCGACGAGGACGACAGCGAGCCCGTGCT |
| | | GAAGGGCGTGAAGCTGCACTACACCTGATGA |
| 36 | Omicron | MFVFLVLLPLVSSQCVNLITRTQSYTNSFTRGVYYPDKVFRSSVLHSTQDLFLPFFSNVTWFHAIH |
| | XBB.1.5 | VSGTNGTKRFDNPALPFNDGVYFASTEKSNIIRGWIFGTTLDSKTQSLLIVNNATNVVIKVCEFQF |
| | 2P Spike | CNDPFLDVYQKNNKSWMESEFRVYSSANNCTFEYVSQPFLMDLEGKEGNFKNLREFVFKNIDG |
| | Protein | YFKIYSKHTPINLERDLPQGFSALEPLVDLPIGINITRFQTLLALHRSYLTPVDSSSGWTAGAAAYY |
| | | VGYLQPRTFLLKYNENGTITDAVDCALDPLSETKCTLKSFTVEKGIYQTSNFRVQPTESIVRFPNIT |
| | | NLCPFHEVFNATTFASVYAWNRKRISNCVADYSVIYNFAPFFAFKCYGVSPTKLNDLCFTNVYAD |
| | | SFVIRGNEVSQIAPGQTGNIADYNYKLPDDFTGCVIAWNSNKLDSKPSGNYNYLYRLFRKSKLKP |
| | | FERDISTEIYQAGNKPCNGVAGPNCYSPLQSYGFRPTYGVGHQPYRVVVLSFELLHAPATVCGP |
| | | KKSTNLVKNKCVNFNFNGLTGTGVLTESNKKFLPFQQFGRDIADTTDAVRDPQTLEILDITPCSF |
| | | GGVSVITPGTNTSNQVAVLYQGVNCTEVPVAIHADQLTPTWRVYSTGSNVFQTRAGCLIGAEY |
| | | VNNSYECDIPIGAGICASYQTQTKSHRRARSVASQSIIAYTMSLGAENSVAYSNNSIAIPTNFTISV |
| | | TTEILPVSMTKTSVDCTMYICGDSTECSNLLLQYGSFCTQLKRALTGIAVEQDKNTQEVFAQVKQ |
| | | IYKTPPIKYFGGFNFSQILPDPSKPSKRSFIEDLLFNKVTLADAGFIKQYGDCLGDIAARDLICAQKF |
| | | NGLTVLPPLLTDEMIAQYTSALLAGTITSGWTFGAGAALQIPFAMQMAYRFNGIGVTQNVLYE |
| | | NQKLIANQFNSAIGKIQDSLSSTASALGKLQDVVNHNAQALNTLVKQLSSKFGAISSVLNDILSRL |
| | | DPPEAEVQIDRLITGRLQSLQTYVTQQLIRAAEIRASANLAATKMSECVLGQSKRVDFCGKGYHL |
| | | MSFPQSAPHGVVFLHVTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHTSPDVDLGDISGINASVV |
| | | NIQKEIDRLNEVAKNLNESLIDLQELGKYEQYIKWPWYIWLGFIAGLIAIVMVTIMLCCMTSCCSC |
| | | LKGCCSCGSCCKFDEDDSEPVLKGVKLHYT |
| 37 | Omicron | ATGTTCGTGTTCCTGGTGCTGCTGCCTCTGGTGTCCAGCCAGTGTGTGAACCTGATCACCCG |
| | XBB.1.5 | GACACAGAGCTACACCAACAGCTTCACCAGGGGCGTGTACTACCCCGACAAGGTGTTCAGA |
| | Codon | TCCAGCGTGCTGCACTCTACCCAGGACCTGTTCCTGCCTTTCTTCAGCAACGTGACCTGGTTC |
| | 2P | CACGCCATCCACGTGTCCGGCACCAATGGCACCAAGAGATTCGACAACCCCGCTCTGCCCTT |
| | Strategy | CAACGACGGGGTGTACTTTGCCAGCACCGAGAAGTCCAACATCATCAGAGGCTGGATCTTC |
| | 1 (TF) | GGCACCACACTGGACAGCAAGACCCAGAGCCTGCTGATCGTGAACAACGCCACCAACGTG |
| | | GTCATCAAAGTGTGCGAGTTCCAGTTCTGCAACGACCCCTTCCTGGACGTGTACCAGAAGA |
| | | ACAACAAGAGCTGGATGGAAAGCGAGTTCCGGGTGTACAGCAGCGCCAACAACTGCACCT |
| | | TCGAGTACGTGTCCCAGCCTTTCCTGATGGACCTGGAAGGCAAAGAGGGCAACTTCAAGAA |
| | | CCTGCGCGAGTTCGTGTTTAAGAACATCGACGGCTACTTCAAGATCTACAGCAAGCACACCC |
| | | CTATCAACCTGGAACGGGATCTGCCTCAGGGCTTCTCTGCTCTGGAACCCCTGGTGGATCTG |
| | | CCCATCGGCATCAACATCACCCGGTTTCAGACACTGCTGGCCCTGCACCGGTCTTACCTGAC |
| | | ACCTGTGGATAGCAGCAGCGGATGGACAGCTGGCGCCGCTGCTTACTATGTGGGCTACCTG |
| | | CAGCCTCGGACCTTCCTGCTGAAGTACAACGAGAACGGCACCATCACCGACGCCGTGGATT |
| | | GTGCTCTGGATCCTCTGAGCGAGACAAAGTGCACCCTGAAGTCCTTCACCGTGGAAAAGGG |
| | | CATCTACCAGACCAGCAACTTCCGGGTGCAGCCCACCGAATCCATCGTGCGGTTCCCCAATA |
| | | TCACCAATCTGTGCCCCTTCCACGAGGTGTTCAATGCCACCACATTCGCCAGCGTGTACGCC |
| | | TGGAACCGGAAGAGAATCAGCAACTGCGTGGCCGACTACAGCGTGATCTACAACTTCGCCC |
| | | CATTCTTCGCCTTCAAGTGCTACGGCGTGTCCCCTACCAAGCTGAACGACCTGTGCTTCACCA |
| | | ATGTGTACGCCGACAGCTTCGTGATCCGGGGCAATGAGGTGTCCCAGATTGCCCCTGGACA |
| | | GACCGGCAATATCGCCGATTACAACTACAAGCTGCCCGACGACTTCACCGGCTGTGTGATC |
| | | GCCTGGAATAGCAACAAGCTGGACTCCAAGCCTAGCGGCAACTACAACTACCTGTACCGGC |
| | | TGTTCAGAAAGAGCAAGCTGAAGCCCTTCGAGCGGGACATCTCCACCGAGATCTATCAGGC |
| | | CGGCAACAAGCCCTGCAATGGCGTGGCAGGCCCTAACTGTTATAGCCCACTGCAGAGCTAC |
| | | GGCTTCAGACCCACATACGGCGTGGGCCACCAGCCTTATAGAGTGGTGGTGCTGAGCTTCG |
| | | AGCTGCTGCATGCTCCTGCCACAGTGTGCGGCCCTAAGAAAAGCACCAACCTCGTGAAGAA |
| | | CAAATGCGTGAACTTCAACTTCAACGGCCTGACCGGCACCGGCGTGCTGACAGAGAGCAAC |
| | | AAGAAGTTCCTGCCATTCCAGCAGTTCGGCCGGGACATTGCCGATACCACAGATGCTGTCA |
| | | GAGATCCCCAGACACTGGAAATCCTGGACATCACCCCTTGCAGCTTCGGCGGAGTGTCTGT |
| | | GATCACCCCTGGCACCAACACCAGCAATCAGGTGGCCGTTCTGTACCAGGGCGTGAACTGT |
| | | ACAGAGGTGCCAGTGGCCATTCACGCCGATCAGCTGACCCCTACTTGGCGGGTGTACTCCA |
| | | CAGGCAGCAATGTGTTTCAGACCAGAGCCGGCTGTCTGATTGGCGCCGAGTATGTGAACAA |
| | | CAGCTACGAGTGCGACATCCCCATCGGAGCCGGCATCTGTGCCAGCTACCAGACACAGACC |
| | | AAGAGCCACAGACGGGCTAGAAGCGTGGCCAGCCAGAGCATCATTGCCTACACAATGTCTC |
| | | TGGGCGCCGAGAACAGCGTGGCCTACAGCAACAACTCTATCGCTATCCCCACCAACTTCACC |
| | | ATCAGCGTGACCACAGAGATCCTGCCTGTGTCCATGACCAAGACCAGCGTGGACTGCACCA |
| | | TGTACATCTGCGGCGATAGCACCGAGTGCTCCAACCTGCTGCTGCAGTACGGCAGCTTCTGC |
| | | ACCCAGCTGAAGAGAGCCCTGACAGGGATTGCCGTGGAACAGGACAAGAACACCCAAGAG |
| | | GTGTTCGCCCAAGTGAAGCAGATCTACAAGACCCCTCCTATCAAGTACTTCGGCGGGTTCAA |
| | | CTTCTCCCAGATTCTGCCCGATCCTAGCAAGCCCAGCAAGCGGAGCTTCATCGAGGACCTGC |
| | | TGTTCAACAAAGTGACACTGGCCGACGCCGGCTTCATCAAGCAGTATGGCGATTGCCTGGG |
| | | CGACATTGCAGCCAGGGATCTGATTTGCGCCCAGAAGTTTAACGGACTGACCGTGCTGCCT |
| | | CCTCTGCTGACCGATGAGATGATCGCCCAGTACACATCTGCCCTGCTGGCCGGCACAATCAC |
| | | AAGCGGCTGGACATTTGGAGCTGGCGCTGCCCTGCAGATCCCCTTTGCTATGCAGATGGCC |
| | | TACCGGTTCAACGGCATCGGAGTGACCCAGAACGTGCTGTACGAGAACCAGAAGCTGATC |
| | | GCCAACCAGTTCAACAGCGCCATCGGCAAGATCCAGGACAGCCTGAGCAGTACAGCCAGC |
| | | GCTCTGGGAAAGCTGCAGGACGTGGTCAACCACAATGCCCAGGCACTGAACACCCTGGTCA |
| | | AGCAGCTGAGCAGCAAGTTCGGCGCCATCTCCTCCGTGCTGAACGATATCCTGAGCAGACT |
| | | GGACCCTCCTGAGGCCGAGGTGCAGATCGACAGACTGATCACAGGACGGCTGCAGTCCCT |
| | | GCAGACCTACGTTACACAGCAGCTGATCAGAGCCGCCGAGATTAGAGCCTCTGCCAATCTG |
| | | GCCGCCACCAAGATGTCTGAGTGTGTGCTGGGCCAGAGCAAGAGAGTGGACTTTTGCGGC |
| | | AAGGGCTACCACCTGATGAGCTTCCCTCAGTCTGCACCACACGGCGTGGTGTTTCTGCACGT |
| | | GACATACGTGCCCGCTCAAGAGAAGAATTTCACCACCGCTCCAGCCATCTGCCACGACGGC |
| | | AAAGCCCACTTTCCTAGAGAAGGCGTGTTCGTGTCCAACGGCACCCATTGGTTCGTGACCCA |
| | | GCGGAACTTCTACGAGCCCCAGATCATCACCACCGACAACACCTTCGTGTCTGGCAACTGCG |
| | | ACGTCGTGATCGGCATTGTGAACAATACCGTGTACGACCCTCTGCAGCCCGAGCTGGACAG |
| | | CTTCAAAGAGGAACTGGATAAGTACTTTAAGAACCACACAAGCCCCGACGTGGACCTGGGC |
| | | GATATCAGCGGAATCAATGCCAGCGTCGTGAACATCCAGAAAGAGATCGACCGGCTGAAC |
| | | GAGGTGGCCAAGAATCTGAACGAGAGCCTGATCGACCTGCAAGAACTGGGGAAGTACGAG |
| | | CAGTATATCAAGTGGCCTTGGTACATCTGGCTGGGCTTTATCGCCGGACTGATTGCCATCGT |
| | | GATGGTCACAATCATGCTGTGCTGCATGACCAGCTGCTGCTCTTGCCTGAAGGGCTGCTGTA |
| | | GCTGTGGCTCCTGCTGCAAGTTCGACGAGGACGATTCTGAGCCTGTGCTGAAAGGCGTGAA |
| | | GCTGCACTACACATGA |
| 38 | Omicron | ATGTTCGTGTTTCTGGTTCTGCTTCCACTGGTATCCTCTCAATGCGTCAACTTGATTACTCGA |
| | XBB.1.5 | ACACAGTCATATACTAATTCTTTTACTCGCGGGGTGTATTATCCAGATAAGGTATTTCGCTCC |
| | 2P | AGCGTACTTCATTCTACACAAGACCTCTTCTTACCTTTCTTTTCCAACGTTACGTGGTTTCATG |
| | Codon | CAATCCATGTCAGCGGTACGAACGGAACTAAACGGTTCGACAATCCAGCTCTCCCTTTTAAC |
| | Strategy | GACGGCGTTTATTTCGCCTCTACCGAAAAGTCCAATATAATACGCGGGTGGATATTTGGCAC |
| | 2 (TW) | TACTCTGGATAGCAAAACTCAATCCTTGCTCATTGTGAATAACGCAACAAATGTCGTAATAA |
| | | AGGTTTGCGAATTCCAATTTTGTAATGATCCGTTTCTCGACGTCTACCAGAAGAACAACAAG |
| | | TCCTGGATGGAGAGTGAGTTTAGGGTGTACAGCAGTGCCAACAATTGTACTTTCGAGTATG |
| | | TTAGTCAACCATTTCTTATGGACCTTGAGGGGAAAGAGGGCAACTTTAAGAATCTGAGGGA |
| | | GTTCGTGTTTAAGAATATTGACGGTTATTTCAAAATTTATTCTAAGCATACCCCCATAAATCT |
| | | CGAGCGAGACCTTCCCCAAGGCTTTTCCGCGCTGGAACCGCTCGTCGATCTCCCTATTGGAA |
| | | TTAACATAACACGGTTCCAAACTCTGCTCGCATTACACCGCTCATACCTCACGCCCGTTGACT |
| | | CCAGCAGTGGTTGGACTGCCGGCGCCGCCGCGTACTACGTAGGCTATCTCCAACCTAGAAC |
| | | TTTTCTGCTCAAATATAATGAAAACGGTACTATCACAGACGCTGTGGATTGTGCCTTAGATC |
| | | CTCTGAGCGAGACTAAATGCACCCTCAAAAGCTTTACGGTAGAAAAGGGGATCTATCAAAC |
| | | ATCCAATTTCCGAGTCCAGCCTACCGAAAGTATTGTGCGGTTCCCAAACATCACCAATCTCT |
| | | GTCCCTTTCACGAAGTGTTTAATGCTACAACTTTTGCGAGCGTGTATGCATGGAATCGAAAG |
| | | AGAATTTCCAATTGTGTTGCGGACTACTCTGTTATTTACAATTTCGCACCTTTCTTCGCCTTTA |
| | | AATGTTATGGCGTCAGCCCGACTAAACTCAACGATCTTTGTTTTACAAACGTTTACGCCGATT |
| | | CCTTCGTGATTAGGGGTAACGAAGTCAGCCAGATTGCACCCGGACAAACTGGAAATATAGC |
| | | AGATTATAACTATAAATTGCCAGACGATTTCACTGGCTGCGTTATCGCATGGAATAGCAACA |
| | | AGCTTGACTCCAAACCTAGTGGAAATTATAATTATCTTTACCGGCTGTTTCGGAAATCCAAA |
| | | CTGAAGCCATTTGAGCGCGATATCAGCACAGAGATTTATCAAGCGGGGAACAAGCCATGTA |
| | | ATGGAGTTGCTGGGCCAAATTGCTACAGTCCCCTTCAATCTTACGGGTTTCGCCCTACCTAT |
| | | GGAGTGGGCCATCAACCTTACCGCGTGGTGGTTTTGAGTTTTGAACTGCTCCATGCCCCCGC |
| | | CACGGTGTGCGGCCCTAAGAAGTCCACCAATCTGGTTAAGAATAAGTGCGTCAACTTTAACT |
| | | TTAATGGACTTACAGGAACTGGGGTTCTGACCGAATCTAACAAGAAATTCCTTCCATTCCAA |
| | | CAATTTGGTAGGGATATTGCTGATACCACTGATGCCGTAAGGGACCCCCAAACACTGGAGA |
| | | TTCTCGATATCACCCCTTGCTCCTTTGGCGGCGTCTCTGTTATCACACCCGGTACTAATACTTC |
| | | AAATCAAGTGGCCGTCCTGTATCAGGGCGTAAACTGCACAGAGGTACCTGTAGCGATTCAC |
| | | GCCGATCAACTCACCCCAACCTGGCGGGTGTATTCTACGGGGAGTAATGTGTTTCAGACAC |
| | | GCGCGGGGTGTTTGATCGGGGCCGAATACGTAAATAACTCATACGAGTGCGACATACCTAT |
| | | TGGAGCTGGCATCTGTGCATCTTATCAAACGCAAACTAAATCCCACCGGCGGGCAAGATCT |
| | | GTGGCATCTCAATCAATTATCGCCTATACTATGAGCTTAGGCGCTGAAAATAGTGTGGCATA |
| | | CTCAAATAACAGTATCGCAATCCCAACCAATTTTACCATAAGTGTTACCACCGAAATACTGCC |
| | | TGTCTCTATGACGAAAACCTCAGTGGACTGCACTATGTATATTTGTGGCGATTCTACGGAGT |
| | | GCTCCAACCTGCTGCTCCAGTACGGCTCCTTTTGCACCCAGTTGAAACGCGCACTCACTGGA |
| | | ATTGCTGTAGAACAGGACAAGAATACACAAGAGGTGTTTGCCCAAGTCAAGCAAATCTATA |
| | | AAACACCGCCCATCAAGTATTTCGGCGGCTTCAATTTTAGCCAAATTCTTCCAGATCCCTCAA |
| | | AGCCTTCCAAACGAAGCTTTATTGAAGACTTGTTATTTAATAAGGTTACTCTGGCGGATGCT |
| | | GGGTTCATTAAACAATATGGAGATTGTTTGGGCGATATCGCCGCACGAGATCTGATCTGCG |
| | | CTCAGAAATTCAATGGGCTTACCGTGCTGCCGCCCTTGTTAACAGATGAAATGATCGCGCAA |
| | | TACACTAGCGCCTTGCTTGCAGGCACCATTACTTCCGGTTGGACGTTCGGGGCAGGTGCCG |
| | | CACTGCAAATTCCTTTCGCCATGCAAATGGCCTATAGGTTCAATGGTATAGGGGTGACTCAG |
| | | AATGTACTCTACGAGAATCAAAAGTTAATAGCAAATCAGTTCAATAGTGCCATAGGCAAGA |
| | | TTCAGGATTCCCTCAGTAGCACAGCAAGTGCACTCGGCAAATTGCAGGATGTTGTGAACCA |
| | | CAATGCCCAAGCACTGAACACGCTCGTAAAACAACTGAGCTCAAAGTTTGGAGCTATTTCAA |
| | | GCGTACTCAATGATATACTGTCCCGATTGGATCCACCCGAGGCTGAGGTTCAAATTGATAGA |
| | | CTGATTACCGGAAGGTTACAGTCTTTGCAGACCTACGTTACCCAACAGCTTATTCGCGCCGC |
| | | TGAAATTCGTGCTTCTGCCAACCTCGCGGCAACTAAAATGTCCGAATGCGTCCTTGGTCAAA |
| | | GTAAAAGGGTCGATTTCTGTGGGAAGGGGTATCACCTCATGTCCTTTCCGCAATCTGCTCCC |
| | | CACGGGGTAGTGTTTCTCCACGTCACGTACGTTCCAGCACAAGAGAAGAACTTTACAACAG |
| | | CACCTGCTATCTGTCATGACGGCAAAGCCCATTTTCCACGAGAAGGAGTGTTTGTCTCCAAC |
| | | GGGACGCATTGGTTTGTTACTCAGAGGAATTTCTACGAACCGCAGATCATTACTACTGATAA |
| | | CACTTTTGTTTCCGGAAATTGTGATGTGGTCATCGGTATTGTTAACAACACTGTGTATGACCC |
| | | TCTCCAGCCTGAACTGGACAGCTTTAAGGAAGAGCTCGATAAGTATTTCAAGAACCATACTA |
| | | GCCCCGACGTGGACCTGGGCGATATCAGTGGGATTAATGCGTCAGTAGTTAACATACAGAA |
| | | AGAAATCGACCGGCTTAATGAAGTTGCAAAGAACTTAAATGAGTCTCTGATTGATTTGCAA |
| | | GAACTCGGAAAGTACGAGCAATACATTAAATGGCCCTGGTACATATGGCTCGGGTTTATCG |
| | | CTGGCCTCATCGCAATAGTCATGGTGACGATTATGCTGTGTTGCATGACCAGTTGTTGCAGC |
| | | TGTTTGAAAGGATGTTGCTCATGTGGGAGCTGCTGCAAGTTTGATGAAGACGACTCAGAAC |
| | | CTGTTTTGAAAGGCGTTAAATTACATTACACGTAA |
| 39 | Omicron | ATGTTTGTGTTCTTGGTGTTATTGCCTCTCGTCTCCTCCCAGTGTGTGAATCTGATCACAAGA |
| | XBB.1.5 | ACTCAAAGCTACACCAACAGCTTTACTCGCGGAGTTTATTACCCAGACAAAGTGTTCAGAAG |
| | 2P | CAGCGTCTTGCACAGCACTCAAGACCTGTTCCTACCTTTCTTTAGCAATGTCACATGGTTCCA |
| | Codon | CGCAATCCATGTGAGCGGTACAAACGGGACAAAACGGTTCGATAACCCTGCACTCCCCTTC |
| | Strategy | AATGATGGAGTTTATTTCGCGAGCACGGAAAAGAGCAATATAATTAGAGGCTGGATCTTCG |
| | 3 (GS) | GCACCACGTTAGACTCCAAGACCCAATCTCTGCTGATCGTCAACAACGCGACCAACGTGGT |
| | | GATCAAGGTGTGTGAGTTCCAATTCTGTAACGACCCCTTTCTGGATGTCTACCAAAAAAACA |
| | | ACAAGTCCTGGATGGAGAGCGAATTCCGGGTGTACTCCTCAGCCAATAACTGCACCTTTGA |
| | | GTACGTTAGCCAGCCGTTCCTCATGGACCTGGAGGGGAAGGAAGGAAACTTCAAGAACTTA |
| | | CGGGAATTTGTCTTTAAAAACATTGATGGATATTTCAAAATCTACAGCAAACACACACCTAT |
| | | TAACCTGGAGAGAGACCTCCCACAGGGATTCAGTGCTCTCGAGCCGCTGGTGGACCTGCCC |
| | | ATCGGCATCAACATCACCAGATTTCAGACGCTGCTGGCTCTGCACCGGAGTTACCTGACTCC |
| | | TGTCGATAGCAGCTCGGGGTGGACAGCCGGCGCTGCTGCGTACTATGTCGGCTATCTGCAG |
| | | CCAAGAACCTTTCTGCTGAAGTATAATGAAAACGGTACCATCACCGATGCCGTGGACTGCG |
| | | CCCTGGATCCACTGAGCGAAACTAAGTGCACGCTGAAAAGCTTCACTGTCGAAAAAGGAAT |
| | | CTACCAAACATCTAACTTCCGAGTCCAGCCTACCGAGAGTATCGTGAGGTTCCCTAACATCA |
| | | CAAACCTTTGCCCCTTTCATGAAGTGTTCAATGCCACAACCTTCGCATCTGTGTACGCATGGA |
| | | ACAGGAAGAGAATCAGCAATTGTGTCGCCGATTACTCCGTCATCTATAACTTCGCCCCCTTC |
| | | TTCGCCTTCAAATGTTACGGCGTAAGTCCCACCAAGCTAAACGATCTATGCTTCACCAATGT |
| | | GTATGCTGACAGCTTCGTGATTCGTGGCAATGAAGTATCCCAGATCGCGCCTGGACAGACG |
| | | GGTAATATTGCAGACTACAACTACAAGCTGCCGGACGACTTCACTGGCTGCGTCATCGCCTG |
| | | GAATTCCAACAAGCTCGACAGCAAACCTTCAGGAAACTACAATTACTTGTACCGCTTATTTA |
| | | GAAAAAGCAAGCTGAAGCCCTTTGAAAGGGATATCTCTACGGAGATCTACCAAGCCGGCAA |
| | | CAAGCCCTGCAACGGGGTGGCAGGCCCCAACTGTTACAGCCCTCTGCAATCGTACGGCTTT |
| | | AGACCCACGTACGGCGTAGGGCACCAGCCCTACCGGGTGGTAGTGCTGAGCTTCGAGTTAC |
| | | TACATGCCCCAGCCACCGTGTGTGGACCCAAGAAAAGCACCAACCTGGTGAAGAATAAATG |
| | | TGTTAACTTCAACTTCAATGGCCTGACAGGAACAGGTGTGCTGACCGAGAGCAACAAGAAG |
| | | TTCCTTCCATTCCAGCAGTTTGGCAGGGATATTGCTGACACAACCGACGCCGTGAGAGATCC |
| | | CCAGACACTTGAGATCCTGGACATCACTCCTTGCAGTTTCGGCGGTGTGTCTGTGATCACCC |
| | | CTGGCACAAACACCTCTAACCAGGTGGCTGTGCTGTATCAGGGCGTGAACTGCACGGAGGT |
| | | GCCTGTGGCTATCCACGCCGACCAGTTGACCCCAACCTGGAGAGTGTACAGCACCGGCAGC |
| | | AACGTGTTCCAAACCCGCGCTGGCTGTCTTATCGGCGCGGAGTACGTTAATAACTCCTACGA |
| | | GTGCGACATCCCAATCGGGGCAGGGATCTGCGCTTCATATCAGACCCAGACAAAGTCCCAC |
| | | AGACGGGCACGATCTGTGGCGTCCCAGTCAATCATTGCATATACAATGTCCTTGGGAGCTG |
| | | AGAATAGTGTGGCCTACTCCAATAACAGCATCGCCATTCCCACCAACTTCACCATCTCTGTCA |
| | | CCACTGAGATCCTGCCGGTGTCTATGACCAAGACATCTGTGGACTGCACCATGTACATTTGT |
| | | GGAGATTCCACGGAATGCAGTAATCTGCTGTTACAGTACGGCTCCTTCTGCACGCAGCTTAA |
| | | AAGAGCCTTGACCGGAATCGCCGTGGAGCAGGACAAGAACACCCAGGAAGTGTTTGCGCA |
| | | GGTCAAGCAAATTTACAAAACACCACCTATTAAGTACTTCGGTGGCTTTAATTTCAGCCAGA |
| | | TTCTGCCAGACCCATCCAAGCCATCCAAGAGATCGTTCATCGAGGACCTGCTGTTCAACAAG |
| | | GTCACCCTGGCCGACGCCGGCTTTATTAAACAGTATGGAGACTGCCTGGGGGACATCGCCG |
| | | CCCGAGATCTGATCTGCGCCCAAAAGTTCAATGGGCTGACCGTGCTTCCTCCTCTGCTGACA |
| | | GATGAGATGATCGCCCAGTACACTAGCGCCCTTCTGGCTGGTACCATTACAAGCGGCTGGA |
| | | CTTTCGGCGCTGGGGCTGCACTTCAGATTCCCTTTGCCATGCAGATGGCCTACAGATTCAAC |
| | | GGAATAGGCGTGACACAGAATGTACTTTACGAAAATCAAAAGCTGATCGCGAACCAGTTCA |
| | | ATTCTGCTATCGGTAAGATACAGGACAGCCTTTCATCGACGGCCTCGGCCCTGGGCAAACT |
| | | GCAAGACGTGGTTAACCACAACGCTCAGGCCCTCAACACTTTGGTAAAGCAGCTAAGTAGT |
| | | AAATTTGGGGCCATCAGCTCAGTGCTGAATGACATACTCTCTCGTCTGGACCCTCCTGAGGC |
| | | TGAGGTCCAGATAGATAGACTGATCACCGGCCGGCTCCAGTCCCTGCAGACTTATGTGACC |
| | | CAGCAGCTGATCAGAGCCGCCGAGATCCGGGCCTCTGCTAACCTTGCCGCCACCAAAATGT |
| | | CTGAATGTGTATTGGGCCAAAGCAAGCGTGTAGACTTTTGTGGTAAGGGCTACCATCTGAT |
| | | GTCTTTCCCTCAGAGCGCCCCTCACGGGGTGGTTTTCTTGCACGTGACGTACGTACCTGCAC |
| | | AGGAGAAGAACTTTACAACCGCCCCCGCCATATGCCACGATGGGAAGGCTCACTTCCCGCG |
| | | GGAGGGGGTCTTCGTCTCTAACGGCACCCACTGGTTTGTGACTCAGAGAAATTTTTATGAG |
| | | CCCCAGATCATCACCACCGACAACACATTTGTTTCCGGCAACTGTGATGTGGTAATTGGGAT |
| | | CGTTAACAACACCGTGTATGACCCTCTGCAGCCTGAACTGGACAGTTTTAAAGAAGAGTTG |
| | | GATAAATACTTTAAGAACCACACCTCCCCTGATGTTGACCTGGGAGATATTAGCGGCATCAA |
| | | TGCCTCCGTGGTGAACATCCAGAAGGAGATTGACCGGCTGAACGAGGTCGCCAAGAACCTC |
| | | AACGAGTCCCTCATCGATCTCCAAGAGCTGGGCAAATATGAGCAGTATATAAAGTGGCCCT |
| | | GGTACATCTGGCTCGGCTTCATCGCAGGCCTGATAGCCATAGTGATGGTGACAATCATGCT |
| | | GTGCTGCATGACGTCGTGCTGCTCCTGCTTGAAGGGCTGCTGTTCCTGCGGCAGCTGTTGCA |
| | | AGTTTGACGAGGATGATAGTGAGCCTGTGCTGAAAGGGGTGAAGCTACACTACACAtga |
| 40 | Omicron | ATGTTCGTGTTCCTGGTGCTGCTGCCCCTGGTGAGCTCTCAGTGCGTGAACCTGATCACAAG |
| | XBB.1.5 | AACACAGAGCTACACCAACAGCTTCACAAGAGGCGTGTACTACCCCGACAAGGTGTTCAGA |
| | 2P | AGCAGCGTGTTGCATTCTACCCAAGACCTGTTTCTGCCCTTCTTCAGCAACGTGACCTGGTTC |
| | Codon | CACGCCATCCACGTGAGCGGCACCAACGGCACCAAGAGATTCGACAACCCCGCCCTGCCCT |
| | Strategy | TCAACGACGGCGTGTACTTCGCTAGCACCGAGAAGAGCAACATCATCAGAGGCTGGATCTT |
| | 4 (AZ) | CGGCACCACCCTCGACAGCAAAACACAGAGCCTGCTGATCGTGAACAATGCCACCAACGTG |
| | | GTGATCAAGGTGTGCGAGTTTCAGTTCTGCAACGACCCCTTCCTGGACGTGTATCAGAAGA |
| | | ACAACAAGAGCTGGATGGAGAGCGAGTTCCGTGTGTATAGCTCCGCTAACAACTGCACCTT |
| | | CGAGTACGTGTCTCAGCCCTTCCTGATGGACCTGGAGGGCAAGGAGGGCAACTTCAAAAAC |
| | | CTGAGAGAGTTCGTGTTCAAGAACATCGACGGCTACTTCAAGATCTACAGCAAGCACACCC |
| | | CCATCAACCTGGAGAGAGACCTGCCCCAAGGCTTCAGCGCCCTGGAGCCCCTGGTGGACCT |
| | | GCCCATCGGCATCAACATCACAAGATTTCAGACCCTGCTGGCCCTGCACAGATCATACCTTA |
| | | CTCCTGTGGATAGCAGCTCGGGTTGGACCGCCGGTGCCGCCGCCTACTACGTGGGCTACCT |
| | | GCAGCCTAGAACCTTCCTGCTGAAGTACAACGAGAACGGTACGATTACTGACGCCGTGGAC |
| | | TGTGCCCTGGACCCCCTGAGCGAGACCAAGTGCACCCTGAAGAGCTTCACCGTGGAGAAG |
| | | GGCATCTATCAGACAAGCAACTTCAGAGTGCAGCCCACCGAGAGCATCGTGAGATTCCCCA |
| | | ACATCACCAACCTGTGCCCCTTCCACGAGGTGTTCAACGCCACCACCTTCGCTAGCGTGTAC |
| | | GCCTGGAACAGAAAGAGAATCAGCAACTGCGTGGCCGACTACAGCGTGATCTACAACTTCG |
| | | CCCCCTTCTTCGCCTTCAAGTGTTACGGCGTGAGCCCCACCAAGCTGAACGACCTGTGCTTC |
| | | ACCAACGTGTACGCCGACAGCTTCGTGATCAGAGGCAACGAGGTGTCTCAGATCGCCCCCG |
| | | GGCAGACCGGCAACATCGCCGACTACAACTACAAGCTGCCCGACGACTTCACCGGCTGCGT |
| | | GATCGCTTGGAACAGCAATAAGCTGGACAGCAAGCCGTCCGGCAACTACAACTACCTGTAC |
| | | AGACTGTTCAGAAAGAGCAAGCTGAAGCCCTTCGAGAGAGACATCAGCACCGAGATCTACC |
| | | AAGCCGGCAACAAGCCCTGCAACGGCGTGGCCGGCCCCAACTGCTACAGCCCCCTGCAGA |
| | | GCTACGGCTTCAGACCCACCTACGGCGTGGGCCATCAGCCCTACAGAGTGGTGGTGCTGAG |
| | | CTTCGAGCTGCTGCACGCCCCCGCTACCGTGTGCGGCCCCAAGAAGAGCACCAACCTGGTG |
| | | AAGAACAAGTGCGTGAACTTCAACTTTAACGGTCTGACGGGCACCGGCGTGCTGACCGAGA |
| | | GCAACAAGAAGTTCCTGCCATTTCAGCAATTCGGCAGAGACATCGCCGACACCACCGATGC |
| | | CGTGCGTGACCCTCAGACCCTGGAGATCCTGGACATCACCCCCTGTAGTTTCGGAGGCGTG |
| | | AGCGTGATCACCCCCGGCACCAACACAAGCAACCAAGTGGCCGTGCTGTACCAAGGCGTGA |
| | | ACTGCACCGAGGTGCCCGTGGCCATCCACGCCGATCAGCTGACCCCCACCTGGAGAGTATA |
| | | TAGCACCGGCAGCAACGTGTTTCAGACAAGAGCCGGCTGCCTGATCGGCGCCGAGTACGT |
| | | GAACAACAGCTACGAGTGCGACATCCCCATCGGCGCCGGCATCTGCGCTAGCTATCAGACA |
| | | CAGACCAAGAGCCACAGAAGAGCTAGAAGCGTGGCTTCTCAGAGCATCATCGCCTACACCA |
| | | TGAGCCTGGGCGCCGAGAACAGCGTGGCCTACAGCAACAACAGCATCGCCATCCCCACCAA |
| | | CTTCACCATCAGCGTGACCACCGAGATTCTGCCTGTGAGCATGACCAAGACAAGCGTGGAC |
| | | TGCACCATGTACATCTGCGGCGACAGCACCGAGTGCAGCAACCTGCTGCTGCAGTACGGCA |
| | | GCTTCTGCACACAGCTGAAGAGAGCCCTGACCGGCATCGCCGTGGAGCAAGACAAGAACA |
| | | CCCAAGAGGTGTTCGCCCAAGTGAAGCAGATCTACAAGACCCCCCCCATCAAGTACTTCGG |
| | | CGGCTTCAACTTCAGCCAAATCCTGCCCGACCCTAGCAAGCCTAGCAAGCGTAGCTTCATCG |
| | | AGGACCTGCTGTTCAACAAGGTGACCCTGGCCGACGCCGGCTTCATCAAGCAGTACGGCGA |
| | | CTGCCTGGGCGACATTGCCGCTAGAGACCTGATCTGCGCTCAGAAGTTCAATGGGTTAACG |
| | | GTGCTCCCCCCCCTGCTGACCGACGAGATGATCGCTCAGTACACTAGCGCTCTGCTGGCTGG |
| | | CACTATCACCTCCGGTTGGACCTTCGGAGCCGGCGCGGCCCTGCAAATCCCCTTCGCCATGC |
| | | AGATGGCCTACAGATTCAACGGCATCGGCGTGACACAGAACGTGCTGTACGAGAATCAGA |
| | | AGCTGATCGCCAATCAGTTCAACAGCGCCATCGGCAAGATCCAAGACAGCCTGAGCAGCAC |
| | | CGCTAGCGCCCTGGGCAAGCTGCAAGACGTGGTGAACCACAACGCCCAAGCCCTGAACACC |
| | | CTGGTGAAGCAGCTGAGCAGCAAGTTCGGCGCCATCAGCAGCGTGTTAAATGACATCCTGA |
| | | GCAGACTGGACCCCCCCGAGGCCGAGGTGCAGATCGACAGACTGATCACCGGCAGACTGC |
| | | AGAGCCTGCAGACCTACGTGACACAGCAGCTGATCAGAGCCGCCGAGATCAGAGCTAGCG |
| | | CCAACCTGGCCGCCACCAAGATGAGCGAGTGCGTGCTGGGGCAGAGCAAGAGAGTGGACT |
| | | TCTGCGGCAAGGGCTACCACCTGATGAGCTTCCCTCAGAGCGCCCCCCACGGCGTGGTGTT |
| | | CCTGCACGTGACCTACGTGCCCGCCCAAGAGAAGAACTTCACTACGGCACCTGCCATCTGCC |
| | | ACGACGGCAAGGCCCACTTCCCTAGAGAGGGCGTGTTCGTGAGCAACGGCACCCACTGGTT |
| | | CGTGACACAGAGAAACTTCTACGAGCCTCAGATCATCACCACCGACAACACCTTCGTGAGC |
| | | GGCAACTGCGACGTGGTGATCGGTATCGTTAATAATACCGTGTACGACCCCCTGCAGCCTG |
| | | AGCTGGACAGCTTCAAGGAGGAGCTGGACAAGTACTTCAAGAACCATACTAGCCCCGACGT |
| | | GGACCTAGGCGACATTAGCGGCATCAACGCTAGCGTGGTGAACATTCAGAAGGAGATCGA |
| | | CCGGCTGAATGAGGTGGCCAAGAACCTGAACGAGAGCCTGATCGACCTGCAAGAGCTGGG |
| | | CAAGTACGAGCAGTACATCAAGTGGCCCTGGTACATCTGGCTGGGCTTCATCGCCGGCCTG |
| | | ATCGCCATCGTGATGGTGACCATCATGCTGTGCTGCATGACAAGCTGTTGCAGCTGCCTGAA |
| | | AGGTTGCTGCAGCTGTGGCTCGTGCTGCAAGTTCGACGAGGACGACAGCGAGCCCGTGCT |
| | | GAAGGGCGTGAAGCTGCACTACACCTGATGA |

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1—Prototyping of DNA Vaccine Candidates with the FAST-PLV Platform for Intracellular Delivery

A mammalian codon-optimized DNA sequence encoding the entire open reading frame (ORF) of Wuhan SARS-CoV-2 Spike glycoprotein was synthesized based on the publicly available amino acid sequence (GenBank Protein Accession: QHD43416; FIG. 1B) (Wu et al., 2020). Similarly, DNA encoding the spike receptor binding domain (RBD) and secreted receptor binding domain (sRBD), RBD fused to the Kappa light chain signal peptide, were synthesized as additional SARS-CoV-2 antigen candidates (FIG. 1B). These antigens were cloned into two different plasmid backbones, the optimized p10 plasmid and a Nanoplasmid. The Nanoplasmid backbone is small, non-immunogenic, and capable of potent expression of encoded gene products. To compare expression of the antigen payloads plasmid candidates were formulated in the fusion associated small transmembrane small transmembrane associated protein-proteolipid vehicle (FAST-PLV) delivery platform and added directly to the media of 293T cells. Spike expression was determined by Western-Blot. Expression of Spike protein from the Nanoplasmid backbone far exceeded that of the p10 backbone at the same dose of DNA (FIG. 1C); expression was dose-dependent, and full-length Spike was efficiently post translationally processed to S1/S2 fragments (FIG. 1D). Expression of sRBD from the Nanoplasmid was also readily detected (FIG. 1E). Therefore, the FAST-PLV platform is sufficient for intracellular delivery, robust expression, and proper post-translational processing of plasmid DNA encoded SARS-Cov2 Spike antigens.

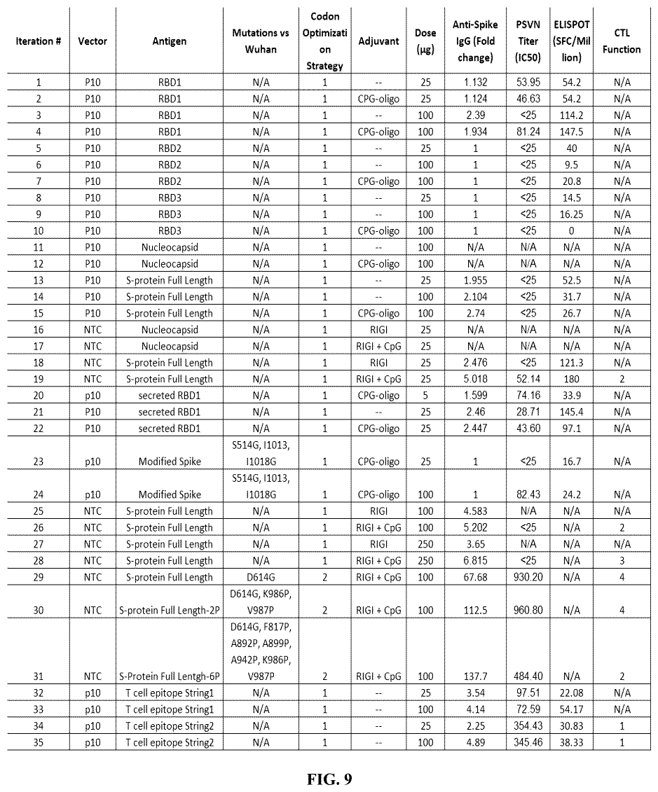

The FAST-PLV platform leverages the case and flexibility of recombinant DNA manipulation by enabling efficient intracellular delivery of plasmid DNAs. Five prototype vaccine candidates were generated encoding either full length SARS-CoV-2 Spike, RBD, or sRBD, and adjuvants. NP-S-CpG-RIGI encodes Spike on a Nanoplasmid backbone with two genetic adjuvants; an RNA Pol III driven retinoic acid-inducible gene 1 (RIG-I) double-stranded RNA agonist element, eRNA41H, and CpG motifs included in the 3′-UTR of the Spike mRNA transcribed by a pol II promoter. An analogous vector to the vector of NP-S-CpG-RIGI is shown in FIG. 10. Compared to the vector of FIG. 10, the NP-S-CpG-RIGI vector comprises a different polynucleotide sequence encoding the Spike protein. In the NP-S-CpG-RIGI vector, the polynucleotide sequence encoding the Spike protein encodes the spike protein of SEQ ID NO: I using a codon sequence optimized using the Integrated DNA Technologies (IDT) codon optimization web tool (https://www.idtdna.com/pages/tools/codon-optimization-tool). NP-S-RIGI encodes Spike on the Nanoplasmid backbone with the RIG-I agonist genetic adjuvant only. A full-length Spike gene in the optimized p10 plasmid backbone, denoted p10-S, lacks all encoded genetic adjuvants. Similarly, p10-RBD encodes the RBD from the SARS-CoV-2 Spike protein, and p10-sRBD encodes a secreted RBD on the optimized p10 backbone without genetic adjuvants (FIG. 1B). These five DNA vaccine prototypes were formulated into FAST-PLVs and screened for immunogenicity in mice.

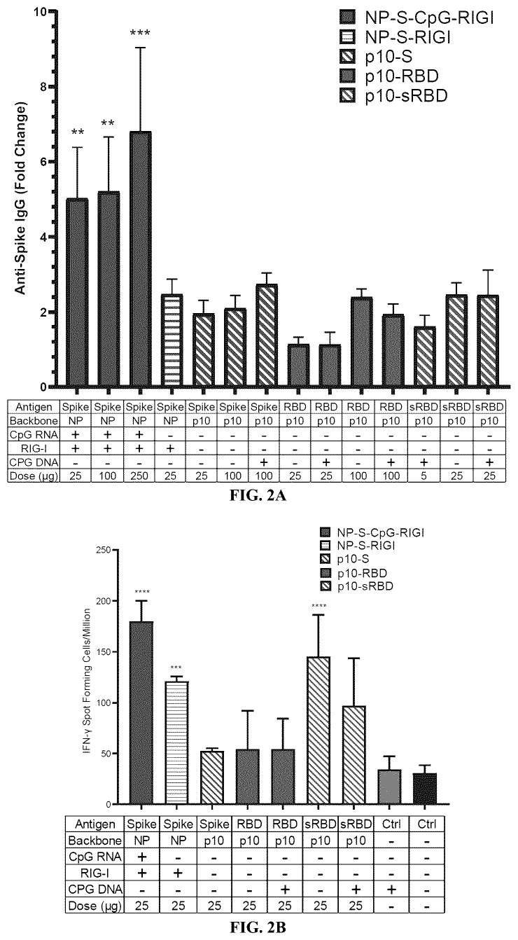

Mice were immunized intra-muscularly (IM) with each candidate using a prime (day 0)-boost (day 14) regimen. At seven days post-boost (day 21), an indirect electro-chemiluminescence immunoassay (ECLIA) was used to measure the binding of anti-Spike IgG to recombinant SARSCoV-2 Spike protein, reported as the fold change from control (vehicle) treated animals. NP-S-CpG-RIGI elicited dose-dependent anti-Spike antibody responses that were approximately two- to three-fold as great as those induced by NP-S-RIGI lacking the CpG RNA element or the other vaccine candidates, with the 250 μg dose inducing the greatest response (FIG. 2A). Neither increasing the dose of the other candidates nor co-formulating them with CpG DNA oligonucleotides achieved responses greater than NP-S-CpG-RIGI (FIG. 2A). Addition of the IgG signal peptide to generate secreted RBD (p10-sRBD) improved antibody production, yielding equivalent responses as p10-RBD, but at lower DNA doses that were not improved by coformulation with CpG DNA (FIG. 2A). Thus, NP-S-CpG-RIGI encoding full length Spike with the addition of two genetic adjuvants generated the most robust antibody responses.

To assess T cell responses elicited by the DNA vaccine candidates, mice were immunized IM with a 25 μg dose of the candidates following the same prime-boost protocol used to elicit humoral responses. At 21 days post-immunization (7 days following the booster), the frequency of IFN-γ spot forming cells (SFC) per million splenocytes was quantified by an enzyme-linked immune absorbent spot (ELISpot) assay following ex vivo stimulation with overlapping peptide pools (15 mers overlapping by 11 amino acids) spanning the entire SARS-CoV-2 Spike protein ORF. NP-S-CpG-RIGI vaccination stimulated a potent T cell response indicated by increased frequencies of T cells expressing IFN-γ, reaching a frequency greater than 170 SFCs/million splenocytes at day 21 (FIG. 2B). NP-S-RIGI and p10-sRBD also elicited a significant increase in the frequency of IFN-g SFCs compared to control immunized mice (FIG. 2B). There was no detectable difference in the responses to p10-Spike and p10-RBD compared to control immunized mice, and the addition of co-formulated CpG to the p10-RBD did not improve the response.

Taken together the combination of full-length Spike with both RIG-I and CpG genetic adjuvants on a Nanoplasmid backbone (NP-S-CpG-RIGI) provided the best overall immunogenicity upon initial screening. The Nanoplasmid backbone formulated in with the FAST-PLV platform also demonstrates durable stability for up to one year at 2-8° C. (data not shown). Therefore, this configuration was selected for further optimization including the introduction of the ubiquitous 614G mutation (NP-S) as well as generation of 2P (K986P and V987P) fusion stabilization mutant (NP-S-2P) for further evaluation. Codon optimization of the NP-S and NP-S-2P variants was performed using the Thermo Fisher web tool (https://www.thermofisher.com/us/en/home/life-science/cloning/gene-synthesis/geneart-gene-synthesis.html). Compared with the codon optimized sequence of the NP-S-CpG-RIGI encoded spike protein (SEQ ID NO: 6), the codon optimized encoding sequence of the Spike protein of the NP-S-2P vector (SEQ ID NO: 5) displays only a 77.1% sequence alignment despite the amino acid sequences differing by only 3 amino acids out of the 1273 amino acids of the full length spike protein (>99.7% sequence identity based on amino acid sequence).

### Example 2—FAST-PLV Vaccine Candidates Encoding Both Spike and Genetic Adjuvants Induce Humoral Immunity in Mice

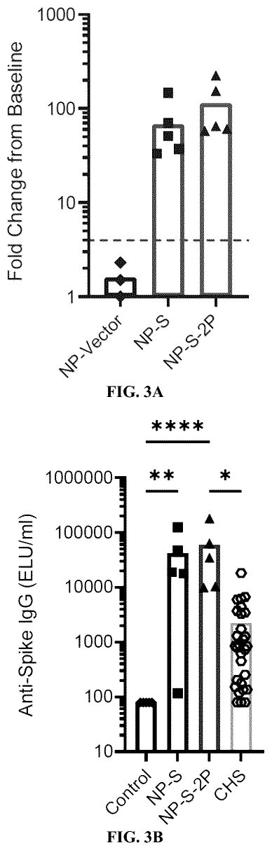

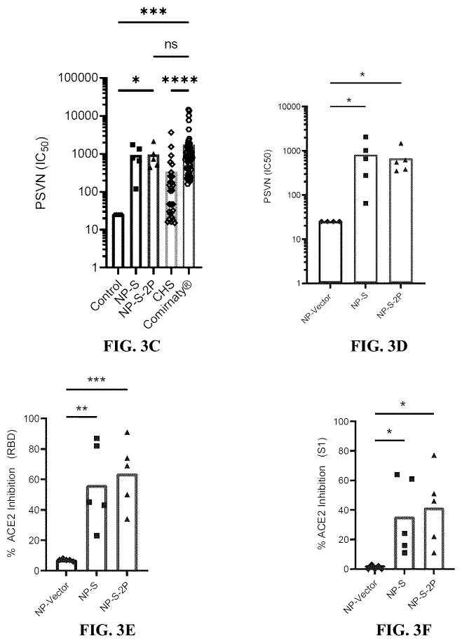

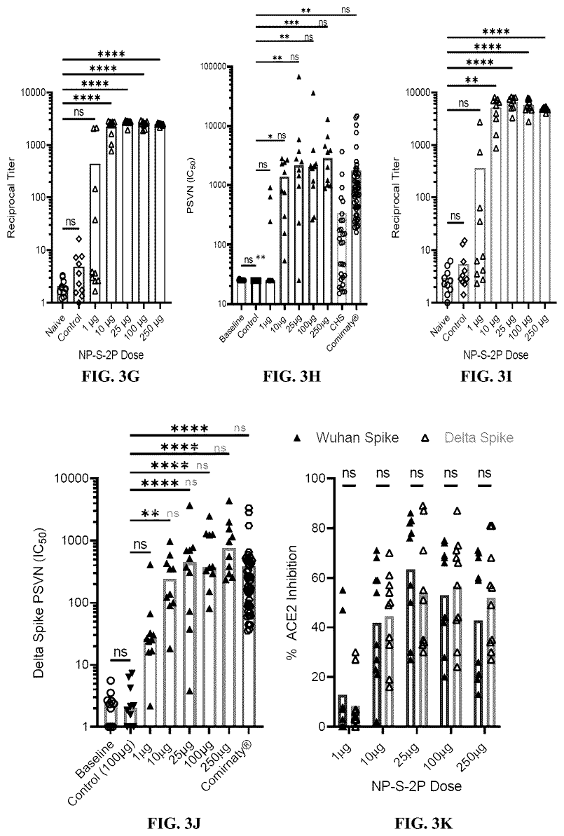

To assess immunogenicity of the optimized NP-S and NP-S-2P PLV-vaccine candidates, mice were injected IM with 100 μg of encapsulated DNA on day 0 (prime) and again on day 21 (boost). Two weeks after the boost (day 35) serum was collected and anti-Spike antibody production was determined by ECLIA. NP-S generated a 67-fold increase in baseline signal and NP-S-2P generated 112-fold change—a nearly two-fold increase over NP-S (FIG. 3A). A qualified SARS-Cov2 Spike binding ELISA determined NP-S and NP-S-2P induced serum anti-Spike IgG concentrations up to 125,518 U/ml and 179,413, respectively (average 41,745 or 59,497 respectively, FIG. 3B), and NP-S-2P generated a higher anti-Spike antibody titer compared to a panel (n=28) of convalescent human serum collected 14 days post symptoms resolving (FIG. 3B). FAST-PLV plasmid DNA vaccines induce robust antigen specific antibody production.

### Example 3A—Vaccine Candidates Encoding Spike Protein and Genetic Adjuvants Induce Robust Neutralizing Antibody Responses in Mice

To assess the functionality of the humoral responses generated by the vaccine candidates, a pseudovirus neutralization assay (PSVN) using luciferase-expressing lentivirus pseudotyped with SARS-CoV-2 Spike was used to infect permissive 293T-ACE2 cells. Serum containing neutralizing antibodies (nAb) against SARS-CoV-2 blocks infection of the pseudotyped lentivirus, resulting in a dose-dependent decrease in luciferase signal. Consistent with total anti-Spike IgG responses, NP-S and NP-S-2P induced robust neutralizing activity, equivalent to the neutralizing activity of SARS-CoV2 convalescent patient serum (FIG. 3C). NP-S-2P generated PSVN titers indistinguishable from a panel of Comirnaty® vaccinated individuals (n=52, serum collected two weeks after the second dose). The robust neutralizing activity generated by NP-S and NP-S-2P versus empty vector control (NP-Vector) were confirmed using a similar qualified PSVN assay performed blinded by a reference lab (FIG. 3D) as well as an RBD-Ace2 inhibition assay (FIG. 3E) and a S1-Ace2 inhibition assay (FIG. 3F). Thus, NP-S and NP-S-2P PLV vaccines elicit a potent nAb response against their target antigens.

### Example 3B—NP-S-2P Vaccine Candidate Induces Significant Anti-Spike IgG Levels and Neutralizing Titers at Low Doses

NP-S-2P was further examined for the impact of PLV-DNA vaccine dose on immunogenicity. Groups of 10 mice were immunized on day—and boosted on day 21 with 1, 10, 25, 100, or 250 microgram of NP-S-2P or NP-Vector control (100 microgram). Serum was collected on day 35, 14 days after the second dose and assessed for antibody titers against Wuhan and Delta variant (B.1.617.2 and AY lineages) Spike. NP-S-2P at PLV doses greater than 1 microgram stimulated robust anti-Spike antibody titers compared to naïve and control treated animals (P<0.0001, FIG. 3G). There was no detectable difference between naïve and control treated animals (P>0.05, FIG. 3G). Antibody responses at does of 10 micrograms and above were indistinguishable, indicating that a threshold dose of 10 micrograms is sufficient to elicit maximal anti-Spike antibody production in mice (FIG. 3G). Furthermore, the group response variability decreased inversely with dose response, the 1 microgram dose group had the most variability that decreased dramatically by increasing the dose to 10 microgram and minimized at the highest dose of 250 microgram (FIG. 3G). Consistent with the robust antibody responses mice vaccinated with two doses ≥10 10 microgram of NP-S-2P generated significant PSVN titers against Wuhan Spike compared to vector control (P<0.01, FIG. 3H). There was no detectable difference between the PSVN titer generated by NP-S-2P at doses ≥10 micrograms and a panel of 52 Comirnaty® vaccinated individuals (P<0.05, FIG. 3H). Thus, PLV based DNA vaccines encoding Wuhan Spike-2P elicit indistinguishable humoral response in mice compared to the mRNA based Comirnaty®.

Notably, a dose ≥10 μg of NP-S-2P was also sufficient to induce maximal antibody titers against the Delta variant Spike (B.1.617.2 and AY lineages) (FIG. 3I). Two doses ≥10 μg of NP-S-2P also generated significant PSVN titers against Delta spike compared to vector control (P<0.01, FIG. 3J). Again, there was no detectable difference between the Delta PSVN titer generated by NP-S-2P at doses ≥10 μg and a panel of 52 Comirnaty® vaccinated individuals (P<0.05, FIG. 3J). Consistent with Wuhan Spike, PSVN titers against vector control was unchanged from baseline (P>0.05, FIG. 3H, J). The neutralizing antibody responses against Wuhan and Delta Spike generated by NP-S-2P were further supported by significant ACE2 inhibition compared to vector control that at doses ≥10 μg (P<0.01, FIG. 3K). Disruption of ACE2 binding to Wuhan vs Delta Spike by serum from vaccinated mice was indistinguishable at all doses tested (P>0.05, FIG. 3K). A threshold dose of 10 μg is sufficient to induce robust antibody responses against Wuhan and Delta Spike proteins using a prime-boost regimen of a PLV-DNA vaccine candidate encoding Spike 614G_2P and comparable to the response generated by Comirnaty®.

### Example 3C—Omicron-2P Vaccine Candidates are Predicted to Induce Significant Anti-Spike IgG Levels and Neutralizing Titers at Low Doses

An experiment substantially as described above is carried out for vaccine candidates which encode an omicron variant spike protein (e.g., as in SEQ ID NO: 3, SEQ ID NO: 31, or SEQ ID NO: 36, such as with a polynucleotide sequence of one of SEQ ID NOs: 4, 32-35, and/or 37-40). It is expected that such vaccine candidates will display similar results to those described above, but with enhanced anti-omicron activity (e.g., will elicit a greater anti-Omicron spike IgG levels).

### Example 4A—Vaccine Candidates Encoding Spike Protein and Genetic Adjuvants Elicit a Robust Cellular Immune Response

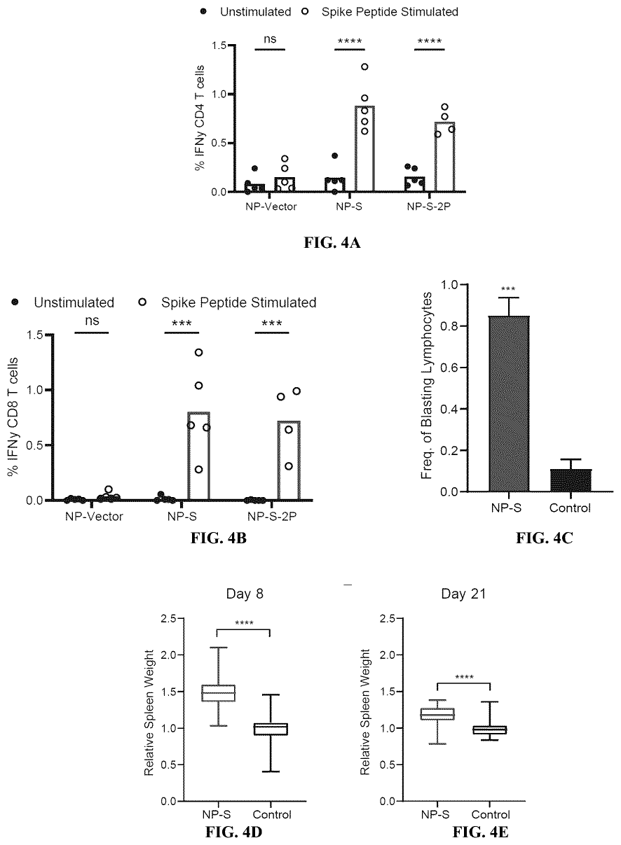

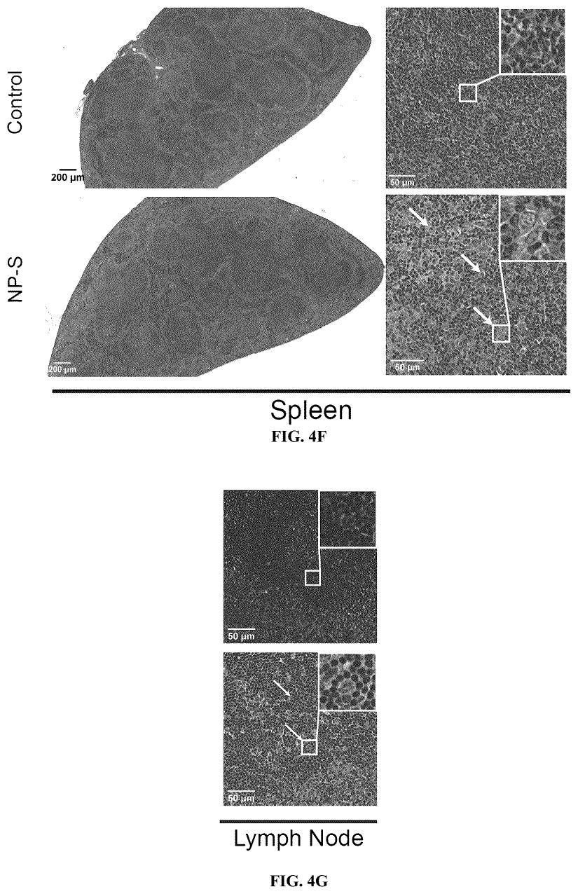

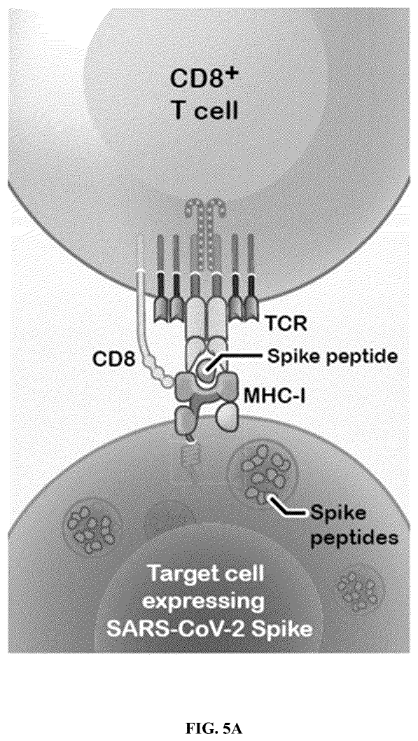

To assess cell mediated immunity of NP-S and NP-S-2P PLV-vaccine candidates, mice were injected IM with 100 μg of encapsulated DNA on day 0 and day 21 as before and Spike protein specific T cell responses were characterized two weeks after the boost (day 35). Compared to empty vector (NP-Vector) control, vaccination with NP-S and NP-S-2P induced significant increases in the frequency of Interferon-gamma (IFN-γ) producing CD4+ and CD8+ T cells following stimulation with SARS-CoV-2 Spike peptide pools (FIG. 4A, FIG. 4B). Consistent with robust immune cell activation, NP-S induced a 4-fold increase in the frequency of blasting lymphocytes in the spleen, and significant splenomegaly compared to control 7 days after each immunization (FIG. 4C, FIG. 4D, FIG. 4E). Features characteristic of lymphoid hyperplasia, including increased size and number of follicles in the spleens and draining lymph nodes was observed at day 8 post-immunization of mice with NP-S (FIG. 4F, FIG. 4G). Thus, PLV-encapsulated plasmid DNA vaccines readily induce functional and histological hallmarks of cell-mediated immunity.

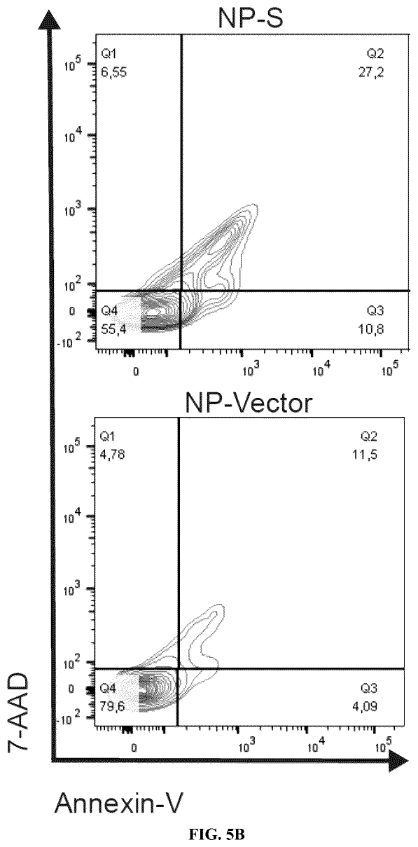

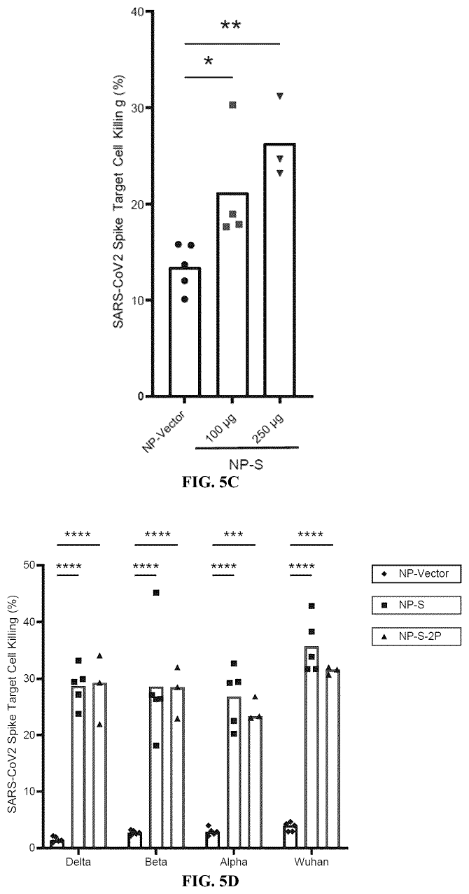

Cytotoxic T lymphocytes (CTLs) are an important effector T cell type to eliminate virus infected cells. Once activated and fully licensed, CTLs target and kill infected cells presenting viral epitopes (peptides) in the context of major histocompatibility complex I (MHC-I) (FIG. 5A). To confirm that CD8+ T cells stimulated by NP-S and NP-S-2P had the capacity to kill relevant target cells, a syngeneic target cell line using B16 melanoma cells from C57BL/6 mice stably expressing SARS-CoV-2 Spike protein was established. Splenocytes from NP-S immunized mice showed increased, dose-dependent killing of these co-cultured syngeneic target cells compared to splenocytes from control mice (FIG. 5B, FIG. 5C). A panel of syngeneic B16 Melanoma target cells expressing the spike protein from three variants of concern (VOC), Alpha, Beta and Delta was then generated. Purified CD8+ T cells from animals vaccinated with NP-S and NPS-2 were able to efficiently kill target cells expressing WT (Wuhan) Spike, and all three VOC Spike proteins compared to CD8+ T cells from animals immunized with empty vector (FIG. 5D). Together these findings indicate that NP-S and NP-S-2P elicit a spike-specific T cell immune responses that includes a Spike-specific CTL response targeting WT and VOC Spike expressing cells with the capacity to functionally kill virally infected target cells.

### Example 4B—Vaccine Candidates Encoding Codon Optimized Omicron Spike Protein and Genetic Adjuvants are Predicted to Elicit a Robust Cellular Immune Response

An experiment substantially as described above is carried out for vaccine candidates which encode an omicron variant spike protein (e.g., as in SEQ ID NO: 3, SEQ ID NO: 31, or SEQ ID NO: 36, such as with a polynucleotide sequence of one of SEQ ID NOs: 4, 32-35, and/or 37-40). It is expected that such vaccine candidates will display similar results to those described above, but with enhanced anti-omicron activity (e.g., will elicit a stronger CD8+ T cell response against B16 melanoma cells from C57BL/6 mice stably expressing a SARS-CoV-2 Omicron Spike protein).

### Example 5A—NP-S was Well Tolerated and Induces Robust Neutralizing Activity in Non-Human Primates (NHPs

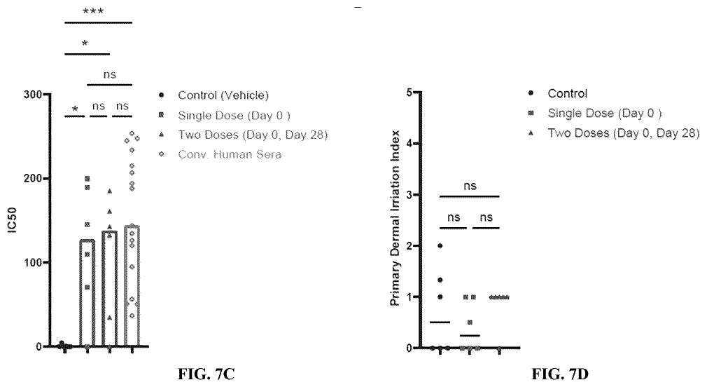

To assess the safety of the NP-S vaccine candidate and its ability to induce anti-Spike neutralizing activity in a translational preclinical model, adult African green monkeys (Chlorocebus sabaeus) were immunized IM with the full human dose of 250 μg NP-S plasmid DNA formulated in FAST-PLV or with saline as a control (FIG. 7A). NHPs were separated into 3 groups and received either (i) saline alone (Control), (ii) one dose of 250 μg NP-S at day 0 (Single Dose), or (iii) two doses of 250 μg NP-S at days 0 and 28 (Two Doses) (FIG. 7A). Sera obtained from NHPs at baseline and at weekly intervals were assessed for their capacity to neutralize SARS-CoV-2 Spike using the earlier described PSVN assay (FIG. 3A). Both one- and two-dose groups demonstrated effective and comparable neutralization of SARS-CoV-2 Spike pseudotyped lentivirus, which increased greatly with time post-vaccination (FIG. 7B), and after 42 days, demonstrated similar neutralization to convalescent human sera, whereas sera from control animals demonstrated no neutralization capacity (FIG. 7C). Local tolerance to the intramuscular administration of NP-S PLV-vaccine was evaluated by Draize scoring, which captures the manifestation and severity of both erythema and edema at the site of the injection, to generate a Primary Dermal Irritation Index. No detectable differences were observed in the primary dermal irritation index between control animals and either of the NP-S dose groups indicating that the repeat dosing (full human dose, IM) in NHP is well tolerated (FIG. 7D). Thus, PLV formulated NP-S plasmid DNA vaccine is well tolerated in NHP and importantly a single dose is sufficient to generate a neutralizing antibody (nAb) response.

### Example 5B—NP-S-2P Produces Anti-Spike IgG and Neutralizing Titers Against Wuhan and Delta Variants

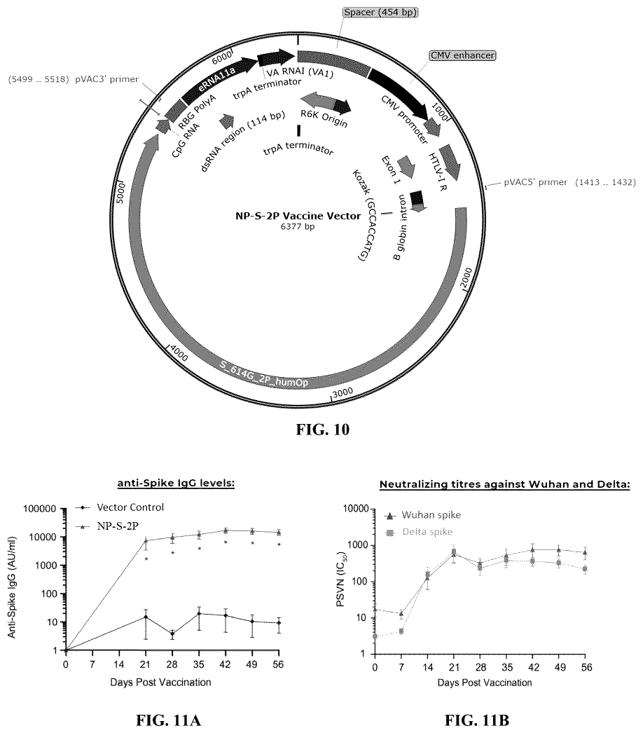

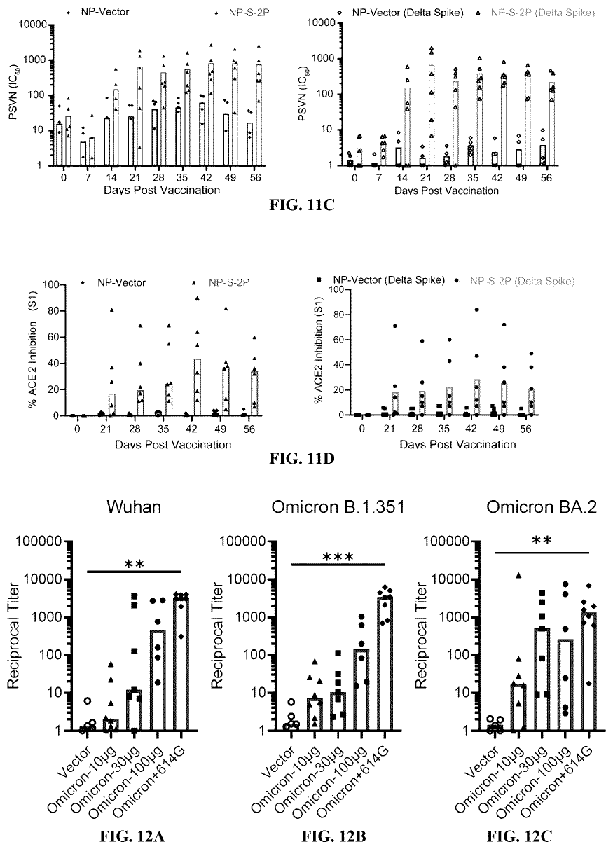

In a second study analogous to that discussed supra, efficacy of the NP-S-2P non-human primates were administered 250 microgram doses of the NP-S-2P vaccine at day 0 (prime) and day 28 (boost). Control animals received a dose of empty vector control. Anti-spike IgG levels were measured at 3, 4, 5, 6, 7, and 8 weeks post priming dose (FIG. 11A), and neutralizing titers against Wuhan and Delta virus variants were measured at 1, 2, 3, 4, 5, 6, 7, and 8 weeks post priming dose (FIG. 11B). The neutralizing titers for Wuhan and Delta variants were also measured using the PSVN assay relative to NP-Vector control (FIG. 11C, left and right, respectively) at the indicated time points. Similarly, neutralizing titers for Wuhan and Delta variants were measured for the inhibition of binding for the viral S1 domain to ACE2 at the indicated time points (FIG. 11D, left and right, respectively).

### Example 5C—Codon Optimized Omicron Spike Vaccine Candidates Produce Enhanced Anti-Spike IgG Titers Against Omicron Variant

A third study substantially as described above is carried out for vaccine candidates which encode an omicron variant spike protein (e.g., as in SEQ ID NO: 3, SEQ ID NO: 31, or SEQ ID NO: 36, such as with a polynucleotide sequence of one of SEQ ID NOs: 4, 32-35, and/or 37-40). It is expected that such vaccine candidates will display similar results to those described above (e.g., similar tolerability), but with enhanced anti-omicron activity (e.g., will elicit a stronger anti-Omicron).

### Example 6A—NP-S Protects Hamsters from SARS-CoV-2 Infection in a Challenge Model

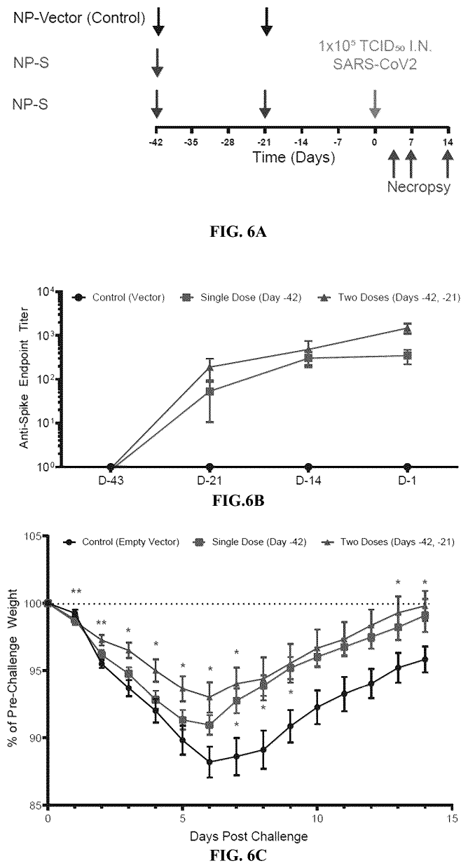

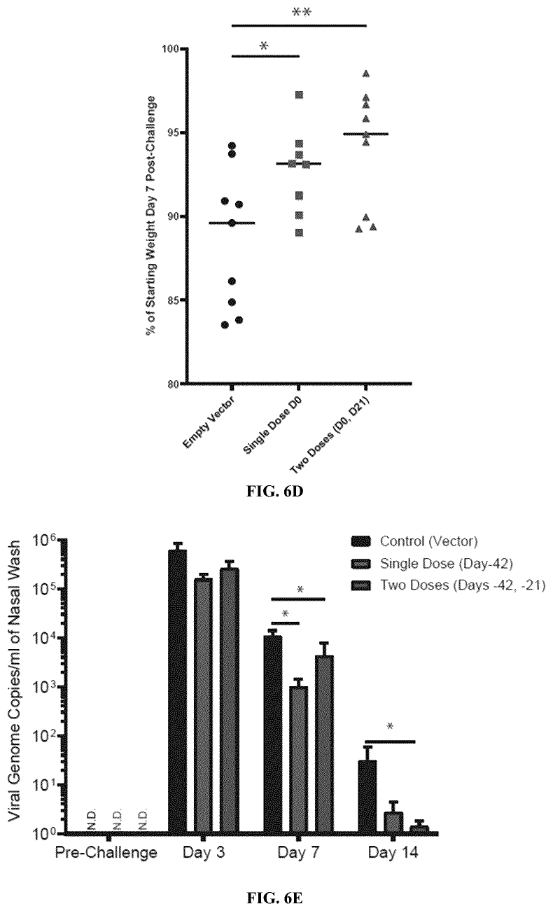

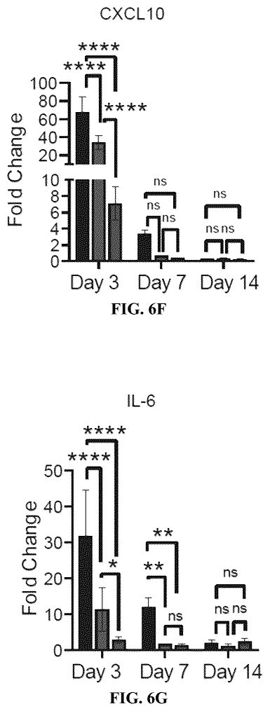

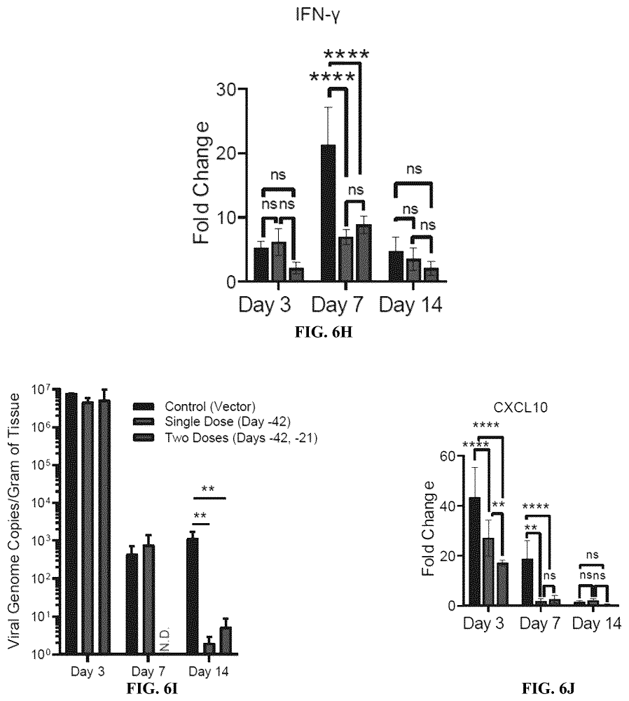

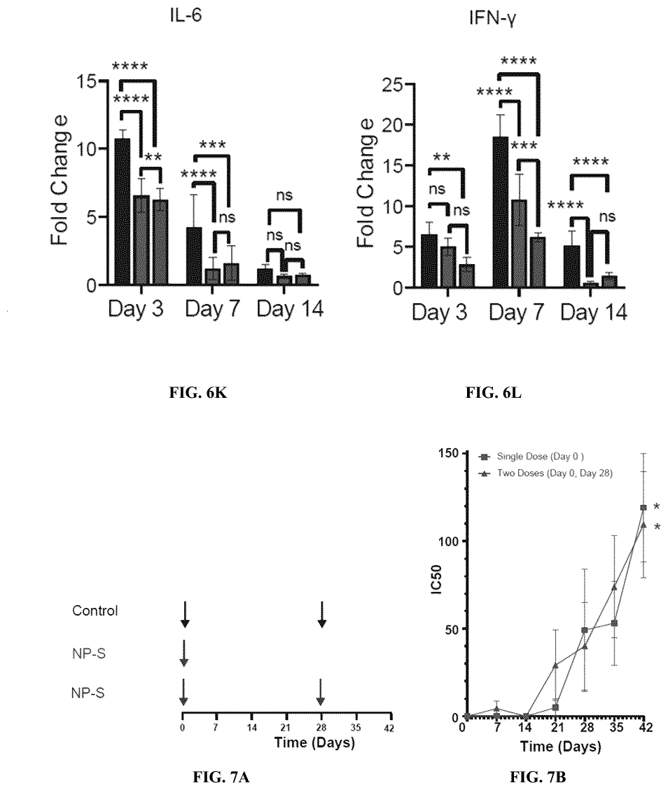

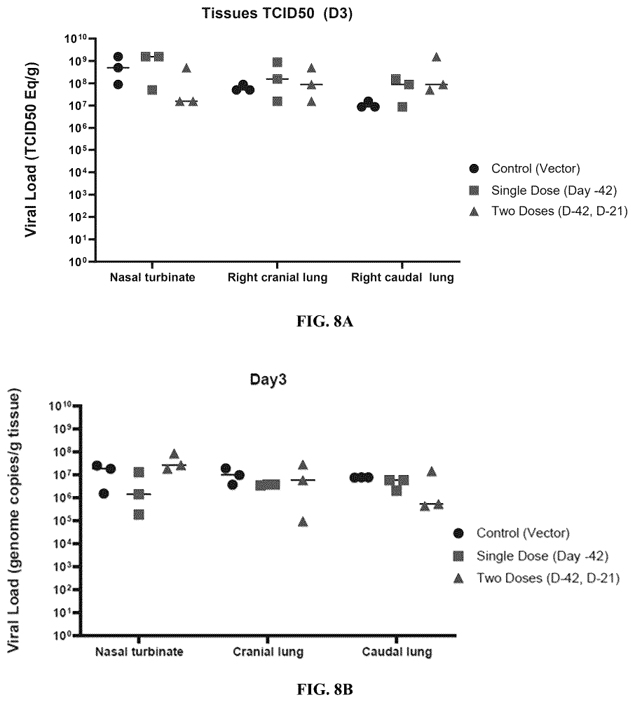

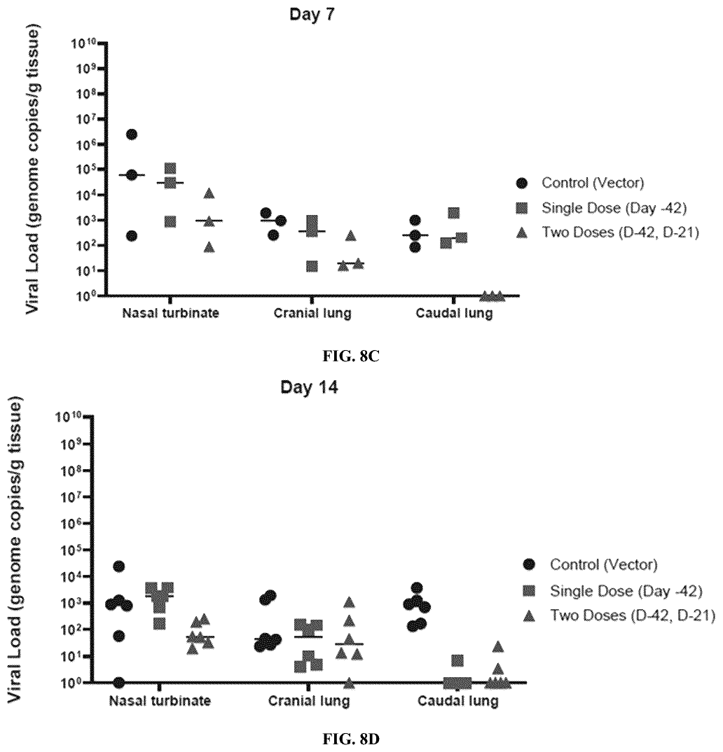

To evaluate the protective efficacy of FAST-PLVs as a COVID-19 vaccine, a SARS-CoV-2 challenge model in Golden Syrian Hamsters was used. Golden Syrian Hamsters were immunized IM 42 days before challenge (priming) with 100 μg of either PLV formulated NP-S or PLV formulated empty vector encoding only the genetic adjuvants (NP-Vector). Twenty-one days before challenge, a second 100 μg dose of NP-S was given to the two-dose group (boosting). Control animals were also given a second dose of NP-Vector at Day-21 (FIG. 6A). Anti-Spike antibody titers were shown to persist to Day-1 prior to challenge (FIG. 6B). Animals were challenged intranasally with a sublethal dose of SARS-CoV-2 (Day 0) and followed for 14 days (FIG. 6A). The two-dose NP-S group showed significantly reduced weight loss as early as Day 1 post-challenge and through to the peak weight loss at Day 7 (FIG. 6C). Although a single dose of NP-S did not significantly decrease weight loss over the first six days, these animals recovered significantly faster than control animals. Weight loss in the single dose group was indistinguishable from the two-dose group at Day 8 post-challenge, with both vaccinated groups showing significantly faster body weight recovery compared to control vaccinated animals (FIG. 6C, FIG. 6D). While no changes to viral loads in nasal washes or lungs were detected by qRT-PCR at three days post-challenge (FIG. 6E, FIG. 8A, FIG. 8B), the proinflammatory factors, CXCL10 and IL-6, were reduced 2-fold in nasal turbinates in the single-dose group and almost 10-fold in the two-dose group (FIG. 6F, FIG. 6G, FIG. 6H). Both groups of vaccinated animals had reduced viral loads in nasal washes 7 and 14-days post-challenge compared to control as determined by qRT-PCR (FIG. 6E). Vaccinated animals showed reduced IL-6 and IFN-g in nasal turbinates 7 days post-challenge with no detectable difference between single- and two-dose groups (FIG. 6G, FIG. 6H). NP-S vaccinated animals that received two doses had undetectable viral loads in the lungs seven days post-challenge, and viral loads in the lungs of both vaccinated groups at day 14 were decreased by 2-3 logs, to near background levels (FIG. 6I, FIG. 8C, FIG. 8D). Inflammatory cytokine expression in the lung was also decreased in both vaccinated animal groups. Both CXCL10 and IL-6 showed decreases in the lung on day 3 with a greater effect observed with 2 doses for CXCL10 (FIG. 6K). No detectable difference between single- and two-dose groups was observed on day 7, and this inflammatory signal was resolved by day 14 in all animals, consistent with IL-6 levels (FIG. 6J, FIG. 6K). IFN-γ expression in the lung was decreased in the two-dose group on day 3 and in both groups on day 7 compared to the control and remained significantly lower than the controls through day 14 (FIG. 6L). Thus, vaccination with FAST-PLVs encapsulating NP-S stimulates a protective immune response that decreases animal morbidity, reduces tissue inflammation, and leads to decreased viral shedding and more rapid viral clearance from the lungs in the hamster challenge model.

### Example 6B—Codon Optimized Omicron Vaccine Candidates are Predicted to Protect Hamsters from SARS-CoV-2 Infection in a Challenge Model

An experiment substantially as described above is carried out for vaccine candidates which encode an omicron variant spike protein (e.g., as in SEQ ID NO: 3, SEQ ID NO: 31, or SEQ ID NO: 36, such as with a polynucleotide sequence of one of SEQ ID NOs: 4, 32-35, and/or 37-40). It is expected that such vaccine candidates will display similar results to those described above, but with enhanced anti-omicron activity (e.g., will better prevent infection from an omicron variant SARS-CoV-2 infection challenge).

### Example 7A—Bi-Valent FAST-PLV Vaccine Candidates Encoding Both Omicron and Wuhan Spike Induce Broad and Robust Antibody Responses

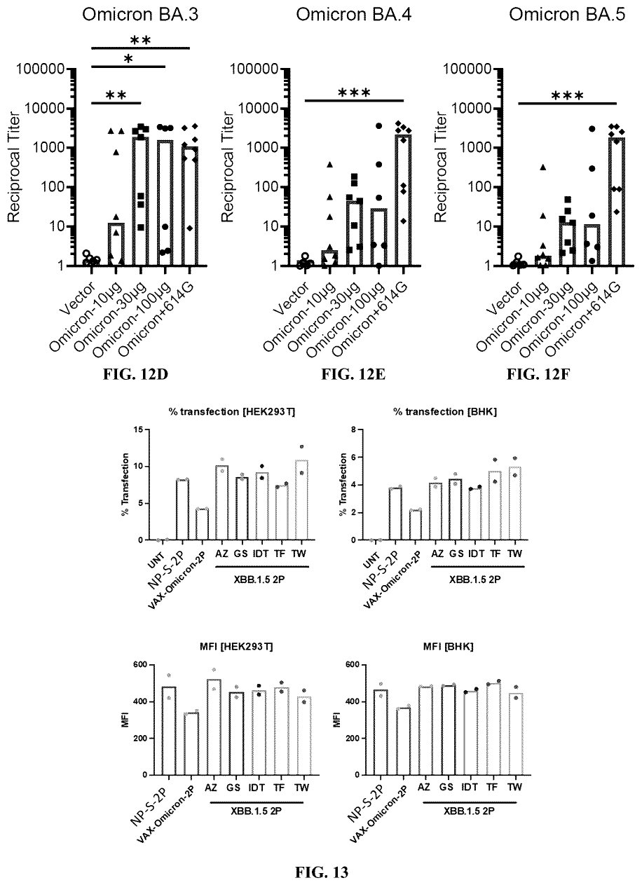

The emergence of SARS-CoV-2 variants with mutations in the Spike protein limiting the effectiveness of Wuhan Spike-based vaccines has driven a need for new booster vaccine strategies to keep pace with SARS-CoV-2 evolution. A bi-valent strategy comprising the use of Wuhan Spike-based vaccine vector (NP-S-2P) in combination with an Omicron-specific vector was proposed. To evaluate immunogenicity of the Omicron Spike (2P mutant based on Omicron B.1.1.529 variant, SEQ ID NO: 3, encoded by the polynucleotide sequence of SEQ ID NO: 4), we immunized mice with 10, 30 or 100 micrograms of Omicron Spike-2P or 100 microgram of a bi-valent vaccine co-formulated with Omicron Spike-2P and Wuhan Spike-2P in a 1:1 ratio (termed “Omicron+614G” herein). Omicron Spike as a monovalent vaccine did not elicit robust or consistent antibody titers against Wuhan Spike (FIG. 12A), Omicron B.1.351 (FIG. 12B), Omicron BA.2 (FIG. 12C), Omicron BA.3 (FIG. 12D), Omicron BA.4 (FIG. 12E), or Omicron BA.5 (FIG. 12F). While 30 and 100 microgram Omicron doses reached significance against Omicron BA.3 (P<0.05), the response in these dose groups was divided (FIG. 12D) with a notable number of low-responder animals. The bi-valent Omicron and Wuhan PLV vaccine generated robust and significant anti-Omicron Spike antibody titers (FIGS. 12A-12F). Therefore, PLV-based DNA vaccines can elicit robust immunity against emerging variants through a bi-valent vaccine strategy.

### Example 7B—Bi-Valent FAST-PLV Vaccine Candidates Encoding Codon Optimized Omicron and/or Wuhan Spike and/or Other SARS-CoV-2 Proteins are Predicted to Induce Broad and Robust Antibody Responses

Additional bi-valent vaccine candidates are also assessed according to the methods described above. Exemplary bi-valent configurations which can be tested are described in the table below. Other combinations of bi-valent vaccine candidates are also contemplated as described herein. In the table below, the first and second SARS-CoV-2 proteins can be encoded on an identical plasmid backbone (e.g., containing all of the same promoter elements, enhancer elements, encoded adjuvants, etc.). Preferably, the plasmid backbone can be that of the NP-S-2P vector described herein (depicted in FIG. 10). It is predicted that these additional bi-valent formats will also exhibit enhanced activity upon administration, including higher antibody titers compared to monovalent vaccines (both for the encoded spike proteins and other variants) and increased neutralizing ability of the antibodies generated. It is also predicted that T cell responses will be further enhanced by the bi-valent vaccines compared to monovalent spike protein vaccines alone.

| | Nucleotide Sequence | | |
|---|---|---|---|
| | Encoding First | | Nucleotide Sequence |
| First SARS- | SARS-CoV-2 | Second SARS-CoV-2 | Encoding Second |
| CoV-2 Protein | Protein | Protein | SARS-CoV-2 Protein |
| Wuhan Spike | SEQ ID NO: 5 | Omicron Spike (SEQ | SEQ ID NO: 4, 32-35, |
| (SEQ ID NO: 2) | | ID NO: 3, 31, or 36) | or 37-40 |
| Wuhan Spike | SEQ ID NO: 5 | Membrane Protein | SEQ ID NO: 42 |
| (SEQ ID NO: 2) | | (SEQ ID NO: 41) | |
| Wuhan Spike | SEQ ID NO: 5 | Nucleocapsid protein | |
| (SEQ ID NO: 2) | | (SEQ ID NO: 43) | |
| Wuhan Spike | SEQ ID NO: 5 | Envelope Protein | |
| (SEQ ID NO: 2) | | (SEQ ID NO: 44) | |
| Omicron Spike | SEQ ID NO: 4, 32- | Membrane Protein | SEQ ID NO: 42 |
| (SEQ ID NO: 3, | 35, or 37-40 | (SEQ ID NO: 41) | |
| 31, or 36) | | | |
| Omicron Spike | SEQ ID NO: 4, 32- | Nucleocapsid protein | |
| (SEQ ID NO: 3, | 35, or 37-40 | (SEQ ID NO: 43) | |
| 31, or 36) | | | |
| Omicron Spike | SEQ ID NO: 4, 32- | Envelope Protein | |
| (SEQ ID NO: 3, | 35, or 37-40 | (SEQ ID NO: 44) | |
| 31, or 36) | | | |
| Omicron Spike | SEQ ID NO: 4 | Omicron Spike (BA.5 | SEQ ID NO: 32-35, or |
| (B.1.1.52) (SEQ | | or XBB.1.5) (SEQ ID | 37-40 |
| ID NO: 3) | | NO: 31 or 36) | |

### Example 8—Methods

The following methods were used to perform the experiments and generate the results described in Examples 1-7 above.

### DNA Vaccine Expression Plasmids

The NTC9385R Nanoplasmid expression plasmid contains a bacterial backbone comprising a 140 bp RNA-based sucrose selectable antibiotic-free marker (RNA-OUT) and a 300 bp R6K origin. Nanoplasmids NTC9385R-eRNA41H and NTC9385R-eRNA41H-CpG RNA are derivatives that co-express RNA adjuvants with the DNA encoded antigen. The D type CpG RNA sequence is 5′GGTGCATCGATGCAGGGGGG 3′ (SEQ ID NO: 8). The RIG-I agonist sequence is: 5′AAAACAGGTCCTCCCCATACTCTTTCATTGTACACACCGCAAGCTCGACAATCA TCGGATTGAAGCATTGTCGCACACATCTTCCACACAGGATCAGTACCTGCTTTCG CTTTT 3′ (SEQ ID NO: 9). Nanoplasmid uses the chimeric CMV promoter to drive transgene expression, with a rabbit beta-globin intron and splice enhancer for efficient RNA export. Rabbit beta-globin polyA signal was used for mRNA transcriptional termination and polyadenylation. Nanoplasmids were manufactured by the Nature Technology Corporation (Lincoln, NE). NP-S-CpG-RIGI comprised the Nanoplasmid NTC9395R-eRNA41H-CpG RNA plasmid with the 3822 bp human codon optimized SARS-CoV-2 Spike gene (synthesized by Integrated DNA Technology; Iowa, USA) cloned in using SalI and BglII restriction sites. NP-S-RIGI comprised the Nanoplasmid NTC9385R-CRNA41H backbone with the 3822 bp human codon-optimized SARS-CoV-2 Spike gene insert. p10-S, p10-RBD, and p10-sRBD candidates used the optimized p10 backbone (Brown et al., 2021) with no encoded RIG-I agonist or CpG sequences. p10-S carried the same Spike gene that was cloned into NP-S-CpG-RIGI and NP-S-RIGI. p10-RBD had the 243 bp RBD sequence from the SARS-CoV-2 Spike gene as the DNA encoded antigen. p10-sRBD carried the RBD sequence fused to the 60 bp sized murine Ig Kappa-chain signal peptide sequence to create a secreted RBD.

### DNA Vaccine Formulation

To manufacture the FAST-PLV DNA vaccines, the antigen-encoded plasmid DNA species were encapsulated within FAST-PLVs as payload. Plasmid DNA was diluted in 10 mM sodium acetate buffer (pH 4.0) containing 5 nM FAST protein. Separately, the PLV lipid components were dissolved in ethanol. Mixing the DNA-protein fraction with the lipid fraction was performed in the NanoAssemblr Benchtop microfluidics instrument (Precision Nanosystems Inc, Vancouver, BC) at a 3:1 ratio and a flow rate of 12 mL/min. Formulations were dialyzed in 8000 MWCO dialysis membranes (product code 12757486, BioDesign, Carmel, New York) against phosphate buffered saline (pH 7.4) for 3 hours with three buffer changes, then concentrated using Amicon ultracentrifuge filters (EMD Millipore, Burlington, Massachusetts) before passage through a 0.22 μm filter (GSWP04700, EMD Millipore). The resulting FAST-PLV DNA vaccine candidates were stored at 4° C. until used.

### PLV Characteristics and Encapsulation Efficiency

PLVs made by NanoAssemblr Benchtop were diluted 1:50 to 1:20,000, depending on concentration, with twice 0.2 μm syringe-filtered PBS buffer. Particle size, polydispersity index (PDI), and zeta potential were measured on final samples using the Malvern Zetasizer Range and a Universal ‘Dip’ Cell Kit (ZEN1002, Malvern) following the manufacturer's instructions. A modified Quant-IT PicoGreen dsDNA assay (P7589, Thermo Fisher Scientific, Edmonton, Canada) was used according to the Assay Kit instructions to calculate the nucleic acid encapsulation efficiency, with the following modifications. PLVs were mixed 1:1 with TE plus Triton (2%) to obtain the Total DNA Concentration, or with TE alone to obtain the Unencapsulated DNA Concentration. The DNA standards were also diluted in TE plus Triton (2%), then together with the samples were incubated at 37° C. for 10 min, then diluted a final time with TE plus Triton (1%) or TE alone, plated in a black 96 well flat-bottomed plate, and measured with a FLUOstar Omega plate reader (415-1147, BMG Labtech). Encapsulation efficiency was calculated by using the following equation:

$$Encapsulation Efficiency = \frac{Total DNA Concentration - Unencapsulated DNA Concentration}{Total DNA Concentration} \times 100$$

### Western Blot Analysis

Cell lysates from HEK293T cells (CRC-3216 ATCC) transfected with Nanoplasmid or the p10 backbone encoding Spike or secreted RBD, or with PLVs carrying SARS-CoV-2 Nucleocapsid as a control, were analyzed via Western blot. Thirty micrograms of cell lysate were loaded per lane and electrophoresed through a 4-20% gradient gel, then transferred to 0.22 μm nitrocellulose at 80V for one hour at 4° C. The nitrocellulose blot was blocked using fluorescent blocking buffer (MB-070, Rockland Immunochemicals, Limerick, United States) or 1% BSA for one hour at ambient temperature then probed with either a 1:2000 dilution of rabbit anti-spike (Catalogue No. 3223, ProSci Antibodies) or mouse anti-Spike RBD (MAB10540, R&D Systems) overnight followed by 1:10000 dilution HRP-conjugated anti-mouse (HAF018, R&D Systems) or Goat anti-rabbit Alexa Fluor 750 (A-21039, Thermo Fisher Scientific) IgG secondary antibody for one hour. Primary mouse anti-alpha tubulin (MAB9344, R&D Systems) or mouse anti-alpha smooth muscle actin (MAB1420, R&D Systems) antibodies were also used as loading controls.

### Rodent Experiments: Ethics and Study Design

All rodent studies were carried out according to the Canadian Council on Animal Care (CCAC) guidelines and approved by the University of Alberta Animal Care and Use Committee. In vivo studies were done using 8-20 weeks old, 25 to 35 g body weight, male and female C57BL/6 mice (Charles River Laboratories, Saint-Constant, QC, Canada). The mice were group-housed in IVCs under SPF conditions, with constant temperature and humidity and with lighting on a fixed light/dark cycle (12-hours/12-hours). Mice were immunized intramuscularly in the musculus tibialis with 50 μl of the test agent unless otherwise stated. Baseline blood was collected via tail vein before immunization. Blood was collected again 14 days after initial immunization, immediately before booster administration. Twenty-one days after immunization, mice were euthanized, and terminal blood was collected via cardiac puncture.

### Non-Human Primate Experiments: Ethics and Study Design

All in-life NHP procedures were carried out by Virscio, Inc, under the guidance of the Institutional Animal Care and Use Committee (IACUC) of the St. Kitts Biomedical Research Foundation (SKBRF), St Kitts, West Indies. SKBRF research facility is fully accredited by the Association for Assessment and Accreditation of Laboratory Animal Care International (AAALAC International). African green monkeys (Chlorocebus sabacus) are an invasive species on the island of St. Kitts and were procured locally using approved practices with IACUC oversight. Groups of 6 animals (3 male and 3 female) per group were enrolled. For all immunization and blood collection procedures, animals were anesthetized with ketamine/xylazine (8.0 mg/kg ketamine [Fort Dodge]/1.6 mg·kg xylazine [Lloyd Lab]) as a sterile, mixed cocktail. General well-being was assessed before, during, and after sedation. All animals were single housed. Baseline plasma was collected immediately prior to intramuscular immunization in the deltoid muscle with the full human dose 250 μg NP-S-CpG-RIGI plasmid DNA formulated in FAST-PLV in 0.5 ml sterile PBS or saline control. Blood draws were collected weekly for 6 weeks and stored at −80° C. until analysis. Individual dose sites were scored according to the Draize system with assessments performed daily post-dosing. On procedure days, animals were sedated with ketamine and xylazine (8 mg/kg: 1.6 mg/kg IM), removed from their cages, and injection site assessed. The classification of the irritancy was obtained by adding the average erythema and edema scores for each assessment and dividing by the number of evaluations. The resulting primary dermal irritation index (PDII) score was classified as follows: PDII<0.5=no irritation; 0.5-2.0=slightly irritating; 2.1-5.0=moderately irritating; >5.0=severely irritating.

### Anti-Spike Antibody Indirect ELISA

Recombinant SARS-CoV-2 S1 Protein (RPO1262, ABclonal, Wuhan, China) was coated on the standard binding plate (Meso Scale Discovery; MSD, Rockville, MD) at 1 μg/mL for 1 h at ambient temperature with shaking. The plate was washed three times with 0.05% Tween-20 in PBS (PBS-T) followed by the addition of Blocker A blocking buffer (R93AA-2, Meso Scale Discovery). After 30 min of incubation, the plate was re-washed with PBS-T. DNA vaccine candidate-immunized mouse serum samples were diluted 1:100 in Blocker A blocking buffer. The plate was rewashed with PBS-T followed by the addition of 1 μg/mL Sulfo-tag Anti-Mouse Secondary Antibody (R32AC-1, Meso Scale Discovery, Rockville, MD). Read Buffer (R92TG-2, Meso Scale Discovery, Rockville, MD) was added to the plate after washing with PBS-T, and the plate was read using the MESO QuickPlex SQ 120 (Meso Scale Discovery; MSD, Rockville, MD). Fold change in anti-Spike IgG was calculated for each animal relative to untreated animals in the same experiment.

### Preparation of VSV-Spike and Lentivirus-Spike Pseudotyped Virus

Neutralizing antibody responses were assessed using either a vesicular stomatitis virus (VSV) encoding GFP but lacking the VSV G protein and pseudotyped with the SARS-CoV-2 Spike protein, or Spike-pseudotyped lentivirus encoding luciferase. To generate the Spike-pseudotyped VSV, 293T cells were transfected with plasmid pMD2.G (pMD2.G was a gift from Didier Trono (Addgene plasmid #12259; RRID: Addgene_12259) expressing the VSV-G protein and 24 h later infected with VSV-ΔG-GFP (Whitt, 2010). The supernatant from cells containing VSV was harvested at 24 h post-transduction, clarified by centrifugation at 500×g for 5 min, and aliquots stored at −80° C. This virus was used to infect 293T cells transfected the previous day with pcDNA3 expressing a codon-optimized version of the SARS-CoV-2 Spike protein (accession #QHD43416.1) with a truncated cytoplasmic tail missing the C-terminal 19 amino acids (pcDNA3-Spike-AC19). Virus inoculum was removed after 1 h at 37° C., cells were washed twice with PBS, incubated in growth medium for 24 h, and the supernatant removed and clarified by centrifugation at 500×g for 5 min before freezing the stock aliquots at −80° C. The Spike-pseudotyped VSV virion stocks were subsequently used for pseudotyped antibody neutralization assays.

To generate spike pseudotyped lentivirus, 293T cells were transfected with pSPAX2 (pSPAX2 was a gift from Didier Trono (Addgene plasmid #12260; RRID: Addgene 12260)), a lentiviral vector encoding Luc2 (based on pLJM1-EGFP, a gift from David Sabatini (Addgene plasmid #19319; RRID: Addgene_19319), and pcDNA3-Spike-ΔC19. After 48 h, the supernatant was removed, filtered at 0.45 μm, aliquoted, and stored at −80° C.

### Pseudotyped Virus Antibody Neutralization Assay

Mouse serum from vaccinated animals and convalescent serum from SARS-CoV-2 infected patients, previously heat-inactivated at 560° C. for 30 min and stored at −80° C., was serially diluted in serum-free media (1:5, 1:10.1:30, 1:80, 1:150 for pseudotyped VSV and 1:12.5-1:1280 for pseudotyped lentivirus) and 50 ul aliquots mixed with 50 ul of Spike-pseudotyped VSV or lentivirus. VSV-antibody mixtures were incubated at 370° C. for 1 h, added to ACE2-expressing Vero E6 cells (African green monkey kidney epithelial cells, ATCC CRL-1586) for 1 h at 37° C.; then the virus inoculum was removed, cells were washed with PBS, and incubated in growth medium for 10 h at 370° C. Cell monolayers were imaged at 200× using an EVOS model cell imaging system (ThermoFisher Scientific, Waltham, MA), and the number of fluorescent cells in 3 random fields from each well quantified using ImageJ software. Lentivirus-antibody mixtures were incubated at 370° C. for 1 h on ACE2-expressing 293A cells (ThermoFisher Scientific #R705). The cells were lysed after 24 h using reporter lysis buffer (Promega #E4030) and stored at −80° C. Luciferase activity was measured using the luciferase assay system (Promega #E1501) on a FLUOstar Omega plate reader (415-1147, BMG Labtech).

### Interferon-Gamma ELISpot

Spleens from immunized animals were dissociated into single-cell suspensions using Spleen Dissociation Kit (130-095-926, Miltenyi Biotec, CA) following the manufacturer's protocol. Single-cell suspensions were counted and resuspended at 2×10⁶ cells/ml for use in the ELISpot assay. Mouse IFN-g ELISpot Kit (EL485, R&D Systems, Minneapolis, MN) according to the manufacture's directions. Briefly, 96-well ELISpot plates pre-coated with capture antibody were hydrated with complete media for 2 hours. Mouse splenocytes were plated at 2.0×10⁵ per well and stimulated at 37° C. for 18 hours with PepMix™ SARS-Cov-2 Spike peptide pools (PM-WCPV-S-1, JPT, Berlin, Germany) at a final concentration of 1.5 nM. PepMix™ peptide pools consist of 15-mer peptides overlapping by 11 amino acids spanning the entire ORF of SARS-CoV-2 Spike divided into two peptide sub-pools. Unstimulated cells served as control. Splenocytes were removed after incubation, and plates were stained with detection antibody for 2 hours at ambient temperature, following which Streptavidin-Alkaline Phosphatase was added for an additional 2 hours. BCIP/NBT substrate (ab7468, Abcam) was added for 1 hour at room temperature. Plates were washed 4× between each staining procedure. Plates were dried for 2 days following BCIP/NBT addition then spots were scanned and quantified using the ImmunoSpot S6 MACRO Analyzer (CTL, Cleveland, United States). Assays were performed in duplicate (for each donor mouse). To calculate Spot-Forming Cells (SFC) per million±SEM for each donor animal mean spot counts were summed for each peptide sub-pool. Control unstimulated splenocytes were subtracted from the representative SARS-CoV-2 Spike-stimulated splenocyte sample for each donor spleen.

### Flow Cytometry Analysis and Immune Phenotyping

Spleen and lymph nodes were isolated and dispersed into single-cell suspensions using mechanical dispersion through 70 μm wire mesh. Red blood cells were lysed using ammonium chloride buffer, followed by a wash with PBS with 2% FBS/PBS. Lymphoid and myeloid populations were examined by flow cytometry. The following antibodies were used: fluorescein isothiocyanate-labeled TCRb (clone H57-597, Biolegend, Cat #109206); Peridinin-chlorophyll-protein Complex:

CY5.5 conjugate-labeled TCRB (clone H57-597, eBioscience, Cat #-45-5961-82), CD8a (clone 53-6.7, Biolegend, Cat #100734); Phycoerythris: Cy-7 tandem conjugate-labeled CXCR5 (clone L138D7, eBioscience, Cat #145515); Allophycocyanin-labelled CD62L (clone MEL-14, Biolegend, Cat #104412); Allophycocyanin-efluor 780-labelled CD69 (clone H1.2F3, eBiosciences, Cat #47-0691-82). Analysis was performed using a three-laser FACSCelesta and Flowjo software (BD Biosciences).

### CTL Killing Assay

Splenocytes were isolated from vaccinated and control mice and co-cultured at a 50:1 ratio with B16-F10 cells stably expressing Sars-CoV-2 Spike protein for 18 hrs at 37° C., 5% CO2 in Dulbecco's Modified Eagle's Medium (DMEM) (Corning, Cat #10-013-CV) supplemented with 10% fetal bovine serum (FBS) (Gibco, Cat #12484028), 100 units/mL of Penicillin (Hyclone, Cat #16777-164), and 100 μg/mL of Streptomycin (Hyclone, Cat #16777-164). B16-F10 cells were washed twice and resuspended in Annexin V binding buffer (Biolegend, Cat #422201). B16-F10 cells were stained with Allophycocyanin-labeled annexin V (BioLegend, Cat #640941) and 7-amino-actinomycin D (BioLegend, Cat #420404) for 15 mins at RT and examined by flow cytometry using a three-laser FACSCelesta. For CTL experiments with purified CD8 T cells, CD8+ T cells were isolated from splenocytes using APC anti-CD8alpha (clone 53-6.7) (Biolegend) and the APC selection kit II (Stem Cell).

### Histology

Spleens and lymph nodes isolated from immunized and control animals were weighed then formalin fixed, and paraffin embedded. 4-6 μm sections were generated. Sections were dewaxed in xylene and rehydrated using graded ethanol to water washes. Sections were stained in Harris Modified Hematoxylin (SH30-4D, Fisher Chemical) for 8 minutes, briefly differentiated in acid alcohol and blued with Scott's Tap Water (pH 8). Slides were then stained in acidified cosin for 30 seconds and dehydrated, cleared, and then mounted. Whole slide images were generated using Panoramic SCAN (3D Histech, Budapest, Hungary) and reviewed by a certified veterinary pathologist for pathology and immune cell proliferation.

SARS-CoV-2 Challenge Study in Hamsters Vaccinated with NP-S-CpG-RIGI

A SARS-Cov2 challenge model in Golden Syrian Hamsters was performed by the Vaccine and Infectious Disease Organization (VIDO) at the University of Saskatchewan. Thirty-six male hamsters were purchased from Charles River Laboratories, Inc. The age of the hamsters at first vaccination was approximately 7-8 weeks. Animals were micro-chipped and randomly assigned to one of 3 groups (n=12/group). The FAST-PLV vaccines were administered intramuscularly (100 μL in each flank). Animals were immunized on Day-42 and animals receiving two-dose regimens were given a second dose on day-21. Hamsters were challenged intranasally (i.n.) with 2×10⁵ TCID50 units (50 μL/nare total dose volume=100 μL) of SARS-CoV-2/Canada/ON/VIDO-01/2020/Vero′76/p.2 (Seq. available at GISAID-EPI_ISL_413015) on Day 0. The same virus stocks were used for challenge and for subsequent assays. Clinical signs, body weights, and body temperature of animals were measured. Animals were observed daily for general health conditions throughout the study period. Animals were euthanized as follows: 3 per Group 3 days postchallenge, 3 per group 7 days post-challenge and 6 per group 14 days post-challenge. At necropsy blood, lung tissues, and nasal turbinates, were collected for assessment of lesions, viral RNA load, infectious virus quantification, virus neutralization assay, and ELISA.

Assessment of Viral Load by qRT-PCR

Extraction of RNA from nasal washes was performed using Qiagen reagents (QIAamp Viral RNA Mini Kit Cat No. 52906) following the manufacturers protocol. Extraction of RNA from lung lobes and nasal turbinates was completed using approximately 100 μg of tissue. The tissues were homogenized in 600 μL of lysis buffer (RLT Qiagen) with a sterile stainless-steel bead in the TissueLyserII (Qiagen) for 6 min, at 30 Hz. The solution was centrifuged at 5000×g for 5 min. Supernatant was transferred to a fresh tube containing 600 μL of 70% ethanol, and the tube was incubated at room temperature for 10 min. Viral RNA was then purified using Qiagen RNeasy Mini Kit (Cat No 74106) and eluted with 50 μL elution buffer.

Viral qRT-PCR Reaction

The qRT-PCR assays were performed on isolated RNA from samples of nasal washes, lung tissues and nasal turbinates using the following SARS-CoV-2 primer-probe combination: Forward Primer (Fwd) 5′-ACAGGTACGTTAATAGTTAATAGCGT-3′ (SEQ ID NO: 16), Reverse Primer (Rev) 5′-ATATTGCAGCAGTACGCACACA-3′ (SEQ ID NO: 17), Labelled Probe 5′-ACACTAGCCATCCTTACTGCGCTTCG-3′ (SEQ ID NO: 18).

### Determination of Infectious Virus by Cell Culture TCID50

Assays to examine biological samples for infectious virus were performed on those samples with a positive qRT-PCR reaction. The assays were conducted in 96-well plates using Vero′76 cells (ATCC CRL-1587). TCID50 was determined by microscopic observation of the cytopathogenic effect (CPE) of cells.

### Hamster Anti-Spike Antibody Determination by Enzyme-Linked Immunosorbent Assay (ELISA)

ELISA assays were performed on serum samples. Plates were coated with SARS-CoV-2 S1 protein antigen at a concentration of 1 μg/mL. Recombinant SARS-CoV-2 S1 protein was produced at VIDO. Plates were blocked with 5% non-fat skim milk powder in PBS containing 0.05% Tween 20. Fourfold dilutions of serum were used. Goat anti-Hamster IgG HRP from ThermoFisher (PA1-29626) was used as the secondary antibody at 1:7000. Plates were developed with OPD peroxidase substrate (0.5 mg/ml) (Thermo Scientific Pierce 34006). The reaction was stopped with 2.5 M sulfuric acid and absorbance was measured at 490 nm. Throughout the assay, plates were washed with PBS containing 0.05% Tween 20. The assay was performed in duplicate. The titers were reported as the end point of the dilutions.

Cytokine Expression in Hamster Tissues by qRT-PCR

Changes in tissue cytokine expression following challenge were determined by qRT-PCR as described previously (Francis et al., 2021).

### Statistical Analyses

The statistics were calculated using GraphPad Prism 5.0 program. Statistical differences between the control and experimental groups were analysed using one-way ANOVA and Dunnet's multiple comparison test and were considered significant for p<0.05. Non-parametric comparisons were performed using the Mann-Whitney test, p<0.05 was considered significant.

### Example 9—Screening of Additional Vaccine Candidates

In addition to the experiments performed on vaccine candidates described in Examples 1-7, additional candidate vaccines were also screened. Results of various experiments performed on these candidates are shown in FIG. 9. Experimental details of how the data in FIG. 9 were generated are found below. In FIG. 9, codon optimization strategy 1 refers to using a codon sequence optimized using the Integrated DNA Technologies (IDT) codon optimization web tool (https://www.idtdna.com/pages/tools/codon-optimization-tool). Codon optimization strategy 2 refers to using a codon sequence optimized using a web tool provided by Thermo Fisher. An exemplary codon optimized sequence using codon optimization strategy 2 is found in SEQ ID NO: 5, which encodes the spike protein used in the NP-S-2P vector.

### Rodent Experiments: Ethics and Study Design

All rodent studies were carried out according to the Canadian Council on Animal Care (CCAC) guidelines and approved by the University of Alberta Animal Care and Use Committee. In vivo studies were done using 8-20 weeks old, 25 to 35 g body weight, male and female C57BL/6 mice (Charles River Laboratories, Saint-Constant, QC, Canada). The mice were group-housed in IVCs under SPF conditions, with constant temperature and humidity and with lighting on a fixed light/dark cycle (12-hours/12-hours). Mice were immunized intramuscularly in the musculus tibialis with 50 μl of the test agent unless otherwise stated. Baseline blood was collected via tail vein before immunization. Blood was collected again 14 days after initial immunization, immediately before booster administration. Twenty-one days after immunization, mice were euthanized, and terminal blood was collected via cardiac puncture.

### Anti-Spike Antibody Indirect ELISA

Recombinant SARS-CoV-2 S1 Protein (RPO1262, ABclonal, Wuhan, China) was coated on the standard binding plate (Meso Scale Discovery; MSD, Rockville, MD) at 1 μg/mL for 1 h at ambient temperature with shaking. The plate was washed three times with 0.05% Tween-20 in PBS (PBS-T) followed by the addition of Blocker A blocking buffer (R93AA-2, Meso Scale Discovery). After 30 min of incubation, the plate was re-washed with PBS-T. DNA vaccine candidate-immunized mouse serum samples were diluted 1:100 in Blocker A blocking buffer. The plate was rewashed with PBS-T followed by the addition of 1 μg/mL Sulfo-tag Anti-Mouse Secondary Antibody (R32AC-1, Meso Scale Discovery, Rockville, MD). Read Buffer (R92TG-2, Meso Scale Discovery, Rockville, MD) was added to the plate after washing with PBS-T, and the plate was read using the MESO QuickPlex SQ 120 (Meso Scale Discovery; MSD, Rockville, MD). Fold change in anti-Spike IgG was calculated for each animal relative to pooled baseline serum from animals in the same experiment.

### Pseudotyped Virus Antibody Neutralization Assay (PSVN Titer)

Mouse serum from vaccinated animals and convalescent serum from SARS-CoV-2 infected patients, previously heat-inactivated at 56° C. for 30 min and stored at −80° C., was serially diluted in serum-free media 1:12.5-1:1280 for pseudotyped lentivirus and 50 ul aliquots mixed with 50 ul of Spike-pseudotyped lentivirus. Lentivirus-antibody mixtures were incubated at 37° C. for 1 h on ACE2-expressing 293A cells (ThermoFisher Scientific #R705). The cells were lysed after 24 h using reporter lysis buffer (Promega #E4030) and stored at −80° C. Luciferase activity was measured using the luciferase assay system (Promega #E1501) on a FLUOstar Omega plate reader (415-1147, BMG Labtech). IC50 values, as defined by the reciprocal of the dilution needed to achieve 50% neutralization, were determined by fitting a 4-parameter inhibitor curve with the following two constraints: top and bottom values were constants based on virus-only and no virus controls, respectively.

### Interferon-Gamma ELISpot

Spleens from immunized animals were dissociated into single-cell suspensions using Spleen Dissociation Kit (130-095-926, Miltenyi Biotec, CA) following the manufacturer's protocol. Single-cell suspensions were counted and resuspended at 2×10⁶ cells/ml for use in the ELISpot assay. Mouse IFN-γ ELISpot assays were performed using the Mouse IFN-γ ELISpot Kit (EL485, R&D Systems, Minneapolis, MN) according to the manufacture's directions. Briefly, 96-well ELISpot plates pre-coated with capture antibody were hydrated with complete media for 2 hours. Mouse splenocytes were plated at 2.0×10⁵ per well and stimulated at 37° C. for 18 hours with PepMix™ SARS-Cov-2 Spike peptide pools (PM-WCPV-S-1, JPT, Berlin, Germany) at a final concentration of 1.5 nM. PepMix™ peptide pools consist of 15-mer peptides overlapping by 11 amino acids spanning the entire ORF of SARS-CoV-2 Spike divided into two peptide sub-pools. Unstimulated cells served as control. Splenocytes were removed after incubation, and plates were stained with detection antibody for 2 hours at ambient temperature, following which Streptavidin-AP was added for an additional 2 hours. BCIP/NBT substrate (ab7468, Abcam) was added for 1 hour at room temperature. Plates were washed 4× between each staining procedure. Plates were dried for 2 days following BCIP/NBT addition then spots were scanned and quantified using the ImmunoSpot S6 MACRO Analyzer (CTL, Cleveland, United States). Assays were performed in duplicate (for each donor mouse). To calculate Spot-Forming Cells (SFC) per million±SEM for each donor animal mean spot counts were summed for each peptide sub-pool. Counts from control unstimulated splenocytes were subtracted from each sample.

### CTL Killing Assay (CTL FUNCTION)

Splenocytes were isolated from vaccinated and control mice and co-cultured at a 50:1 ratio with B16-F10 cells stably expressing Sars-CoV-2 Spike protein for 18 hrs at 37° C., 5% CO2 in Dulbecco's Modified Eagle's Medium (DMEM) (Corning, Cat #10-013-CV) supplemented with 10% fetal bovine serum (FBS) (Gibco, Cat #12484028), 100 units/mL of Penicillin (Hyclone, Cat #16777-164), and 100 μg/mL of Streptomycin (Hyclone, Cat #16777-164). B16-F10 cells were washed twice and resuspended in Annexin V binding buffer (Biolegend, Cat #422201). B16-F10 cells were stained with Allophycocyanin-labeled annexin V (BioLegend, Cat #640941) and 7-amino-actinomycin D (BioLegend, Cat #420404) for 15 mins at RT and examined by flow cytometry using a three-laser FACSCelesta. For CTL experiments with purified CD8 T cells, CD8+ T cells were isolated from splenocytes using APC anti-CD8alpha (clone 53-6.7) (Biolegend) and the APC selection kit II (Stem Cell). Note: In the summary chart, results were graded on 0-4 scale where 0 indicates no detectable CTL function ranging to 4 which was maximum CTL function.

### Example 10—Development of Omicron Variant XBB.1.5 Vaccine Construct

### In Vitro Characterization

To develop a vaccine construct capable of recognizing Omicron variant XBB.1.5, we designed five novel construct sequences encoding the XBB.1.5 spike protein with the 2P mutations included. These constructs encode the same protein sequence (SEQ ID NO: 36) but differ in their codon usage; they are termed XBB.1.5-2P_AZ (“AZ”) (SEQ ID NO: 40), XBB.1.5-2P_GS (“GS”) (SEQ ID NO: 39), XBB.1.5-2P_IDT (“IDT”), XBB.1.5-2P_TF (“TF”) (SEQ ID NO: 37), and XBB.1.5-2P_TW (“TW”) (SEQ ID NO: 38). Each construct was cloned into the NTC nanoplasmid backbone with CpG motifs and RIG-I encoded agonist, equivalent to the NP-S-2P vector backbone described herein. Initially the five constructs were tested in vitro by transfection of HEK293T and BHK cell lines with 250 ng DNA. Cells were harvested 24 hours post-transfection and assessed for Spike protein expression via flow cytometry. All five constructs showed robust expression in vitro (FIG. 13), however constructs XBB.1.5-2P_AZ, XBB.1.5-2P_GS, and XBB.1.5-2P_TF were prioritized for in vivo characterization.

### In Vivo Validation of XBB.1.5 Constructs

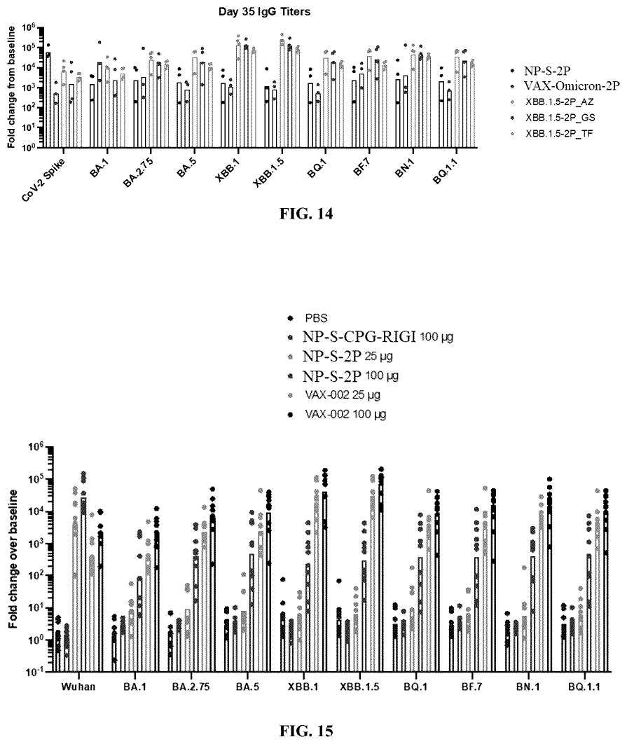

Constructs XBB.1.5-2P_AZ, XBB.1.5-2P_GS, and XBB.1.5-2P_TF were characterized for in vivo immunogenicity in C57BL/6 mice, along with NP-S-2P and VAX-Omicron-2P (analogous to the NP-S-2P vaccine but encoding the Omicron variant spike protein of SEQ ID NO: 3 using the nucleotide sequence of SEQ ID NO: 4) as controls. Each construct was given as two I.M. doses of 100 μg on day 0 and day 21, and antibody titers were measured on Day 35 (FIG. 14). All novel XBB.1.5 constructs showed high immunogenicity, however XBB.1.5-2P_AZ was determined to have the highest antibody titers against more recent circulating strains, and was advanced as “VAX-002” for additional development.

### Induction of Humoral Immunity by VAX-002

To evaluate the humoral response elicited by VAX-002, an immunogenicity and safety bridging study was conducted with VAX-002 and included vaccine candidates (i.e., NP-S-CPG-RIGI vaccine as described herein and NP-S-2P as described herein), at one or two dose levels. Group assignment and doses were as indicated in the table below. Immunization was done with a single IM injection on Day 1, and evaluation of anti-SARS-CoV-2 Spike immunoglobulin G (IgG) antibody production and neutralization potency was determined from serum samples collected on Day 36.

TABLE 3

| | Dose | Dose Volume | Route of | No. of |
|---|---|---|---|---|
| Test Article | (μg) | (mL/animal) | Administration | Animals |
| Control (PBS) | 0 | 0.05 | IM | 10M/10F |
| NP-S-CPG-RIGI | 100 | 0.05 | IM | 10M/10F |
| NP-S-2P | 25 | 0.05 | IM | 10M/10F |
| NP-S-2P | 100 | 0.05 | IM | 10M/10F |
| VAX-002 | 25 | 0.05 | IM | 10M/10F |
| VAX-002 | 100 | 0.05 | IM | 10M/10F |

Following immunization, the anti-Spike IgG titer was determined by indirect electrochemiluminescence immunoassay (ECLIA). A threshold for seroconversion of >4-fold increase in anti-Spike IgG antibody titer over baseline (pre-immunization) serum was established to be consistent with our proposed clinical studies. At Day 36, the serum concentration of anti-Spike IgG against a panel of circulating variants elicited by VAX-002 was determined using a validated assay (Meso Scale Discovery). VAX-002 induced a robust increase in serum anti-Spike IgG levels, at a dose level of 25 μg (geometric mean titer [GMT] of 31,945.9 against XBB.1.5 Spike) and 100 μg (GMT 73,095.7) and 100% seroconversion of vaccinated animals at both dose levels (FIG. 15). NP-S-2P vaccine induced high titers against Wuhan strain at both dose levels (GMT 3,951.1 and 27,428.5 for 25 and 100 μg doses, respectively), but induced much lower titers against variants from the Omicron lineage (FIG. 15). This observation matches with reports of immune evasion by Omicron variants, and justifies the switch of cargo from NP-S-2P to VAX-002 to induce antibodies against variants of concern in the Omicron lineage.

Neutralizing Antibody Production Following Immunization with VAX-002

The utility of using BSL2 pseudo-virion based neutralization (PsVN) assays as a readout for functional, protective humoral responses in animal models has been demonstrated in the assessment of multiple COVID-19 vaccine candidates. PsVN assays do not require any species-specific reagents, thus values can be compared directly across species. Early challenge studies in NHPs clearly indicate that a half maximal inhibitory concentration (IC50) of 1:50 to 1:100 is sufficient to protect against SARS-CoV-2 challenge. Vaccines that induce a nAb titer greater than 1:100 in nonclinical models have also proven to be efficacious in the human population. The production of nAbs following administration of VAX-002 was evaluated using a validated PsVN assay performed by an independent laboratory (Nexelis, Inc). Briefly, SARS-CoV-2 Spike protein-pseudotyped lentivirus expressing luciferase was treated with serial dilutions of immunized mouse serum and added to Vero E6 cells stably expressing the human ACE2 receptor. The pseudotyped lentivirus displays SARS-CoV-2 Spike protein on its surface, which enables infection of ACE2-expressing cells. The amount of luciferase produced in the infected ACE2-expressing cells correlates directly with the number of non-neutralized virus particles (after incubation with serum samples). The luciferase activity is measured to determine the efficiency of infection neutralization capacity of the sera, and is used to calculate the dilution at which 50% neutralization is achieved (IC50).

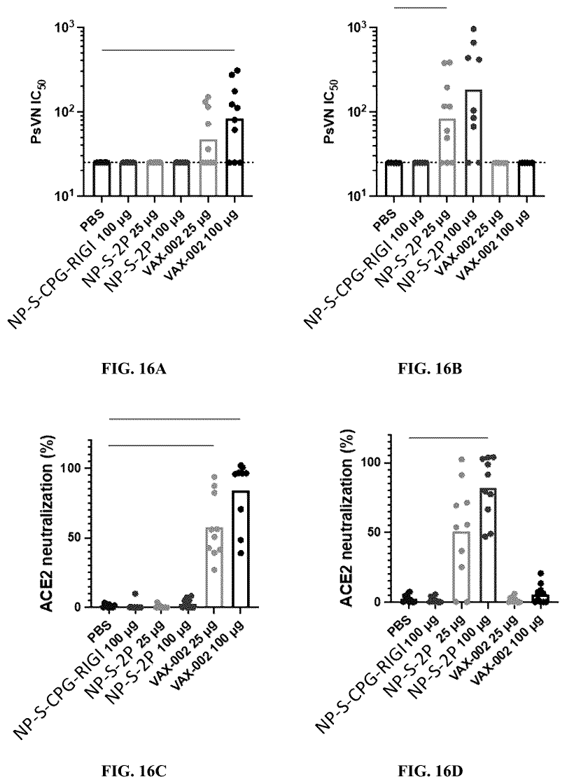

Scrum from animals immunized with VAX-002 generated a robust neutralization response against pseudovirus displaying XBB.1.5 Spike protein, with IC50 values reaching 875 (GMT=88, P<0.01 versus control), with no other vaccine candidates showing neutralization against this strain (FIG. 16A). Serum from NP-S-2P immunized animals was able to neutralize pseudovirus displaying the D614G but not XBB.1.5 Spike protein (FIG. 16B), confirming our hypothesis that the potency of NP-S-2P against Omicron lineage viruses would be suboptimal, and that a novel vaccine cargo (VAX-002) was needed to address currently circulating variants. Another plasmid DNA-based COVID-19 vaccine candidate generated IC₅₀ values ranging from 43 to 147 using a prime-boost two-dose regimen.

As a second measure of nAb production induced by VAX-002, an ACE2 inhibition assay was performed. Briefly, with this approach the antigen (SARS-CoV-2 Spike) is attached to an electrochemiluminescence immunoassay (ECLIA) plate and detected by binding recombinant labeled ACE2 protein. Unlabeled recombinant ACE2 protein is used to calibrate the assay and determine percent neutralization of serum samples. Serum samples were derived from mice immunized as indicated in Table 3. Scrum from VAX-002 vaccinated animals showed significant inhibition of ACE2 binding to Spike protein from XBB.1.5 at both dose levels (mean inhibition of 57.6% and 84.2% for 25 and 100 μg doses, respectively) (FIG. 16C). Serum from NP-S-2P immunized animals was able to inhibit ACE2 binding to Spike protein of Wuhan strain but not XBB.1.5, indicating that the shift in cargo from NP-S-2P to VAX-002 was necessary to confer neutralizing capacity against more recent circulating variants (FIG. 16D). Together, these results demonstrate that administration of a single dose of VAX-002 in mice elicits a nAb response well within the established range to confer protection against SARS-CoV-2 challenge and consistent with neutralizing responses in convalescent patients (Yu 2020).

Induction of SARS-CoV-2 Spike-Specific T Cell Responses with VAX-002

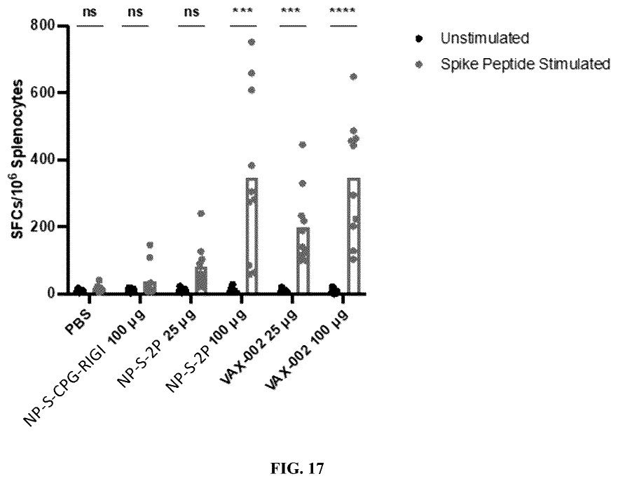

SARS-CoV-2 Spike-specific cell-mediated immunity was evaluated in splenocytes using interferon-gamma (IFN-γ)-based enzyme-linked immunospot assay (ELIspot). As above, animals were immunized as indicated in Table 3. On Day 36, splenocytes were isolated following necropsy. Splenocytes were stimulated ex vivo with an overlapping peptide pool (15 mers overlapping by 11 amino acids) spanning the entire SARS-CoV-2 Spike protein open reading frame (ORF), strain XBB.1.5. SARS-CoV-2 Spike-specific IFN-γ release was then measured by ELISpot. Splenocytes from VAX-002-treated animals showed an increased frequency of IFN-γ positive T-cells (FIG. 17 FIG. 5, P<0.0001) upon stimulation, compared with control treated animals. This data suggests that VAX-002 invokes robust cell-mediated immunity in addition to the neutralizing antibody response.

### Safety of VAX-002 in Mice

A non-GLP safety study (23-AES008) was conducted in mice to evaluate the safety characteristics of VAX-002, and compare to the vaccine candidates NP-S-CPG-RIGI and NP-S-2P. C57BL6 mice (N=10/sex/group) were given a single dose of either NP-S-CPG-RIGI (100 μg), NP-S-2P (25 or 100 μg), or VAX-002 (25 or 100 μg), as indicated in Table 3. The dosing regimen was selected to be in accordance with the proposed clinical strategy of a single dose booster, as opposed to earlier studies which used a two-dose regimen.

There was no treatment-related mortality/morbidity observed at any dose level following IM administration. Transient decreases in percent body weight relative to pre-dose were observed following dosing in each study arm, which rebounded to pre-dose levels by Day 3 post-injection.

Clinical chemistry parameters were measured in serum on Day 2 and Day 36 post-dosing. VAX-002-related findings included changes in clinical chemistry parameters (decreased albumin, glucose, alkaline phosphatase (ALP), and blood urea nitrogen (BUN) and increased potassium, and globulin), within the range of historical controls. Complete blood counts were evaluated on Day 2 and Day 36 post-dosing, and no significant alteration in hematology parameters was seen across all vaccines and doses. Histopathological evaluation of the injection site (quadricep), spleen, and lymph nodes showed no significant treatment-related adverse effects, with the only observation being mild inflammation in the quadricep within the range of expected findings for an intramuscular injection. Histopathology was conducted on liver and lungs as well, but data was inconclusive due to processing-related artifacts in samples independent of treatment group.

The acute phase proteins alpha-2-macroglobulin (A2M) and alpha-1-acid glycoprotein (AGP) were measured at baseline (pre-immunization), 24 hours (Day 2), 48 hours (Day 3), and 168 hours (Day 8) post-injection. A2M levels were transiently elevated in all groups, with significant differences vs PBS control on Day 3 (P<0.05), but differences between PBS control were insignificant by Day 8. AGP levels were similarly elevated in a transient manner, with groups receiving a high dose (100 μg) showing significant differences vs PBS control (P<0.05) on Days 2 and 3 but not on Day 8. The magnitude in changes for acute phase proteins was low, with a 2-3-fold increase in A2M at its peak (Day 3) and a 4-6-fold increase in AGP at the peak (Day 3). Such increases in A2M and AGP are indicative of transient activation of the innate immune system.

Proinflammatory cytokines in serum were similarly measured at baseline (pre-immunization), 24 hours (Day 2), 48 hours (Day 3), and 168 hours (Day 8) post-injection. Compared to PBS control group, significant changes in levels of several cytokines (IFN-γ, IL-1B, IL-5, IL-6, KC/GRO, IL-10, and TNF-α) were observed. Cytokine levels peaked at Day 2, and in all cases were not significantly different than the control group by Day 8. The observed changes were generally small in magnitude, with cytokine levels increasing less than 5-fold in all groups, with the exceptions of IFN-γ and IL-6. IFN-γ levels, which were elevated by up to 25-fold at peak on Day 2, and IL-6 levels were elevated by up to 13-fold at peak on Day 2. The increases in IFN-γ are consistent with a robust T-cell response, similar to what was observed in the immunogenicity assays from this study.

### Example 11—Phase 1/2 Clinical Trial of VAX-002

A Phase 1/2 clinical trial is performed based on VAX-002 as a therapeutic intervention based on that of its predecessors VAX-001 (NP-S-CpG-RIGI as described herein) and VAX-001b (NP-S-2P as described herein). Previous clinical studies of VAX-001 and VAX-001b determined that both high (250 microgram) and low (100 microgram) doses of these constructs were safe with no serious adverse events reported. It is predicted that VAX-002 will display a similar safety profile.

This study will investigate the safety and immunogenicity of VAX-002 when given as a booster dose to healthy adults who have received another authorized COVID-19 vaccine previously. It is predicted that VAX-002 will produce substantially enhanced humoral immunity against new circulating variants of SARS-CoV-2 (e.g., omicron variants). Phase 1 will contain a dose-finding/safety evaluation part (100 microgram and 250 microgram doses administered), with Phase 2 being adaptive to evaluate the safety and efficacy of VAX-002 at the optimal dose determined in Phase 1.

In Phase 1 up to 50 participants are planned to be randomized 1:1 into one of two groups: either 100 μg intramuscular (IM) injection or 250 μg IM injection. Participants are to receive a single 0.5 mL IM injection of VAX-002 100 μg or 250 μg on Day 0. Follow-up visits will occur on Days 7, 14, 17 (phone call visit), 21, 28, 42, and 180. An interim analysis is planned once all participants in Phase 1 have completed their Day 28 visit to evaluate dose-response and safety to support optimal dose selection for Phase 2.

In Phase 2 approximately 250 participants will be enrolled and will receive a single 0.5 mL IM injection of the optimal VAX-002 dose (determined in the Phase 1 interim analysis) on Day 0. Follow-up visits will occur on Days 7, 14, 17 (phone call visit), 21, 28, 42, and 180.

The dose regimen for both Phase 1 and Phase 2 studies will include a single injection (0.5 mL) of either 100 or 250 micrograms of VAX-002 via intramuscular administration.

The primary objective of the Phase 1 study is to evaluate the safety of VAX-002, with the primary objective of Phase 2 being further evaluation of safety and to assess the efficacy in providing protection against the SARS-CoV-2 virus. To assess safety, endpoints will include 1) Frequency and grade of each solicited local (injection site) and systemic reactogenicity adverse event (AE) from Day 0 through Day 28; 2) Frequency and grade of unsolicited AEs, significant AEs (SAEs), and medically attended AEs (MAAEs); and 3) Frequency, type, and grade of SAEs related to administration, MAAEs related to administration, or COVID-19 related illness from administration through the end of the study. It is predicted that all doses of VAX-002 will be well tolerated with minimal adverse events.

The secondary objective is to assess SARS-CoV-2 neutralizing antibody response from baseline (Day 0) through Day 28. As secondary endpoints 1) Antibody responses at specified timepoints from baseline through Day 28 and end of study will be assessed; and 2) seroconversion rate (% of participants who seroconvert, defined as a 4-fold or greater increase in neutralizing antibody titers, as measured be SARS-CoV-2 neutralization assay at specified timepoints from baseline through end of study. It is predicted that all or substantially all subjects will generate therapeutically relevant levels of antibody response and all or substantially all subjects will seroconvert.

Exploratory objectives of the study are to assess the humoral immune response of VAX-002 booster to the SARS-CoV-2 S protein; to evaluate dose-response immunity; to evaluate antigen specific B and T cell response by interferon-gamma ELIspot assay with overlapping peptide pools (15 mers overlapping by 11 residues) of vaccine antigens and by immunophenotyping T cells by flow cytometry (samples will be bio-banked for future analysis depending on the immune response); to evaluate anti-FAST protein antibody; and to evaluate anti dsDNA antibodies. This will be accomplished by measuring immune response at specified timepoint from baseline through the end of study (IgG antibody titers against S protein by ELISA or ECLIA; percentage seroconversion; neutralizing antibody titers against the pseudo-virion as measured by PSVN assay); assessing persistence of IgG antibody titers as measured as discussed previously; measuring spot forming cells per million PBMC and the differential expression of T cellular markers measured at specified timepoint from baseline through end of study; performing immunophenotyping flow cytometry at specified timepoints, measuring anti-FAST protein antibodies at day 0, 28, and 42 (e.g., by ELISA or ECLIA), and measuring anti-dsDNA antibodies (e.g., by chemiluminescent immunoassay (CIA)) at days 0, 28, and 42. It is predicted that VAX-002 will result in robust IgG antibody titer and strong immune response.

The above study will be performed on healthy adults 18 years or older who have previously completed a prior COVID-19 primary vaccination course or booster at least 3 months prior to enrollment or recent confirmed SARS-CoV-2 infection in the past three months but less than one month from the start of enrollment. Exclusion criteria include history of anaphylaxis to key vaccine ingredients, pregnancy or likelihood of becoming pregnant during the study, positive test result for HIV or hepatitis B or C, history of seizure disorder, encephalopathy, or psychosis, standard laboratory tests outside of normal range (e.g., complete blood count, prothrombin time, partial thromboplastin time, alanine aminotransferase, aspartate aminotransferase, alkaline phosphatase, total bilirubin, creatinine, lipase, blood glucose), active infectious disease, unstable underlying conditions, history of Guillain-Barre Syndrome or other degenerative neurological disease, history of autoimmune, inflammatory, or potential immune-mediated diseases, history of serious cardiovascular diseases, history of immunodeficiency, asplenia, or functional asplenia, history of platelet or other bleeding disorder, heavy smoker, vaper, or cannabis user, history or diagnosis of coagulopathies, prior receipt of immunosuppressive medication, cytotoxic therapy, or systemic corticosteroids within 6 months, recent receipt of blood products, administration of other investigational drugs within 3 months, or other condition which may interfere with compliance, evaluation, or informed consent process.

The VAX-002 doses will be packaged as sterile, single-use, preservative free suspension in a USP Type I glass contain. The composition will include the plasmid, FAST protein, Fusogenix™ lipid mix in a buffer of 5% sucrose in PBS solution. Dose will be 0.5 mL of either 100 microgram or 250 microgram of VAX-002 to be injected by intramuscular injection into the deltoid muscle of the subject on Day 0. VAX-002 doses will be stored at 2-8° C., maintained during transit and at all relevant times prior to administration.

During the study, Scrum samples will be taken and assessed by SARS-CoV-2 neutralizing antibody assay at Days 0, 7, 14, 21, 28, 42, and 180. Serum samples will also be assessed by ELISA or ECLIA and PVN at Days 0, 7, 14, 21, 28, 42, and 180. Blood samples will be tested for cell mediated immune response (PBMC and ELISpot) at Days 0, 7, 14, 21, 28, 42, and 180. Blood samples will also be tested for cell-mediated immune response (ICS) at Days 0, 7, 14, 21, 28, 42, and 180. Serum samples will also be tested for Anti-FAST protein antibodies and anti-dsDNA (e.g., plasmid) antibodies at days 28 and 42.

It is predicted that the study described above will show that VAX-002 at both doses will result in robust immune response and protection against SARS-CoV-2 infection over the period of the study, including a robust immune response against a wide variety of virus variants.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A SARS-CoV-2 DNA vaccine, comprising:
a) a first DNA vector comprising a polynucleotide sequence encoding a first SARS-CoV-2 spike protein or a portion thereof, wherein the first SARS-CoV-2 spike protein or portion thereof comprises 1) at least one amino acid substitution selected from a D614G amino acid substitution, a K986P amino acid substitution and a V987P amino acid substitution, and 2) an additional amino acid substitution at another residue of the first SARS-CoV-2 spike protein, wherein residue position numbering is based on SEQ ID NO: 1 as a reference sequence;
b) an adjuvant or a polynucleotide sequence encoding an adjuvant;
wherein the first DNA vector is encapsulated in proteolipid vehicles that comprise a fusogenic membrane protein.

2. A SARS-CoV-2 DNA vaccine, comprising:
a) a first DNA vector comprising a polynucleotide sequence encoding a first SARS-CoV-2 spike protein or a portion thereof,
b) a second DNA vector comprising a polynucleotide sequence encoding a second SARS-CoV-2 protein or a portion thereof; and
wherein the first DNA vector and the second DNA vector are encapsulated in proteolipid vehicles that comprise a fusogenic membrane protein, optionally comprising an adjuvant or a polynucleotide sequence encoding an adjuvant.

3. A SARS-CoV-2 DNA vaccine, comprising:
a) a first DNA vector comprising a polynucleotide sequence encoding a first SARS-CoV-2 spike protein or a portion thereof, wherein the SARS-CoV-2 spike protein or portion thereof comprises a D614G amino acid substitution, a K986P amino acid substitution, a V987P amino acid substitution, or any combination thereof, wherein residue position numbering is based on SEQ ID NO: 1 as a reference sequence;
b) a second DNA vector comprising a polynucleotide sequence encoding a second SARS-CoV-2 protein or a portion thereof; and
c) an adjuvant or a polynucleotide sequence encoding an adjuvant;
wherein the first DNA vector and the second DNA vector are encapsulated in proteolipid vehicles that comprise a fusogenic membrane protein.

4. The SARS-CoV-2 DNA vaccine of any one of the preceding claims, wherein the first SARS-CoV-2 spike protein or portion thereof comprises a D614G amino acid substitution, a K986P amino acid substitution, and a V987P amino acid substitution.

5. The SARS-CoV-2 DNA vaccine of any one of the preceding claims, wherein the first SARS-CoV-2 spike protein or the portion thereof is a full-length SARS-CoV-2 spike protein.

6. The SARS-CoV-2 DNA vaccine of any one of the preceding claims, wherein the first SARS-CoV-2 spike protein comprises an amino acid sequence having at least 90%, at least 95%, at least 96%, at least 97%, at least 98%, or at least 99% sequence identity with the sequence set forth in SEQ ID NO: 1.

7. The SARS-CoV-2 DNA vaccine any one of the preceding claims, wherein the first SARS-CoV-2 spike protein further comprises a T19I, 24-26Del, A27S, A67V, 69-70Del, T95I, 137-145Del, G142D, 143-145Del, Y145H, 211Del, L212I, V213G, ins214EPE, ins214TDR, A222V, G339D, G339H, R346K, R346S, V367F, L368I, S371F, S371L, S373P, S375F, T376A, P384L, N394S, D405N, R408S, Q414K, K417N, K417T, N439K, N440K, V445P, G446S, Y449H, Y449N, N450K, L452R, L452Q, N460K, S477N, T478K, V483A, E484A, E484K, E484Q, E484Del, F486P, F486V, F490R, F490S, Q493K, Q493R, S494P, G496S, Q498R, N501T, N501Y, Y505H, E516Q, T547K, Q613H, A653V, H655Y, G669S, Q677H, N679K, ins679GIAL, P681H, P681R, A701V, S704L, N764K, D796Y, N856K, Q954H, N969K, or L981F modification, or any combination thereof.

8. The SARS-CoV-2 DNA vaccine of any one of the preceding claims, wherein the first SARS-CoV-2 spike protein comprises an amino acid sequence of SEQ ID NO: 2, 3, 31, or 36.

9. The SARS-CoV-2 DNA vaccine of any one of the preceding claims, wherein the polynucleotide sequence encoding the first SARS-CoV-2 spike protein comprises a polynucleotide sequence having at least 80%, at least 85%, at least 90%, at least 95%, at least 97%, at least 98%, or at least 99% sequence identity with the sequence set forth in SEQ ID NO: 4, 5, 32-35, or 37-40.

10. The SARS-CoV-2 DNA vaccine of any one of the preceding claims, wherein the polynucleotide sequence encoding the first SARS-CoV-2 spike protein comprises a polynucleotide sequence of SEQ ID NO: 4, 5, 32-35, or 37-40.

11. The SARS-CoV-2 DNA vaccine of any one of the preceding claims, wherein the first SARS-CoV-2 spike protein comprises an amino acid sequence having at least 80%, 85%, 90%, 95%, 96%, 97%, 98%, 99%, 99.5%, or 100% sequence identity to the sequence set forth in SEQ ID NO: 36, and wherein the polynucleotide sequence encoding the first SARS-CoV-2 spike protein comprises a polynucleotide sequence having at least at least 80%, at least 85%, at least 90%, at least 95%, at least 97%, at least 98%, or at least 99%, or 100% sequence identity to the sequence set forth in SEQ ID NO: 40.

12. The SARS-CoV-2 DNA vaccine of any one of the preceding claims, wherein the second SARS-CoV-2 protein is a second SARS-CoV-2 spike protein.

13. The SARS-CoV-2 DNA vaccine of claim 12, wherein the second SARS-CoV-2 spike protein comprises at least 5, at least 10, at least 15, at least 20, at least 25, or at least 30 amino acid modifications relative to the first SARS-CoV-2 spike protein.

14. The SARS-CoV-2 DNA vaccine of claim 12 or 13, wherein the second SARS-CoV-2 spike protein further comprises a T19I, 24-26Del, A27S, A67V, 69-70Del, T95I, 137-145Del, G142D, 143-145Del, Y145H, 211Del, L212I, V213G, ins214EPE, ins214TDR, A222V, G339D, G339H, R346K, R346S, V367F, L368I, S371F, S371L, S373P, S375F, T376A, P384L, N394S, D405N, R408S, Q414K, K417N, K417T, N439K, N440K, V445P, G446S, Y449H, Y449N, N450K, L452R, L452Q, N460K, S477N, T478K, V483A, E484A, E484K, E484Q, E484Del, F486P, F486V, F490R, F490S, Q493K, Q493R, S494P, G496S, Q498R, N501T, N501Y, Y505H, E516Q, T547K, Q613H, D614G, A653V, H655Y, G669S, Q677H, N679K, ins679GIAL, P681H, P681R, A701V, S704L, N764K, D796Y, N856K, Q954H, N969K, L981F, K986P, or V987P modification, or any combination thereof.

15. The SARS-CoV-2 DNA vaccine of any one claims 12-14, wherein the second SARS-CoV-2 spike protein comprises a K986P amino acid substitution and a V987P amino acid substitution.

16. The SARS-CoV-2 DNA vaccine of any one of claims 12-15, wherein the second SARS-CoV-2 spike protein comprises an amino acid sequence having at least 90%, at least 95%, at least 96%, at least 97%, at least 98%, or at least 99% sequence identity with the sequence set forth in SEQ ID NO: 31 or 36.

17. The SARS-CoV-2 DNA vaccine of any one of claims 12-16, wherein the second SARS-CoV-2 spike protein comprises an amino acid sequence of SEQ ID NO: 31 or 36.

18. The SARS-CoV-2 DNA vaccine of any one of claims 12-17, wherein the polynucleotide sequence encoding the second SARS-CoV-2 spike protein comprises a polynucleotide sequence having at least 80%, at least 85%, at least 90%, at least 95%, at least 97%, at least 98%, or at least 99% sequence identity with the sequence set forth in SEQ ID NO: 4, 32-35, or 37-40.

19. The SARS-CoV-2 DNA vaccine of any one of claims 12-18, wherein the polynucleotide sequence encoding the second SARS-CoV-2 spike protein comprises a polynucleotide sequence of SEQ ID NO: 4, 32-35 or 37-40.

20. The SARS-CoV-2 DNA vaccine of any one of claims 12-19, wherein the first SARS-CoV-2 spike protein and the second SARS-CoV-2 spike protein are derived from different variants of the SARS-CoV-2 virus.

21. The SARS-CoV-2 DNA vaccine of any one of the preceding claims, wherein the first SARS-CoV-2 spike protein is derived from the Wuhan strain of SARS-CoV-2.

22. The SARS-CoV-2 DNA vaccine of any one of claims 12-21, wherein the second SARS-CoV-2 spike protein is derived from an omicron variant of SARS-CoV-2.

23. The SARS-CoV-2 DNA vaccine of any one of claims 1-11, wherein the second SARS-CoV-2 protein comprises a SARS-CoV-2 envelope protein, a SARS-CoV-2 membrane protein, or a SARS-CoV-2 nucleocapsid protein, or a portion of any of these.

24. The SARS-CoV-2 DNA vaccine of claim 23, wherein the second SARS-CoV-2 protein is a SARS-CoV-2 envelope protein, or a portion thereof.

25. The SARS-CoV-2 DNA vaccine of claim 24, wherein the second SARS-CoV-2 protein is a full-length SARS-CoV-2 envelope protein.

26. The SARS-CoV-2 DNA vaccine of claim 24 or 25, wherein the SARS-CoV-2 envelope protein comprises an amino acid sequence having at least about 80%, 85%, 90%, 95%, 97%, or 98% sequence identity to the sequence
(SEQ ID NO: 44)
MYSFVSEETGTLIVNSVLLFLAFVVFLLVTLAILTALRLCAYCCNIVNV
SLVKPSFYVYSRVKNLNSSRVPDLLV (Uniprot ID P0DTC4).

27. The SARS-CoV-2 DNA vaccine of any one of claims 24-26, wherein the SARS-CoV-2 envelope protein comprises the sequence
(SEQ ID NO: 44)
MYSFVSEETGTLIVNSVLLFLAFVVFLLVTLAILTALRLCAYCCNIVNV
SLVKPSFYVYSRVKNLNSSRVPDLLV.

28. The SARS-CoV-2 DNA vaccine of claim 23, wherein the second SARS-CoV-2 protein is a SARS-CoV-2 membrane protein, or a portion thereof.

29. The SARS-CoV-2 DNA vaccine of claim 28, wherein the second SARS-CoV-2 protein is a full-length SARS-CoV-2 membrane protein.

30. The SARS-CoV-2 DNA vaccine of claim 28 or 29, wherein the SARS-CoV-2 membrane protein comprises an amino acid sequence having at least about 80%, 85%, 90%, 95%, 97%, 98%, or 99% sequence identity to the sequence
(SEQ ID NO: 41)
MADSNGTITVEELKKLLEQWNLVIGFLFLTWICLLQFAYANRNRFLYII
KLIFLWLLWPVTLACFVLAAVYRINWITGGIAIAMACLVGLMWLSYFIA
SFRLFARTRSMWSFNPETNILLNVPLHGTILTRPLLESELVIGAVILRG
HLRIAGHHLGRCDIKDLPKEITVATSRTLSYYKLGASQRVAGDSGFAAY
SRYRIGNYKLNTDHSSSSDNIALLVQ (UniProt ID P0DTC5).

31. The SARS-CoV-2 DNA vaccine of any one of claims 28-30, wherein the SARS-CoV-2 membrane protein comprises the sequence
(SEQ ID NO: 41)
MADSNGTITVEELKKLLEQWNLVIGFLFLTWICLLQFAYANRNRFLYII
KLIFLWLLWPVTLACFVLAAVYRINWITGGIAIAMACLVGLMWLSYFIA
SFRLFARTRSMWSFNPETNILLNVPLHGTILTRPLLESELVIGAVILRG
HLRIAGHHLGRCDIKDLPKEITVATSRTLSYYKLGASQRVAGDSGFAAY
SRYRIGNYKLNTDHSSSSDNIALLVQ.

32. The SARS-CoV-2 DNA vaccine of claim 23, wherein the second SARS-CoV-2 protein is a SARS-CoV-2 nucleocapsid protein, or a portion thereof.

33. The SARS-CoV-2 DNA vaccine of claim 32, wherein the second SARS-CoV-2 protein is a full-length SARS-CoV-2 nucleocapsid protein.

34. The SARS-CoV-2 DNA vaccine of claim 32 or 33, wherein the SARS-CoV-2 nucleocapsid protein comprises an amino acid sequence having at least about 80%, 85%, 90%, 95%, 97%, 98%, or 99% sequence identity to the sequence
(SEQ ID NO: 43)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA (UniProt ID P0DTC9).

35. The SARS-CoV-2 DNA vaccine of any one of claims 32-34, wherein the SARS-CoV-2 nucleocapsid protein comprises the sequence
(SEQ ID NO: 43)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA.

36. The SARS-CoV-2 DNA vaccine of any one of claims 23-35, wherein the first SARS-CoV-2 spike protein comprises an amino acid sequence having at least 90%, at least 95%, at least 96%, at least 97%, at least 98%, or at least 99% sequence identity with the sequence set forth in SEQ ID NO: 3, 31, or 36.

37. The SARS-CoV-2 DNA vaccine of any one of claim 36, wherein the second SARS-CoV-2 spike protein comprises an amino acid sequence of SEQ ID NO: 3, 31, or 36.

38. The SARS-CoV-2 DNA vaccine of any one of claims 23-37, wherein the first SARS-CoV-2 spike protein is derived from an omicron variant of SARS-CoV-2.

39. The SARS-CoV-2 DNA vaccine of any one of the preceding claims, wherein the DNA vectors are each double-stranded DNA vectors.

40. The SARS-CoV-2 DNA vaccine of any one of the preceding claims, wherein the DNA vectors are each plasmids.

41. The SARS-CoV-2 DNA vaccine of any one of the preceding claims, wherein the DNA vectors are present in the vaccine in about equal amounts.

42. The SARS-CoV-2 DNA vaccine of any one of the preceding claims, wherein the proteolipid vehicle comprises an ionizable lipid.

43. The SARS-CoV-2 DNA vaccine of any one of the preceding claims, wherein the fusogenic membrane protein is a fusion-associated small transmembrane (FAST) protein.

44. The SARS-CoV-2 DNA vaccine of claim 43, wherein the FAST protein comprises domains from one or more FAST proteins selected from p10, p14, p15, and p22.

45. The SARS-CoV-2 DNA vaccine of claim 43 or 44, wherein the FAST protein comprises an amino acid sequence having at least 80% sequence identity to the sequence:
(SEQ ID NO: 7)
MGSGPSNFVNHAPGEAIVTGLEKGADKVAGTISHTIFVEIVSSSTGIII
AVGIFAFIFSFLYKLLQWYNRKSKNKKRKEQIREQIELGLLSYGAGVAS
LPLLNVIAHNPGSVISATPIYKGPCTGVPNSRLLQITSGTAEENTRILN
HDGRNPDGSINV.

46. The SARS-CoV-2 DNA vaccine of any one of the preceding claims, wherein the first DNA vector and/or the second DNA vector comprise the polynucleotide sequence encoding the adjuvant.

47. The SARS-CoV-2 DNA vaccine of any one of the preceding claims, wherein both the first DNA vector and the second DNA vector comprises copies of the polynucleotide sequence encoding the adjuvant.

48. The SARS-CoV-2 DNA vaccine of claim 46 or 47 wherein the polynucleotide encoding the adjuvant is positioned on the DNA vector in which it is comprised such that the adjuvant is included in the 3′-UTR of an mRNA transcribed from the polynucleotide encoding the SARS-CoV-2 protein.

49. The SARS-CoV-2 DNA vaccine of any one of claims 46-48, wherein the adjuvant comprises a pathogen-associated molecular pattern (PAMP).

50. The SARS-CoV-2 DNA vaccine of any one of claims 46-49, wherein the adjuvant comprises an RNA CpG motif, a retinoic acid-inducible gene I (RIGI) agonist, a melanoma differentiation-associated protein 5 (MDA5) agonist, or a combination thereof.

51. The SARS-CoV-2 DNA vaccine of any one of claims 46-50, wherein the first DNA vector and/or the second DNA vector comprise a second polynucleotide sequence encoding a second adjuvant.

52. The SARS-CoV-2 DNA vaccine of claims 46-51, wherein both the first DNA vector and the second DNA vector comprises copies of a second polynucleotide sequence encoding a second adjuvant.

53. The SARS-CoV-2 DNA vaccine of claim 51 or 52, wherein the second polynucleotide encoding the second adjuvant is positioned on the DNA vector such that the second adjuvant is included in the 3′-UTR of an mRNA transcribed from the polynucleotide encoding the SARS-CoV-2 protein.

54. The SARS-CoV-2 DNA vaccine of any one of claims 51-53, wherein the adjuvant and the second adjuvant each independently comprise PAMPs.

55. The SARS-CoV-2 DNA vaccine of any one of claims 51-54, wherein the adjuvant comprises an RNA CpG motif and the second adjuvant comprises a RIGI agonist.

56. The SARS-CoV-2 DNA vaccine of any one of the preceding claims, wherein the SARS-CoV-2 DNA vaccine is stable at a temperature of from about 2° C. to about 8° C. for a period of at least about 1 month, at least about 2 months, at least about 3 months, at least about 6 months, at least about 9 months, or at least about 12 months.

57. The SARS-CoV-2 DNA vaccine of any one of the preceding claims, wherein the SARS-CoV-2 DNA vaccine is stable at room temperature for a period of at least about 7 days, at least about 14 days, at least about 21 days, or at least about 28 days.

58. The SARS-CoV-2 DNA vaccine of any one of the preceding claims, wherein administration of the SARS-CoV-2 DNA vaccine to a subject induces at least 2-fold, at least 3-fold, at least 4-fold, at least 5-fold, at least 6-fold, at least 7-fold, at least 8-fold, at least 9-fold, or at least 10-fold higher anti-spike protein antibody concentration as compared to a SARS-CoV-2 DNA vaccine which contains only one of the vectors.

59. The SARS-CoV-2 DNA vaccine of claim 58, wherein the higher anti-spike protein antibody concentration is determined as against the spike protein from which the second SARS-CoV-2 spike protein is derived.

60. The SARS-CoV-2 DNA vaccine of claim 59, wherein the higher anti-spike protein concentration is determined as against a third SARS-CoV-2 spike protein.

61. The SARS-CoV-2 DNA vaccine of claim 60, wherein the third SARS-CoV-2 spike protein comprises at least 1, at least 2, at least 3, at least 4, or at least 5 amino acid modification as compared to the first and second SARS-CoV-2 spike proteins.

62. The SARS-CoV-2 DNA vaccine of claim 60 or 61, wherein the third SARS-CoV-2 spike protein is a different sub-variant of the same variant from which the first or second SARS-CoV-2 spike protein is derived.

63. A method of eliciting an anti-SARS-CoV-2 immune response in a subject, the method comprising administering to the subject a therapeutically effective amount of the SARS-CoV-2 DNA vaccine of any one of the preceding claims to the subject.

64. The method of claim 63, wherein the administering comprises administering two doses of the therapeutically effective amount of the SARS-CoV-2 DNA vaccine.

65. The method of claim 64, wherein the two doses are administered at an interval of from about 2 weeks to about 8 weeks apart.

66. The method of any one of claims 63-65, wherein the therapeutically effective amount of the SARS-CoV-2 DNA vaccine is from about 0.050 mg to about 0.500 mg.

67. The method of any one of claims 63-66, wherein the SARS-CoV-2 DNA vaccine is administered intramuscularly.

68. The method of any one of claims 63-67, wherein the SARS-CoV-2 DNA vaccine is administered without electroporation.

69. The method of any one of claims 63-68, wherein the subject is a mammal.

70. The method of any one of claims 63-69, wherein the subject is a human.

71. The method of any one of claims 63-70, wherein antibodies induced by the administration of the SARS-CoV-2 DNA vaccine exhibit specific binding for two or more variants of the SARS-CoV-2 spike protein.
